(19) United States
(12) Patent Application Publication
(10) Pub. No.: US 2024/0115566 A1
(43) Pub. Date: Apr. 11, 2024

(54) METHODS OF TREATING DISORDERS ASSOCIATED WITH CASTOR

(71) Applicant: ST. JUDE CHILDREN'S RESEARCH HOSPITAL, INC., Memphis, TN (US)

(72) Inventors: Suzanne JACKOWSKI, Bartlett, TN (US); Charles O. ROCK, Bartlett, TN (US); Matthew W. FRANK, Memphis, TN (US)

(21) Appl. No.: 18/257,680

(22) PCT Filed: Dec. 16, 2021
(86) PCT No.: PCT/US2021/063717

§ 371 Date: Jun. 15, 2023

Related U.S. Application Data
(60) Provisional application No. 63/126,462, filed on Dec. 16, 2020.
(60) Provisional application No. 63/164,484, filed on Mar. 22, 2021.

(51) Int. Cl. A61K 31/501; A61P 3/00; A61P 25/00; G01N 33/50

(52) U.S. Cl. CPC A61K 31/501; A61P 3/00; A61P 25/00; G01N 33/5008; G01N 2800/52

(57) ABSTRACT

The present disclosure relates to methods of identifying subjects in need of treatment for, e.g., a coenzyme A reduction, elevation, sequestration, toxicity, or redistribution (CASTOR) disease such as, for example, defects in fatty acid oxidation enzymes, methylmalonic acidemia, glutaric acidemia, propionic academia, and HMG-CoA lyase, via small molecule modulators of CoA levels. The methods may comprise assessing levels of carnitines, CoA, and/or various metabolites and biomarkers and administration of therapeutics useful in the treatment of CASTOR disorders, metabolic diseases, and/or neurological diseases. This abstract is intended as a scanning tool for purposes of searching in the particular art and is not intended to be limiting of the present inventions.

### CROSS-REFERENCES TO RELATED APPLICATIONS

This application claims priority to U.S. Provisional Application No. 63/126,462 filed Dec. 16, 2020 and U.S. Provisional Application No. 63/164,484 filed Mar. 22, 2021, each of which is incorporated herein in its entirety for all purposes.

### STATEMENT AS TO RIGHTS TO INVENTIONS MADE UNDER FEDERALLY SPONSORED RESEARCH AND DEVELOPMENT

This inventions described herein were made with government support under grant number GM034496 awarded by the National Institutes of Health. The government has certain rights in the inventions.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Coenzyme A (CoA) is a cofactor derived from vitamin B₅ (pantothenate) that is covalently bound to the organic acids in cells, thereby enabling the organic acids to participate in the biochemical reactions that govern energy production and lipid metabolism. CoA is essential for hundreds of metabolic reactions including the tricarboxylic acid cycle, fatty acid oxidation and synthesis, amino acid metabolism, and neurotransmitter synthesis. The CoA-bound organic acids, called acyl-CoAs, constitute a small, but significant portion of the total CoA pool under normal healthy conditions. A number of inborn errors of metabolism result from the genetic deficiency of one of the enzymes acting on acyl-CoAs, leading to the accumulation of acyl-CoAs to high levels. ‘CASTOR’ is the term that has been given to these diseases and stands for CoA sequestration, toxicity or redistribution (Mitchell et al. (2008) Mol. Genet. Metab. 94:4-15). Acyl-CoAs are known feedback inhibitors of pantothenate kinase (PANK) enzymes that catalyze the first step in CoA biosynthesis (Leonardi et al. (2005) Prog. Lipid Res. 44:125-153). Under CASTOR conditions the synthesis of CoA becomes inhibited and energy production and lipid metabolism are limited as a result.

CASTOR diseases are numerous and include, for example, organic acidemias, HMG-CoA lyase deficiency, and defects in fatty acid oxidation enzymes. Organic acidemias are genetic metabolic disorders characterized by a defect in protein metabolism that results in an essential enzyme malfunctioning or being absent. Examples of organic acidemias include, but are not limited to, propionic acidemia, methylmalonic acidemia, and isovaleric acidemia. Propionic acidemia (PA) is a rare autosomal-recessive metaolic disease that arises from missense mutations in one of the mitochondrial propionyl-CoA carboxylase (PCC) (E.C. 6.4.1.3) genes (PCCA or PCCB) that produce proteins with diminished catalytic activity that compromises the catabolism of propionyl-CoA (C3-CoA) and disrupts multiple metabolic processes. PA arises from a devastating inborn error of metabolism that results in substantial morbidity and mortality. The CoA-dependent tricarboxylic acid (TCA) cycle (also referred to as the citric acid cycle (CAC)) activity is dysfunctional in PA patients leading to TCA cycle intermediates being excreted in urine. Reduced PCC activity can lead to the accumulation of intracellular C3-CoA, which is converted to propionyl-carnitine (C3-carnitine) that is released into the plasma and excreted into the urine. The severity of PA varies considerably depending on the impact of the missense or nonsense mutations on PCC catalytic activity and the plasma C3:C2-carnitine ratio is a key biomarker used to screen newborns. C3-carnitine is not considered a toxic metabolite but rather a mechanism to release non-esterified CoA (CoASH) and eliminate excess propionate from the body. Methylcitrate formed by the condensation of C3-CoA with oxaloacetate is another mechanism to release CoASH from C3-CoA. Methylcitrate is not a substrate for ATP:citrate lyase or aconitase and its excretion in urine eliminates excess propionate from the body.

CoASH functions as a cofactor in numerous reactions in intermediary metabolism and is a critical substrate for two key steps in mitochondrial energy metabolism: pyruvate and α-ketoglutarate dehydrogenases. The concept of CoASH sequestration or trapping as a metabolic basis for disease was advanced in the 1990s to explain the cellular toxicity of xenobiotic carboxylic acids, like valproate, pivalate, and benzoic acid. These compounds are converted to their respective intracellular CoA thioesters leading to a reduction in CoASH needed to support intermediary metabolism and TCA cycle function. CoASH sequestration was proposed as an underlying cause for metabolic dysfunction in acidemia diseases based on the inhibition of pyruvate oxidation, fatty acid oxidation, ureagenesis, and gluconeogenesis in ex-vivo cultures treated with propionate. Treatment of hepatocytes with propionate leads to a decrease in CoASH and C2-CoA concomitant with a rise in C3-CoA.

The cellular levels of CoA are controlled by the activity of pantothenate kinase (PanK), the first and rate-controlling step in CoASH biosynthesis. A rare, life-threatening neurological disorder known as pantothenate kinase-associated neurodegeneration (PKAN) arises from mutations in the human PANK2 gene leading to a prominent extrapyramidal movement disorder and a characteristic deposition of iron in the basal ganglia. Disruption to the PANK1,2 gene can lead to a reduction in CoA synthesis. Pyruvate produced from glycolysis requires CoA to form acetyl-CoA, the substrate for the mitochondrial TCA cycle, and reduced CoA can lead to disrupted TCA cycle and hence reduce glutamate levels in the neurons and may cause cell death.

PanK activity is potently inhibited by CoA thioesters. The PanK⋅acetyl-CoA complex crystal structure shows CoA thioesters stabilize a PanK conformation that is not capable of interacting with ATP. Both active sites in each protomer are intimately linked and simultaneously switch between the inactive, acyl-CoA bound conformation to the active, ATP:Mg²⁺ bound conformation. Inhibition of PanK in liver can lead to mitochondrial dysfunction highlighted by reduced fatty acid β-oxidation capacity and gluconeogenesis. Analysis of the Pank1^{-/-} mice shows that the reduced liver CoA in these animals prevents normal fuel switching to mitochondrial fatty acid oxidation during fasting. The Pank1^{-/-}Pank2^{-/-} and Pank1^{-/-}Syn-Pank2^{-/-} mice are more severely compromised and succumb to metabolic crisis that results in death. Thus, a reduction in PanK activity can lead to reduced CoA that in turn has a major impact on metabolism.

Currently, there are not sufficient approved therapies to treat organic acidemias such as PA or PKAN. PA patients are placed on low-protein or synthetic diets to reduce C3-CoA formation. Human liver transplantation results in improvement in the quality of life and lower levels of PA biomarkers, although the patients are still at risk of developing debilitating complications. Moreover, despite the documented association of PanK with acyl-CoAs, methods of treating CASTOR diseases using small molecule modulators of CoA levels have yet to be realized. Thus, there remains a need for methods of treating CASTOR diseases via modulation of CoA levels. These needs and others are met by the present disclosure.

### SUMMARY

The present disclosure relates to compositions, methods, systems, and kits for use in the prevention and treatment of disorders associated with CoA sequestration, toxicity or redistribution (CASTOR) and/or pantothenate kinase activity such as, for example, CASTOR diseases such as organic acidemias (e.g., propionic acidemia, methylmalonic acidemia, glutaric acidemia, isovaleric acidemia, HMG-CoA lyase deficiency, and defects in fatty acid oxidation enzymes), pantothenate kinase-associated neurodegeneration (PKAN), and diabetes. The present disclosure provides methods of identifying subjects in need of treatment with a therapeutic agent effective in the treatment of a metabolic disease, neurological disorder (e.g., PKAN), or coenzyme A reduction, elevation, sequestration, toxicity, or redistribution (CASTOR) disorder and method of treating the same. The present disclosure also provides methods of assessing or monitoring subjects having or suspected of having such diseases as well as therapeutic regimens comprising administration of a therapeutic agent effective in the treatment of a metabolic disease, neurological disorder (e.g., PKAN), or CASTOR disease. In some embodiments, the methods, systems, and kits provided herein involve evaluating a level of an analyte, such as a tricarboxylic acid (TCA) cycle metabolite. In some embodiments, the methods, systems, and kits provided herein involve the use of a magnetic resonance method (e.g., to quantify a change in a level of a metabolite, such as glutamate/glutamine (Glx), γ-aminobutyric acid (GABA), inositol, choline, taurine, or N-acetyl aspartate. In some embodiments, a subject has undergone or is undergoing treatment for a metabolic disease, neurological disorder (e.g., PKAN), or CASTOR disorder.

In an aspect, the present disclosure provides a method comprising: (a) providing a subject having abnormal levels of one or more tricarboxylic acid (TCA) cycle metabolites; and (b) based at least in part on (a), identifying the subject as being in need of a treatment with a therapeutic agent useful in the treatment of a disorder associated with Coenzyme A (CoA) reduction, elevation, sequestration, toxicity, or redistribution (CASTOR), a neurological disorder, and/or a metabolic disorder. In some embodiments, the method further comprises administering (e.g., orally administering) the therapeutic agent to the subject.

In another aspect, the present disclosure provides a method comprising: (a) providing a subject having abnormal levels of one or more tricarboxylic acid (TCA) cycle metabolites; and (b) based at least in part on (a), administering a therapeutically effective amount of a therapeutic agent useful in the treatment of a disorder associated with Coenzyme A (CoA) reduction, elevation, sequestration, toxicity, or redistribution (CASTOR), a neurological disorder, and/or a metabolic disorder to the subject.

In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites comprise malate. In some embodiments, the level at least one of the one or more TCA cycle metabolites is elevated. In some embodiments, the malate level is elevated. In some embodiments, the level of at least one of the one or more TCA cycle metabolites is depressed. In some embodiments, the levels of TCA cycle metabolites of the one or more TCA cycle metabolites are urinary levels. In some embodiments, the levels of TCA cycle metabolites of the one or more TCA cycle metabolites are plasma levels. In some embodiments, the method further comprises assessing a sample (e.g., plasma and/or urine sample) from the subject to determine the levels of the one or more TCA cycle metabolites.

In some embodiments, the subject has an elevated C3-carnitine level, a depressed C2-carnitine level, a depressed carnitine level, and/or an elevated C3:C2-carnitine ratio in plasma. In some embodiments, the subject has an elevated C3-carnitine level, a depressed C2-carnitine level, a depressed carnitine level, and/or an elevated C3:C2-carnitine ratio in the liver. In some embodiments, the subject has an elevated C3:C2-Coenzyme A (CoA) level in the liver. In some embodiments, the subject has an elevated C3-CoA level in the liver and/or heart. In some embodiments, the subject is a mammal. In some embodiments, the subject is a human subject.

In some embodiments, the subject is diagnosed with a disorder associated with CASTOR or Pantothenate kinase-associated neurodegeneration (PKAN). In some embodiments, the subject is diagnosed with propionic acidemia, defects in fatty acid oxidation enzymes, methylmalonic acidemia, glutaric acidemia, or HMG-CoA lyase deficiency. In some embodiments, diagnosed with Pantothenate kinase-associated neurodegeneration (PKAN). In some embodiments, the subject has previously undergone a therapeutic regimen for treatment of a disorder associated with CASTOR or pantothenate kinase-associated neurodegeneration (PKAN). In some embodiments, the subject has previously been treated with pantothenate, carnitine, pantothenic acid, or a combination thereof. In some embodiments, the method further comprises identifying the subject as in need of treatment with pantothenate, carnitine, pantothenic acid, or a combination thereof. In some embodiments, the method further comprises administering pantothenate, carnitine, pantothenic acid, or a combination thereof to the subject. In some embodiments, the method further comprises use of a protein restricted diet, antibiotics, and/or sodium benzoate.

In an aspect, the present disclosure provides a method comprising: (a) providing a first analysis of a first sample derived from a subject at a first time, wherein the first analysis provides first levels of one or more tricarboxylic acid (TCA) cycle metabolites; (b) providing a second analysis of a second sample derived from the subject at a second time after the first time, wherein the second analysis provides second levels of the one or more TCA cycle metabolites; (c) assessing a difference between a second level and a first level of at least one of the one or more TCA cycle metabolites; and (d) based at least in part on (c), identifying the subject as being in need of a treatment with a therapeutic regimen comprising administration of a therapeutic agent useful in the treatment of a disorder associated with Coenzyme A (CoA) reduction, elevation, sequestration, toxicity, or redistribution (CASTOR), a neurological disorder, and/or a metabolic disorder. In some embodiments, the method further comprises administering (e.g., orally administering) the therapeutic agent to the subject.

In another aspect, the present disclosure provides a method comprising: (a) providing a first analysis of a first sample derived from a subject at a first time, wherein the first analysis provides first levels of one or more tricarboxylic acid (TCA) cycle metabolites; (b) providing a second analysis of a second sample derived from the subject at a second time, wherein the second analysis provides second levels of the one or more TCA cycle metabolites, wherein the second time is after the first time, and wherein the subject has undergone treatment with a therapeutic regimen comprising administration of a first amount of a therapeutic agent useful in the treatment of a disorder associated with Coenzyme A (CoA) reduction, elevation, sequestration, toxicity, or redistribution (CASTOR), a neurological disorder, and/or a metabolic disorder at a first frequency between the first time and the second time; (c) assessing a difference between a second level and a first level of at least one of the one or more TCA cycle metabolites; and (d) based at least in part on (c), (i) identifying the subject as being in need of a change in the therapeutic regimen if the difference in (c) exceeds or does not meet a threshold level, wherein the change comprises changing the amount of the therapeutic agent administered from the first amount to a second amount and/or changing the frequency of administration of the therapeutic agent to the subject from the first frequency to a second frequency, or (ii) identifying the subject as not being in need of a change in the therapeutic regimen in the difference in (c) does not exceed the threshold level.

In some embodiments, (d) comprises decreasing a dosage of the therapeutic agent if the difference in (c) exceeds the threshold level. In some embodiments, (d) comprises increasing a dosage of the therapeutic agent if the difference in (c) does not meet the threshold level. In some embodiments, (d) comprises not changing the therapy regimen if the difference in (c) does not exceed the threshold level. In some embodiments, the subject is diagnosed with a disorder associated with CASTOR, a neurological disorder, and/or a metabolic disorder (i) after performance of (a) but before performance of (b); (ii) before performance of (a); or (iii) after performance of (b). In some embodiments, the subject has undergone treatment with the therapeutic regimen prior to (a). In some embodiments, the first time is at least one week before the second time. In some embodiments, the first time is at least one month before the second time. In some embodiments, the first time is at least six months before the second time.

In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites comprise malate.

In some embodiments, the second level at least one of the one or more TCA cycle metabolites is lower than the first level of the at least one of the one or more TCA cycle metabolites. In some embodiments, the second level of malate level is lower than the first level of malate. In some embodiments, the second level at least one of the one or more TCA cycle metabolites is higher than the first level of the at least one of the one or more TCA cycle metabolites. In some embodiments, the first sample and the second sample are urine samples. In some embodiments, the first sample and the second sample are plasma samples.

In some embodiments, prior to (b), the subject has an elevated C3-carnitine level, a depressed C2-carnitine level, a depressed carnitine level, and/or an elevated C3:C2-carnitine ratio in plasma. In some embodiments, prior to (b), the subject has an elevated C3-carnitine level, a depressed C2-carnitine level, a depressed carnitine level, and/or an elevated C3:C2-carnitine ratio in the liver. In some embodiments, prior to (b), the subject has an elevated C3:C2-Coenzyme A (CoA) level in the liver. In some embodiments, prior to (b), the subject has an elevated C3-CoA level in the liver and/or heart. In some embodiments, subsequent to treatment with the therapeutic regimen, the C3-carnitine level and/or the C3:C2-carnitine ratio in the plasma and/or liver of the subject decreases. In some embodiments, subsequent to treatment with the therapeutic regimen, the C3:C2-Coenzyme A (CoA) level in the liver of the subject decreases. In some embodiments, the subject is a human subject.

In some embodiments, prior to (a), the subject is diagnosed with a disorder associated with CASTOR or Pantothenate kinase-associated neurodegeneration (PKAN). In some embodiments, prior to (a), the subject is diagnosed with propionic acidemia, defects in fatty acid oxidation enzymes, methylmalonic acidemia, glutaric acidemia, or HMG-CoA lyase deficiency. In some embodiments, prior to (a), the subject was treated with pantothenate, carnitine, pantothenic acid, or a combination thereof. In some embodiments, the method further comprises identifying the subject as in need of treatment with pantothenate, carnitine, pantothenic acid, or a combination thereof. In some embodiments, the method further comprises administering pantothenate, carnitine, pantothenic acid, antibiotics, sodium benzoate, or a combination thereof to the subject.

In some embodiments of any of the above aspects, the therapeutic agent is a compound provided herein. In some embodiments of any of the above aspects, the therapeutic agent is a compound having a structure represented by a formula:

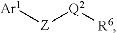

wherein Z is selected from A(C═O), COCH₂,

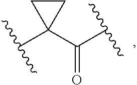 [SMILES: *C(=O)C1(*)CC1]

CO, NHCO, NHCS, CH₂SO₂, and SO₂; wherein A is selected from O, CO, CH₂, CF₂, NH, N(CH₃), and CH(OH); wherein Q² is a structure selected from:

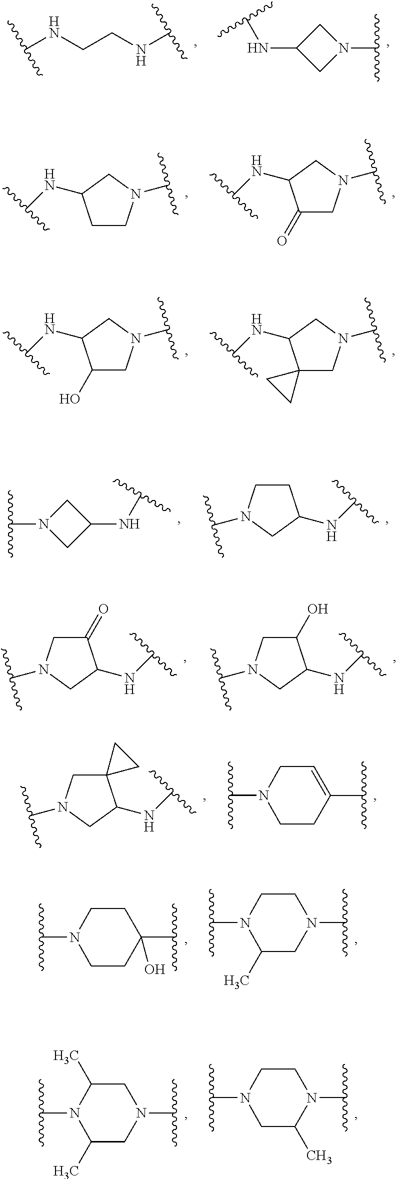

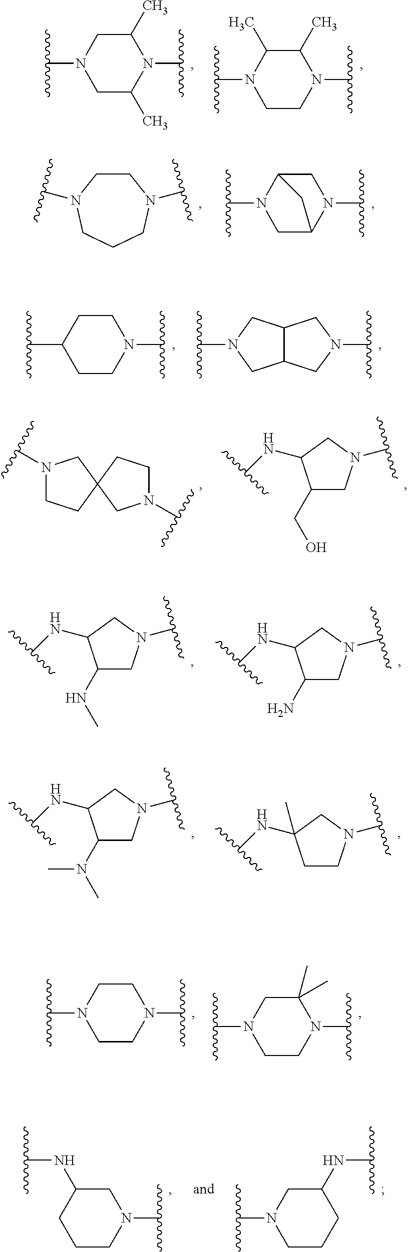

wherein Ar¹ is selected from aryl and heteroaryl and substituted with 0, 1, 2, or 3 groups independently selected from halogen, —NO₂, —CN, —OH, —SH, —NH₂, C1-C8 thioalkyl, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), C1-C8 alkoxyhaloalkyl, and cyclopropyl, cyclobutyl, and oxetane, wherein the cyclopropyl, cyclobutyl, and oxetane are optionally substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl); wherein R⁶ is selected from —NHCH₂C₆H₅ and Ar²; wherein Ar² is a structure represented by a formula selected from:

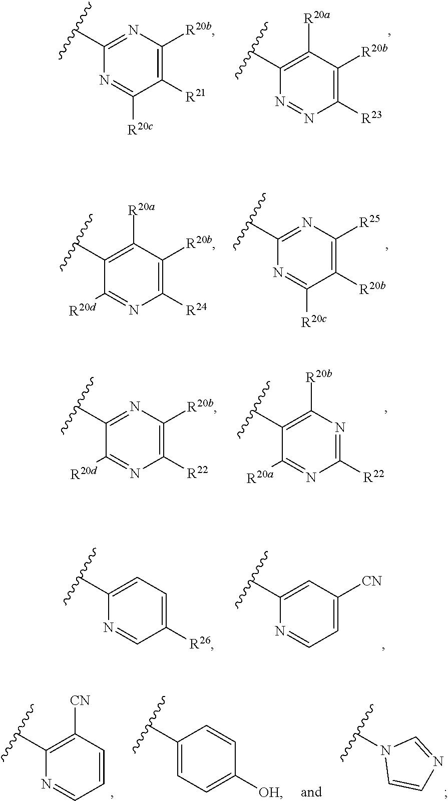

wherein each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; wherein R²¹, when present, is selected from hydrogen, halogen, —CN, —NO₂, —SO₂NH₂, —SO₂CH₃, —SO₂CF₃, and Cy¹; wherein Cy¹, when present, is selected from cycle, heterocycle, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino; wherein R²², when present, is selected from —CN, halogen, —NO₂, SO₂NH₂, SO₂CH₃, and SO₂CF₃; wherein R²³, when present, is selected from hydrogen, halogen, —CN, —NO₂, —SO₂NH₂, —SO₂CH₃, —SO₂CF₃, cyclohexyl,

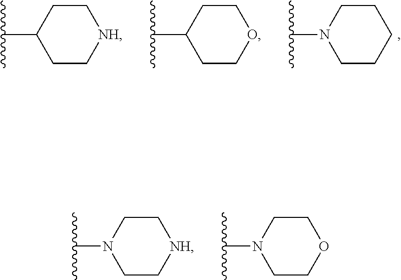

and Cy¹; wherein R²⁴, when present, is selected from —CN, halogen, —NO₂, SO₂NH₂, SO₂CH₃, and SO₂CF₃; wherein R²⁵, when present, is selected from —CN, —NO₂, SO₂NH₂, SO₂CH₃, and SO₂CF₃; wherein R²⁶, when present, is selected from —Br, —Cl, —F, —CN, —NO₂, —CF₃, and methyl; or a pharmaceutically acceptable salt thereof.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles described herein.

FIGS. 1A-1L show CoASH sequestration in PA mice. Wild-type (WT) and Pcca^{-/-}PCCA(A138T)^{tg/0} (PA) mice were maintained on a standard rodent chow and samples were harvested at day 68-71. (1A) Plasma carnitine. (1B) Plasma C2-carnitine. (1C) Plasma C3-carnitine. (1D) Plasma C3:C2-carnitine ratio. (1E) Liver carnitine. (1F) Liver C2-carnitine. (1G) Liver C3-carnitine. (1H) Liver C3:C2-carnitine ratio. (1I) Liver CoASH (non-esterified CoA). (1J) Liver C2-CoA. (1K) Liver C3-CoA. (1L) Liver C3:C2-CoA ratio. Male mice are blue and female mice are red. [¹³C]C2-CoA was used as the internal standard (FIG. 9). Statistical significance was determined using the two-tailed Student's t test (GraphPad/Prism software). ns means not significant (p>0.01) and the significant p values are reported in red. Number of animals in each group are shown in parenthesis.

FIGS. 2A-2B show transgene expression and TCA cycle metabolites in PA mice. FIG. 2A shows Western blot analysis of PCCA(A138T) transgene expression in tissues of WT and PA mice. This blot illustrates the tissue-specific distribution of Pcca in WT mice (10 micrograms per lane (μg/lane)) compared to the transgene PCCA(A138T) protein level in the PA mice (60 μg/lane). Western blots for each tissue were obtained from triplicate mice (FIGS. 10A-10E), and a fourth blot was performed for this figure. The red asterisk indicates a non-specific band. FIG. 2B shows a metabolomics screen of TCA cycle metabolites in plasma (upper panel) and urine (lower panel) of PA mice. Three males and three females were used for this analysis. Statistical significance was determined using the two-tailed Student's t test (GraphPad/Prism software). ns means not significant (p>0.01) and the significant p values are reported in red.

FIGS. 3A-3F show properties of PZ-3022 and its impact on CoA levels in PA liver. (3A) Chemical structures and relevant properties of PZ-2891 and PZ-3022. Purity and NMR spectra of PZ-3022 is shown in FIGS. 14A-14B and 15A-15B. (3B) Inhibition of PANK3 by PZ-3022. The EC₅₀ was calculated in GraphPad using the Morrison equation. (3C) Crystal structure of the PanK3⋅AMPPNP⋅Mg²⁺⋅PZ-3022 complex (PDB ID: 6PE6) overlaid on the PZ-2891 structure (PDB ID: 6B3V). The two PANK3 protomers are colored gold and cyan. The Fo-Fc simulated annealing omit map is contoured at 3σ (yellow mesh). (3D) Elevation of total cellular CoA in C3A cells treated with 10 μM of either PZ-2891 or PZ-3022. (3E) Half-life of PZ-2891 compared to PZ-3022 in mice. The complete pharmacokinetic profiles are detailed in Table 5. (3F) Male C57BL/6 mice were orally gavaged every 24 hours (h) with Captisol containing 10 milligrams per kilogram (mg/kg) of either PZ-2891 or PZ-3022 for three days, and the level of liver total CoA was determined 4 h after the last drug dose. Total CoA was determined using fluorescent derivatization assay. Statistical significance was determined using the two-tailed Student's t test (GraphPad/Prism software). The significant p values are reported in red. Numbers of biological replicates are shown in parenthesis.

FIGS. 4A-4H show that acute treatment with PZ-3022 relieves CoASH sequestration in liver. Mice were orally gavaged every 24 h with either 10 or 30 mg/kg PZ-3022 plus 50 mg/kg pantothenate. Control animals received 50 mg/kg pantothenate. Four hours after the third dose, the impact of short-term PZ-3022 treatment on liver CoA pools was determined using mass spectrometry. (4A) CoASH. (4B) C2-CoA. (4C) C3-CoA. (4D) C3:C2-CoA ratio. Male mice are blue and female mice are red. (4E) Elevation of total hepatic CoA in PA mice dosed with drug. (4F) C57BL/6J male mice were administered PZ-3022 by gavage and at the indicated times samples were taken (3 mice per point) to determine plasma PZ-3022 levels and liver total CoA using the fluorescence assay. (4G) Envigo chow containing 1000 parts per million (ppm) pantothenate was formulated with 7.5, 37.5 and 75 ppm PZ-3022. C57BL/6J male mice were maintained on the diets for 1 week and the total liver CoA was determined using the fluorescence assay. (4H) PZ-3022 levels in the plasma and liver as a function of PZ-3022 in the diet. Statistical significance was determined using the two-tailed Student's t test (GraphPad/Prism software). ns means not significant (p>0.01) and the significant p values are reported in red. Numbers of mice are shown in parenthesis.

FIGS. 5A-5L show metabolic parameters in mice treated with PZ-3022 for 70 days. Animals were maintained on a defined diet supplemented with 1000 ppm pantothenate either with or without 75 ppm PZ-3022 beginning at weaning on day 21. On day 68-70, levels of liver CoAs and carnitines were determined by mass spectrometry. Male mice are blue and female mice are red. (5A) Liver CoASH. (5B) Liver C2-CoA. (5C) Liver C3-CoA. (5D) Liver C3:C2-CoA ratio. (5E) Liver carnitine. (F) Liver C2-carnitine. (5G) Liver C3-carnitine. (5H) Liver C3:C2-carnitine ratio. (5I) Plasma carnitine. (5J) Plasma C2-carnitine. (5K) Plasma C3-carnitine. (5L) Plasma C3-C2-carnitine ratio. Statistical significance was determined using the two-tailed Student's t test (GraphPad/Prism software). ns means not significant (p>0.01) and the significant p values are reported in red. Numbers of mice in each group are shown in parenthesis.

FIGS. 6A-6B show TCA cycle intermediates in plasma and urine of treated PA mice. Urinary TCA cycle metabolites eliminated over 24 h were quantified by mass spectrometry and normalized to mouse body weight. FIG. 6A shows the effect of PZ-3022 therapy on the TCA cycle intermediate levels in plasma. FIG. 6B shows the effect of PZ-3022 therapy on the levels of TCA cycle intermediates in urine. Statistical significance was determined using the two-tailed Student's t test (GraphPad/Prism software). ns means not significant (p>0.01) and the significant p values are reported in red. Three male (blue) and three female (red) mice were used for each determination.

FIG. 7 shows the CoASH and the TCA cycle in PA. PA arises from the reduced capability to metabolize C3-CoA (green) by PCC (red X) leading to the accumulation of C3-CoA and the suppression of pantothenate kinase activity (red bar) and CoASH biosynthesis. Metabolites in yellow highlight are TCA cycle intermediates/metabolites measured in plasma and urine. CoASH and CoA thioesters are highlighted in salmon. The biochemical reactions that form methylmalonate, methylcitrate and C3-carnitine (not shown) all lead to the release of CoASH and eliminate excess propionate from the body. CoASH is a key substrate for two irreversible reactions in the cycle: pyruvate and α-ketoglutarate dehydrogenases. In PA, reduced availability of CoASH for these reactions slows the TCA cycle leading to the release of cycle intermediates into plasma and urine. PZ-3022 therapy relieves feedback inhibition of pantothenate kinase by C3-CoA, elevates CoA biosynthesis, increases intracellular CoASH and improves TCA cycle function based on the decrease in C3:C2-carnitine ratio in plasma and the 80% reduction of urinary malate.

FIG. 8 shows total plasma acyl-carnitine profile in WT and PA mice. The upper scan shows the plasma carnitine profile for WT mice and the reflection plot shows the profile in PA mice. Heavy internal standards (Carnitine Standards Set B; Cambridge Isotope Labs) are colored blue, all carnitine species are black except for C3-carnitine shown in red. C3-carnitine increases dramatically in PA mice, while carnitine and C2-carninte are reduced. Other species constitute a small percentage of the pool.

FIG. 9 shows mass spectrometry calibration curves. CoASH, C2-CoA and C3-CoA levels were quantified by LC-MS/MS using [¹³C]C2-CoA as the internal standard. Calibration curves of CoASH and C3-CoA with respect to [¹³C]C2-CoA are shown. These curves show that the CoASH was detected approximately 2.4-fold less efficiently than the C2-CoA, whereas C3-CoA detection was 80% efficient compared to C2-CoA. Thus, CoASH is more abundant than it appears when using [¹³C]C2-CoA as the calibrator.

FIGS. 10A-10E show propionyl-CoA levels in PA mouse tissues and CoA thioester pool composition of the PA mouse heart. Wild-type and PA mice were maintained on the Envigo diet, and at 70 days of age, the CoA thioester compositions of the tissues were determined by mass spectrometry using a [¹³C]acetyl-CoA internal standard. FIG. 10A shows levels of propionyl-CoA (C3-CoA) in liver, heart quadriceps muscle and brain from the PA mouse. FIG. 10B shows heart non-esterified CoA (CoASH) levels. FIG. 10C shows heart acetyl-CoA (C2-CoA) levels. FIG. 10D shows heart propionyl-CoA levels. FIG. 10E shows the heart C3:C2-CoA ratio. Males are blue and females are red. The number of mice in each group is shown in parenthesis in each bar. Values were compared using Student's t-test using GraphPad/Prism software (mean±S.E.) and the p values are shown in the panels.

FIGS. 11A-11B show tissue distributions of murine Pcca and transgene-expressed human PCCA proteins in WT and PA mice. The tissues from three male and three female WT and PA mice were collected and the level of Pcca protein present was estimated by western blotting using an antibody that reacts with both human and mouse alpha subunits of propionyl-CoA carboxylase. Gapdh antibody was also included in the western blot as a loading control. See Methods for details. The levels of Pcca in WT tissues were easily detected using 10 μg of protein per lane, whereas 60 μg per lane of protein was required to detect the expression of the transgene in the same tissues. FIG. 11A: Tissues from male WT and PA mice. FIG. 11B: Tissues from female WT and PA mice. The red asterisks indicate non-specific, cross-reacting protein bands.

FIGS. 12A-12B show liver fatty acid composition of WT and PA mice. FIG. 12A shows total fatty acid composition of male livers from wild-type and PA mice. FIG. 12B shows total fatty acid composition of female livers from wild-type and PA mice. Fatty acids were quantified by gas-liquid chromatography, and methyl esters present at less than 0.05% weight percent are not reported. Odd chain fatty acids were below this level of detection in wild-type mice, and rose to approximately 8.8% of the total in the PA mice. Five mice per group.

FIG. 13 shows amino acids in plasma and urine of WT and PA mice. Amino acid levels were determined by mass spectrometry of the native amino acids using warfarin as an internal standard, and the ratio of the abundances in the wild-type (WT) and PA mice are plotted. Glycine was measured as its benzoyl derivative. Upper panel, Plasma; Lower panel, Urine. Statistical significance of the relative change in each metabolite was determined using the two-tailed Student's t test (GraphPad/Prism software). p values<0.01 are shown in red. ns means not significant (p>0.01). Three male (blue) and three female (red) mice per genotype were used in this study.

FIGS. 14A-14B show purity and mass spectrum of PZ-3022. FIG. 14A shows liquid chromatography to examine purity of PZ-3022 based on ELSD detection, UV detection and total ion current. FIG. 14B shows the spectrum of PZ-3022.

FIGS. 15A-15B show NMR spectra of PZ-3022: ¹H-NMR spectrum of PZ-3022 in [d₆]DMSO (15A); ¹³C-NMR spectrum of PZ-3022 in [d₆]DMSO (15B).

FIGS. 16A-16D show pantazine drug levels in plasma and tissues. FIG. 16A shows a Representative LC/MS detection of PZ-2891 and its hydroxylated metabolites in plasma. The reactive ring is highlighted in the green circle and the structure of the compounds in peaks P1, P2 and P3 are shown. Dehydration of the major metabolite (P3) produced the styrene (P2). FIG. 16B shows a representative spectra of LCMS detection of PZ-3022 and its hydroxylated metabolite (P2) in plasma. For FIGS. 16C-16D, C57BL/6J mice were maintained on chow containing 75 ppm of either PZ-2891 or PZ-3022 for 4 weeks, and the parent drug levels were analyzed in plasma and liver by LC/MS. FIG. 16C shows a comparison of PZ-2891 and PZ-3022 levels in plasma. FIG. 16D shows a comparison of the levels of PZ-2891 and PZ-3022 in liver. Statistical significance was determined using the two-tailed Student's t test (GraphPad software). p values are shown in red.

FIGS. 17A-17C show that PZ-3022 renders PanK resistant to C3-CoA inhibition. FIG. 17A shows inhibition of PANK1b by C3-CoA in the presence and absence of PZ-3022. FIG. 17B shows inhibition of PANK2m (the mature processed form of human PANK2) by C3-CoA in the presence and absence of PZ-3022. FIG. 17C shows inhibition of PANK3 by C3-CoA in the presence and absence of PZ-3022. Human and mouse PanK isoforms are almost identical and respond to pantazines in the same manner. Data is from two independent experiment done in duplicate. Lines were fit using the Morrison equation using GraphPad software.

FIGS. 18A-18H show plasma and liver carnitines following short-term PZ-3022 therapy. Groups of male and female PA mice were gavaged for three days with either 10 or 30 mg/kg PZ-3022 plus 50 mg/kg pantothenate delivered in Captisol. Control PA mice received 50 mg/kg pantothenate. Four hours after the last dose on day 3, plasma and liver samples were analyzed to determine the impact of short-term PZ-3022 treatment on the levels of carnitine and acyl carnitines. FIGS. 18A-18H show plasma carnitine (18A), plasma C2-carnitine (18B), plasma C3-carnitine (18C), plasma C3:C2-carnitine ratio (18D), liver carnitine (18E), liver C2-carnitine (18F), liver C3-carnitine (18G), and liver C3:C2-carnitine ratio (18H). Male mice are blue and female mice are red. Statistical significance was determined using the two-tailed Student's t test (GraphPad/Prism software). p values<0.01 are shown in red. Numbers of mice are shown in parenthesis.

FIGS. 19A-19B show the impact of PZ-3022 therapy on total heart CoA and the heart:body weight ratio. Animals were maintained on PZ-3022 show until 70 days of age. FIG. 19A show total CoA levels in heart in response to treatment. FIG. 19B shows heart:body weight ratios of male and female PA mice treated with PZ-3022. Number of mice in each group is shown in parenthesis. Statistical analyses were performed using the Student's t-text using GraphPad/Prism software and the p values are shown in the figure panels. Male mice are blue and female mice are red. In FIG. 19B, two outliers were eliminated using Grubb' outliers test in GraphPad/Prism.

FIGS. 20A-20E show the effect of PZ-3022 on liver CoA, and plasma and urine amino acids wild-type mice. CoA species were determined using mass spectrometry as described in FIG. 5 comparing wild-type mice that are either untreated or treated with chow containing PZ-3022. FIGS. 20A-20E show liver CoASH (20A), liver C2-CoA (20B), liver C3-CoA (20C), liver C3:C2-CoA ratio (20D), and plasma and urine amino acid levels (20E). Number of mice in each group are shown in parenthesis. Amino acids were measured by mass spectrometry using warfarin as an internal standard except for glycine, which was measured as its benzoyl derivative. Statistical significance was determined using the two-tailed Student's t test (GraphPad/Prism software). ns means not significant (p>0.01) and the significant p values are reported in red. Two male (blue) and three female (red) mice were used for each determination.

FIGS. 21A-21B show the effect of PZ-3022 on TCA cycle intermediates in plasma and urine of wild-type mice. Urinary TCA cycle metabolites eliminated over 24 h were quantified by mass spectrometry and normalized to mouse body weight. FIG. 21A shows the effect of PZ-3022 on the TCA intermediate levels in plasma. FIG. 21B shows the effect of PZ-3022 on the levels of TCA cycle intermediates in urine. Statistical significance was determined using the two-tailed Student's t test (GraphPad/Prism software). ns means not significant (p>0.01) and the significant p values are reported in red. Male (blue) and female (red) mice were used for each determination. Number of mice in each group are shown in parenthesis.

FIG. 22A schematically depicts the effects of loss of CoA synthesis due to Pank1,2 deletion (e.g., disruption of murine Pank1 and Pank2 genes) in neurons. Pyruvate produced from glycolysis requires CoA to form acetyl-CoA, a substrate for the mitochondrial TCA cycle, and CoA limitation can disrupt TCA cycling which, in turn, affects glutamate metabolism thereby causing cell death. CoA loss due to Pank1,2 deletion results in lower neuronal glutamate and NAA. FIG. 22B schematically depicts CoA recovery after treatment with a compound provided herein, or a pharmaceutically acceptable form thereof (e.g., Compound 1), as described in Example 4. Without wishing to be bound by theory, the compound (e.g., a pantazine) allosertically activates the alternate Pank3 isoform that is expressed in murine neurons. Neurometabolic effects of CoA restoration by treatment with the compound may include increased TCA cycling and thus higher glutamate and NAA.

FIGS. 23A-23C depict representative 1H magnetic resonance spectra from three groups of mice studied in Example 4, including wild type (FIG. 23A), Pank1,2 neuronal dKO (FIG. 23B), and Pank1,2 neuronal dKO mouse treated with Compound 1 (FIG. 23C).

FIGS. 24A-24C depict metabolite to total creatine ratios of glutamate+glutamine (Glx) (FIG. 24A), N-acetyl aspartate (NAA) (FIG. 24B), and lactate (Lac) (FIG. 24C); *p<0.05 (WT vs KO); #p<0.05 (KO vs. KO+Compound 1). The box in the box and whisker plot represents the first and third quartiles. The line within the box represents the median while the ‘x’ within the box represents the mean.

FIG. 25 depicts metabolite to total creatine ratio for m-inositol, total choline, and taurine. KO: untreated Pank1/2 neuronal dKO mice. KO+Compound 1: Compound 1-treated Pank1/2 neuronal dKO mice. WT: wild-type.

FIGS. 26A-26C depicts voxel positioning corresponding to FIGS. 23A-23C in a wild-type mouse with viewpoints including horizontal (FIG. 26A), sagittal (FIG. 26B), and coronal (FIG. 26C).

Additional advantages of the inventions provided herein will be set forth in part in the description which follows, and in part will be obvious from the description, or can be learned by practice of the inventions. The advantages of the inventions will be realized and attained by means of the elements and combinations particularly pointed out in the appended claims. It is to be understood that both the foregoing general description and the following detailed description are exemplary and explanatory only and are not restrictive of the inventions, as claimed.

### DETAILED DESCRIPTION

The present disclosure can be understood more readily by reference to the following detailed description and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although methods and materials similar or equivalent to those described herein can be used in the practice or testing of the present disclosure, example methods and materials are now described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure and inventions described herein are not entitled to antedate such publication by virtue of prior invention. Further, the dates of publication provided herein may be different from the actual publication dates, which can require independent confirmation.

### I. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, horse, cat, guinea pig, or rodent. The term “subject” may be a domesticated animal (e.g., cat, dog, etc.), livestock (e.g., cattle, horse, pig, sheep, goat, etc.), or laboratory animal (e.g., mouse, rabbit, rat, guinea pig, fruit fly, etc.). The term does not denote a particular age or sex. Thus, adult and newborn subjects, as well as fetuses, whether male or female, are intended to be covered. In one aspect, the subject is a mammal, such as a human. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of or curing the disease.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to an examination such as a physical examination by a person of skill, for example, a physician, and found to have a condition that can be diagnosed or treated by the compounds, compositions, or methods disclosed herein. Diagnosis may comprise genetic analysis, physical examination, laboratory analysis, qualitative analysis, etc.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex, comorbidities, and diet of the patient; the time of administration; the route of administration; the rate of excretion of the specific compound employed; the duration of the treatment; drugs used in combination or coincidental with the specific compound employed and like factors. For example, treatment of a subject with a compound described herein may start with doses lower than those required to achieve a desired therapeutic effect and gradually increase until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs, and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes, without limitation, medicaments; vitamins; mineral supplements; substances used for the treatment, prevention, diagnosis, cure, or mitigation of disease or illness; or substances which affect the structure or function of the body; or prodrugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH₂, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula (CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldeyhyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptenyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” or “CO” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ-, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula N₃.

The term “nitro” as used herein is represented by the formula —NO₂.

The term “nitrile” or “cyano” as used herein is represented by the formula —CN or —C═N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the present disclosure may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘}may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

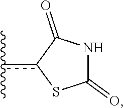

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present disclosure unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the present disclosure includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of synthetic procedures used to prepare such compounds, or in using racemization or epimerization procedures, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the present disclosure include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this present disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

The compounds described in the present disclosure can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the present disclosure to form solvates and hydrates. Unless stated to the contrary, the present disclosure includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

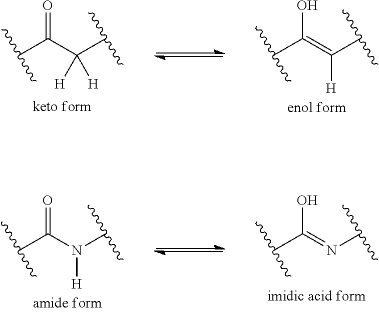 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

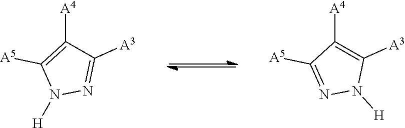

Unless stated to the contrary, the present disclosure includes all such possible tautomers.

Certain chemical substances form solids which are present in different states of order which are termed polymorphic forms or modifications. The different modifications of a polymorphic substance can differ greatly in their physical properties. The compounds according to the present disclosure can be present in different polymorphic forms, with it being possible for particular modifications to be metastable. Unless stated to the contrary, the present disclosure includes all such possible polymorphic forms.

In some aspects, a structure of a compound can be represented by a formula:

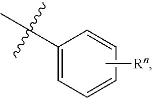

which is understood to be equivalent to a formula:

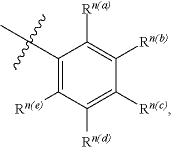

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the present disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the present disclosure. Thus, if there are a variety of additional steps that can be performed it is understood that each of these additional steps can be performed with any specific embodiment or combination of embodiments of the methods of the present disclosure.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### II. IDENTIFYING CASTOR DISEASES

The present disclosure provides methods of identifying and/or selecting subject (e.g., human subjects) in need of treatment with a therapeutic regimen (e.g., a therapeutic regimen comprising administration of a therapeutic agent useful in the treatment of a metabolic disorder, neurological disorder (e.g., pantothenate kinase-associated neurodegeneration (PKAN)), coenzyme A reduction, elevation, sequestration, toxicity, or redistribution (CASTOR) disorder (e.g., defects in fatty acid oxidation enzymes, HMG-CoA lyase deficiency, or organic acidemia such as methylmalonic acidemia, glutaric acidemia, or propionic acidemia), such as a compound provided herein), or adjustment to the same. The subject may have a metabolic disorder, neurological disorder (e.g., PKAN), or a CASTOR disorder, as described herein. The methods may comprise analysis of one or more biomarkers, including CoA levels, carnitine levels, and/or tricarboxylic acid (TCA) cycle metabolite levels, or proxies or ratios thereof. The one or more biomarkers may be plasma and/or urine biomarkers. Alternatively or in addition, the methods may comprise analysis of one or more metabolites (e.g., cerebral metabolites) such as glutamate/glutamine (Glx), GABA, lactate, inositol, choline, taurine, and N-acetyl aspartate. The methods may comprise collection of one or more samples of, e.g., plasma and/or urine from a subject and assessment of the one or more biomarkers in the one or more samples. Alternatively or in addition, the methods may comprise the use of magnetic resonance methods to assess (e.g., quantify) changes in metabolites. Assessment of one or more biomarkers and/or metabolites may comprise comparison against standard or normal levels accepted for healthy subjects. One or more samples from a subject may be collected at one or more different times, such as at a first time prior to commencement of any therapeutic regimen for a CASTOR disorder, metabolic disorder, or PKAN and at a second time subsequent to commencement of a therapeutic regimen for a CASTOR disorder, metabolic disorder, or PKAN. Similarly, a magnetic resonance method may be applied at one or more different times, such as at a first time prior to commencement of any therapeutic regimen for a CASTOR disorder, metabolic disorder, or PKAN and at a second time subsequent to commencement of a therapeutic regimen for a CASTOR disorder, metabolic disorder, or PKAN. Additional subject information, including the subject's age, weight, general health, symptoms, national origin, family medical history, genetic profile, appetite, muscle tone, energy levels, intellectual development, and any other details, may also be collected and/or assessed. The methods provided herein may comprise monitoring or evaluating a therapeutic regimen the subject is currently undergoing (e.g., a therapeutic regimen for the treatment of a CASTOR disorder, metabolic disorder, or PKAN), recommending a therapeutic regimen for administration to the subject, administration of a therapeutic agent (e.g., as described herein) to the subject, and/or recommending a change to a therapeutic regimen the subject is currently undergoing, such as a change in dose amount and/or frequency.

In an aspect, the present disclosure provides a method for identifying and/or selecting a subject (e.g., a human subject) in need of treatment with a therapeutic regimen (e.g., a therapeutic regimen comprising administration of a therapeutic agent useful in the treatment of a CASTOR disorder (e.g., an organic acidemia, such as propionic acidemia), such as a compound provided herein), PKAN, or metabolic disorder, or adjustment to the same. The methods may comprise analysis of one or more biomarkers, including CoA levels, carnitine levels, and/or tricarboxylic acid (TCA) cycle metabolite levels, or proxies or ratios thereof. The one or more biomarkers may be plasma and/or urine biomarkers. The methods may comprise collection of one or more samples of, e.g., plasma and/or urine from a subject and assessment of the one or more biomarkers in the one or more samples. Such assessment may comprise comparison against standard or normal levels accepted for healthy subjects. The one or more samples may be collected at one or more different times, such as at a first time prior to commencement of any therapeutic regimen for a CASTOR disorder and at a second time subsequent to commencement of a therapeutic regimen for a CASTOR disorder. Additional subject information, including the subject's age, weight, general health, symptoms, national origin, family medical history, genetic profile, appetite, muscle tone, energy levels, intellectual development, and any other details, may also be collected and/or assessed. The methods provided herein may comprise recommending a therapeutic regimen for administration to the subject and/or administration of a therapeutic agent (e.g., as described herein) to the subject. The methods provided herein can be used to identify and/or select patients for therapy (e.g., as described herein); to demonstrate, evince, or quantify one or more therapeutic effects, such as improvement of a patient's condition and/or a reduction or improvement in one or more symptoms of a disorder for which a patient is treated (e.g., as described herein); and/or for identifying an appropriate dose of a compound, or a pharmaceutically acceptable form thereof, for therapy (e.g., dose titration).

A subject identified or selected according to the methods provided herein may be a human subject. The subject may be a child (e.g., less than 18 years of age). For example, the subject may be no older than 18 years, 16 years, 14 years, 12 years, 10 years, 8 years, 6 years, 5 years, 4 years, 3 years, 2 years, 1 year, 6 months, or less. The subject may be an adult. For example, the subject may be older than 18 years, such as at least 20 years, 25 years, 30 years, 35 years, 40 years, or older. The subject may have been diagnosed with a CASTOR disorder such as propionic acidemia (e.g., as described herein). The subject may have been diagnosed with a metabolic disorder. The subject may have been diagnosed with a neurological disorder (e.g., PKAN).

One or more biomarkers may be assessed. Propionic acidemia (PA) is a rare autosomal-recessive metabolic disease that arises from mutations in propionyl-CoA (C3-CoA) carboxylase. Reduced enzyme activity blocks C3-CoA metabolism leading to an elevated plasma C3:C2-carnitine ratio, the hallmark biomarker of PA. The metabolic imbalances experienced in PA are poorly defined, and here we use a hypomorphic PA mouse model to demonstrate that C3-CoA accumulation results in a significant reduction in liver non-esterified CoA (CoASH) and acetyl-CoA (C2-CoA). Tricarboxylic acid (TCA) cycle intermediates that are normally metabolized accumulate in the urine providing direct evidence for compromised mitochondrial function in PA. Accordingly, the one or more biomarkers assessed may include one or more TCA cycle intermediates or metabolites. In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, malate may be assessed. In some embodiments, a subject has abnormal levels of one or more biomarkers (e.g., as described herein). In some embodiments, a subject has abnormal levels of one or more TCA cycle metabolites (e.g., prior to commencement of a therapeutic regimen, as described herein). In some embodiments, a subject has elevated levels of one or more TCA cycle metabolites (e.g., prior to commencement of a therapeutic regimen, as described herein). In some embodiments, a subject has depressed levels of one or more TCA cycle metabolites (e.g., prior to commencement of a therapeutic regimen, as described herein). In some embodiments, a subject has an elevated level of a first TCA cycle metabolite and a depressed level of a second TCA cycle metabolite (e.g., prior to commencement of a therapeutic regimen, as described herein).

Carnitine and CoA levels may also be assessed. In some embodiments, the subject has an elevated C3-carnitine level, a depressed C2-carnitine level, a depressed carnitine level, and/or an elevated C3:C2-carnitine ratio in plasma. In some embodiments, the subject has an elevated C3-carnitine level, a depressed C2-carnitine level, a depressed carnitine level, and/or an elevated C3:C2-carnitine ratio in the liver. In some embodiments, the subject has an elevated C3:C2-Coenzyme A (CoA) level in the liver. In some embodiments, the subject has an elevated C3-CoA level in the liver and/or heart.

A therapeutic regimen suitable for a subject having, e.g., abnormal of one or more biomarkers for a CASTOR disorder or other disorder (e.g., as described herein) may comprise treatment with a pantothenate kinase (PanK) activator. PanK catalyzes the rate-controlling step in CoA biosynthesis and its inhibition by C3-CoA prevents a compensatory increase in CoA biosynthesis to alleviate CoASH sequestration. The compounds provided herein include allosteric PanK activators that counteracts C3-CoA inhibition. Administration of a compound provided herein may thus increase hepatic CoASH and C2-CoA, and decrease C3-CoA, leading to significant improvement in the intracellular C3:C2-CoA ratio and the clinically relevant biomarker, the plasma C3:C2-carnitine ratio. As described in the Examples, elevated urinary malate is a major component of the metabolic signature for TCA cycle dysfunction in the PA mouse and the 80% reduction in urine malate upon administration of a compound provided herein (e.g., PZ-3022) indicates the restoration of mitochondrial function. Thus, CoASH sequestration in PA leads to reduced TCA cycle activity that is relieved following administration of a compound provided herein providing preclinical proof of concept for PanK activators as a therapy to attenuate the underlying mitochondrial defect in PA.

In an aspect, the present disclosure provides a method comprising: (a) providing a subject (e.g., a human subject, such as a human child) having abnormal levels of one or more tricarboxylic acid (TCA) cycle metabolites (e.g., malate, glucose, etc.); and (b) based at least in part on (a), identifying the subject as being in need of a treatment with a therapeutic agent (e.g., a compound provided herein) useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN). In some embodiments, the method further comprises administering (e.g., orally administering) the therapeutic agent to the subject (e.g., for at least one administration, such as for at least one day, one week, one month, two months, three months, four months, five months, six months, one year, or longer).

In another aspect, the present disclosure provides a method comprising: (a) providing a subject (e.g., a human subject, such as a human child) having abnormal levels of one or more tricarboxylic acid (TCA) cycle metabolites (e.g., malate, glucose, etc.); and (b) based at least in part on (a), administering (e.g., orally administering) a therapeutically effective amount of a therapeutic agent (e.g., a compound provided herein) useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN) to the subject (e.g., for at least one administration, such as for at least one day, one week, one month, two months, three months, four months, five months, six months, one year, or longer).

In an additional aspect, the present disclosure provides a compound (e.g., a compound provided herein), or a pharmaceutically acceptable salt thereof, for use in treating a CASTOR disorder (e.g., propionic acidemia) in a subject (e.g., a human subject, such as a human child) comprising: (a) providing the subject having abnormal levels of one or more tricarboxylic acid (TCA) cycle metabolites (e.g., malate, glucose, etc.); and (b) based at least in part on (a), identifying the subject as being in need of a treatment with the compound or salt thereof useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN). In some embodiments, the use further comprises administering a therapeutically effective amount of the compound to the subject.

In a related aspect, the present disclosure provides a compound (e.g., a compound provided herein), or a pharmaceutically acceptable salt thereof, for use in treating a CASTOR disorder (e.g., propionic acidemia) in a subject (e.g., a human subject, such as a human child) comprising: (a) providing the subject having abnormal levels of one or more tricarboxylic acid (TCA) cycle metabolites (e.g., malate, glucose, etc.); and (b) based at least in part on (a), administering (e.g., orally administering) a therapeutically effective amount of the compound or salt thereof useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN) to the subject (e.g., for at least one administration, such as for at least one day, one week, one month, two months, three months, four months, five months, six months, one year, or longer).

In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites comprise malate. In some embodiments, the level at least one of the one or more TCA cycle metabolites is elevated. In some embodiments, the malate level is elevated. In some embodiments, the level of at least one of the one or more TCA cycle metabolites is depressed. In some embodiments, the levels of TCA cycle metabolites of the one or more TCA cycle metabolites are urinary levels. In some embodiments, the levels of TCA cycle metabolites of the one or more TCA cycle metabolites are plasma levels. In some embodiments, the method further comprises assessing a plasma and/or urine sample from the subject to determine the levels of the one or more TCA cycle metabolites.

In some embodiments, the subject has an elevated C3-carnitine level, a depressed C2-carnitine level, a depressed carnitine level, and/or an elevated C3:C2-carnitine ratio in plasma. In some embodiments, the subject has an elevated C3-carnitine level, a depressed C2-carnitine level, a depressed carnitine level, and/or an elevated C3:C2-carnitine ratio in the liver. In some embodiments, the subject has an elevated C3:C2-Coenzyme A (CoA) level in the liver. In some embodiments, the subject has an elevated C3-CoA level in the liver and/or heart.

In some embodiments, the subject has elevated malate, methylcitrate, citrate, α-ketoglutarate, succinate, glycine, and methylmalonate levels in urine and/or plasma and an elevated C3:C2-carnitine ratio in plasma and/or the liver. In some embodiments, the subject has elevated malate levels in urine and an elevated C3:C2-carnitine ratio in plasma. In some embodiments, the subject has elevated malate, methylcitrate, citrate, α-ketoglutarate, succinate, glycine, and methylmalonate levels in urine and/or plasma and an elevated C3:C2-Coenzyme A (CoA) level in the liver. In some embodiments, the subject has elevated malate levels in urine and an elevated C3:C2-Coenzyme A (CoA) level in the liver.

In some embodiments, the subject is a mammal. In some embodiments, the subject is a human subject, such as a human child (e.g., as described herein).

In some embodiments, the subject is diagnosed with a disorder associated with Coenzyme A (CoA) reduction, elevation, sequestration, toxicity, or redistribution (CASTOR). In some embodiments, the subject is diagnosed with propionic acidemia, defects in fatty acid oxidation enzymes, methylmalonic acidemia, glutaric acidemia, or HMG-CoA lyase deficiency. In some embodiments, the subject is diagnosed with propionic acidemia. In some embodiments, the subject has previously undergone a therapeutic regimen for treatment of a disorder associated with Coenzyme A (CoA) reduction, elevation, sequestration, toxicity, or redistribution (CASTOR). In some embodiments, the subject is diagnosed with a metabolic disorder. In some embodiments, the subject has previously undergone a therapeutic regimen for treatment of a metabolic disorder. In some embodiments, the subject is diagnosed with a neurological disorder such as PKAN. In some embodiments, the subject has previously undergone a therapeutic regimen for treatment of a neurological disorder such as PKAN. In some embodiments, the subject has previously been treated with pantothenate, carnitine, pantothenic acid, a protein restricted diet, antibiotics, sodium benzoate, or a combination thereof (e.g., for at least one administration, such as for at least one day, one week, one month, two months, three months, four months, five months, six months, one year, or longer). In some embodiments, the subject has not previously been treated with pantothenate, carnitine, pantothenic acid, a protein restricted diet, antibiotics, sodium benzoate, or a combination thereof. In some embodiments, the method further comprises identifying the subject as in need of treatment with pantothenate, carnitine, pantothenic acid, a protein restricted diet, antibiotics, sodium benzoate, or a combination thereof. In some embodiments, the method further comprises administering pantothenate, carnitine, pantothenic acid, a protein restricted diet, antibiotics, sodium benzoate, or a combination thereof to the subject (e.g., for at least one administration, such as for at least one day, one week, one month, two months, three months, four months, five months, six months, one year, or longer).

In another aspect, the present disclosure provides a method comprising: (a) providing a subject (e.g., a human subject, such as a human child) having (i) abnormal levels of one or more tricarboxylic acid (TCA) cycle metabolites (e.g., malate, glucose, etc.) and/or (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart; and (b) based at least in part on (a), identifying the subject as being in need of a treatment with a therapeutic agent (e.g., a compound provided herein) useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN). In some embodiments, the method further comprises administering (e.g., orally administering) the therapeutic agent to the subject (e.g., for at least one administration, such as for at least one day, one week, one month, two months, three months, four months, five months, six months, one year, or longer).

In another aspect, the present disclosure provides a method comprising: (a) providing a subject (e.g., a human subject, such as a human child) having (i) abnormal levels of one or more tricarboxylic acid (TCA) cycle metabolites (e.g., malate, glucose, etc.) and/or (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart; and (b) based at least in part on (a), administering (e.g., orally administering) a therapeutically effective amount of a therapeutic agent (e.g., a compound provided herein) useful in the treatment of a disorder associated with CoA reduction, elevation, sequestration, toxicity, or redistribution (CASTOR) (e.g., propionic acidemia), a metabolic disorder, or a neurological disorder (e.g., PKAN) to the subject (e.g., for at least one administration, such as for at least one day, one week, one month, two months, three months, four months, five months, six months, one year, or longer).

In another aspect, the present disclosure provides a compound (e.g., a compound provided herein), or a pharmaceutically acceptable salt thereof, for use in the treatment of a CASTOR disorder (e.g., propionic acidemia) in a subject (e.g., a human subject, such as a human child) comprising: (a) providing the subject having (i) abnormal levels of one or more tricarboxylic acid (TCA) cycle metabolites (e.g., malate, glucose, etc.) and/or (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart; and (b) based at least in part on (a), identifying the subject as being in need of a treatment with the compound or salt thereof useful in the treatment of a disorder associated with CASTOR (e.g., propionic acidemia), a metabolic disorder, or a neurological disorder (e.g., PKAN). In some embodiments, the use further comprises administering (e.g., orally administering) the compound or salt thereof to the subject (e.g., for at least one administration, such as for at least one day, one week, one month, two months, three months, four months, five months, six months, one year, or longer).

In another aspect, the present disclosure provides a compound (e.g., a compound provided herein), or a pharmaceutically acceptable salt thereof, for use in the treatment of a CASTOR disorder (e.g., propionic acidemia) in a subject (e.g., a human subject, such as a human child) comprising: (a) providing the subject having (i) abnormal levels of one or more tricarboxylic acid (TCA) cycle metabolites (e.g., malate, glucose, etc.) and/or (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart; and (b) based at least in part on (a), administering (e.g., orally administering) a therapeutically effective amount of the compound or salt thereof useful in the treatment of a disorder associated with CASTOR (e.g., propionic acidemia), a metabolic disorder, or a neurological disorder (e.g., PKAN) to the subject (e.g., for at least one administration, such as for at least one day, one week, one month, two months, three months, four months, five months, six months, one year, or longer).

In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites comprise malate. In some embodiments, the level at least one of the one or more TCA cycle metabolites is elevated. In some embodiments, the malate level is elevated. In some embodiments, the level of at least one of the one or more TCA cycle metabolites is depressed. In some embodiments, the levels of TCA cycle metabolites of the one or more TCA cycle metabolites are urinary levels. In some embodiments, the levels of TCA cycle metabolites of the one or more TCA cycle metabolites are plasma levels. In some embodiments, the method further comprises assessing a plasma and/or urine sample from the subject to determine the levels of the one or more TCA cycle metabolites.

In some embodiments, the subject has an elevated C3-carnitine level, a depressed C2-carnitine level, a depressed carnitine level, and/or an elevated C3:C2-carnitine ratio in plasma. In some embodiments, the subject has an elevated C3-carnitine level, a depressed C2-carnitine level, a depressed carnitine level, and/or an elevated C3:C2-carnitine ratio in the liver. In some embodiments, the subject has an elevated C3:C2-Coenzyme A (CoA) level in the liver. In some embodiments, the subject has an elevated C3-CoA level in the liver and/or heart.

In some embodiments, the subject has (i) abnormal levels of one or more tricarboxylic acid (TCA) cycle metabolites (e.g., malate, glucose, etc.) and (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart. In some embodiments, the subject has elevated levels of one or more tricarboxylic acid (TCA) cycle metabolites (e.g., malate, glucose, etc.) and (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart. In some embodiments, the subject has depressed levels of one or more tricarboxylic acid (TCA) cycle metabolites (e.g., malate, glucose, etc.) and (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart. In some embodiments, the subject has elevated levels of malate (e.g., urinary malate) and (ii) one or more of an elevated C3-carnitine plasma level, a depressed C2-carnitine plasma level, a depressed carnitine plasma level, an elevated C3:C2-carnitine ratio in plasma, an elevated C3-carnitine level in the liver, a depressed C2-carnitine level in the liver, a depressed carnitine level in the liver, an elevated C3:C2-carnitine ratio in the liver, an elevated C3:C2-Coenzyme A (CoA) level in the liver, and an elevated C3-CoA level in the liver and/or heart. In some embodiments, the subject has elevated malate, methylcitrate, citrate, α-ketoglutarate, succinate, glycine, and methylmalonate levels in urine and/or plasma and an elevated C3:C2-carnitine ratio in plasma and/or the liver. In some embodiments, the subject has elevated malate levels in urine and an elevated C3:C2-carnitine ratio in plasma. In some embodiments, the subject has elevated malate, methylcitrate, citrate, α-ketoglutarate, succinate, glycine, and methylmalonate levels in urine and/or plasma and an elevated C3:C2-Coenzyme A (CoA) level in the liver. In some embodiments, the subject has elevated malate levels in urine and an elevated C3:C2-Coenzyme A (CoA) level in the liver.

In some embodiments, the subject is a mammal. In some embodiments, the subject is a human subject.

In some embodiments, the subject is diagnosed with a disorder associated with Coenzyme A (CoA) reduction, elevation, sequestration, toxicity, or redistribution (CASTOR). In some embodiments, the subject is diagnosed with propionic acidemia, defects in fatty acid oxidation enzymes, methylmalonic acidemia, glutaric acidemia, or HMG-CoA lyase deficiency. In some embodiments, the subject is diagnosed with propionic acidemia. In some embodiments, the subject has previously undergone a therapeutic regimen for treatment of a disorder associated with Coenzyme A (CoA) reduction, elevation, sequestration, toxicity, or redistribution (CASTOR). In some embodiments, the subject is diagnosed with a metabolic disorder. In some embodiments, the subject has previously undergone a therapeutic regimen for treatment of a metabolic disorder. In some embodiments, the subject is diagnosed with a neurological disorder such as PKAN. In some embodiments, the subject has previously undergone a therapeutic regimen for treatment of a neurological disorder such as PKAN. In some embodiments, the subject has previously been treated with pantothenate, carnitine, pantothenic acid, a protein restricted diet, antibiotics, sodium benzoate, or a combination thereof (e.g., for at least one administration, such as for at least one day, one week, one month, two months, three months, four months, five months, six months, one year, or longer). In some embodiments, the method further comprises identifying the subject as in need of treatment with pantothenate, carnitine, pantothenic acid, a protein restricted diet, antibiotics, sodium benzoate, or a combination thereof. In some embodiments, the method further comprises administering pantothenate, carnitine, pantothenic acid, a protein restricted diet, antibiotics, sodium benzoate, or a combination thereof to the subject (e.g., for at least one administration, such as for at least one day, one week, one month, two months, three months, four months, five months, six months, one year, or longer).

In a further aspect, the present disclosure provides a method comprising: (a) providing a first analysis of a first sample (e.g., urine sample) derived from a subject (e.g., human subject, such as a human child) at a first time, wherein the first analysis provides first levels of one or more tricarboxylic acid (TCA) cycle metabolites (e.g., malate, glucose, etc.); (b) providing a second analysis of a second sample (e.g., urine sample) derived from the subject at a second time, wherein the second analysis provides second levels of the one or more TCA cycle metabolites; (c) assessing a difference between a second level and a first level of at least one of the one or more TCA cycle metabolites; and (d) based at least in part on (c), identifying the subject as being in need of a treatment with a therapeutic regimen comprising administration of a therapeutic agent (e.g., a compound provided herein) useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN). In some embodiments, the method further comprises administering the therapeutic agent to the subject (e.g., for at least one administration, such as for at least one day, one week, one month, two months, three months, four months, five months, six months, one year, or longer).

In another aspect, the present disclosure provides a method comprising: (a) providing a first analysis of a first sample (e.g., urine sample) derived from a subject (e.g., human subject, such as a human child) at a first time, wherein the first analysis provides first levels of one or more tricarboxylic acid (TCA) cycle metabolites (e.g., malate, glucose, etc.); (b) providing a second analysis of a second sample (e.g., urine sample) derived from the subject at a second time, wherein the second analysis provides second levels of the one or more TCA cycle metabolites, and wherein the subject has undergone treatment with a therapeutic regimen comprising administration of a therapeutic agent (e.g., a compound provided herein) useful in the treatment of a disorder associated with Coenzyme A (CoA) reduction, elevation, sequestration, toxicity, or redistribution (CASTOR) (e.g., propionic acidemia), a metabolic disorder, or a neurological disorder (e.g., PKAN) between the first time and the second time; (c) assessing a difference between a second level and a first level of at least one of the one or more TCA cycle metabolites; and (d) based at least in part on (c), (i) identifying the subject as being in need of a change in a therapeutic regimen if the difference in (c) exceeds or does not meet a threshold level, wherein the change comprises altering an amount of the therapeutic agent administered and/or the frequency of administration of the therapeutic agent to the subject, or (ii) identifying the subject as not being in need of a change in the therapeutic regimen in the difference in (c) does not exceed the threshold level.

In a further aspect, the present disclosure provides a compound (e.g., a compound provided herein), or a pharmaceutically acceptable salt thereof, for us in the treatment of a CASTOR disorder (e.g., propionic acidemia) in a subject (e.g., a human subject, such as a human child), comprising: (a) providing a first analysis of a first sample (e.g., urine sample) derived from the subject at a first time, wherein the first analysis provides first levels of one or more tricarboxylic acid (TCA) cycle metabolites (e.g., malate, glucose, etc.); (b) providing a second analysis of a second sample (e.g., urine sample) derived from the subject at a second time, wherein the second analysis provides second levels of the one or more TCA cycle metabolites; (c) assessing a difference between a second level and a first level of at least one of the one or more TCA cycle metabolites; and (d) based at least in part on (c), identifying the subject as being in need of a treatment with a therapeutic regimen comprising administration of the compound or salt thereof useful in the treatment of a disorder associated with CASTOR (e.g., propionic acidemia), a metabolic disorder, or a neurological disorder (e.g., PKAN). In some embodiments, the use further comprises administering the compound or salt thereof to the subject (e.g., for at least one administration, such as for at least one day, one week, one month, two months, three months, four months, five months, six months, one year, or longer).

In another aspect, the present disclosure provides a compound (e.g., a compound provided herein), or a pharmaceutically acceptable salt thereof, for us in the treatment of a CASTOR disorder (e.g., propionic acidemia), a metabolic disorder, or a neurological disorder (e.g., PKAN) in a subject (e.g., a human subject, such as a human child), comprising: (a) providing a first analysis of a first sample (e.g., urine sample) derived from the subject at a first time, wherein the first analysis provides first levels of one or more tricarboxylic acid (TCA) cycle metabolites (e.g., malate, glucose, etc.); (b) providing a second analysis of a second sample (e.g., urine sample) derived from the subject at a second time, wherein the second analysis provides second levels of the one or more TCA cycle metabolites, and wherein the subject has undergone treatment with a therapeutic regimen comprising administration of the compound or salt thereof useful in the treatment of a disorder associated with CASTOR (e.g., propionic acidemia), a metabolic disorder, or a neurological disorder (e.g., PKAN) between the first time and the second time; (c) assessing a difference between a second level and a first level of at least one of the one or more TCA cycle metabolites; and (d) based at least in part on (c), (i) identifying the subject as being in need of a change in a therapeutic regimen if the difference in (c) exceeds or does not meet a threshold level, wherein the change comprises altering an amount of the therapeutic agent administered and/or the frequency of administration of the therapeutic agent to the subject, or (ii) identifying the subject as not being in need of a change in the therapeutic regimen in the difference in (c) does not exceed the threshold level.

In some embodiments, (d) comprises decreasing a dosage of the therapeutic agent if the difference in (c) exceeds the threshold level. In some embodiments, (d) comprises increasing a dosage of the therapeutic agent if the difference in (c) does not meet the threshold level. In some embodiments, (d) comprises not changing the therapy regimen if the difference in (c) does not exceed the threshold level. In some embodiments, (d) comprises changing the frequency of administration of the therapeutic agent if the difference in (c) exceeds the threshold level. In some embodiments, (d) comprises changing the dosage and the frequency of administration of the therapeutic agent if the difference in (c) exceeds the threshold level. In some embodiments, the frequency is increased. In some embodiments, the frequency is decreased.

In some embodiments, the first time precedes the second time, and the subject is diagnosed with a disorder associated with CASTOR (e.g., propionic acidemia), a metabolic disorder, or a neurological disorder (e.g., PKAN) after performance of (a) but before performance of (b). In some embodiments, the first time precedes the second time, and the subject is diagnosed with a disorder associated with CASTOR (e.g., propionic acidemia), a metabolic disorder, or a neurological disorder (e.g., PKAN) before performance of (a). In some embodiments, the first time precedes the second time, and the subject is diagnosed with a disorder associated with CASTOR (e.g., propionic acidemia), a metabolic disorder, or a neurological disorder (e.g., PKAN) after performance of (b). In some embodiments, the subject has undergone treatment with the therapeutic regimen (e.g., administration of a compound provided herein) prior to (a). In some embodiments, the first time is at least one week before the second time. In some embodiments, the first time is at least two weeks before the second time. In some embodiments, the first time is at least one month before the second time. In some embodiments, the first time is at least six months before the second time.

In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites comprise malate.

In some embodiments, prior to administration with a compound useful in the treatment of a CASTOR disorder, a metabolic disorder, or a neurological disorder (e.g., PKAN), the subject has elevated malate, methylcitrate, citrate, α-ketoglutarate, succinate, glycine, and methylmalonate levels in urine and/or plasma and an elevated C3:C2-carnitine ratio in plasma and/or the liver. In some embodiments, prior to administration with a compound useful in the treatment of a CASTOR disorder, a metabolic disorder, or a neurological disorder (e.g., PKAN), the subject has elevated malate levels in urine and an elevated C3:C2-carnitine ratio in plasma. In some embodiments, prior to administration with a compound useful in the treatment of a CASTOR disorder, a metabolic disorder, or a neurological disorder (e.g., PKAN), the subject has elevated malate, methylcitrate, citrate, α-ketoglutarate, succinate, glycine, and methylmalonate levels in urine and/or plasma and an elevated C3:C2-Coenzyme A (CoA) level in the liver. In some embodiments, prior to administration with a compound useful in the treatment of a CASTOR disorder, the subject has elevated malate levels in urine and an elevated C3:C2-Coenzyme A (CoA) level in the liver.

In some embodiments, the second level at least one of the one or more TCA cycle metabolites is lower than the first level of the at least one of the one or more TCA cycle metabolites. In some embodiments, the second level of malate level is lower than the first level of malate. In some embodiments, the second level at least one of the one or more TCA cycle metabolites is higher than the first level of the at least one of the one or more TCA cycle metabolites. In some embodiments, the first sample and the second sample are urine samples. In some embodiments, the first sample and the second sample are plasma samples.

In some embodiments, prior to (b), the subject has an elevated C3-carnitine level, a depressed C2-carnitine level, a depressed carnitine level, and/or an elevated C3:C2-carnitine ratio in plasma. In some embodiments, prior to (b), the subject has an elevated C3-carnitine level, a depressed C2-carnitine level, a depressed carnitine level, and/or an elevated C3:C2-carnitine ratio in the liver. In some embodiments, prior to (b), the subject has an elevated C3:C2-Coenzyme A (CoA) level in the liver. In some embodiments, prior to (b), the subject has an elevated C3-CoA level in the liver and/or heart. In some embodiments, subsequent to treatment with the therapeutic regimen, the C3-carnitine level and/or the C3:C2-carnitine ratio in the plasma and/or liver of the subject decreases. In some embodiments, subsequent to treatment with the therapeutic regimen, the C3:C2-Coenzyme A (CoA) level in the liver of the subject decreases. In some embodiments, the subject is a human subject.

In some embodiments, prior to (a), the subject is diagnosed with a disorder associated with Coenzyme A (CoA) reduction, elevation, sequestration, toxicity, or redistribution (CASTOR). In some embodiments, prior to (a), the subject is diagnosed with propionic acidemia, defects in fatty acid oxidation enzymes, methylmalonic acidemia, glutaric acidemia, or HMG-CoA lyase deficiency. In some embodiments, the subject is diagnosed with propionic acidemia. In some embodiments, prior to (a), the subject has previously undergone a therapeutic regimen for treatment of a disorder associated with Coenzyme A (CoA) reduction, elevation, sequestration, toxicity, or redistribution (CASTOR). In some embodiments, the subject is diagnosed with a metabolic disorder. In some embodiments, the subject has previously undergone a therapeutic regimen for treatment of a metabolic disorder. In some embodiments, the subject is diagnosed with a neurological disorder such as PKAN. In some embodiments, the subject has previously undergone a therapeutic regimen for treatment of a neurological disorder such as PKAN. In some embodiments, prior to (a), the subject was treated with pantothenate, carnitine, pantothenic acid, a protein restricted diet, antibiotics, sodium benzoate, or a combination thereof (e.g., for at least one administration, such as for at least one day, one week, one month, two months, three months, four months, five months, six months, one year, or longer). In some embodiments, the method further comprises identifying the subject as in need of treatment with pantothenate, carnitine, pantothenic acid, a protein restricted diet, antibiotics, sodium benzoate, or a combination thereof. In some embodiments, the method further comprises administering pantothenate, carnitine, pantothenic acid, a protein restricted diet, antibiotics, sodium benzoate, or a combination thereof to the subject (e.g., for at least one administration, such as for at least one day, one week, one month, two months, three months, four months, five months, six months, one year, or longer).

The present disclosure also provides methods and systems for identifying subjects having a metabolic disorder, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency). A method for identifying a subject having a disease or disorder described herein may comprise the use of magnetic resonance methods to quantify changes in metabolites (e.g., cerebral metabolites) such as glutamate/glutamine (Glx), GABA, lactate, inositol, choline, taurine, and N-acetyl aspartate. The present disclosure also provides methods and systems for monitoring subjects undergoing treatment or who have undergone treatment for and/or evaluating the efficacy of a treatment program for a metabolic disease, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency). For example, a subject may undergo a first assessment (e.g., magnetic resonance-based analysis, as described herein) at a first time (e.g., prior to commencement of a therapeutic program) and a second assessment at a second time (e.g., during the course of or upon completion of a therapeutic program), and the assessments may be compared. Comparison of the first and second assessments may be used as a basis for alterations to ongoing therapeutic programs (e.g., changes in dosing, etc.) and/or for recommendations for future therapeutic programs. The methods provided herein can be used to identify and/or select patients for therapy (e.g., as described herein); to demonstrate, evince, or quantify one or more therapeutic effects, such as improvement of a patient's condition and/or a reduction or improvement in one or more symptoms of a disorder for which a patient is treated (e.g., as described herein); and/or for identifying an appropriate dose of a compound, or a pharmaceutically acceptable form thereof, for therapy (e.g., dose titration). For example, one or more substances described herein may be useful as a biomarker, such as a neural pharmacodynamic biomarker, and may correlate with symptomatic improvement. For example, an improvement in N-acetyl aspartate (NAA) levels may be suggestive of more neurons/less neurodegeneration; rescue of Glx may be suggestive that there are increased neurotransmitters and neural firing, etc.

In an aspect, the present disclosure provides a method for identifying a subject in need of treatment with a therapeutic agent (e.g., a compound provided herein) useful in the treatment of a CASTOR disorder (e.g., propionic acidemia), a metabolic disorder, or a neurological disorder (e.g., PKAN), comprising: (a) using a magnetic resonance technique (e.g., magnetic resonance imaging) to measure a substance in the brain of the subject, and (b) based at least in part on the amount of the substance, identifying the subject as being in need of the treatment, wherein the therapeutic agent is a compound provided herein, or a pharmaceutically acceptable form thereof.

In some embodiments, the magnetic resonance technique is magnetic resonance imaging, such as ¹H magnetic resonance imaging.

In some embodiments, the substance is glutamate/glutamine (Glx), GABA, lactate, inositol, choline, taurine, or N-acetyl aspartate. In some embodiments, the Glx/tCr ratio is assessed. In some embodiments, the GABA/tCr ratio is assessed.

In some embodiments, the method comprises treatment of a subject with a therapeutic agent (e.g., a compound provided herein) useful in the treatment of a CASTOR disorder (e.g., propionic acidemia), a metabolic disorder, or a neurological disorder (e.g., PKAN).

In some embodiments, the subject has been diagnosed with a disorder associated with Coenzyme A (CoA) reduction, elevation, sequestration, toxicity, or redistribution (CASTOR). In some embodiments, the subject has been diagnosed with propionic acidemia, defects in fatty acid oxidation enzymes, methylmalonic acidemia, glutaric acidemia, or HMG-CoA lyase deficiency. In some embodiments, the subject has been diagnosed with propionic acidemia. In some embodiments, the subject has previously undergone a therapeutic regimen for treatment of a disorder associated with Coenzyme A (CoA) reduction, elevation, sequestration, toxicity, or redistribution (CASTOR). In some embodiments, the subject has been diagnosed with a metabolic disorder. In some embodiments, the subject has previously undergone a therapeutic regimen for treatment of a metabolic disorder. In some embodiments, the subject has been diagnosed with a neurological disorder such as PKAN. In some embodiments, the subject has previously undergone a therapeutic regimen for treatment of a neurological disorder such as PKAN. In some embodiments, the subject has been diagnosed with a metabolic disease, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency). In some embodiments, the subject is undergoing, plans to undergo, or has previously undergone therapy for a metabolic disease, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency).

In some embodiments, the subject has been treated with pantothenate, carnitine, pantothenic acid, a protein restricted diet, antibiotics, sodium benzoate, or a combination thereof (e.g., for at least one administration, such as for at least one day, one week, one month, two months, three months, four months, five months, six months, one year, or longer). In some embodiments, the method further comprises identifying the subject as in need of treatment with pantothenate, carnitine, pantothenic acid, a protein restricted diet, antibiotics, sodium benzoate, or a combination thereof. In some embodiments, the method further comprises administering pantothenate, carnitine, pantothenic acid, a protein restricted diet, antibiotics, sodium benzoate, or a combination thereof to the subject (e.g., for at least one administration, such as for at least one day, one week, one month, two months, three months, four months, five months, six months, one year, or longer).

In another aspect, the present disclosure provides a method for assessing a therapeutic regimen for a subject (e.g., a human subject, such as a human child), comprising: (a) using a magnetic resonance technique (e.g., magnetic resonance imaging) to measure a substance in the brain of the subject at a first time; (b) using the magnetic resonance technique to measure the substance in the brain of the subject a second time; (c) assessing the difference between the amount of the substance at the first time and the second time; and (d) based at least in part on (c), changing the therapeutic regimen if the amount in (b) or the difference in (c) exceeds or does not meet a threshold level or not changing the therapeutic regimen if the amount in (b) or the difference in (c) does not exceed the threshold level, wherein the therapeutic regimen comprises administration of a compound provided herein, or a pharmaceutically acceptable form thereof.

In some embodiments, the first time precedes the second time. In some embodiments, the subject is diagnosed with the disorder before performance of (a). In some embodiments, the subject is diagnosed with the disorder after performance of (a) but before performance of (b). In some embodiments, the subject is diagnosed with the disorder after performance of (a) and (b).

In some embodiments, the subject is undergoing the therapeutic regimen prior to performance of (a). In some embodiments, the subject is undergoing the therapeutic regimen prior to performance of (b).

In some embodiments, (d) comprises decreasing a dosage of the compound, or the pharmaceutically acceptable form thereof (e.g., if the difference in (c) exceeds a first threshold level). In some embodiments, (d) comprises increasing a dosage of the compound, or the pharmaceutically acceptable form thereof (e.g., if the difference in (c) does not meet a second threshold level). In some embodiments, (d) comprises decreasing or increasing a dosage of the compound if the amount in (b) exceeds or does not meet a threshold level. In some embodiments, (d) comprises decreasing a dosage of the compound, or the pharmaceutically acceptable form thereof, if the amount in (b) exceeds a first threshold level. In some embodiments, (d) comprises increasing a dosage of the compound, or the pharmaceutically acceptable form thereof, if the amount in (b) does not meet a second threshold level. In some embodiments, (d) comprises not changing the therapeutic regimen if the amount in (b) does not exceed a threshold level and/or if the difference in (c) does not exceed a threshold level.

In some embodiments, the magnetic resonance technique is magnetic resonance imaging, such as ¹H magnetic resonance imaging.

In some embodiments, the substance is glutamate/glutamine (Glx), GABA, lactate, inositol, choline, taurine, or N-acetyl aspartate (NAA). In some embodiments, the Glx/tCr ratio is assessed. In some embodiments, the GABA/tCr ratio is assessed.

In some embodiments, a threshold level is expressed as a ratio, such as a ratio of the metabolite to total creatine (tCr). In some embodiments, a threshold level of Glx/tCr is between about 0.5-2, such as between about 0.6-2, 0.8-2, 1-2, 1-1.8, 1-1.5, 1.2-1.8, or any range therein. some embodiments, a threshold level of Glx/tCr is between about 1-1.8. In some embodiments, a threshold level of NAA/tCr is between about 0.1-2, such as between about 0.1-1.5, 0.1-1, 0.5-1, 0.5-0.9, 0.6-0.9, 0.5-2, 1-2, or any range therein. In some embodiments, a threshold level of NAA/tCr is between about 0.55-0.85. In some embodiments, a threshold level of lactate/tCr is between about 0.01-1, such as between about 0.01-0.8, 0.01-0.5, 0.01-0.4, 0.05-0.4, or any range therein. In some embodiments, a threshold level of lactate/tCr is between about 0.05-0.4. In some embodiments, a threshold level of inositol/tCr is between about 0.1-2, such as between about 0.1-1.5, 0.1-1.2, 0.1-1.1, 0.4-1.1, 0.4-1.2, 0.5-1.1, or any range therein. In some embodiments, a threshold level of inositol/tCr is between about 0.5-1.1. In some embodiments, a threshold level of total choline/tCr is between about 0.05-0.5, such as between about 0.05-0.4, 0.05-0.25, 0.1-0.25, or any range therein. In some embodiments, a threshold level of total choline/tCr is between about 0.1-0.25. In some embodiments, a threshold level of taurine/tCr is between about 0.5-2, such as between about 0.5-1.8, 0.5-1.6, 0.5-1.5, 0.6-1.6, 0.8-1.5, or any range therein. In some embodiments, a threshold level of taurine/tCr is between about 0.8-1.5.

In some embodiments, the method comprises treatment of a subject with a therapeutic agent (e.g., a compound provided herein) useful in the treatment of a CASTOR disorder (e.g., propionic acidemia), a metabolic disorder, or a neurological disorder (e.g., PKAN).

In some embodiments, the subject has been diagnosed with a disorder associated with Coenzyme A (CoA) reduction, elevation, sequestration, toxicity, or redistribution (CASTOR). In some embodiments, the subject has been diagnosed with propionic acidemia, defects in fatty acid oxidation enzymes, methylmalonic acidemia, glutaric acidemia, or HMG-CoA lyase deficiency. In some embodiments, the subject has been diagnosed with propionic acidemia. In some embodiments, the subject has previously undergone a therapeutic regimen for treatment of a disorder associated with Coenzyme A (CoA) reduction, elevation, sequestration, toxicity, or redistribution (CASTOR). In some embodiments, the subject has been diagnosed with a metabolic disorder. In some embodiments, the subject has previously undergone a therapeutic regimen for treatment of a metabolic disorder. In some embodiments, the subject has been diagnosed with a neurological disorder such as PKAN. In some embodiments, the subject has previously undergone a therapeutic regimen for treatment of a neurological disorder such as PKAN. In some embodiments, the subject has been diagnosed with a metabolic disease, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency). In some embodiments, the subject is undergoing, plans to undergo, or has previously undergone therapy for a metabolic disease, neurologic disorder (e.g., PKAN), or a CASTOR disorder (e.g., defects in fatty acid oxidation enzymes, methylmalonic acidemia, glutaric acidemia, propionic acidemia, and HMG-CoA lyase deficiency).

In some embodiments, the subject has been treated with pantothenate, carnitine, pantothenic acid, a protein restricted diet, antibiotics, sodium benzoate, or a combination thereof (e.g., for at least one administration, such as for at least one day, one week, one month, two months, three months, four months, five months, six months, one year, or longer). In some embodiments, the method further comprises identifying the subject as in need of treatment with pantothenate, carnitine, pantothenic acid, a protein restricted diet, antibiotics, sodium benzoate, or a combination thereof. In some embodiments, the method further comprises administering pantothenate, carnitine, pantothenic acid, a protein restricted diet, antibiotics, sodium benzoate, or a combination thereof to the subject (e.g., for at least one administration, such as for at least one day, one week, one month, two months, three months, four months, five months, six months, one year, or longer).

In some embodiments of any of the above aspects, the therapeutic agent is a compound provided herein, or a form thereof (e.g., a pharmaceutically acceptable salt thereof).

In some embodiments of any of the above aspects, one or more TCA cycle metabolites (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine) are present in a bodily sample (e.g., urine, plasma, and/or blood sample) taken from a subject prior to treatment with a compound provided herein, or a pharmaceutically acceptable form thereof.

In some embodiments of any of the above aspects, the subject has a normal level of one or more TCA cycle metabolites or other analytes. Normal or reference levels for various analytes are included in Tables 1A and 1B below (adapted from Haijes et al. Orphanet J. Rare Dis. 2020, which is herein incorporated by reference in its entirety), in which N refers to the number of samples, SD to standard deviation, Min to a minimum value, Max to a maximum value, TH to therapy related, and DI-HRMS to direct-infusion high-resolution mass spectrometry. In Tables 1A and 1B, results of targeted metabolic assays in plasma are included in micromoles per liter (μmol/L), and results of targeted metabolic assays in urine are presented in millimoles per mole of creatinine. Tables 1C and 1D include additional reference ranges for various analyes in plasma/serum or urine.

TABLE 1A
Analyte levels for propionic acidemia patients.

| | | | | | | | Reference | |
|---|---|---|---|---|---|---|---|---|
| Analyte | Matrix | N | Median | S | Min | Max | Range | Known? |
| Targeted metabolic assays | | | | | | | | |
| Propionylcarnitine | Plasma | 34 | 54.4 | 19.6 | 23 | 99.5 | 0.00-0.81 | Yes |
| Glycine | Plasma | 73 | 1381 | 362 | 350 | 1962 | 166-330 | Yes |
| Isoleucine | Plasma | 73 | 21 | 9 | 7 | 48 | 34-106 | Yes, TH |
| Valine | Plasma | 73 | 66 | 26 | 20 | 183 | 155-343 | Yes, TH |
| Leucine | Plasma | 73 | 59 | 23 | 31 | 152 | 86-206 | Yes, TH |
| Threonine | Plasma | 73 | 72 | 29 | 29 | 212 | 102-246 | Yes, TH |
| Acetylcarnitine | Plasma | 34 | 10.1 | 7.5 | 1.6 | 42.2 | 0.69-9.72 | Yes |
| Methylcitric acid | Urine | 3 | 697 | 383 | 426 | 968 | — | Yes |
| 3-Hydroxy-propionic | Urine | 10 | 232 | 99 | 89 | 349 | 0-20 | Yes |
| acid | | | | | | | | |
| Histidine | Plasma | 73 | 65 | 14 | 32 | 107 | 68-108 | No |
| Methylmalonic acid | Urine | 12 | 2 | 3.1 | 1 | 10 | 0-20 | — |
| Methylmalonic acid | Plasma | 0 | — | — | — | — | 0.12-0.29 | — |
| Methylmalonylcarnitine | Plasma | 26 | 0.01 | 0.01 | 0.01 | 0.04 | 0.00-0.07 | — |
| Methylcitric acid | Plasma | 0 | — | — | — | — | 0.00-0.83 | — |
| Glutamine | Plasma | 73 | 494 | 105 | 252 | 793 | 457-857 | — |
| Untargeted DI-HRMS analysis | | | | | | | P-value | |
| 2-Methylcitric acid | Plasma | 23 | 57.4 | 43.1 | −1.8 | 146.9 | <0.0001 | Yes |
| (3 isomers) | | | | | | | | |
| 3-Dehydroxycarnitine | Plasma | 23 | 15.9 | 53.2 | 0.4 | 203.4 | <0.0001 | Yes |
| Propionylcarnitine | Plasma | 23 | 12 | 80.8 | −2.8 | 362.6 | <0.0001 | Yes |
| Fructoseglycine | Plasma | 23 | 7.6 | 5.7 | −0.8 | 25.8 | <0.0001 | No |
| Isoleucyl-Isoleucine | Plasma | 23 | 5.7 | 3.8 | −0.6 | 13.9 | <0.0001 | No |
| (3 isomers) | | | | | | | | |
| LysoPC(15:0) | Plasma | 23 | 5.5 | 4.6 | −2.9 | 16 | <0.0001 | No |
| (2 isomers) | | | | | | | | |
| Glucosamine | Plasma | 23 | 5.1 | 8.9 | −2.5 | 31.1 | <0.0001 | No |
| (2 isomers) | | | | | | | | |
| Threonic acid | Plasma | 23 | 2.1 | 1.6 | −2.8 | 3.6 | 0.0009 | No |
| DL-2-Aminooctanoic | Plasma | 23 | −1.6 | 0.1 | −1.7 | −1.3 | <0.0001 | No |
| acid | | | | | | | | |
| 2-Methyl-3-ketovaleric | Plasma | 23 | −1.8 | 0.3 | −2.8 | −1.3 | <0.0001 | No |
| acid (7 isomers) | | | | | | | | |
| L-Glutamine | Plasma | 23 | −1.8 | 0.1 | −1.9 | −1.6 | <0.0001 | No |
| (4 isomers) | | | | | | | | |
| L-Methionine | Plasma | 23 | −2 | 5 | −2.5 | −0.5 | <0.0001 | No |
| L-Histidine | Plasma | 23 | −2.8 | 1.4 | −5.6 | −0.6 | <0.0001 | No |
| Propionylglycine | Plasma | 23 | 28.5 | 29.3 | −3 | 91.1 | <0.0001 | Yes |
| (9 isomers) | | | | | | | | |
| Glycine | Plasma | 23 | 9.5 | 4.8 | −2.4 | 18.5 | <0.0001 | Yes |
| 2-Amino-3- | Plasma | 23 | 6.2 | 6.5 | −1.9 | 23.7 | <0.0001 | No |
| phosphonopropionic | | | | | | | | |
| acid | | | | | | | | |
| 1-(sn-Glycero-3- | Plasma | 23 | 5.3 | 3.9 | −0.6 | 10.8 | <0.0001 | No |
| phospho)-1D-myo- | | | | | | | | |
| inositol | | | | | | | | |
| LysoPC(17:0) | Plasma | 23 | 4.1 | 5.3 | −2.3 | 17.3 | <0.0001 | No |
| (2 isomers) | | | | | | | | |
| Homocysteine | Plasma | 23 | −1.7 | 1.2 | −3.5 | 2.1 | 0.0002 | No |
| L-Isoleucine | Plasma | 23 | −1.7 | 0.7 | −2.8 | 0.1 | <0.0001 | Yes |
| (6 isomers) | | | | | | | | |
| Methylmalonic acid | Plasma | 23 | 0.1 | 1.7 | −2.2 | 7 | — | — |
| (3 isomers) | | | | | | | | |
| Propionic acid | Plasma | 23 | −0.3 | 4.4 | −2.4 | 20.1 | — | — |
| (2 isomers) | | | | | | | | |
| 3-Hydroxy-9- | Plasma | 23 | 3.1 | 3.1 | −1.7 | 8.8 | — | — |
| hexadecenoylcarnitine | | | | | | | | |

TABLE 1B
Analyte levels for methylmalonic acidemia patients.

| | | | | | | | Reference | |
|---|---|---|---|---|---|---|---|---|
| Analyte | Matrix | N | Median | SD | Min | Max | Range | Known? |
| Targeted metabolic assays | | | | | | | | |
| Propionylcarnitine | Plasma | 68 | 21 | 21.7 | 2.7 | 83.7 | 0.00-0.81 | Yes |
| Glycine | Plasma | 77 | 492 | 369 | 168 | 1916 | 166-330 | Yes |
| Isoleucine | Plasma | 77 | 32 | 15 | 7 | 84 | 34-106 | Yes, TH |
| Valine | Plasma | 77 | 107 | 42 | 41 | 217 | 155-343 | Yes, TH |
| Leucine | Plasma | 77 | 77 | 39 | 34 | 223 | 86-206 | Yes, TH |
| Threonine | Plasma | 77 | 80 | 31 | 36 | 174 | 102-246 | Yes, TH |
| Acetylcarnitine | Plasma | 68 | 9.2 | 5.8 | 2.1 | 29.9 | 0.69-9.72 | — |
| Methylcitric acid | Urine | 0 | — | — | — | — | — | — |
| 3-Hydroxy-propionic | Urine | 57 | 13 | 19 | 0 | 86 | 0-20 | — |
| acid | | | | | | | | |
| Histidine | Plasma | 77 | 72 | 15 | 45 | 142 | 68-108 | — |
| Methylmalonic acid | Urine | 174 | 727 | 4651 | 64 | 21587 | 0-20 | Yes |
| Methylmalonic acid | Plasma | 52 | 73.8 | 345.8 | 2.9 | 1327.4 | 0.12-0.29 | Yes |
| Methylmalonylcarnitine | Plasma | 68 | 0.34 | 0.52 | 0.1 | 2.69 | 0.00-0.07 | Yes |
| Methylcitric acid | Plasma | 11 | 2.2 | 2.8 | 1 | 10.9 | 0.00-0.83 | Yes |
| Glutamine | Plasma | 77 | 380 | 126 | 190 | 736 | 457-857 | No |
| Untargeted DI-HRMS analysis | | | | | | | P-value | |
| 2-Methylcitric acid | Plasma | 51 | 6.1 | 7.6 | −0.6 | 25.5 | <0.0001 | Yes |
| (3 isomers) | | | | | | | | |
| 3-Dehydroxycarnitine | Plasma | 51 | 3.2 | 7 | −0.6 | 26.6 | <0.0001 | Yes |
| Propionylcarnitine | Plasma | 51 | 27.9 | 80 | −1.4 | 368 | <0.0001 | Yes |
| Fructoseglycine | Plasma | 51 | 2.2 | 2.2 | −1.2 | 7.6 | <0.0001 | No |
| Isoleucyl-Isoleucine | Plasma | 51 | 3.4 | 2.8 | −1 | 10.8 | <0.0001 | No |
| (3 isomers) | | | | | | | | |
| LysoPC(15:0) | Plasma | 51 | 2.5 | 4.1 | −1.7 | 20.5 | <0.0001 | No |
| (2 isomers) | | | | | | | | |
| Glucosamine | Plasma | 51 | 2.2 | 4.8 | −0.7 | 22.2 | <0.0001 | No |
| (2 isomers) | | | | | | | | |
| Threonic acid | Plasma | 51 | 2.5 | 2.4 | −1.4 | 8 | <0.0001 | No |
| DL-2-Aminooctanoic | Plasma | 51 | −1.5 | 0.5 | −1.7 | 0.4 | <0.0001 | No |
| acid | | | | | | | | |
| 2-Methyl-3-ketovaleric | Plasma | 51 | −2.1 | 0.9 | −2.8 | 1.9 | <0.0001 | No |
| acid (7 isomers) | | | | | | | | |
| L-Glutamine | Plasma | 51 | −1.5 | 0.9 | −2.1 | 1.5 | <0.0001 | No |
| (4 isomers) | | | | | | | | |
| L-Methionine | Plasma | 51 | −1.5 | 0.5 | −2.1 | 0 | <0.0001 | No |
| L-Histidine | Plasma | 51 | −1.7 | 1.3 | −5.2 | 1.6 | <0.0001 | No |
| Propionylglycine | Plasma | 51 | 1.1 | 2.4 | −2.4 | 8.7 | — | — |
| (9 isomers) | | | | | | | | |
| Glycine | Plasma | 51 | 1.5 | 2.2 | −1.1 | 8.5 | — | — |
| 2-Amino-3- | Plasma | 51 | 0.8 | 1.3 | −1.3 | 7.1 | — | — |
| phosphonopropionic | | | | | | | | |
| acid | | | | | | | | |
| 1-(sn-Glycero-3- | Plasma | 51 | 0.3 | 4.8 | −2.7 | 25.5 | — | — |
| phospho)-1D-myo- | | | | | | | | |
| inositol | | | | | | | | |
| LysoPC(17:0) | Plasma | 51 | 1.5 | 3.4 | −2.5 | 14.4 | — | — |
| (2 isomers) | | | | | | | | |
| Homocysteine | Plasma | 51 | −0.9 | 1.2 | −3 | 4.1 | — | — |
| L-Isoleucine | Plasma | 51 | −1.3 | 0.9 | −2.8 | 2.6 | — | — |
| (6 isomers) | | | | | | | | |
| Methylmalonic acid (3 | Plasma | 51 | 10 | 50.7 | 0 | 228.6 | <0.0001 | Yes |
| isomers) | | | | | | | | |
| Propionic acid (2 | Plasma | 51 | 6.9 | 79.1 | −1.1 | 344.9 | <0.0001 | No |
| isomers) | | | | | | | | |
| 3-Hydroxy-9- | Plasma | 51 | 3.6 | 7.6 | −0.9 | 40 | <0.0001 | No |
| hexadecenoylcarnitine | | | | | | | | |

TABLE 1C
Reference levels for various analytes in serum or plasma.

| | No | <3 | 3-24 | 1-13 | 13-18 | ≥18 |
|---|---|---|---|---|---|---|
| | specific | months | months | years | years | years |
| Analyte | age | of age | of age | of age | of age | of age |
| lactacte | | ≤3.3 | ≤3.1 | | | 0.5- |
| (mmol/L, plasma) | | | | | | 2.2 |
| malate | | | | | | 3.2- |
| (aka malic acid, μM) | | | | | | 12.0 |
| methyl-malonate | <1 | | | | | |
| (μmol/L, serum) | | | | | | |
| methylmalonic acid | 0-378 | <0.27 | <0.27 | <0.33 | <0.34 | <0.4 |
| (serum or plasma) | nmol/L | μM | μM | μM | μM | μM |
| methylcitrate | <2 | | | | | 0.021- |
| (serum) | μmol/L | | | | | 0.26 |
| | | | | | | μM |
| methylcitrate | ≤0.63 | | | | | |
| (μmol/L, dried blood | | | | | | |
| spot) | | | | | | |
| 3-hydroxypropionate | <11 | | | | 6-8 | 2.3-4 |
| (3-HPA; aka | μmol/L | | | | μM | μM |
| hydroxypropionic acid) | serum | | | | | |
| propionylglycine | 0 | | | | | |
| (μmol/L, serum) | | | | | | |
| 2-ketoisovaleric acid | 3-20 | | | 14.0 | | 11.0 ± |
| | μmol/L | | | (0.0-28.0) | | 1.7 |
| | plasma | | | μM | | μM |
| a-ketoglutarate | 5 | 20.7 ± | | 8.6 ± 2.6- | | 7-8.9 |
| (μM, serum; | | 2.5 | | 9.3 ± 2.3 | | |
| aka oxoglutaric acid) | | | | | | |
| propionic acid | 0.6-2.4 | | | 1.6 ± 1.2 | | 0.9 ± |
| | μmol/L | | | μM | | 1.2 |
| | plasma | | | | | μM |

| | | 8 days- | ≥8 |
|---|---|---|---|
| Acycarnitines | <7 days | 7 years | years |
| (nmol/mL, serum) | of age | of age | of age |
| acetylcarnitine | 2.14- | 2.00- | 2.00- |
| (C2) | 15.89 | 27.57 | 17.83 |
| propionylcarnitine | <0.04 | <0.05 | <0.07 |
| (C3) | | | |
| tiglylcarnitine | <0.05 | <0.09 | <0.11 |
| (C5:1-carn.) | | | |
| hexanoylcarnitine | <0.14 | <0.23 | <0.17 |
| (C6-carn.) | | | |
| 2-hexenoylcarnitine | <0.12 | <0.10 | <0.15 |
| (C6:1-carn.) | | | |
| 3-hydroxytetradecanoyl | <0.04 | <0.05 | <0.08 |
| carnitine | | | |
| (C14-OH-carn.) | | | |
| isovalerylcarnitine | <0.38 | <0.63 | <0.51 |
| (C5-carn.) | | | |
| methylmalonylcarnitine | <0.05 | <0.05 | <0.05 |
| (C4-DC-carn.) | | | |

| Carnitine, free and | | | 8-31 | 32 days- | 13 months- | 7-10 | 11-17 | ≥18 |
|---|---|---|---|---|---|---|---|---|
| total (nmol/mL, | ≤1 day | 2-7 days | days | 12 months | 6 years | years | years | years |
| plasma) | old | old | old | of age | of age | of age | of age | of age |
| total carnitine (TC) | 23-68 | 17-41 | 19-59 | 38-68 | 35-84 | 28-83 | 34-77 | 34-78 |
| free carnitine (FC) | 12-36 | 10-21 | 12-46 | 27-49 | 24-63 | 22-66 | 22-65 | 25-54 |
| acylcarnitine (AC) | 7-37 | 3-24 | 4-15 | 7-19 | 4-28 | 3-32 | 4-29 | 5-30 |
| AC/FC ratio | 0.4-1.7 | 0.2-1.4 | 0.1-0.07 | 0.2-0.5 | 0.1-0.8 | 0.1-0.9 | 0.1-0.9 | 0.1-0.8 |

| Amino acids | 0-30 | 31 days- | 2-15 | |
|---|---|---|---|---|
| (μmol/L, | days | 23 months | years | >15 years |
| plasma) | old | of age | of age | of age |
| lysine | 83.2-334.2 | 70.4-279.2 | 82.7-239.5 | 94.0-278.0 |
| valine | 73.5-309.4 | 84.9-345.0 | 110.0-333.9 | 102.6-345.4 |
| isoleucine | 19.3-127.4 | 25.2-126.4 | 27.7-110.3 | 27.7-112.8 |
| leucine | 44.0-224.5 | 51.1-216.8 | 56.6-193.6 | 54.9-205.0 |
| glycine | 145.6-518.9 | 120.0-365.0 | 129.1-429.7 | 132.0-467.0 |

TABLE 1D
Reference levels for various analytes in urine.

| | No specific | Newborn | Infant | 2 days-1 | >1 year of |
|---|---|---|---|---|---|
| Biomarker | age | (0-30 days) | (0-1 year) | year of age | age |
| lactate | <0.067 | | | | |
| (aka 2-hydroxypropanoic | | | | | |
| acid, mmol/mmol Cr) | | | | | |
| malate | 0-3.1 | 12.4-121.6 | 0-38.9 | | |
| (aka malic acid) | mcg/mg Cr | μmol/mmol | μmol/mmol | | |
| | | Cr | Cr | | |
| tiglylcarnitine (C5:1-carn, | | 0.03-0.16 | | | |
| μmol/mmol Cr) | | | | | |
| hexanoylcarnitine (C6- | | 0.02-0.05 | | | |
| carn, μmol/mmol Cr) | | | | | |
| 2-hexenoylcarnitine (C6:1- | | 0.01-0.02 | | | |
| carn, μmol/mmol Cr) | | | | | |
| isovalerylcarnitine (C5- | | <3.0 | | | |
| carn, μmol/mmol Cr) | | | | | |
| tiglylcarnitine (C5:1-carn, | | 0.03-0.16 | | | |
| μmol/mmol Cr) | | | | | |
| methylmalonylcarnitine | | | | | |
| (C4-DC-carn, μmol/mmol | | | | | |
| Cr | | | | | |
| methyl-malonate | <10 | | | | |
| (mmol/mol Cr) | | | | | |
| methylmalonic acid (urine) | 0-14 ug/mg | 0.42-20 | <47.871 | <11 | <6 |
| | Cr | μmol/mmol | μmol/mmol | μmol/mmol | μmol/mmol |
| | | Cr | Cr | Cr | Cr |
| methyl-citrate (urine, | <10 | 0.7-19.6 | 3.3-28.6 | | |
| μmol/mmol Cr) | | | | | |
| 3-hydroxy | 0-4 ug/mg | 3.4-33.6 | 0.1-48.0 | <5 | <3 |
| propionate (urine) | Cr | μmol/mmol | μmol/mmol | μmol/mmol | μmol/mmol |
| | | Cr | Cr | Cr | Cr |
| tiglylglycine (urine) | 0-10 ug/mg | 0-3.8 | 0-0.1 | | |
| | Cr | μmol/mmol | μmol/mmol | | |
| | | Cr | Cr | | |
| propionylglycine (urine) | 0-4 ug/mg | | | | |
| | Cr | | | | |
| 2-ketoisovaleric | | | 0.16 ± 0.48 | | |
| acid/alpha-ketoisovaleric | | | | | |
| acid (μmol/mmol Cr) | | | | | |
| a-ketoglutarate (urine) | 0-35 ug/mg | 0.083-590 | 0-236.9 | | |
| | Cr | μmol/mmol | (<350) | | |
| | | Cr | mol/mmol | | |
| | | | Cr | | |

| | | | 13-18 | |
|---|---|---|---|---|
| | 1-13 years | 1-18 years | years of | Adult (>18 |
| Biomarker | of age | of age | age | years old) |
| lactate | | | | |
| (aka 2-hydroxypropanoic | | | | |
| acid, mmol/mmol Cr) | | | | |
| malate | 9.0-116.959 | | 2.8-30.1 | 0-2.63 |
| (aka malic acid) | μmol/mmol | | μmol/mmol | μmol/mmol |
| | Cr | | Cr | Cr |
| tiglylcarnitine (C5:1-carn, | | | | 0.05-0.20 |
| μmol/mmol Cr) | | | | |
| hexanoylcarnitine (C6- | | | | 0.03-0.07 |
| carn, μmol/mmol Cr) | | | | |
| 2-hexenoylcarnitine (C6:1- | | | 13.38 ± | 0.009-0.050 |
| carn, μmol/mmol Cr) | | | 0.73 | |
| isovalerylcarnitine (C5- | | | | |
| carn, μmol/mmol Cr) | | | | |
| tiglylcarnitine (C5:1-carn, | | | | 0.05-0.20 |
| μmol/mmol Cr) | | | | |
| methylmalonylcarnitine | | | | 0.05-0.16 |
| (C4-DC-carn, μmol/mmol | | | | |
| Cr) | | | | |
| methyl-malonate | | | | |
| (mmol/mol Cr) | | | | |
| methylmalonic acid (urine) | <9.247 | | 0.17-21.6 | <6 |
| | μmol/mmol | | μmol/mmol | μmol/mmol |
| | Cr | | Cr | Cr |
| methyl-citrate (urine, | 1.4-8.5 | <6.09 | 1.6-6.2 | <15 |
| μmol/mmol Cr) | | | | |
| 3-hydroxy | <52.003 | <15.8 | 1.5-26.1 | <30 |
| propionate (urine) | μmol/mmol | μmol/mmol | μmol/mmol | μmol/mmol |
| | Cr | Cr | Cr | Cr |
| tiglylglycine (urine) | 0.1-6.7 | <15.62 | | 0.1-<7 |
| | μmol/mmol | μmol/mmol | | μmol/mmol |
| | Cr | Cr | | Cr |
| propionylglycine (urine) | | 0 | | 0 |
| 2-ketoisovaleric | | | | 0.07-1.2 |
| acid/alpha-ketoisovaleric | | | | |
| acid (μmol/mmol Cr) | | | | |
| a-ketoglutarate (urine) | 1.548 -< 115 | | 10.2-146.0 | 0.658 -< 150 |
| | μmol/mmol | | μmol/mmol | μmol/mmol |
| | Cr | | Cr | Cr |

| Amino | | | | | | Adult |
|---|---|---|---|---|---|---|
| acids | | 13-35 | | | | (>18 |
| (μmol/L, | ≤1 year of | months of | 3-6 years | 7-8 years of | 9-17 years | years |
| urine) | age | age | of age | ages | of age | old) |
| Lysine | 19-1,988 | 25-743 | 14-307 | 17-276 | 10-240 | 15-271 |
| Valine | 11-211 | 11-211 | <139 | 16-91 | <75 | Nov-61 |
| Isoleucine | <86 | <78 | <62 | <34 | <28 | <22 |
| Leucine | <200 | 15-167 | 12-100 | 13-73 | <62 | <51 |
| Glycine | 362-18,614 | 627-6,914 | 412-5,705 | 449-4,492 | 316-4,249 | 229-2,989 |

In some embodiments, a level of α-ketoglutarate in the serum of a subject is between about 0.1-30 μM, such as between about 0.1-20 μM, 0.1-10 μM, 0.1-9 μM, 0.1-8 μM, 0.1-1.0 μM, 0.5-30 μM, 0.5-20 μM, 0.5-15 μM, 0.5-10 μM, 0.5-9 μM, 0.5-8 μM, 1-30 μM, 1-20 μM, 1-15 μM, 1-10 μM, 1-9 μM, 1-8 μM, 3-30 μM, 3-20 μM, 3-15 μM, 3-10 μM, 3-9 μM, 3-8 μM, 5-30 μM, 5-20 μM, 5-15 μM, 5-10 μM, 5-9 μM, 5-8 μM, or any range therein. In some embodiments, a level of α-ketoglutarate in the urine of a subject is between about 0-600 μmol/mmol creatine (Cr), such as between about 0-500 μmol/mmol Cr, 0-400 μmol/mmol Cr, 0-300 μmol/mmol Cr, 0-200 μmol/mmol Cr, 0-150 μmol/mmol Cr, 0-120 μmol/mmol Cr, 0-100 μmol/mmol Cr, 0-80 μmol/mmol Cr, 0-60 μmol/mmol Cr, 0-40 μmol/mmol Cr, 0-20 μmol/mmol Cr, or any range therein. In some embodiments, a level of citrate (e.g., methylcitrate) in the serum of a subject is between about 0-10 μM, such as between about 0-8 μM, 0-6 μM, 0-4 μM, 0-2 μM, 0.01-10 μM, 0.01-8 μM, 0.01-6 μM, 0.01-4 μM, 0.01-2 μM, 0.05-10 μM, 0.05-8 μM, 0.05-6 μM, 0.05-4 μM, 0.05-2 μM, 0.05-1 μM, or any range therein. In some embodiments, a level of citrate (e.g., methylcitrate) in the urine of a subject is between about 0-50 μmol/mmol Cr, such as between about 0-40 μmol/mmol Cr, 0-30 μmol/mmol Cr, 0-20 μmol/mmol Cr, 0-10 μmol/mmol Cr, 3-50 μmol/mmol Cr, 3-40 μmol/mmol Cr, 3-30 μmol/mmol Cr, 3-20 μmol/mmol Cr, 3-10 μmol/mmol Cr, 5-50 μmol/mmol Cr, 5-40 μmol/mmol Cr, 5-30 μmol/mmol Cr, 5-20 μmol/mmol Cr, 5-10 μmol/mmol Cr, or any range therein. In some embodiments, a level of malate in the plasma or serum of a subject is between about 0-20 μM, such as between about 0-18 μM, 0-16 μM, 0-14 μM, 0-12 μM, 0-10 μM, 1-20 μM, 1-18 μM, 1-16 μM, 1-14 μM, 1-12 μM, 1-10 μM, 1-8 μM, 1-6 μM, 1-5 μM, 2-20 μM, 2-18 μM, 2-16 μM, 2-14 μM, 2-12 μM, 2-10 μM, 2-8 μM, 3-20 μM, 3-18 μM, 3-16 μM, 3-14 μM, 3-12 μM, 3-10 μM, 3-8 μM, 3-6 μM, 5-20 μM, 5-10 μM, or any range therein. In some embodiments, a level of malate in the urine of a sample is between about 0-300 μmol/mml Cr, such as between about 0-200 μmol/mml Cr, 0-150 μmol/mml Cr, 0-120 μmol/mml Cr, 0-100 μmol/mml Cr, 0-80 μmol/mml Cr, 0-60 μmol/mml Cr, 0-40 μmol/mml Cr, 0-20 μmol/mml Cr, 0-15 μmol/mml Cr, 0-10 μmol/mml Cr, 0-5 μmol/mml Cr, 0-3 μmol/mml Cr, 0-2 μmol/mml Cr, 5-300 μmol/mml Cr, 5-200 μmol/mml Cr, 5-100 mol/mml Cr, 5-80 mol/mml Cr, 5-60 mol/mml Cr, 5-40 μmol/mml Cr, 5-20 μmol/mml Cr, or any range therein. In some embodiments, a level of methylmalonate in the serum or plasma of a subject is between about 0-20 μM, such as between about 0-18 μM, 0-16 μM, 0-14 μM, 0-12 μM, 0-10 μM, 0-8 μM, 0-6 μM, 0-4 μM, 0-2 μM, 0-1 μM, 0-0.9 μM, 0-0.8 μM, 0-0.7 μM, 0- 0.6 μM, 0-0.5 μM, 0-0.4 μM, 0-0.3 μM, 0-0.2 μM, 0-0.1 μM, 0-0.05 μM, or any range therein. In some embodiments, a level of methylmalonate in the urine of a subject is between about 0-100 μmol/mmol Cr, such as between about 0-80 μmol/mmol Cr, 0-60 μmol/mmol Cr, 0-50 μmol/mmol Cr, 0-40 μmol/mmol Cr, 0-30 μmol/mmol Cr, 0-20 μmol/mmol Cr, 0-10 μmol/mmol Cr, 0-8 μmol/mmol Cr, 0-6 μmol/mmol Cr, 0-4 μmol/mmol Cr, 0-2 μmol/mmol Cr, 2-50 μmol/mmol Cr, 2-40 μmol/mmol Cr, 2-30 μmol/mmol Cr, 2-20 μmol/mmol Cr, 2-10 μmol/mmol Cr, 5-50 μmol/mmol Cr, 5-40 μmol/mmol Cr, 5-30 μmol/mmol Cr, 5-20 μmol/mmol Cr, 5-10 μmol/mmol Cr, or any range therein. In some embodiments, a level of lactate in the plasma of a subject is between about 0-20 mmol/L, such as between about 0-15 mmol/L, 0-10 mmol/L, 0-8 mmol/L, 0-6 mmol/L, 0-5 mmol/L, 0-4 mmol/L, 0-3 mmol/L, 0-2 mmol/L, 0.5-10 mmol/L, 0.5-8 mmol/L, 0.5-6 mmol/L, 0.5-4 mmol/L, 0.5-3 mmol/L, 0.5-2 mmol/L, 0.5-1 mmol/L, 1-10 mmol/L, 1-8 mmol/L, 1-6 mmol/L, 1-4 mmol/L, 1-3 mmol/L, 1-2 mmol/L, or any range therein. In some embodiments, a level of lactate in the urine of the subject is between about 0-10 mmol/mmol Cr, such as between about 0-8 mmol/mmol Cr, 0-6 mmol/mmol Cr, 0-5 mmol/mmol Cr, 0-4 mmol/mmol Cr, 0-3 mmol/mmol Cr, 0-2 mmol/mmol Cr, 0-1 mmol/mmol Cr, 0-0.5 mmol/mmol Cr, 0-0.4 mmol/mmol Cr, 0-0.3 mmol/mmol Cr, 0-0.2 mmol/mmol Cr, 0-0.1 mmol/mmol Cr, 0-0.08 mmol/mmol Cr, 0-0.07 mmol/mmol Cr, 0-0.06 mmol/mmol Cr, 0-0.05 mmol/mmol Cr, or any range therein.

In some embodiments of any of the above aspects, treatment of a subject having a CASTOR disorder, PKAN, or a metabolic disorder with a compound provided herein, or a pharmaceutically acceptable form thereof, normalizes (e.g., brings into a range normal for a subject not suffering from a CASTOR disorder, PKAN, or a metabolic disorder) levels of one or more TCA cycle metabolites (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine). In some embodiments, normalization of a level of a TCA cycle metabolite is measured using blood, urine, and/or plasma.

In some embodiments of any of the above aspects, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is decreased by at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, or more upon treatment with a compound provided herein, or a pharmaceutically acceptable form thereof. In some embodiments of any of the above aspects, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is decreased by between about 1-5%, 5-10%, 10-15%, 10-20%, 20-25%, 20-30%, 30-35%, 30-40%, 40-45%, 40-50%, 50-55%, 50-60%, 60-65%, 60-70%, 70-75%, 70-80%, 80-85%, 80-90%, 90-95%, 90-100%, 100-125%, 125-150%, 150-175%, 175-200%, 200-225%, 225-250%, 250-275%, 275-300%, or any useful range therein upon treatment with a compound provided herein, or a pharmaceutically acceptable form thereof. In some embodiments, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is decreased by less than one fold, one fold, two fold, three fold, four fold, five fold, or more upon treatment with a compound provided herein, or a pharmaceutically acceptable form thereof. In some embodiments, the ratio of a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder at a second time to the level of the TCA cycle metabolite in the subject at a first time prior to the second time is about 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.025, 0.01, or less, where the first time is prior to treatment of the subject with a compound provided herein, or a pharmaceutically acceptable salt thereof, and the second time is after treatment of the subject with the compound, or the pharmaceutically acceptable salt thereof. In some embodiments, the TCA cycle metabolite is selected from α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the TCA cycle metabolite is selected from α-ketoglutarate, citrate, isocitrate, malate, methylcitrate, methylmalonate, oxaloacetate, succinate, glucose, glycerate, phosphoenolpyruvate (PEP), and choline. In some embodiments, the TCA cycle metabolite is selected from α-ketoglutarate, malate, methylcitrate, methylmalonate, oxaloacetate, and succinate. In some embodiments, the level of the TCA cycle metabolite is measured using blood, urine, and/or plasma. In some embodiments, the change in the level of the TCA cycle metabolite is assessed by measuring the level of the TCA cycle metabolite at a first time prior to treatment with the compound, or the pharmaceutically acceptable form thereof, and at a second time during or after treatment with the compound, or the pharmaceutically acceptable form thereof. In some embodiments, the second time is 1 day, 2 days, 3 days, 4 days, 5 days, 6 days, 1 week, 2 weeks, 3 weeks, 4 weeks, 6 weeks, 8 weeks, 1 month, 2 months, 3 months, 4 months, 5 months, 6 months, 7 months, 8 months, 9 months, 10 months, 11 months, 1 year, or longer after the first dosing of the subject with the compound, or the pharmaceutically acceptable form thereof. In some embodiments, the level of the TCA cycle metabolite is assessed more than one time following the first dosing of the subject with the compound, or the pharmaceutically acceptable form thereof. In some embodiments, the level of the TCA cycle metabolite is assessed at regular intervals over a period of time, such as about every week, every two weeks, every three weeks, every four weeks, every month, every two months, every three months, every six months, or every year.

In some embodiments of any of the above aspects, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is increased by at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, or more upon treatment with a compound provided herein, or a pharmaceutically acceptable form thereof. In some embodiments of any of the above aspects, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is increased by between about 1-5%, 5-10%, 10-15%, 10-20%, 20-25%, 20-30%, 30-35%, 30-40%, 40-45%, 40-50%, 50-55%, 50-60%, 60-65%, 60-70%, 70-75%, 70-80%, 80-85%, 80-90%, 90-95%, 90-100%, 100-125%, 125-150%, 150-175%, 175-200%, 200-225%, 225-250%, 250-275%, 275-300%, or any useful range therein upon treatment with a compound provided herein, or a pharmaceutically acceptable form thereof. In some embodiments, a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder is increased by less than one fold, one fold, two fold, three fold, four fold, five fold, or more upon treatment with a compound provided herein, or a pharmaceutically acceptable form thereof. In some embodiments, the ratio of a level of a TCA cycle metabolite (e.g., α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, or creatine) in a subject having a CASTOR disorder, PKAN, or a metabolic disorder at a second time to the level of the TCA cycle metabolite in the subject at a first time prior to the second time is about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8, 5.0, or more, where the first time is prior to treatment of the subject with a compound provided herein, or a pharmaceutically acceptable salt thereof, and the second time is after treatment of the subject with the compound, or the pharmaceutically acceptable salt thereof. In some embodiments, the TCA cycle metabolite is selected from α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the TCA cycle metabolite is selected from α-ketoglutarate, citrate, isocitrate, malate, methylcitrate, methylmalonate, oxaloacetate, succinate, glucose, glycerate, phosphoenolpyruvate (PEP), and choline. In some embodiments, the TCA cycle metabolite is selected from α-ketoglutarate, malate, methylcitrate, methylmalonate, oxaloacetate, and succinate. In some embodiments, the level of the TCA cycle metabolite is measured using blood, urine, and/or plasma. In some embodiments, the change in the level of the TCA cycle metabolite is assessed by measuring the level of the TCA cycle metabolite at a first time prior to treatment with the compound, or the pharmaceutically acceptable form thereof, and at a second time during or after treatment with the compound, or the pharmaceutically acceptable form thereof. In some embodiments, the second time is 1 day, 2 days, 3 days, 4 days, 5 days, 6 days, 1 week, 2 weeks, 3 weeks, 4 weeks, 6 weeks, 8 weeks, 1 month, 2 months, 3 months, 4 months, 5 months, 6 months, 7 months, 8 months, 9 months, 10 months, 11 months, 1 year, or longer after the first dosing of the subject with the compound, or the pharmaceutically acceptable form thereof. In some embodiments, the level of the TCA cycle metabolite is assessed more than one time following the first dosing of the subject with the compound, or the pharmaceutically acceptable form thereof. In some embodiments, the level of the TCA cycle metabolite is assessed at regular intervals over a period of time, such as about every week, every two weeks, every three weeks, every four weeks, every month, every two months, every three months, every six months, or every year.

In some embodiments of any of the above aspects, a subject has an elevated C3-carnitine level, a depressed C2-carnitine level, a depressed carnitine level, and/or an elevated C3:C2-carnitine ratio in plasma and/or in liver prior to treatment of the subject with a compound provided herein, or a pharmaceutically acceptable salt thereof. In some embodiments, one or more of a C3-carnitine level, a C2-carnitine level, a carnitine level, or a C3:C2-carnitine ratio in plasma and/or in the liver is normalized upon treatment of the subject with a compound provided herein, or a pharmaceutically acceptable salt thereof.

In some embodiments of any of the above aspects, a subject has an elevated C3:C2-Coenzyme A (CoA) level in the liver and/or an elevated C3-CoA level in the liver and/or heart prior to treatment of the subject with a compound provided herein, or a pharmaceutically acceptable salt thereof. In some embodiments, one or more of a C3:C2-Coenzyme A (CoA) level in the liver and/or a C3-CoA level in the liver and/or heart is normalized upon treatment of the subject with a compound provided herein, or a pharmaceutically acceptable salt thereof.

In some embodiments of any of the above aspects, a level of a substance (e.g., glutamate/glutamine (Glx), GABA, inositol, choline, taurine, or N-acetyl aspartate) in the brain of a subject having a CASTOR disorder, PKAN, or a metabolic disorder is normalized upon treatment with a compound provided herein, or a pharmaceutically acceptable form thereof. In some embodiments of any of the above aspects, a level of a substance (e.g., glutamate/glutamine (Glx), GABA, inositol, choline, taurine, or N-acetyl aspartate) in the brain of a subject having a CASTOR disorder, PKAN, or a metabolic disorder is increased by at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, or more upon treatment with a compound provided herein, or a pharmaceutically acceptable form thereof. In some embodiments of any of the above aspects, a level of a substance (e.g., glutamate/glutamine (Glx), GABA, inositol, choline, taurine, or N-acetyl aspartate) in the brain of a subject having a CASTOR disorder, PKAN, or a metabolic disorder is increased by between about 1-5%, 5-10%, 10-15%, 10-20%, 20-25%, 20-30%, 30-35%, 30-40%, 40-45%, 40-50%, 50-55%, 50-60%, 60-65%, 60-70%, 70-75%, 70-80%, 80-85%, 80-90%, 90-95%, 90-100%, 100-125%, 125-150%, 150-175%, 175-200%, 200-225%, 225-250%, 250-275%, 275-300%, or any useful range therein upon treatment with a compound provided herein, or a pharmaceutically acceptable form thereof. In some embodiments, a level of a substance (e.g., glutamate/glutamine (Glx), GABA, inositol, choline, taurine, or N-acetyl aspartate) in the brain of a subject having a CASTOR disorder, PKAN, or a metabolic disorder is increased by less than one fold, one fold, two fold, three fold, four fold, five fold, or more upon treatment with a compound provided herein, or a pharmaceutically acceptable form thereof. In some embodiments, the ratio of a level of a substance (e.g., glutamate/glutamine (Glx), GABA, inositol, choline, taurine, or N-acetyl aspartate) in the brain of a subject having a CASTOR disorder, PKAN, or a metabolic disorder at a second time to the level of the substance in the subject at a first time prior to the second time is about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.2, 3.4, 3.6, 3.8, 4.0, 4.2, 4.4, 4.6, 4.8, 5.0, or more, where the first time is prior to treatment of the subject with a compound provided herein, or a pharmaceutically acceptable salt thereof, and the second time is after treatment of the subject with the compound, or the pharmaceutically acceptable salt thereof. In some embodiments, the substance is glutamate/glutamine (Glx). In some embodiments, the substance is GABA. In some embodiments, the substance is N-acetyl aspartate. In some embodiments, the substance is inositol. In some embodiments, the substance is choline. In some embodiments, the substance is taurine. In some embodiments, the change in the level of the substance is assessed by measuring the level of the substance at a first time prior to treatment with the compound, or the pharmaceutically acceptable form thereof, and at a second time during or after treatment with the compound, or the pharmaceutically acceptable form thereof. In some embodiments, the second time is 1 day, 2 days, 3 days, 4 days, 5 days, 6 days, 1 week, 2 weeks, 3 weeks, 4 weeks, 6 weeks, 8 weeks, 1 month, 2 months, 3 months, 4 months, 5 months, 6 months, 7 months, 8 months, 9 months, 10 months, 11 months, 1 year, or longer after the first dosing of the subject with the compound, or the pharmaceutically acceptable form thereof. In some embodiments, the level of the substance is assessed more than one time following the first dosing of the subject with the compound, or the pharmaceutically acceptable form thereof. In some embodiments, the level of the substance is assessed at regular intervals over a period of time, such as about every week, every two weeks, every three weeks, every four weeks, every month, every two months, every three months, every six months, or every year.

In some embodiments, magnetic resonance analysis of one or more metabolites (e.g., as described herein) is performed in combination with analysis of one or more biomarkers (e.g., as described herein).

### III. METHODS OF TREATING A CASTOR DISEASE IN A SUBJECT

In various aspects, the compounds and compositions disclosed herein are useful for treating, preventing, ameliorating, controlling or reducing the risk of a variety of disorders associated with pantothenate kinase activity, including, for example, PKAN, aging and diabetes. Thus, in one aspect, disclosed are methods of treating a disorder associated with pantothenate kinase activity in a subject, the method comprising administering to the subject an effective amount of at least one disclosed compound or a pharmaceutically acceptable salt thereof.

The present disclosure provides methods comprising treating a coenzyme A reduction, elevation, sequestration, toxicity, or redistribution (CASTOR) disease such as propionic acidemia, a neurological disease (e.g., PKAN), or a metabolic disorder in a subject, the method comprising the step of administering to the subject a therapeutically effective amount of at least one disclosed compound or a pharmaceutically acceptable salt thereof. Examples of CASTOR diseases include, but are not limited to, defects in fatty acid oxidation enzymes, methylmalonic acidemia, glutaric acidemia, propionic academia, and HMG-CoA lyase deficiency.

In some embodiments, the method provided herein involve treating a coenzyme A reduction, elevation, sequestration, toxicity, or redistribution (CASTOR) disease, a neurological disease (e.g., PKAN), or a metabolic disorder in a subject, the method comprising the step of administering to the subject a therapeutically effective amount of a compound provided herein, or a form thereof (e.g., a pharmaceutically acceptable salt thereof).

The disclosed small molecule modulators of pantothenate kinases counteract the feedback inhibition of the PANK enzyme(s) by cellular acyl-coenzyme A molecules (acyl-CoAs), thereby releasing the PANK catalytic capacity to initiate CoA biosynthesis. Treatment with the disclosed small molecules resulted in elevated levels of CoA that can accommodate the acyl-CoA accumulation under CASTOR conditions while maintaining a rate of energy production and lipid metabolism that is sufficient to ameliorate the pathology, morbidity, and mortality that are associated with acyl-CoA imbalances.

In various aspects, the CASTOR disease may be associated with inhibition of one or more pantothenate kinases by one or more acyl Coenzyme A (acyl-CoA) species. In a further aspect, the CASTOR disease is associated with accumulation of one or more acyl Coenzyme A (acyl-CoA) species in a subject having a CASTOR disease in an amount greater than that of a subject not having a CASTOR disease. In a still further aspect, the CASTOR disease is associated with a decrease of free CoA and/or acetyl-CoA in a subject having a CASTOR disease in an amount lower than that of a subject not having the CASTOR disease. In yet a further aspect, the CASTOR disease is associated with impaired or inhibited degradation of the one or more acyl-CoA species in the subject having a CASTOR disease. In an even further aspect, the one or more acyl-CoA species are not acetyl Coenzyme A (acetyl-CoA).

In various aspects, the CASTOR disease is associated with accumulation of one or more fatty acids in a subject having a CASTOR disease in an amount greater than that of a subject not having the CASTOR disease. In a further aspect, the CASTOR disease is associated with impaired or inhibited degradation of the one or more fatty acids in the subject having a CASTOR disease.

In various aspects, the CASTOR disease is selected from medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-Co A: amino acid N-acyltransferase deficiency, bile acid-CoA ligase deficiency, holocarboxylase synthetase deficiency, succinate dehydrogenase deficiency, α-ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, cytosolic acetoacetyl-CoA thiolase deficiency, cytosolic HMG-CoA synthase deficiency, lecithin cholesterol acyltransferase deficiency, choline acetyl transferase deficiency, congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

In various aspects, the CASTOR disease is selected from medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, and PLA2G6-associated neurodegeneration.

In various aspects, the CASTOR disease is selected from glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA: amino acid N-acyltransferase deficiency, bile acid-CoA ligase deficiency, holocarboxylase synthetase deficiency, succinate dehydrogenase deficiency, α-ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, systemic primary carnitine deficiency, carnitine: acylcamitine translocase deficiency I and II, acetyl-CoA carboxylase deficiency, malonyl-CoA decarboxylase deficiency, mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency.), acyl-CoA oxidase deficiency, alpha-methylacyl-CoA racemase (AMACR) deficiency, sterol carrier protein x deficiency, 2,4-dienoyl-CoA reductase deficiency, cytosolic acetoacetyl-CoA thiolase deficiency, cytosolic HMG-CoA synthase deficiency, lecithin cholesterol, acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy.

In various aspects, the CASTOR disease is selected from medium chain acyl-CoA dehydrogenase deficiency, short chain acyl-CoA dehydrogenase deficiency, very long chain acyl-CoA dehydrogenase deficiency, and D-bifunctional protein deficiency. In a further aspect, the CASTOR disease is medium chain acyl-CoA dehydrogenase deficiency. In a still further aspect, the CASTOR disease is short chain acyl-CoA dehydrogenase deficiency. In yet a further aspect, the CASTOR disease is very long chain acyl-CoA dehydrogenase deficiency. For yet another example, the CASTOR disease is D-bifunctional protein deficiency.

In various aspects, the CASTOR disease is selected from glutaric acidemia type 1, methylmalonic academia, propionyl-CoA carboxylase deficiency, propionic academia, 3-methylcrotonyl carboxylase deficiency, and isovaleryl-CoA dehydrogenase deficiency. In a further aspect, the CASTOR disease is Glutaric acidemia type 1. In a still further aspect, the CASTOR disease is methylmalonic academia. In yet a further aspect, the CASTOR disease is propionyl-CoA carboxylase deficiency. In an even further aspect, the CASTOR disease is propionic academia. In a still further aspect, the CASTOR disease is 3-methylcrotonyl carboxylase deficiency. In yet a further aspect, the CASTOR disease is isovaleryl-CoA dehydrogenase deficiency.

In various aspects, the disclosed compounds can be used in combination with one or more other drugs in the treatment, prevention, control, amelioration, or reduction of risk of CASTOR diseases or other disorders associated with PanK activity for which disclosed compounds or the other drugs can have utility, where the combination of the drugs together are safer or more effective than either drug alone. In some embodiments, the compound and another agent administered in combination with the agent have a synergistic effect. Such other drug(s) can be administered, by a route and in an amount commonly used therefor, contemporaneously, concomitantly, or sequentially with a compound of the present disclosure. In some embodiments, the compound and another agent administered in combination with the agent are administered concomitantly. In some embodiments, the compound and another agent administered in combination with the agent are administered sequentially. In some embodiments, the compound and another agent administered in combination with the agent are administered separately. When a compound of the present disclosure is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present disclosure.

In a further aspect, the compound exhibits inhibition of PanK activity. In a still further aspect, the compound exhibits a decrease in PanK activity.

In a further aspect, the compound exhibits inhibition of PanK activity with an IC₅₀ of from about 0.001 μM to about 25 μM. In a still further aspect, the compound exhibits inhibition of PanK activity with an IC₅₀ of from about 0.001 μM to about 15 μM. In yet a further aspect, the compound exhibits inhibition of PanK activity with an IC₅₀ of from about 0.001 μM to about 10 μM. In an even further aspect, the compound exhibits inhibition of PanK activity with an IC₅₀ of from about 0.001 μM to about 5 μM. In a still further aspect, the compound exhibits inhibition of PanK activity with an IC₅₀ of from about 0.001 μM to about 1 μM. In yet a further aspect, the compound exhibits inhibition of PanK activity with an IC₅₀ of from about 0.001 μM to about 0.5 μM. In an even further aspect, the compound exhibits inhibition of PanK activity with an IC₅₀ of from about 0.001 μM to about 0.1 μM. In a still further aspect, the compound exhibits inhibition of PanK activity with an IC₅₀ of from about 0.001 μM to about 0.05 μM. In yet a further aspect, the compound exhibits inhibition of PanK activity with an IC₅₀ of from about 0.001 μM to about 0.01 μM. In an even further aspect, the compound exhibits inhibition of PanK activity with an IC₅₀ of from about 0.001 μM to about 0.005 μM. In a still further aspect, the compound exhibits inhibition of PanK activity with an IC₅₀ of from about 0.005 μM to about 25 μM. In yet a further aspect, the compound exhibits inhibition of PanK activity with an IC₅₀ of from about 0.01 μM to about 25 μM. In an even further aspect, the compound exhibits inhibition of PanK activity with an IC₅₀ of from about 0.05 μM to about 25 μM. In a still further aspect, the compound exhibits inhibition of PanK activity with an IC₅₀ of from about 0.1 μM to about 25 μM. In yet a further aspect, the compound exhibits inhibition of PanK activity with an IC₅₀ of from about 0.5 μM to about 25 μM. In an even further aspect, the compound exhibits inhibition of PanK activity with an IC₅₀ of from about 1 μM to about 25 μM. In a still further aspect, the compound exhibits inhibition of PanK activity with an IC₅₀ of from about 5 μM to about 25 μM. In yet a further aspect, the compound exhibits inhibition of PanK activity with an IC₅₀ of from about 10 μM to about 25 μM. In an even further aspect, the compound exhibits inhibition of PanK activity with an IC₅₀ of from about 15 μM to about 25 μM.

In a further aspect, the subject is a mammal. In a still further aspect, the mammal is human.

In a further aspect, the subject has been diagnosed with a need for treatment of the disorder (e.g., CASTOR disorder, neurological disease such as PKAN, or metabolic disorder) prior to the administering step. In a still further aspect, the subject is at risk for developing the disorder (e.g., CASTOR disorder, neurological disease such as PKAN, or metabolic disorder) prior to the administering step.

In a further aspect, the method further comprises identifying a subject at risk for developing the disorder (e.g., CASTOR disorder, neurological disease such as PKAN, or metabolic disorder) prior to the administering step.

In a further aspect, the method further comprises the step of administering to the subject a therapeutically effective amount of carnitine, pantothenate, and/or pantothenic acid. In a still further aspect, the method further comprises the step of administering to the subject a therapeutically effective amount of carnitine, pantothenate, and pantothenic acid. In yet a further aspect, the method further comprises the step of administering to the subject a therapeutically effective amount of carnitine, pantothenate, or pantothenic acid. In an even further aspect, the method further comprises the step of the step of administering to the subject a therapeutically effective amount of carnitine. In a still further aspect, the method further comprises the step of administering to the subject a therapeutically effective amount of pantothenate. In yet a further aspect, the method further comprises the step of administering to the subject a therapeutically effective amount of pantothenic acid.

In a further aspect, the CASTOR disease is hereditary. In a still further aspect, the CASTOR disease is acquired.

### IV. COMPOUNDS

The method provided herein may comprise the use of compounds useful in treating or preventing a CASTOR disease such as, for example, defects in fatty acid oxidation enzymes, methylmalonic acidemia, glutaric acidemia, propionic academia, and HMG-CoA lyase deficiency. The compounds may be useful in the treatment or prevention of CASTOR diseases in which PanKs or altered levels of CoA and CoA esters are involved, as further described herein.

It is contemplated that each disclosed derivative can be optionally further substituted. It is also contemplated that any one or more derivative can be optionally omitted from the present disclosure. It is understood that a disclosed compound can be provided by the disclosed methods.

It is also understood that the disclosed compounds can be employed in the disclosed methods of using.

### 1. Structure

In some embodiments, a compound (e.g., a compound useful in treating or preventing a CASTOR disease or symptoms thereof) has a structure represented by a formula:

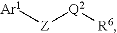

wherein Z is selected from A(C═O), COCH₂,

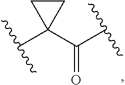 [SMILES: *C(=O)C1(*)CC1]

CO, NHCO, NHCS, CH₂SO₂, and SO₂; wherein A is selected from O, CO, CH₂, CF₂, NH, N(CH₃), and CH(OH); wherein Q² is a structure selected from:

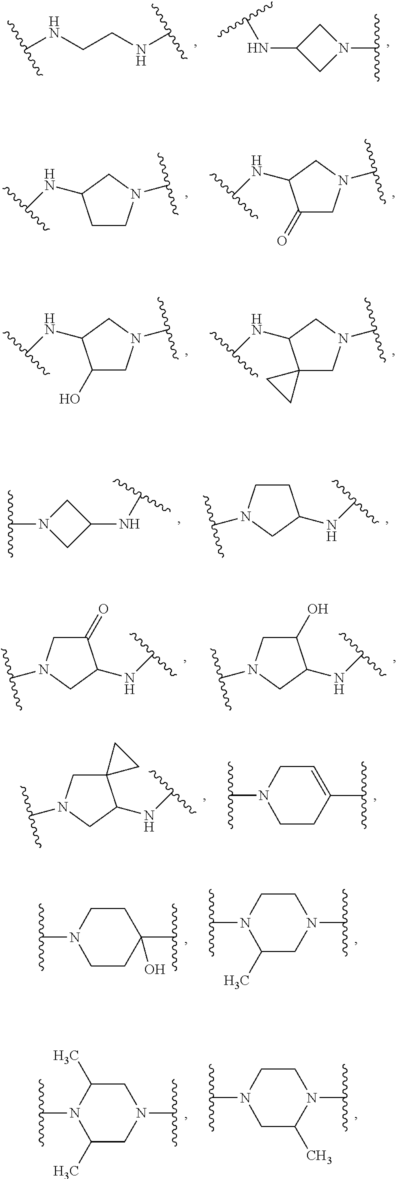

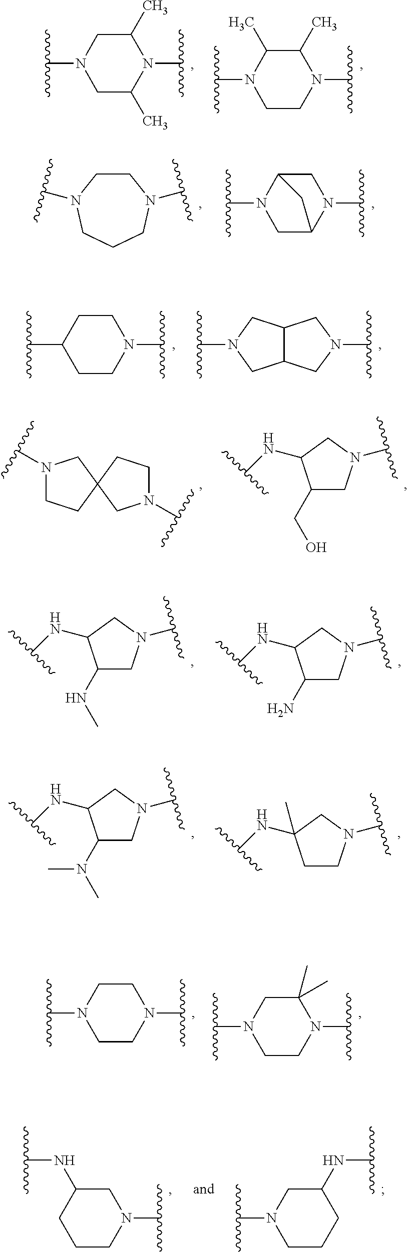

wherein Ar¹ is selected from aryl and heteroaryl and substituted with 0, 1, 2, or 3 groups independently selected from halogen, —NO₂, —CN, —OH, —SH, —NH₂, C1-C8 thioalkyl, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), C1-C8 alkoxyhaloalkyl, and cyclopropyl, cyclobutyl, and oxetane, wherein the cyclopropyl, cyclobutyl, and oxetane are optionally substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl); wherein R⁶ is selected from —NHCH₂C₆H₅ and Ar²; wherein Ar² is a structure represented by a formula selected from:

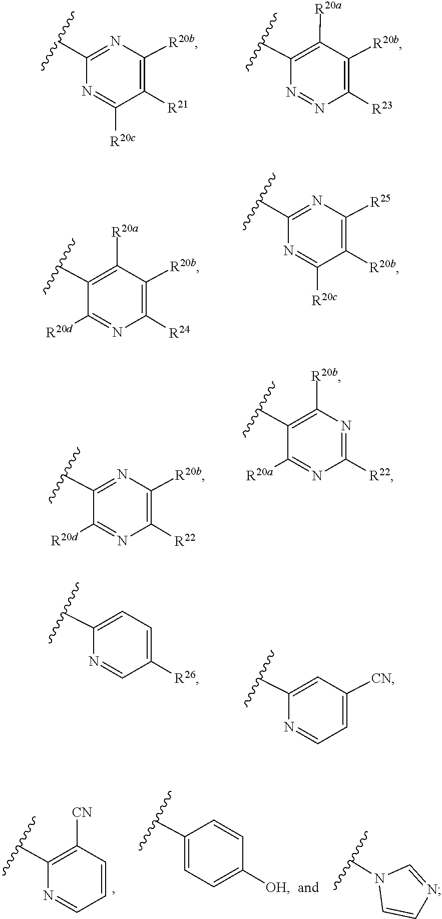

wherein each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; wherein R²¹, when present, is selected from hydrogen, halogen, —CN, —NO₂, —SO₂NH₂, —SO₂CH₃, —SO₂CF₃, and Cy¹; wherein Cy¹, when present, is selected from cycle, heterocycle, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino; wherein R²², when present, is selected from —CN, halogen, —NO₂, SO₂NH₂, SO₂CH₃, and SO₂CF₃; wherein R²³, when present, is selected from hydrogen, halogen, —CN, —NO₂, —SO₂NH₂, —SO₂CH₃, —SO₂CF₃, cyclohexyl,

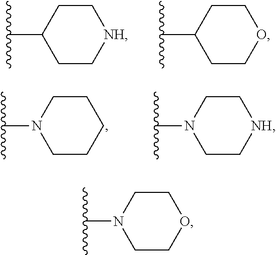

and Cy¹; wherein R²⁴, when present, is selected from —CN, halogen, —NO₂, SO₂NH₂, SO₂CH₃, and SO₂CF₃; wherein R²⁵, when present, is selected from —CN, —NO₂, SO₂NH₂, SO₂CH₃, and SO₂CF₃; wherein R²⁶, when present, is selected from —Br, —Cl, —F, —CN, —NO₂, —CF₃, and methyl; or a pharmaceutically acceptable salt thereof.

In some embodiments, a compound (e.g., a compound useful in treating or preventing a CASTOR disease or symptoms thereof) has a structure represented by a formula:

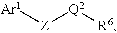

wherein:
Z is A(C═O);
A is CH₂;
Q² is a structure selected from:

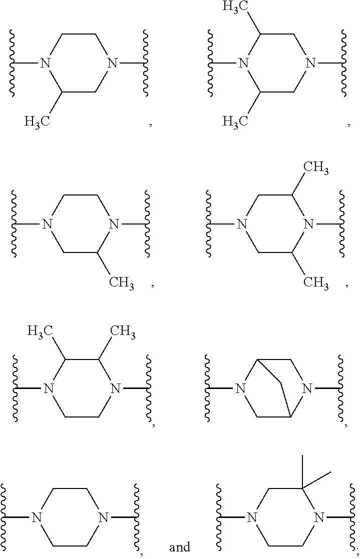

Ar¹ is selected from aryl and heteroaryl and substituted with 0, 1, 2, or 3 groups independently selected from halogen, —NO₂, —CN, —OH, —SH, —NH₂, C1-C8 thioalkyl, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), C1-C8 alkoxyhaloalkyl, and cyclopropyl, cyclobutyl, and oxetane, wherein the cyclopropyl, cyclobutyl, and oxetane are optionally substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl);
R⁶ is Ar²;
Ar² is a structure represented by a formula selected from:

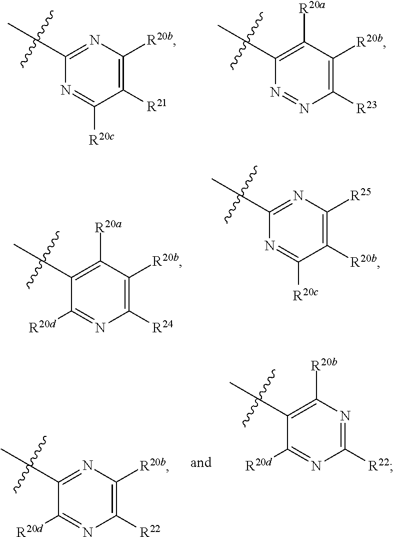

each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl;
R²¹, when present, is selected from hydrogen, halogen, —CN, —NO₂, —SO₂NH₂, —SO₂CH₃, and —SO₂CF₃;
R²², when present, is selected from —CN, halogen, —NO₂, SO₂NH₂, SO₂CH₃, and SO₂CF₃;
R²³, when present, is selected from hydrogen, halogen, —CN, —NO₂, —SO₂NH₂, —SO₂CH₃, —SO₂CF₃, cyclohexyl, and

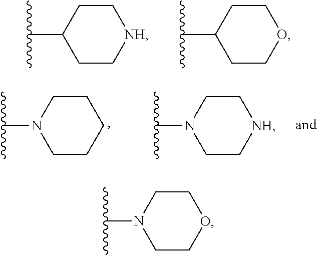

R²⁴, when present, is selected from —CN, halogen, —NO₂, SO₂NH₂, SO₂CH₃, and SO₂CF₃; and
R²⁵, when present, is selected from —CN, —NO₂, SO₂NH₂, SO₂CH₃, and SO₂CF₃.

In a further aspect, disclosed are compounds having a structure represented by a formula:

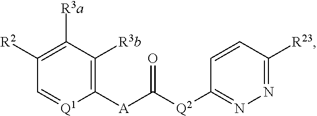

wherein A is selected from O, CO, CH₂, CF₂, NH, N(CH₃), and CH(OH); wherein Q¹ is CH; and wherein R² is selected from —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; or wherein Q¹ is N; and R² is selected from halogen, —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; wherein Q² is a structure selected from:

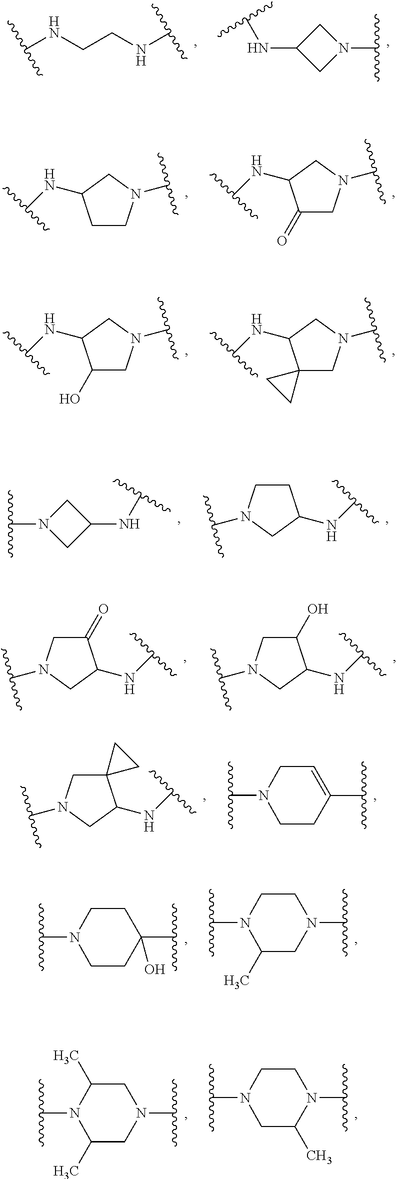

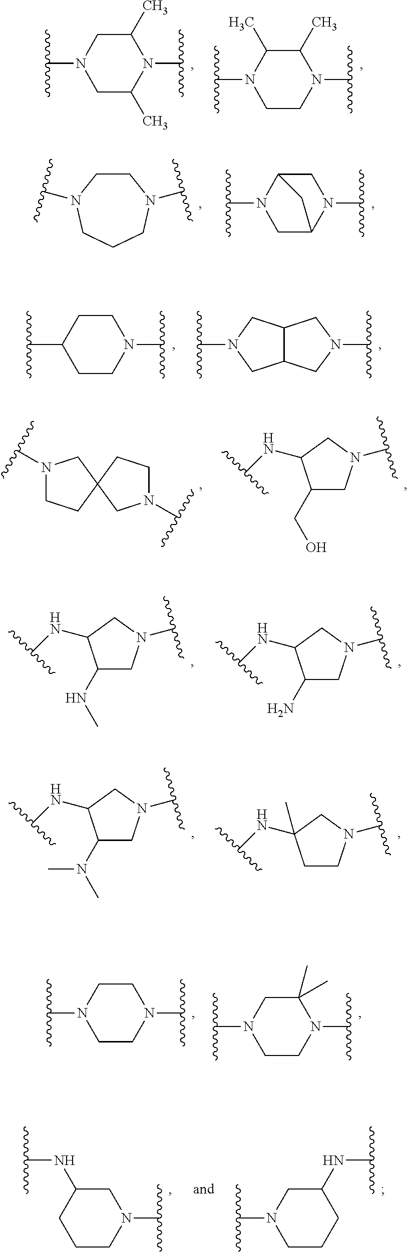

wherein each of R^{3a} and R^{3b} is independently selected from hydrogen, halogen, —OH, C1-C4 alkoxy, and C1-C4 alkyl; and wherein R²³ is selected form hydrogen, halogen, —CN, SO₂NH₂, SO₂CH₃, SO₂CF₃, and NO₂, or a pharmaceutically acceptable salt thereof.

In a still further aspect, the compound has a structure represented by a formula:

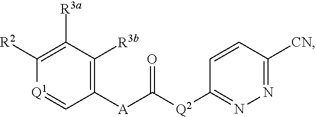

or a pharmaceutically acceptable salt thereof.

In yet a further aspect, the compound has a structure represented by a formula:

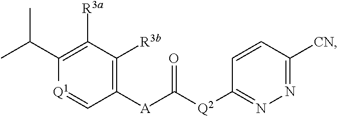

or a pharmaceutically acceptable salt thereof.

In an even further aspect, the compound is selected from:

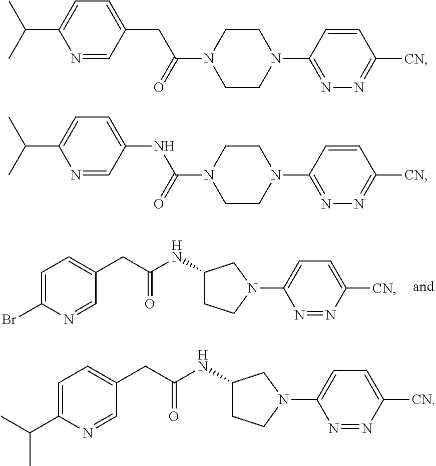 [SMILES: CC(C)c1ccc(CC(=O)N2CCN(c3ccc(C#N)nn3)CC2)cn1 ; CC(C)c1ccc(NC(=O)N2CCN(c3ccc(C#N)nn3)CC2)cn1 ; N#Cc1ccc(N2CC[C@H](NC(=O)Cc3ccc(Br)nc3)C2)nn1 ; CC(C)c1ccc(CC(=O)N[C@H]2CCN(c3ccc(C#N)nn3)C2)cn1]

or a pharmaceutically acceptable salt thereof.

In a still further aspect, the compound is selected from:

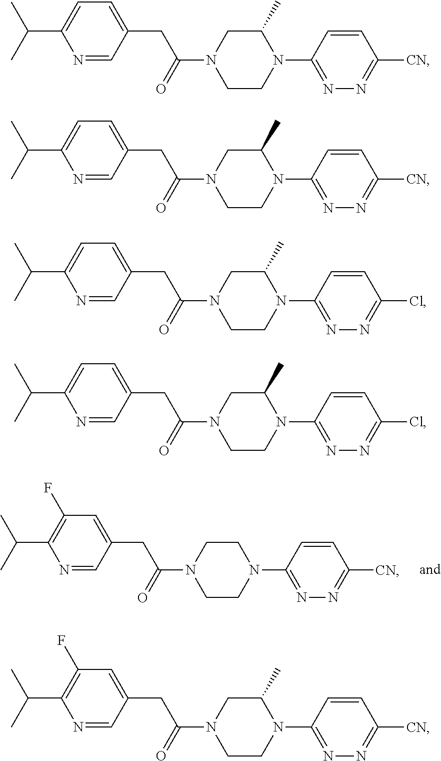

In yet a further aspect, the compound is:

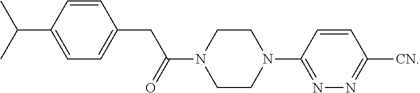 [SMILES: CC(C)c1ccc(CC(=O)N2CCN(c3ccc(C#N)nn3)CC2)cc1]

In yet a further aspect, the compound is:

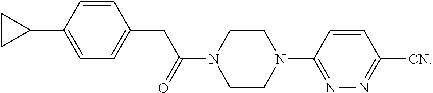 [SMILES: N#Cc1ccc(N2CCN(C(=O)Cc3ccc(C4CC4)cc3)CC2)nn1]

In a further aspect, a compound can be present as one or more of the following structures:

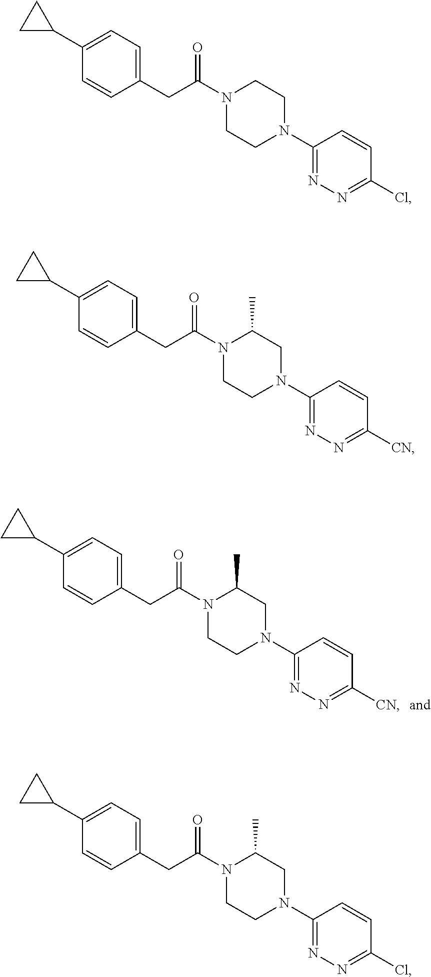 [SMILES: O=C(Cc1ccc(C2CC2)cc1)N1CCN(c2ccc(Cl)nn2)CC1 ; C[C@@H]1CN(c2ccc(C#N)nn2)CCN1C(=O)Cc1ccc(C2CC2)cc1 ; C[C@H]1CN(c2ccc(C#N)nn2)CCN1C(=O)Cc1ccc(C2CC2)cc1 ; C[C@@H]1CN(c2ccc(Cl)nn2)CCN1C(=O)Cc1ccc(C2CC2)cc1]

or a pharmaceutically acceptable salt thereof.

In a further aspect, the compound has a structure a structure represented by a formula:

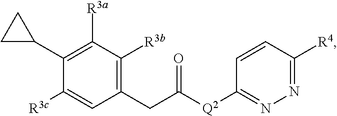

wherein Q² is a structure selected from:

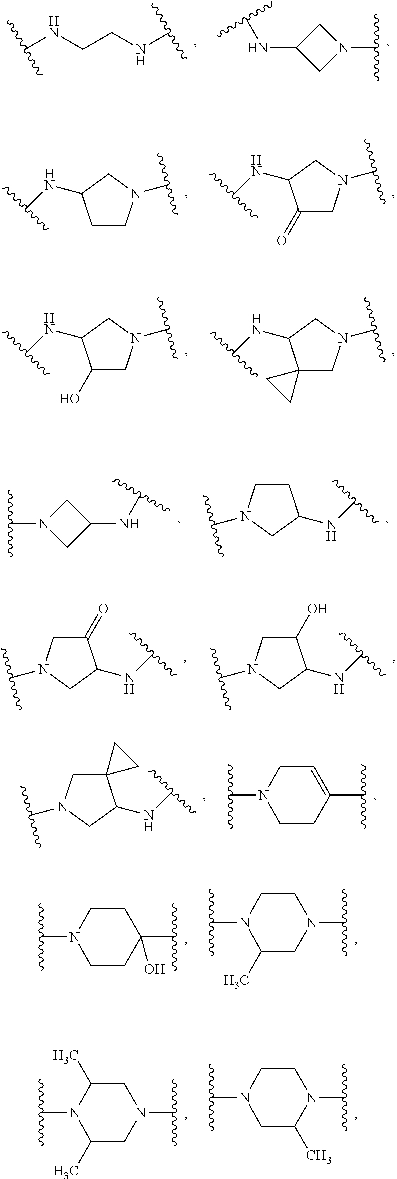

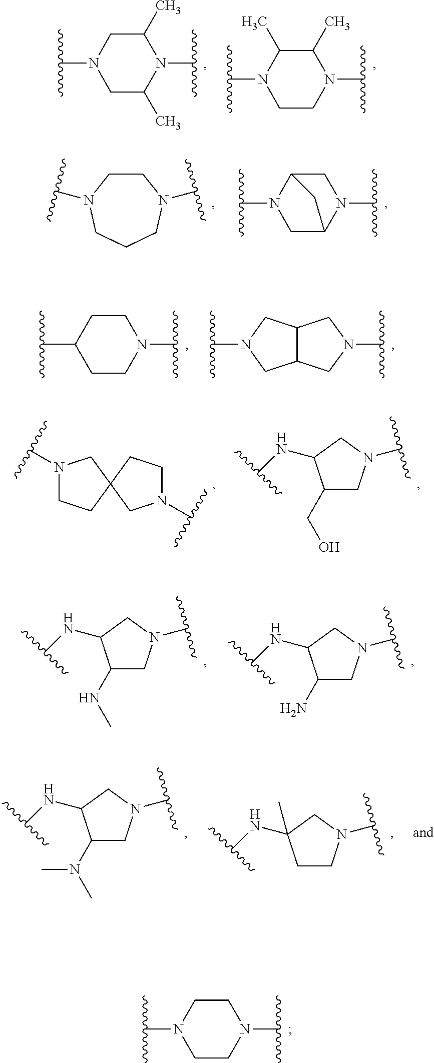

wherein each of R^{3a}, R^{3b}, and R^{3c} is independently selected from hydrogen, halogen, C1-C4 alkoxy and C1-C4 alkyl, provided at least one of R^{3a}, R^{3b}, and R^{3c} is halogen; and wherein R⁴ is selected form hydrogen, halogen, —CN, SO₂NH₂, SO₂CH₃, SO₂CF₃, and NO₂, or a pharmaceutically acceptable salt thereof.

In some embodiments, Q² is a structure:

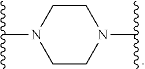 [SMILES: *N1CCN(*)CC1]

In some embodiments, R^{3a} is halogen. In some embodiments, R^{3a} is —F. In some embodiments, R^{3a} is halogen and each of R^{3b} and R^{3c} is hydrogen. In some embodiments, R^{3a} is —F and each of R^{3b} and R^{3c} is hydrogen. In some embodiments, R^{3c} is halogen. In some embodiments, R^{3c} is —F. In some embodiments, each of R^{3a} and R^{3c} is —F and R^{3b} is hydrogen. In some embodiments, R⁴ is —CN. In some embodiments, R⁴ is —Cl.

In some embodiments, the compound has a structure:

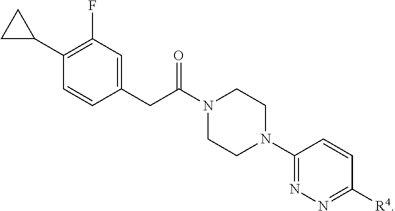

In some embodiments, the compound is selected from:

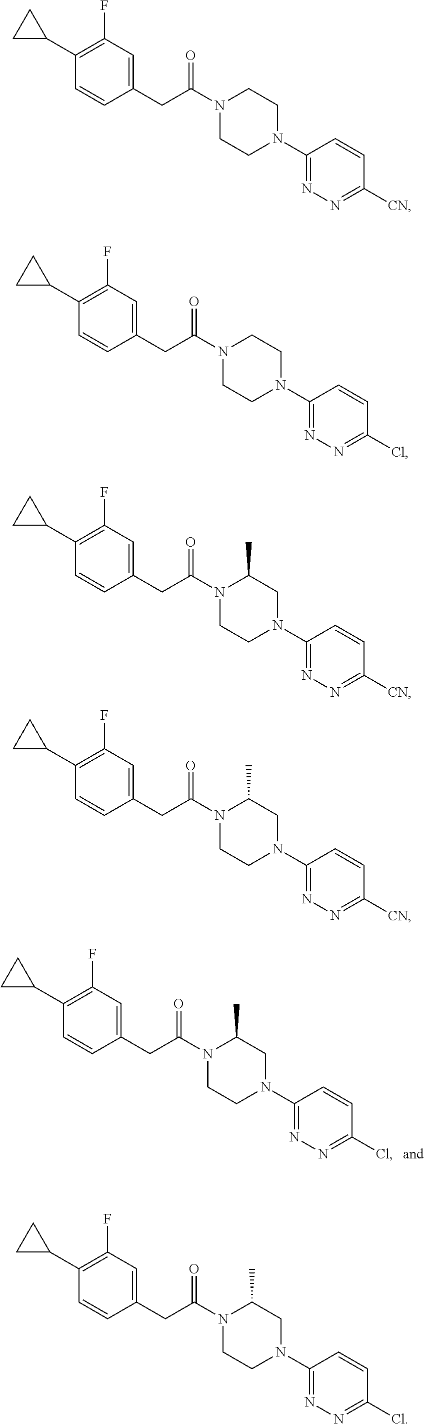 [SMILES: N#Cc1ccc(N2CCN(C(=O)Cc3ccc(C4CC4)c(F)c3)CC2)nn1 ; O=C(Cc1ccc(C2CC2)c(F)c1)N1CCN(c2ccc(Cl)nn2)CC1 ; C[C@H]1CN(c2ccc(C#N)nn2)CCN1C(=O)Cc1ccc(C2CC2)c(F)c1 ; C[C@@H]1CN(c2ccc(C#N)nn2)CCN1C(=O)Cc1ccc(C2CC2)c(F)c1 ; C[C@H]1CN(c2ccc(Cl)nn2)CCN1C(=O)Cc1ccc(C2CC2)c(F)c1 ; C[C@@H]1CN(c2ccc(Cl)nn2)CCN1C(=O)Cc1ccc(C2CC2)c(F)c1]

In some embodiments, the compound is selected from:

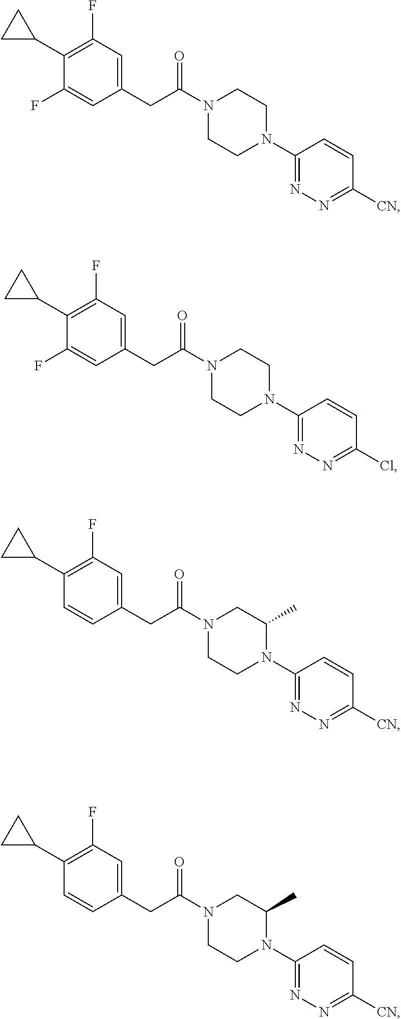 [SMILES: N#Cc1ccc(N2CCN(C(=O)Cc3cc(F)c(C4CC4)c(F)c3)CC2)nn1 ; O=C(Cc1cc(F)c(C2CC2)c(F)c1)N1CCN(c2ccc(Cl)nn2)CC1 ; C[C@H]1CN(C(=O)Cc2ccc(C3CC3)c(F)c2)CCN1c1ccc(C#N)nn1 ; C[C@@H]1CN(C(=O)Cc2ccc(C3CC3)c(F)c2)CCN1c1ccc(C#N)nn1]

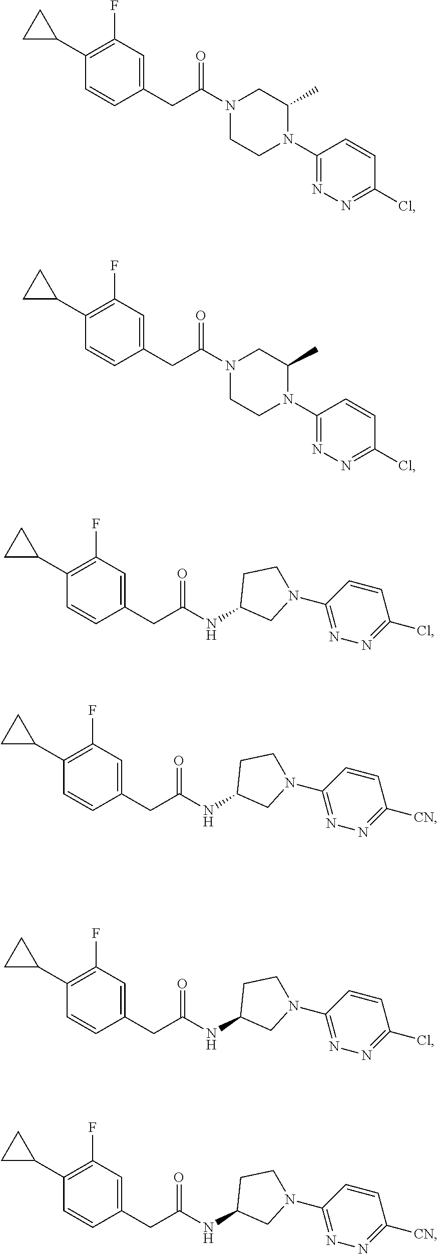 [SMILES: C[C@H]1CN(C(=O)Cc2ccc(C3CC3)c(F)c2)CCN1c1ccc(Cl)nn1 ; C[C@@H]1CN(C(=O)Cc2ccc(C3CC3)c(F)c2)CCN1c1ccc(Cl)nn1 ; O=C(Cc1ccc(C2CC2)c(F)c1)N[C@@H]1CCN(c2ccc(Cl)nn2)C1 ; N#Cc1ccc(N2CC[C@@H](NC(=O)Cc3ccc(C4CC4)c(F)c3)C2)nn1 ; O=C(Cc1ccc(C2CC2)c(F)c1)N[C@H]1CCN(c2ccc(Cl)nn2)C1 ; N#Cc1ccc(N2CC[C@H](NC(=O)Cc3ccc(C4CC4)c(F)c3)C2)nn1]

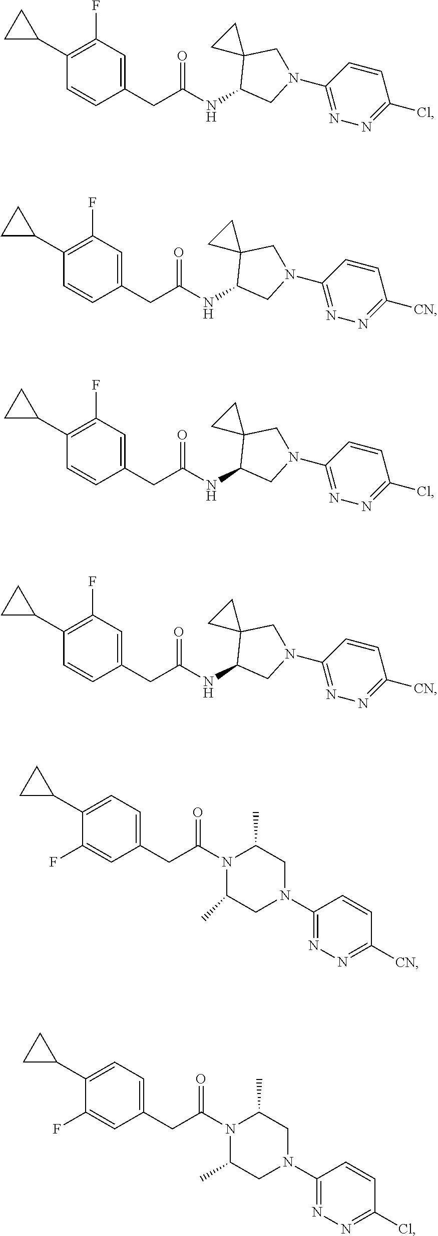 [SMILES: O=C(Cc1ccc(C2CC2)c(F)c1)N[C@H]1CN(c2ccc(Cl)nn2)CC12CC2 ; N#Cc1ccc(N2C[C@H](NC(=O)Cc3ccc(C4CC4)c(F)c3)C3(CC3)C2)nn1 ; O=C(Cc1ccc(C2CC2)c(F)c1)N[C@@H]1CN(c2ccc(Cl)nn2)CC12CC2 ; N#Cc1ccc(N2C[C@@H](NC(=O)Cc3ccc(C4CC4)c(F)c3)C3(CC3)C2)nn1 ; C[C@@H]1CN(c2ccc(C#N)nn2)C[C@H](C)N1C(=O)Cc1ccc(C2CC2)c(F)c1 ; C[C@@H]1CN(c2ccc(Cl)nn2)C[C@H](C)N1C(=O)Cc1ccc(C2CC2)c(F)c1]

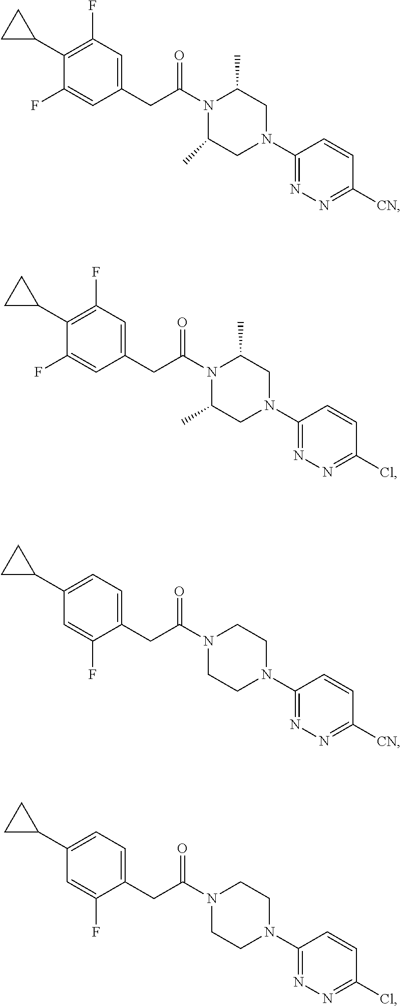 [SMILES: C[C@@H]1CN(c2ccc(C#N)nn2)C[C@H](C)N1C(=O)Cc1cc(F)c(C2CC2)c(F)c1 ; C[C@@H]1CN(c2ccc(Cl)nn2)C[C@H](C)N1C(=O)Cc1cc(F)c(C2CC2)c(F)c1 ; N#Cc1ccc(N2CCN(C(=O)Cc3ccc(C4CC4)cc3F)CC2)nn1 ; O=C(Cc1ccc(C2CC2)cc1F)N1CCN(c2ccc(Cl)nn2)CC1]

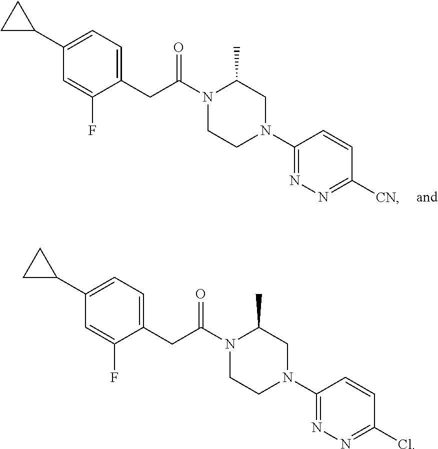 [SMILES: C[C@@H]1CN(c2ccc(C#N)nn2)CCN1C(=O)Cc1ccc(C2CC2)cc1F ; C[C@H]1CN(c2ccc(Cl)nn2)CCN1C(=O)Cc1ccc(C2CC2)cc1F]

In some embodiments, the compound has a structure represented by the formula:

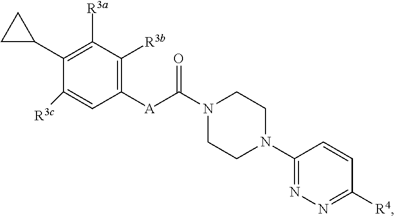

wherein A is selected from O, CO, CH₂, CF₂, NH, N(CH₃), and CH(OH); wherein each of R^{3a}, R^{3b}, and R^{3c} is independently selected from hydrogen, halogen, C1-C4 alkoxy and C1-C4 alkyl, provided at least one of R^{3a}, R^{3b}, and R^{3c} is halogen; and wherein R⁴ is selected form hydrogen, halogen, —CN, SO₂NH₂, SO₂CH₃, SO₂CF₃, and NO₂, or a pharmaceutically acceptable salt thereof.

In some embodiments, R^{3a} is halogen. In some embodiments, R^{3a} is —F. In some embodiments, R^{3a} is halogen and each of R^{3b} and R^{3c} is hydrogen. In some embodiments, R^{3a} is —F and each of R^{3b} and R^{3c} is hydrogen. In some embodiments, R^{3c} is halogen. In some embodiments, R^{3c} is —F. In some embodiments, each of R^{3a} and R^{3c} is —F and R^{3b} is hydrogen.

In some embodiments, R⁴ is —CN. In some embodiments, R⁴ is —Cl.

In some embodiments, the compound is selected from:

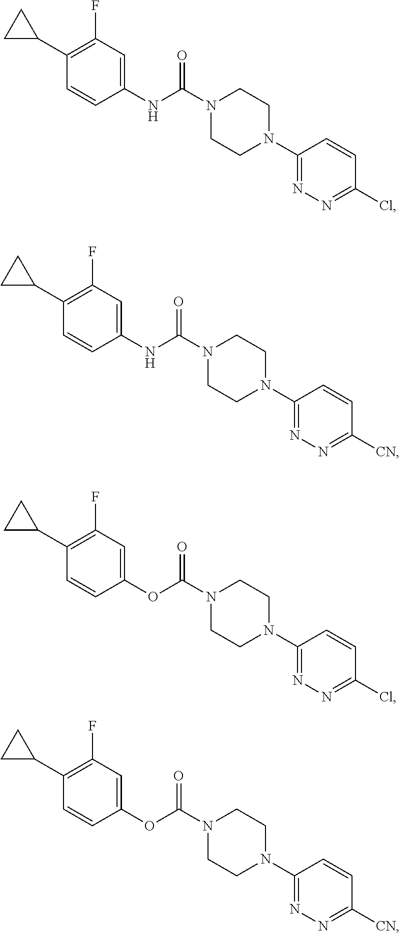 [SMILES: O=C(Nc1ccc(C2CC2)c(F)c1)N1CCN(c2ccc(Cl)nn2)CC1 ; N#Cc1ccc(N2CCN(C(=O)Nc3ccc(C4CC4)c(F)c3)CC2)nn1 ; O=C(Oc1ccc(C2CC2)c(F)c1)N1CCN(c2ccc(Cl)nn2)CC1 ; N#Cc1ccc(N2CCN(C(=O)Oc3ccc(C4CC4)c(F)c3)CC2)nn1]

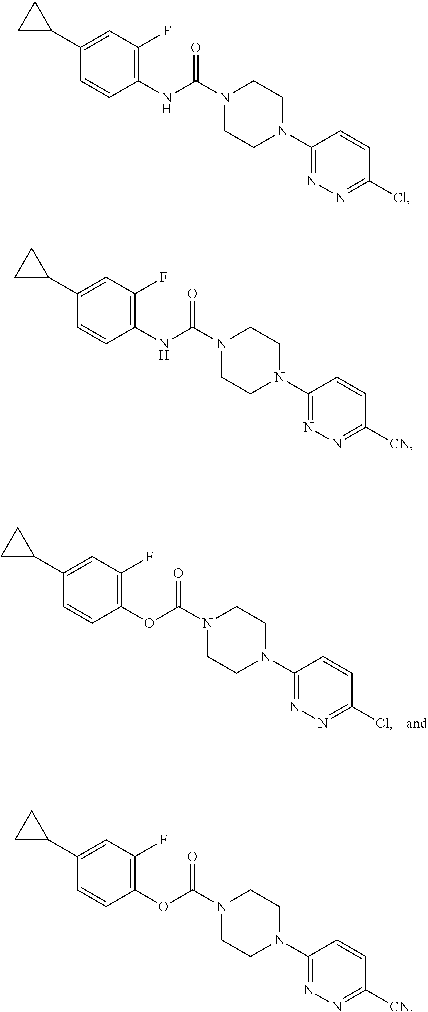 [SMILES: O=C(Nc1ccc(C2CC2)cc1F)N1CCN(c2ccc(Cl)nn2)CC1 ; N#Cc1ccc(N2CCN(C(=O)Nc3ccc(C4CC4)cc3F)CC2)nn1 ; O=C(Oc1ccc(C2CC2)cc1F)N1CCN(c2ccc(Cl)nn2)CC1 ; N#Cc1ccc(N2CCN(C(=O)Oc3ccc(C4CC4)cc3F)CC2)nn1]

In a still further aspect, the compound is selected from:

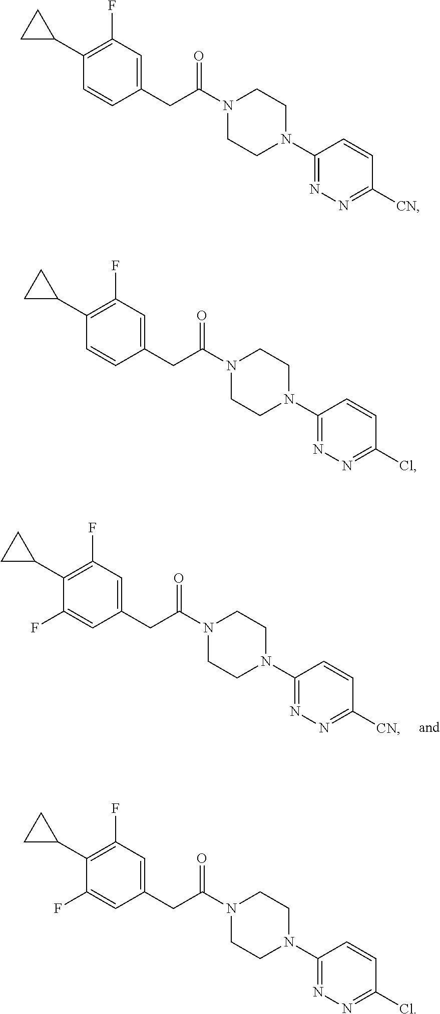 [SMILES: N#Cc1ccc(N2CCN(C(=O)Cc3ccc(C4CC4)c(F)c3)CC2)nn1 ; O=C(Cc1ccc(C2CC2)c(F)c1)N1CCN(c2ccc(Cl)nn2)CC1 ; N#Cc1ccc(N2CCN(C(=O)Cc3cc(F)c(C4CC4)c(F)c3)CC2)nn1 ; O=C(Cc1cc(F)c(C2CC2)c(F)c1)N1CCN(c2ccc(Cl)nn2)CC1]

In a further aspect, a compound can be present as one or more of the following structures:

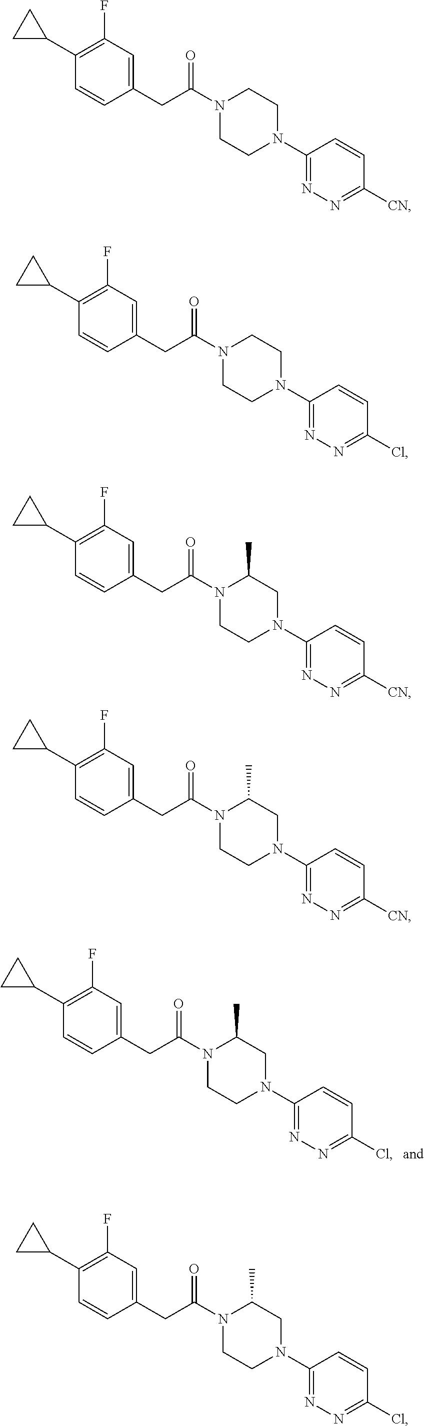 [SMILES: N#Cc1ccc(N2CCN(C(=O)Cc3ccc(C4CC4)c(F)c3)CC2)nn1 ; O=C(Cc1ccc(C2CC2)c(F)c1)N1CCN(c2ccc(Cl)nn2)CC1 ; C[C@H]1CN(c2ccc(C#N)nn2)CCN1C(=O)Cc1ccc(C2CC2)c(F)c1 ; C[C@@H]1CN(c2ccc(C#N)nn2)CCN1C(=O)Cc1ccc(C2CC2)c(F)c1 ; C[C@H]1CN(c2ccc(Cl)nn2)CCN1C(=O)Cc1ccc(C2CC2)c(F)c1 ; C[C@@H]1CN(c2ccc(Cl)nn2)CCN1C(=O)Cc1ccc(C2CC2)c(F)c1]

or a pharmaceutically acceptable salt thereof.

In a further aspect, disclosed are compounds having a structure represented by a formula:

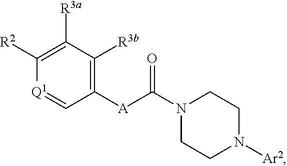

wherein A is selected from O, CO, CH₂, CF₂, NH, N(CH₃) and CH(OH); wherein Q¹ is selected from N and CH; wherein R² is selected from C1-C8 acyclic alkyl, C1-C8 acyclic alkenyl, C1-C8 monohaloalkyl, C1-C8 polyhaloalkyl, (C1-C8)(C1-C8) dialkylamino, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; wherein each of R^{3a} and R^{3b} is independently selected from hydrogen, halogen, C1-C4 alkyl, and C1-C4 alkoxy; wherein Ar² is a structure represented by a formula selected from:

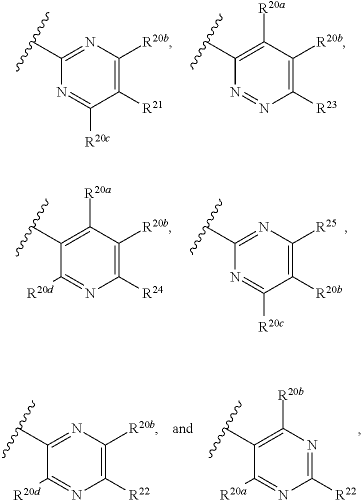

wherein each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; wherein R²¹, when present, is selected from —CN, —NO₂, SO₂NH₂, SO₂CH₃, SO₂CF₃, and Cy¹; wherein Cy¹, when present, is selected from cycle, heterocycle, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino; wherein R²², when present, is selected from —CN, halogen, —NO₂, SO₂NH₂, SO₂CH₃, and SO₂CF₃; wherein R²³, when present, is selected from —CN, —NO₂, SO₂NH₂, SO₂CH₃, SO₂CF₃, cyclohexyl,

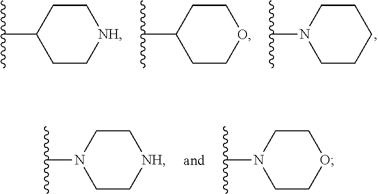

wherein R²⁴, when present, is selected from —CN, halogen, —NO₂, SO₂NH₂, SO₂CH₃, and SO₂CF₃, provided that if A is NH or N(CH₃) then R²⁴ is not —NO₂; and wherein R²⁵, when present, is selected from —CN, —NO₂, SO₂NH₂, SO₂CH₃, and SO₂CF₃; or a pharmaceutically acceptable salt thereof.

In a further aspect, disclosed are compounds having a structure represented by a formula:

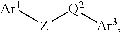

wherein Q² is a structure selected from:

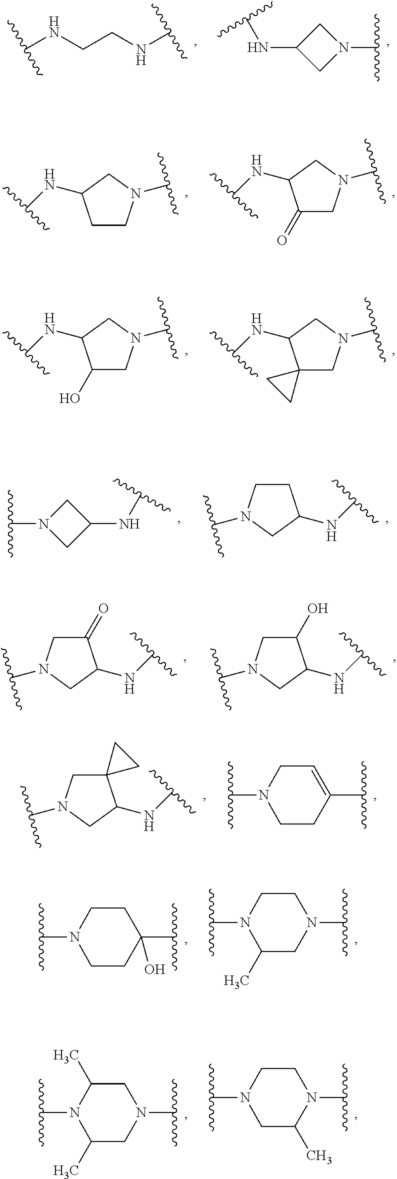

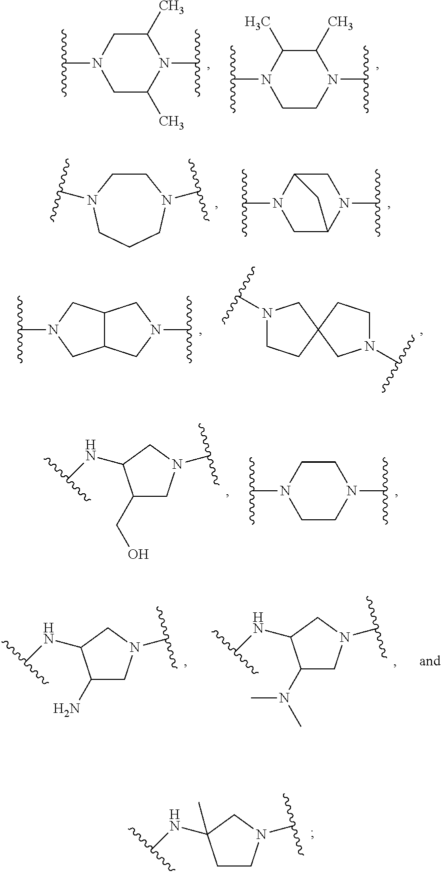

wherein Z is selected from O(C═O), CF₂CO, COCH₂, CH₂CO,

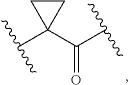 [SMILES: *C(=O)C1(*)CC1]

CO, CH₂SO₂, SO₂, NHCO, N(CH₃)CO, and CH(OH)CO; wherein Ar¹ is selected from aryl and heteroaryl and substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; and wherein Ar³ is a structure selected from:

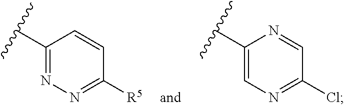

wherein R⁵, when present, is selected from CN, halogen, —NO₂, SO₂NH₂, and SO₂CH₃, provided that if R⁵ is CN and Z is CO then Ar¹ is not substituted with C1-C8 monohaloalkyl or C1-C8 polyhaloalkyl; provided that if R⁵ is halogen then Ar¹ is selected from 5- and 6-membered heteroaryl and Z cannot be CO, or a pharmaceutically acceptable salt thereof.

In a further aspect, disclosed are compounds having a structure represented by a formula:

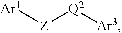

or a pharmaceutically acceptable salt thereof, wherein:
Q² is a structure selected from:

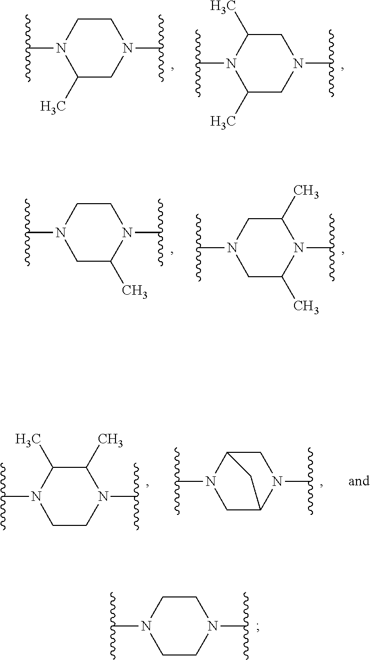

Z is CH₂CO;
Ar¹ is selected from aryl and heteroaryl and substituted with 1, 2, or 3 groups independently selected from halogen, —NO₂, —CN, —OH, —SH, —NH₂, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy;
Ar³ is a structure selected from:

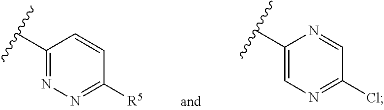

and
R⁵, when present, is selected from CN, halogen, —NO₂, SO₂NH₂, and SO₂CH₃, provided that if R⁵ is halogen then Ar¹ is selected from 5- and 6-membered heteroaryl.

In a still further aspect, Ar¹ is selected from aryl and heteroaryl and substituted with 1, 2, or 3 groups independently selected from halogen, —NO₂, —CN, —OH, —SH, —NH₂, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl.

In yet a further aspect, Ar³ is:

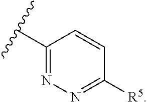

In an even further aspect, R⁵ is CN. In a still further aspect, R⁵ is —Cl. In yet a further aspect, R⁵ is selected from halogen, —NO₂, SO₂NH₂, and SO₂CH₃.

In an even further aspect, the compound is:

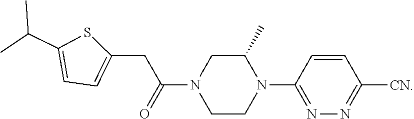 [SMILES: CC(C)c1ccc(CC(=O)N2CCN(c3ccc(C#N)nn3)[C@@H](C)C2)s1]

In a further aspect, disclosed are compounds having a structure represented by a formula:

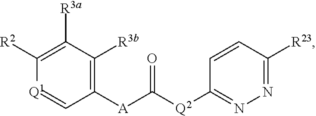

wherein A is selected from O, CO, CH₂, CF₂, NH, N(CH₃), and CH(OH); wherein Q¹ is CH; and wherein R² is selected from —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; or wherein Q¹ is N; and R² is selected from halogen, —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; wherein Q² is a structure selected from:

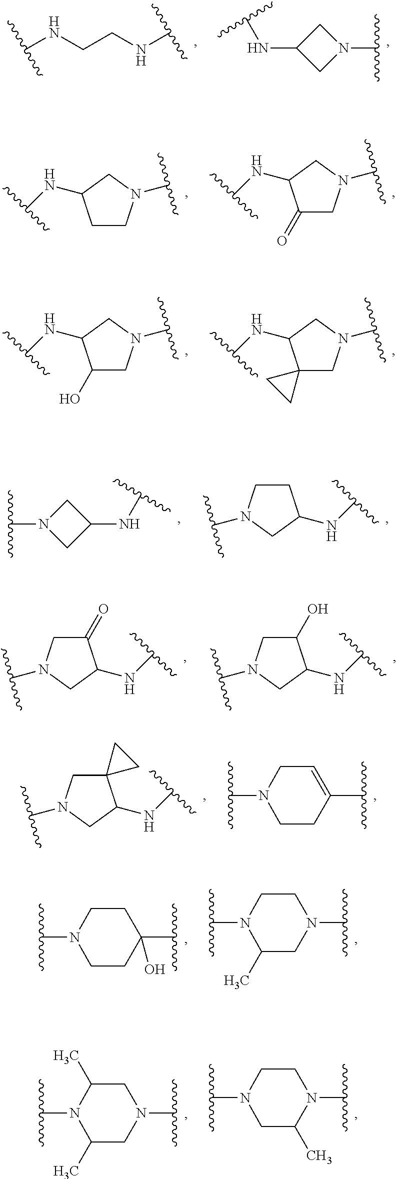

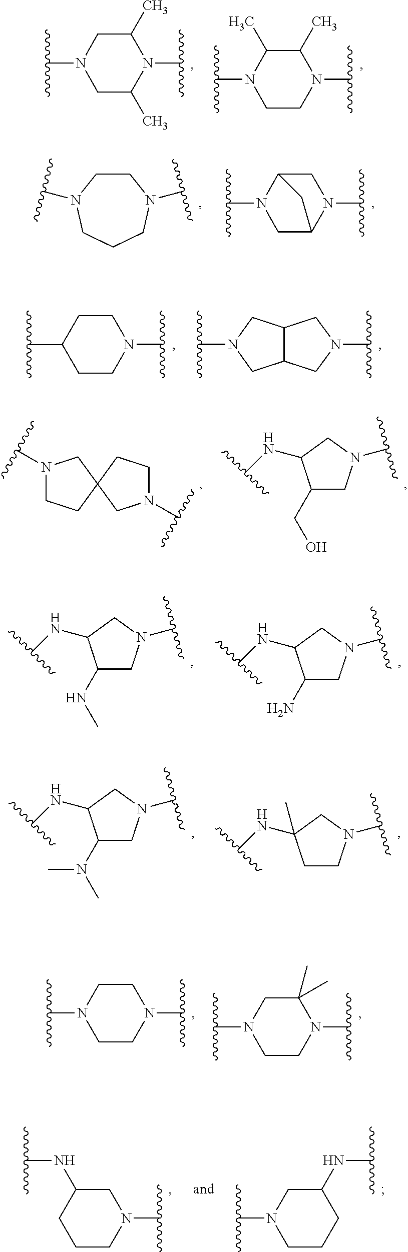

wherein each of R^{3a} and R^{3b} is independently selected from hydrogen, halogen, —OH, C1-C4 alkoxy, and C1-C4 alkyl; and wherein R²³ is selected form hydrogen, halogen, —CN, SO₂NH₂, SO₂CH₃, SO₂CF₃, and NO₂, or a pharmaceutically acceptable salt thereof.

In a further aspect, disclosed are compounds having a structure represented by a formula:

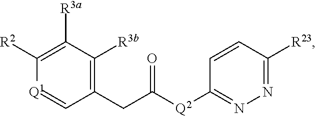

or a pharmaceutically acceptable salt thereof, wherein:
Q¹ is CH; and R² is selected from —SCH₃, C1-C8 acyclic alkyl, C2-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; or
Q¹ is N; and R² is selected from halogen, —SCH₃, C1-C8 acyclic alkyl, C2-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy;
Q² is a structure selected from:

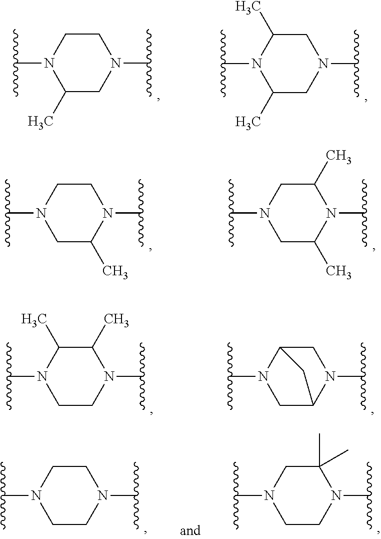

each of R^{3a} and R^{3b} is independently selected from hydrogen, halogen, —OH, C1-C4 alkoxy, and C1-C4 alkyl; and
R²³ is selected from hydrogen, halogen, —CN, SO₂NH₂, SO₂CH₃, SO₂CF₃, and NO₂.

In a further aspect, disclosed are compounds having a structure represented by a formula:

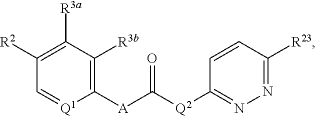

wherein A is selected from O, CO, CH₂, CF₂, NH, N(CH₃), and CH(OH); wherein Q¹ is CH; and wherein R² is selected from C1-C8 acyclic alkyl, C1-C8 acyclic alkenyl, C1-C8 monohaloalkyl, C1-C8 polyhaloalkyl, and cyclopropyl, or wherein A is selected from O, CO, CH₂, CF₂, N(CH₃), and CH(OH); wherein Q¹ is N; and R² is selected from halogen, C1-C8 acyclic alkyl, C1-C8 acyclic alkenyl, C1-C8 monohaloalkyl, C1-C8 polyhaloalkyl, and cyclopropyl; wherein Q² is a structure selected from:

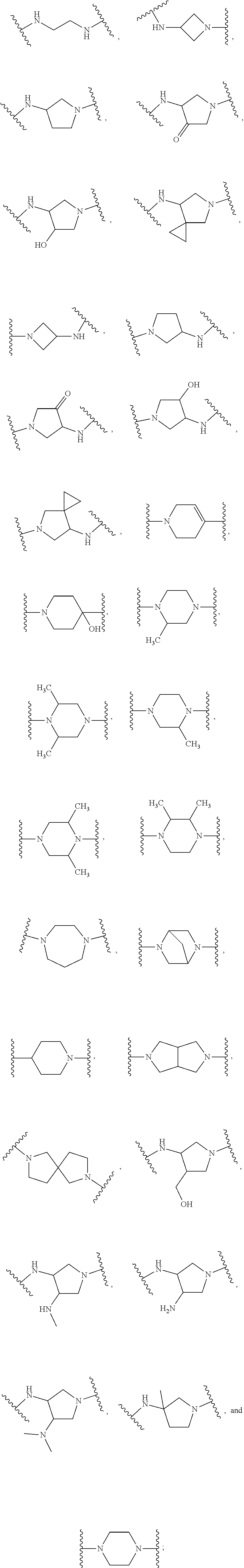

wherein each of R^{3a} and R^{3b} is independently selected from hydrogen, halogen, C1-C4 alkyl, and C1-C4 alkoxy; and wherein R⁴ is selected form hydrogen, halogen, —CN, SO₂NH₂, SO₂CH₃, SO₂CF₃, and NO₂, or a pharmaceutically acceptable salt thereof.

In a further aspect, disclosed are compounds having a structure represented by a formula:

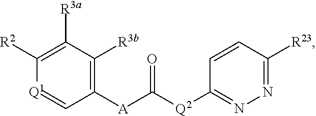

wherein A is selected from O, CO, CH₂, CF₂, NH, N(CH₃), and CH(OH); wherein Q¹ is CH; and wherein R² is selected from C1-C8 acyclic alkyl, C1-C8 acyclic alkenyl, C1-C8 monohaloalkyl, C1-C8 polyhaloalkyl, and cyclopropyl, or wherein A is selected from O, CO, CH₂, CF₂, N(CH₃), and CH(OH); wherein Q¹ is N; and R² is selected from halogen, C1-C8 acyclic alkyl, C1-C8 acyclic alkenyl, C1-C8 monohaloalkyl, C1-C8 polyhaloalkyl, and cyclopropyl; wherein Q² is a structure selected from:

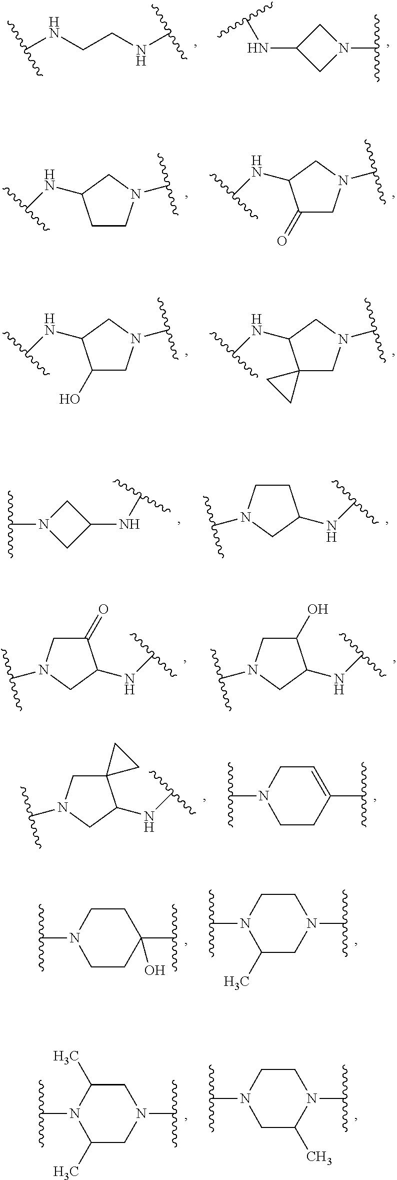

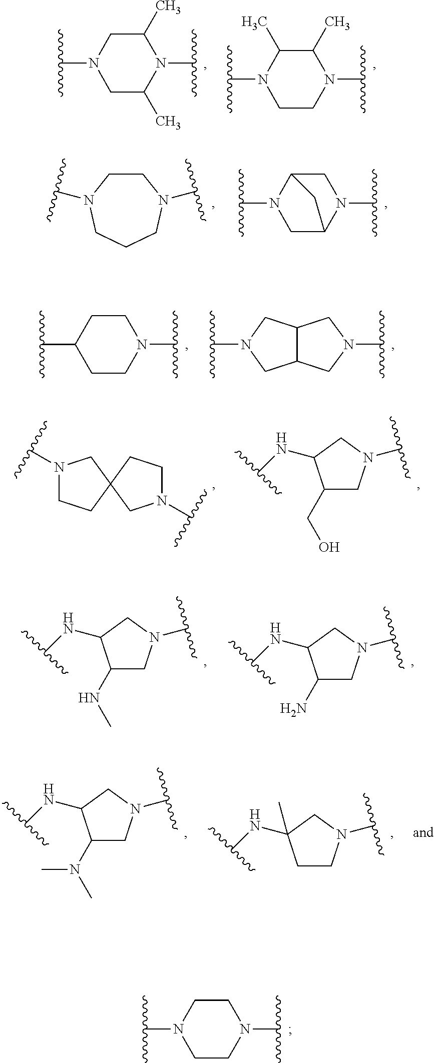

wherein each of R^{3a} and R^{3b} is independently selected from hydrogen, halogen, C1-C4 alkyl, and C1-C4 alkoxy; and wherein R⁴ is selected form hydrogen, halogen, —CN, SO₂NH₂, SO₂CH₃, SO₂CF₃, and NO₂, or a pharmaceutically acceptable salt thereof.

In a further aspect, disclosed are compounds having a structure represented by a formula:

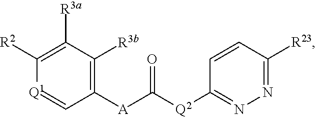

wherein A is CH₂; wherein Q¹ is CH; and wherein R² is selected from C1-C8 acyclic alkyl, C1-C8 acyclic alkenyl, C1-C8 monohaloalkyl, C1-C8 polyhaloalkyl, and cyclopropyl, or wherein A is CH₂; wherein Q¹ is N; and R² is selected from halogen, C1-C8 acyclic alkyl, C1-C8 acyclic alkenyl, C1-C8 monohaloalkyl, C1-C8 polyhaloalkyl, and cyclopropyl; wherein Q² is a structure selected from:

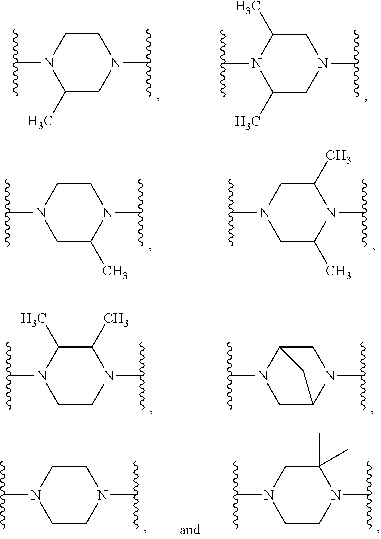

wherein each of R^{3a} and R^{3b} is independently selected from hydrogen, halogen, C1-C4 alkyl, and C1-C4 alkoxy; and wherein R⁴ is selected form hydrogen, halogen, —CN, SO₂NH₂, SO₂CH₃, SO₂CF₃, and NO₂, or a pharmaceutically acceptable salt thereof.

In a further aspect, disclosed are compounds having a structure represented by a formula:

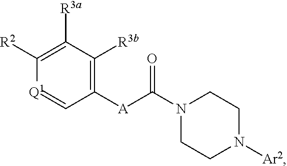

wherein A is selected from O, CO, CH₂, CF₂, NH, N(CH₃) and CH(OH); wherein Q¹ is selected from N and CH; wherein R² is selected from C1-C8 acyclic alkyl, C1-C8 acyclic alkenyl, C1-C8 monohaloalkyl, C1-C8 polyhaloalkyl, (C1-C8)(C1-C8) dialkylamino, and cyclopropyl; wherein each of R^{3a} and R^{3b} is independently selected from hydrogen, halogen, C1-C4 alkyl, and C1-C4 alkoxy; wherein Ar² is a structure represented by a formula selected from:

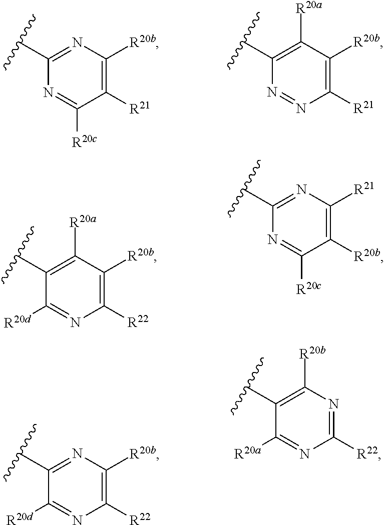

wherein each of R^{20a}, R^{20b}, R^{20c}, and R^{20d}, when present, is independently selected from hydrogen, halogen, —CN, —NO₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; wherein R²¹, when present, is selected from —CN, —NO₂, SO₂NH₂, SO₂CH₃, SO₂CF₃, and Cy¹; and wherein R²², when present, is selected from —CN, halogen, —NO₂, SO₂NH₂, SO₂CH₃, and SO₂CF₃, or a pharmaceutically acceptable salt thereof.

In a further aspect, disclosed are compounds having a structure represented by a formula:

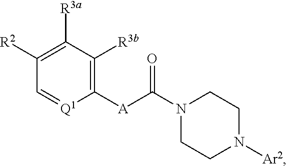

wherein A is selected from O, CO, CH₂, CF₂, NH, N(CH₃) and CH(OH); wherein Q¹ is selected from N and CH; wherein R² is selected from C1-C8 acyclic alkyl, C1-C8 acyclic alkenyl, C1-C8 monohaloalkyl, C1-C8 polyhaloalkyl, (C1-C8)(C1-C8) dialkylamino, and cyclopropyl; wherein each of R^{3a} and R^{3b} is independently selected from hydrogen, halogen, C1-C4 alkyl, and C1-C4 alkoxy; wherein Ar² is a structure represented by a formula selected from:

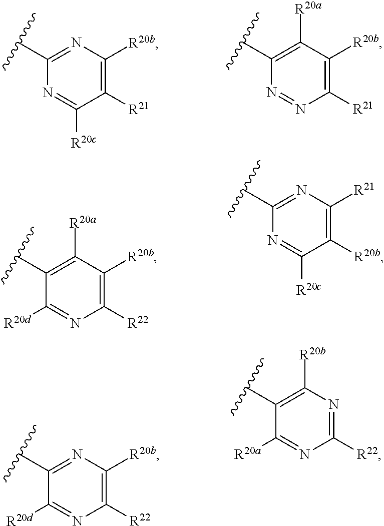

wherein each of R^{20a}, R^{20b}b, R^{20c}, and R^{20d}, when present, is independently selected from hydrogen, halogen, —CN, —NO₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; wherein R²¹, when present, is selected from —CN, —NO₂, SO₂NH₂, SO₂CH₃, SO₂CF₃, and Cy¹; and wherein R²², when present, is selected from —CN, halogen, —NO₂, SO₂NH₂, SO₂CH₃, and SO₂CF₃, or a pharmaceutically acceptable salt thereof.

In a further aspect, disclosed are compounds having a structure represented by a formula:

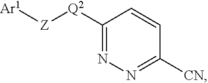

wherein Q² is a structure selected from:

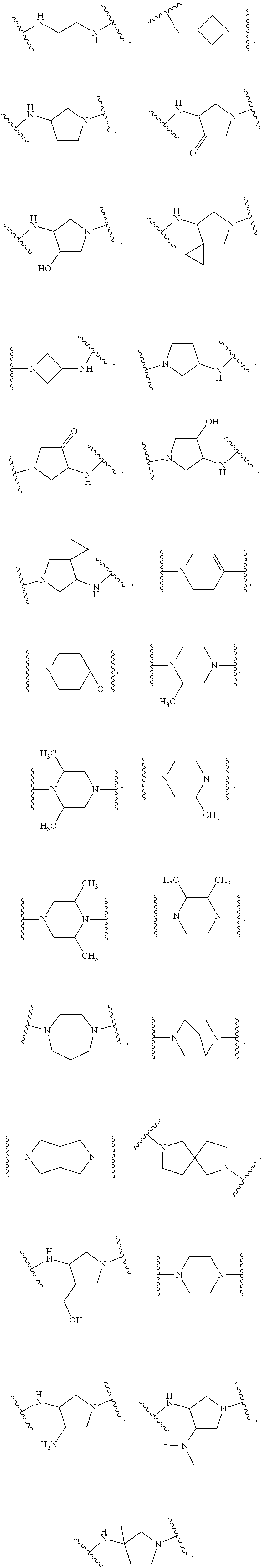

wherein Z is selected from O(C═O), CF₂CO, COCH₂, CH₂CO,

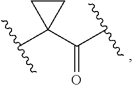 [SMILES: *C(=O)C1(*)CC1]

CO, CH₂SO₂, SO₂, NHCO, and CH(OH)CO; and wherein Ar¹ is selected from aryl and heteroaryl and substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy, or a pharmaceutically acceptable salt thereof.

In a further aspect, disclosed are compounds having a structure represented by a formula:

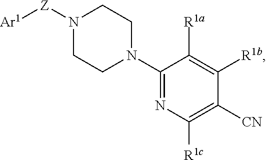

wherein each of R^{1a}, R^{1b}, and R^{1c} is independently selected from hydrogen, halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino; and wherein Z is selected from COCH₂, O(C═O), CF₂CO, and CH(OH)CO; and wherein Ar¹ is selected from aryl and heteroaryl and substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl, or wherein Z is selected from CO,

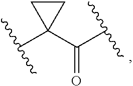 [SMILES: *C(=O)C1(*)CC1]

CH₂CO, COCH₂, NHCO, and NHCS; and wherein Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino, or a pharmaceutically acceptable salt thereof.

In a further aspect, disclosed are compounds having a structure represented by a formula:

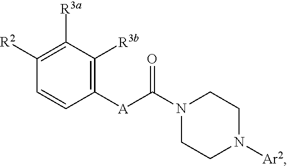

wherein A is selected from O, CO, CH₂, CF₂, NH, and CH(OH); wherein R² is selected from isopropyl and cyclopropyl; wherein Ar² is a structure represented by a formula selected from:

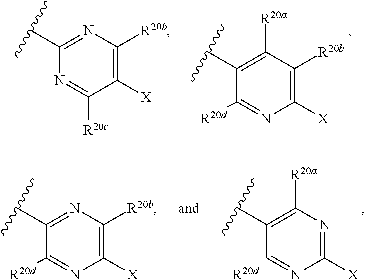

wherein X is halogen; and wherein each of R^{20a}, R^{20b}, R^{20c}, and R^{20d}, when present, is independently selected from hydrogen, halogen, —CN, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl; or wherein A is selected from O, CO, CH₂, CF₂, and CH(OH); and wherein Ar² is a structure represented by a formula:

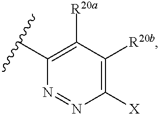

or a pharmaceutically acceptable salt thereof.

Also disclosed are compounds selected from:

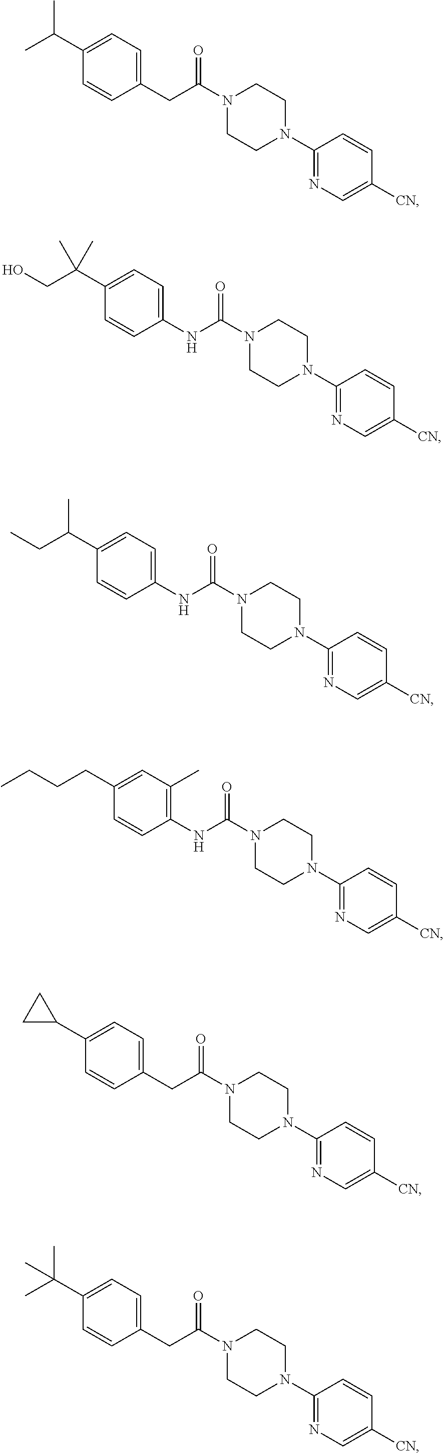 [SMILES: CC(C)c1ccc(CC(=O)N2CCN(c3ccc(C#N)cn3)CC2)cc1 ; CC(C)(CO)c1ccc(NC(=O)N2CCN(c3ccc(C#N)cn3)CC2)cc1 ; CCC(C)c1ccc(NC(=O)N2CCN(c3ccc(C#N)cn3)CC2)cc1 ; CCCCc1ccc(NC(=O)N2CCN(c3ccc(C#N)cn3)CC2)c(C)c1 ; N#Cc1ccc(N2CCN(C(=O)Cc3ccc(C4CC4)cc3)CC2)nc1 ; CC(C)(C)c1ccc(CC(=O)N2CCN(c3ccc(C#N)cn3)CC2)cc1]

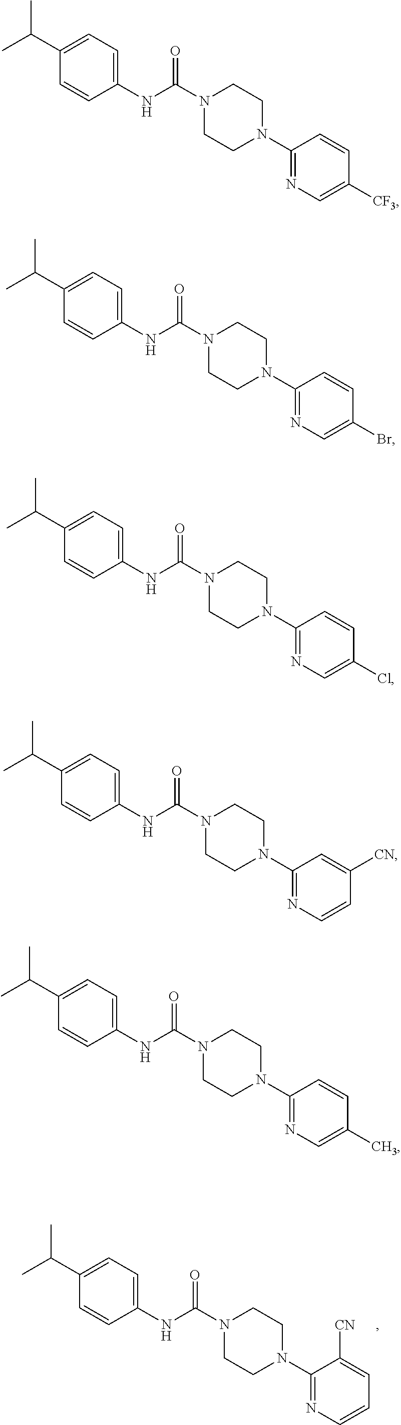 [SMILES: CC(C)c1ccc(NC(=O)N2CCN(c3ccc(C(F)(F)F)cn3)CC2)cc1 ; CC(C)c1ccc(NC(=O)N2CCN(c3ccc(Br)cn3)CC2)cc1 ; CC(C)c1ccc(NC(=O)N2CCN(c3ccc(Cl)cn3)CC2)cc1 ; CC(C)c1ccc(NC(=O)N2CCN(c3cc(C#N)ccn3)CC2)cc1 ; Cc1ccc(N2CCN(C(=O)Nc3ccc(C(C)C)cc3)CC2)nc1 ; CC(C)c1ccc(NC(=O)N2CCN(c3ncccc3C#N)CC2)cc1]

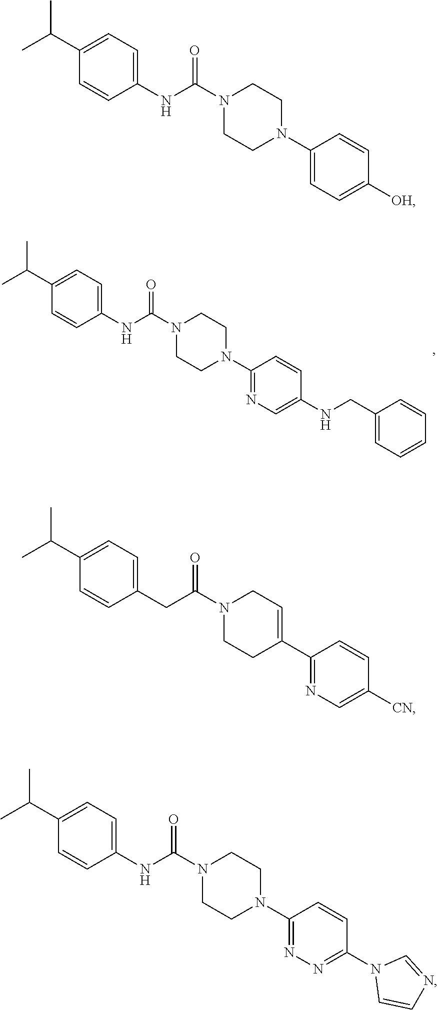 [SMILES: CC(C)c1ccc(NC(=O)N2CCN(c3ccc(O)cc3)CC2)cc1 ; CC(C)c1ccc(NC(=O)N2CCN(c3ccc(NCc4ccccc4)cn3)CC2)cc1 ; CC(C)c1ccc(CC(=O)N2CC=C(c3ccc(C#N)cn3)CC2)cc1 ; CC(C)c1ccc(NC(=O)N2CCN(c3ccc(-n4ccnc4)nn3)CC2)cc1]

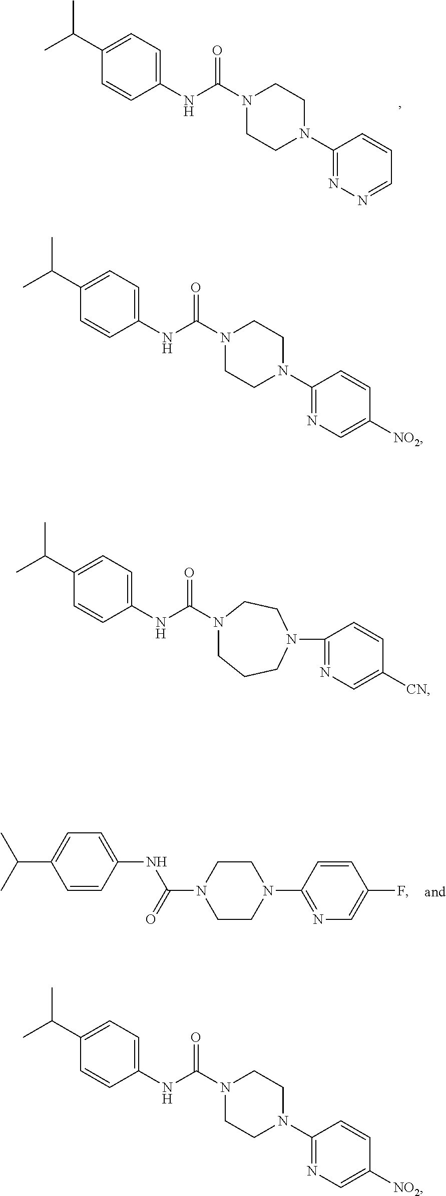 [SMILES: CC(C)c1ccc(NC(=O)N2CCN(c3cccnn3)CC2)cc1 ; CC(C)c1ccc(NC(=O)N2CCN(c3ccc([N+](=O)[O-])cn3)CC2)cc1 ; CC(C)c1ccc(NC(=O)N2CCCN(c3ccc(C#N)cn3)CC2)cc1 ; CC(C)c1ccc(NC(=O)N2CCN(c3ccc(F)cn3)CC2)cc1 ; CC(C)c1ccc(NC(=O)N2CCN(c3ccc([N+](=O)[O-])cn3)CC2)cc1]

or a pharmaceutically acceptable salt thereof.

In a further aspect, Q¹ is CH and R² is selected from C1-C8 acyclic alkyl, C1-C8 acyclic alkenyl, C1-C8 monohaloalkyl, C1-C8 polyhaloalkyl, and cyclopropyl; or Q¹ is N and R² is selected from halogen, C1-C8 acyclic alkyl, C1-C8 acyclic alkenyl, C1-C8 monohaloalkyl, C1-C8 polyhaloalkyl, and cyclopropyl; and Q² is a structure selected from:

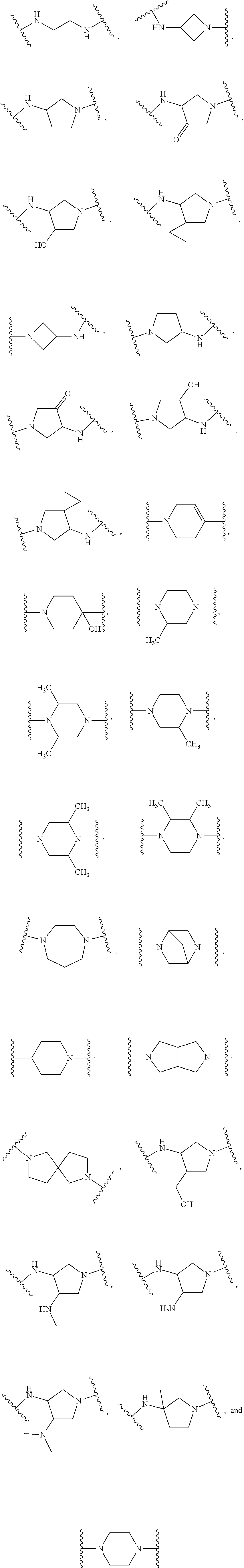

In a further aspect, Q¹ is CH; and R² is selected from C1-C8 acyclic alkyl, C1-C8 acyclic alkenyl, C1-C8 monohaloalkyl, C1-C8 polyhaloalkyl, and cyclopropyl; or Q¹ is N; and R² is selected from halogen, C1-C8 acyclic alkyl, C1-C8 acyclic alkenyl, C1-C8 monohaloalkyl, C1-C8 polyhaloalkyl, and cyclopropyl.

In a further aspect, Q¹ is CH or N; and R² is selected from —SCH₃, C1-C8 alkoxyhaloalkyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy.

In a further aspect, the compound has a structure represented by a formula:

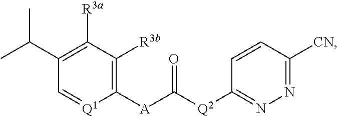

or a pharmaceutically acceptable salt thereof.

In a still further aspect, the compound has a structure represented by a formula selected from:

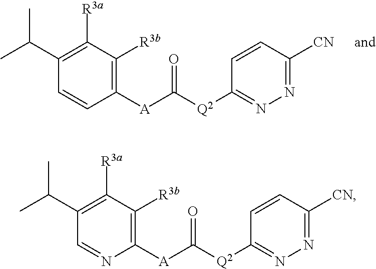

or a pharmaceutically acceptable salt thereof.

In yet a further aspect, the compound has a structure represented by a formula:

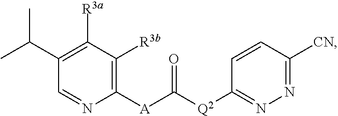

or a pharmaceutically acceptable salt thereof.

In an even further aspect, the compound has a structure represented by a formula:

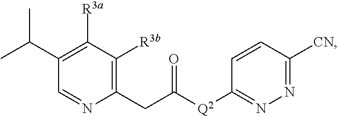

or a pharmaceutically acceptable salt thereof.

In a still further aspect, the compound has a structure represented by a formula:

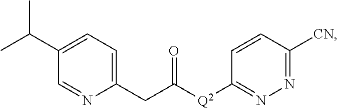

or a pharmaceutically acceptable salt thereof.

In yet a further aspect, the compound is:

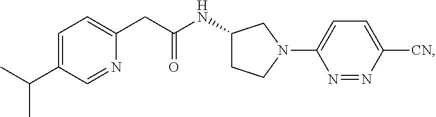 [SMILES: CC(C)c1ccc(CC(=O)N[C@H]2CCN(c3ccc(C#N)nn3)C2)nc1]

or a pharmaceutically acceptable salt thereof.

In a further aspect, the compound has a structure represented by a formula:

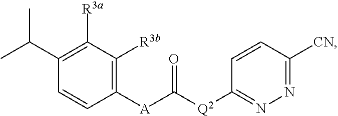

or a pharmaceutically acceptable salt thereof.

In yet a further aspect, the compound has a structure represented by a formula selected from:

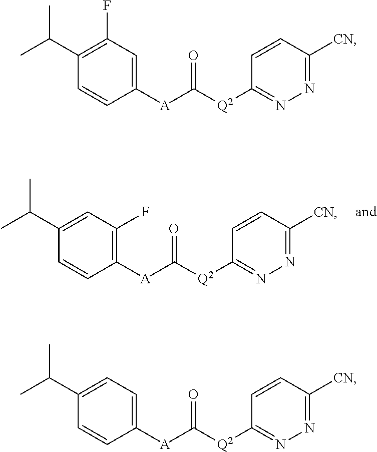

or a pharmaceutically acceptable salt thereof.

In an even further aspect, the compound has a structure represented by a formula selected from:

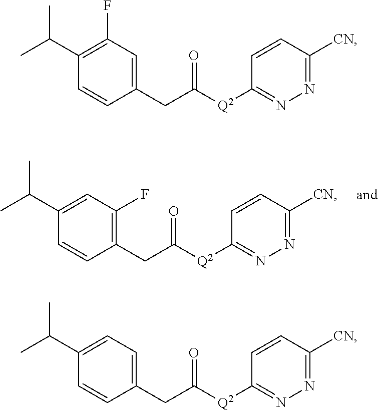

or a pharmaceutically acceptable salt thereof.

In a still further aspect, the compound is selected from:

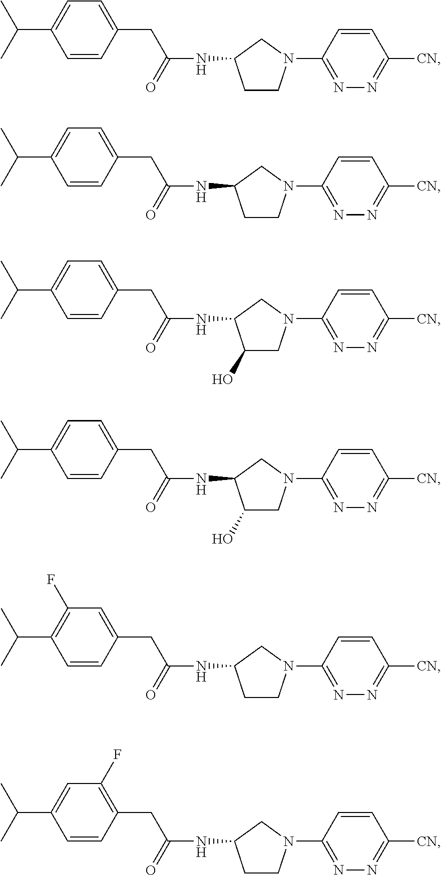 [SMILES: CC(C)c1ccc(CC(=O)N[C@H]2CCN(c3ccc(C#N)nn3)C2)cc1 ; CC(C)c1ccc(CC(=O)N[C@@H]2CCN(c3ccc(C#N)nn3)C2)cc1 ; CC(C)c1ccc(CC(=O)N[C@@H]2CN(c3ccc(C#N)nn3)C[C@H]2O)cc1 ; CC(C)c1ccc(CC(=O)N[C@H]2CN(c3ccc(C#N)nn3)C[C@@H]2O)cc1 ; CC(C)c1ccc(CC(=O)N[C@H]2CCN(c3ccc(C#N)nn3)C2)cc1F ; CC(C)c1ccc(CC(=O)N[C@H]2CCN(c3ccc(C#N)nn3)C2)c(F)c1]

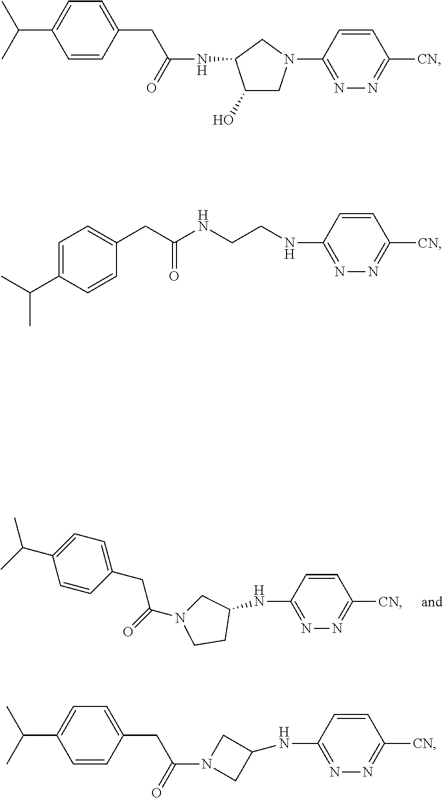 [SMILES: CC(C)c1ccc(CC(=O)N[C@@H]2CN(c3ccc(C#N)nn3)C[C@@H]2O)cc1 ; CC(C)c1ccc(CC(=O)NCCNc2ccc(C#N)nn2)cc1 ; CC(C)c1ccc(CC(=O)N2CC[C@@H](Nc3ccc(C#N)nn3)C2)cc1 ; CC(C)c1ccc(CC(=O)N2CC(Nc3ccc(C#N)nn3)C2)cc1]

or a pharmaceutically acceptable salt thereof.

In yet a further aspect, the compound has a structure represented by a formula selected from:

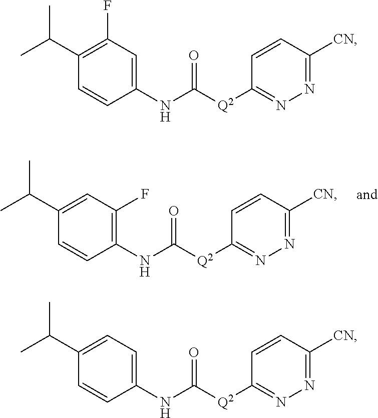

or a pharmaceutically acceptable salt thereof.

In an even further aspect, the compound is selected from:

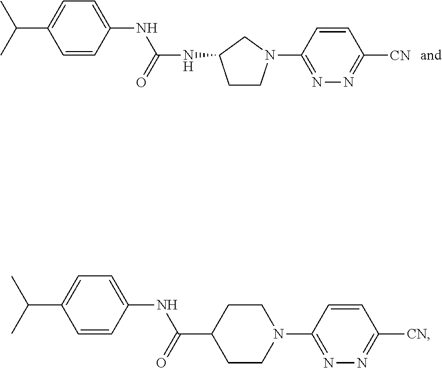 [SMILES: CC(C)c1ccc(NC(=O)N[C@H]2CCN(c3ccc(C#N)nn3)C2)cc1 ; CC(C)c1ccc(NC(=O)C2CCN(c3ccc(C#N)nn3)CC2)cc1]

or a pharmaceutically acceptable salt thereof.

In a further aspect, the compound has a structure represented by a formula:

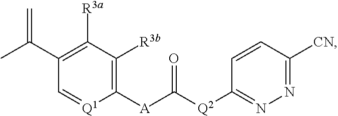

or a pharmaceutically acceptable salt thereof.

In a still further aspect, the compound has a structure represented by a formula selected from:

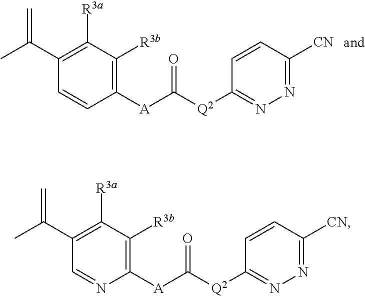

or a pharmaceutically acceptable salt thereof.

In yet a further aspect, the compound has a structure represented by a formula:

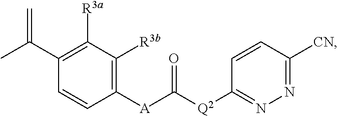

or a pharmaceutically acceptable salt thereof.

In an even further aspect, the compound has a structure represented by a formula selected from:

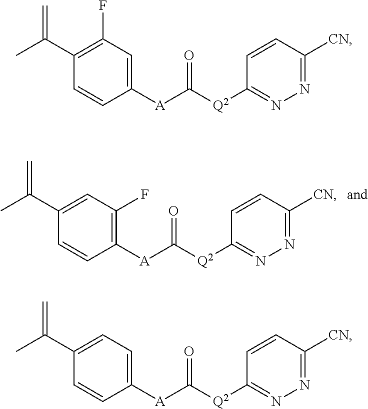

or a pharmaceutically acceptable salt thereof.

In a still further aspect, the compound has a structure represented by a formula selected from:

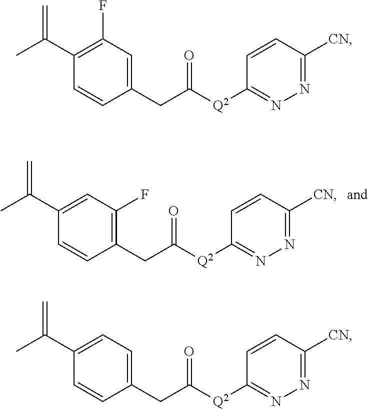

or a pharmaceutically acceptable salt thereof.

In yet further aspect, the compound is selected from:

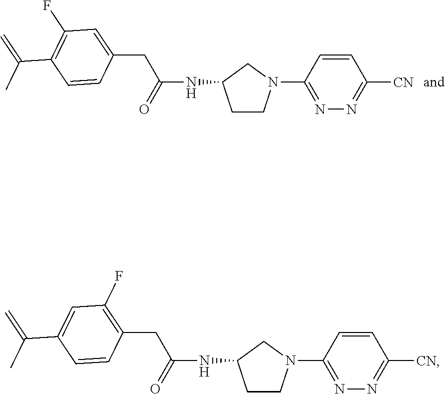 [SMILES: C=C(C)c1ccc(CC(=O)N[C@H]2CCN(c3ccc(C#N)nn3)C2)cc1F ; C=C(C)c1ccc(CC(=O)N[C@H]2CCN(c3ccc(C#N)nn3)C2)c(F)c1]

or a pharmaceutically acceptable salt thereof.

In a further aspect, the compound has a structure represented by a formula:

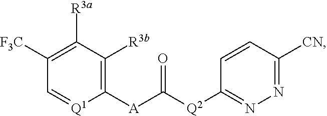

or a pharmaceutically acceptable salt thereof.

In a still further aspect, the compound has a structure represented by a formula selected from:

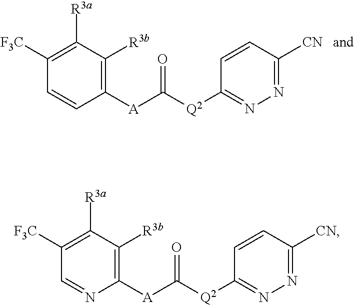

or a pharmaceutically acceptable salt thereof.

In yet a further aspect, the compound has a structure represented by a formula:

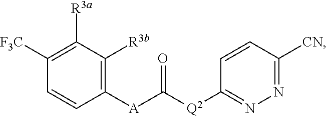

or a pharmaceutically acceptable salt thereof.

In an even further aspect, the compound has a structure represented by a formula selected from:

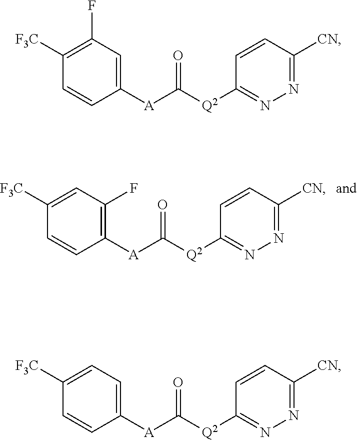

or a pharmaceutically acceptable salt thereof.

In a still further aspect, the compound has a structure represented by a formula:

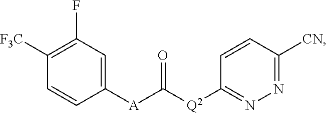

or a pharmaceutically acceptable salt thereof.

In yet a further aspect, the compound is:

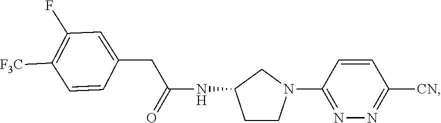 [SMILES: N#Cc1ccc(N2CC[C@H](NC(=O)Cc3ccc(C(F)(F)F)c(F)c3)C2)nn1]

or a pharmaceutically acceptable salt thereof.

In a further aspect, the compound is selected from:

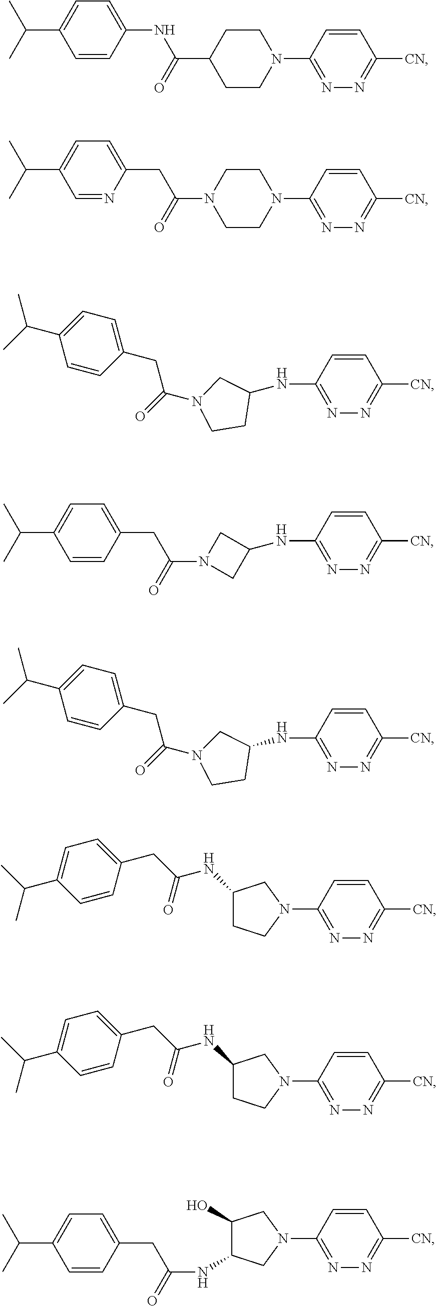

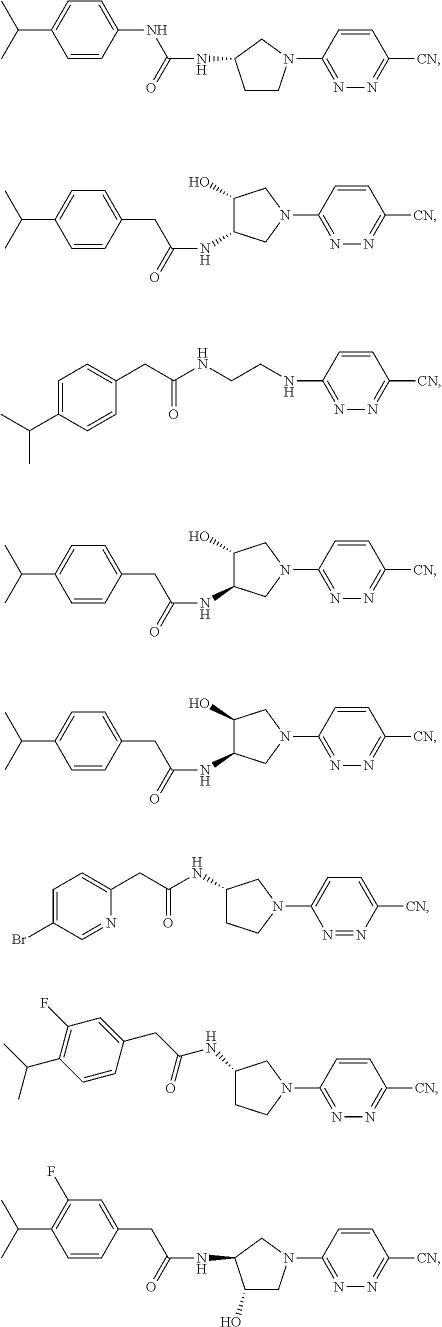

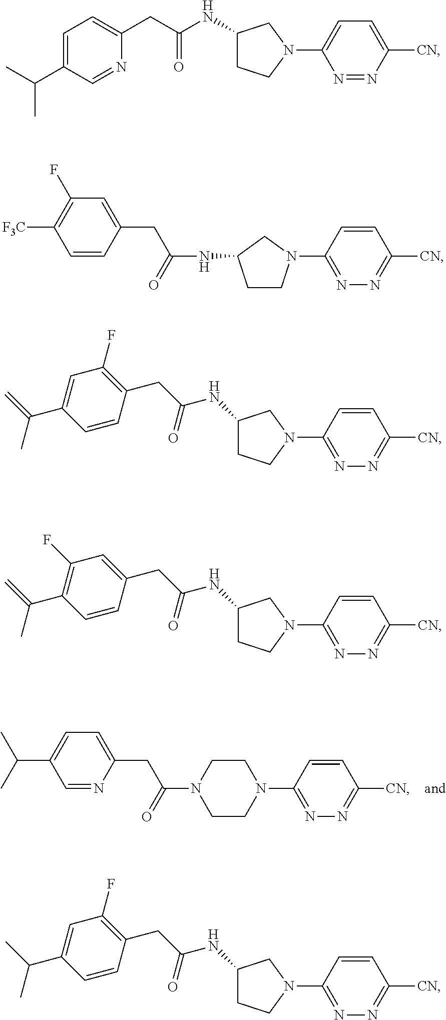 [SMILES: CC(C)c1ccc(CC(=O)N[C@H]2CCN(c3ccc(C#N)nn3)C2)nc1 ; N#Cc1ccc(N2CC[C@H](NC(=O)Cc3ccc(C(F)(F)F)c(F)c3)C2)nn1 ; C=C(C)c1ccc(CC(=O)N[C@H]2CCN(c3ccc(C#N)nn3)C2)c(F)c1 ; C=C(C)c1ccc(CC(=O)N[C@H]2CCN(c3ccc(C#N)nn3)C2)cc1F ; CC(C)c1ccc(CC(=O)N2CCN(c3ccc(C#N)nn3)CC2)nc1 ; CC(C)c1ccc(CC(=O)N[C@H]2CCN(c3ccc(C#N)nn3)C2)c(F)c1]

or a pharmaceutically acceptable salt thereof.

In a further aspect, the compound is selected from:

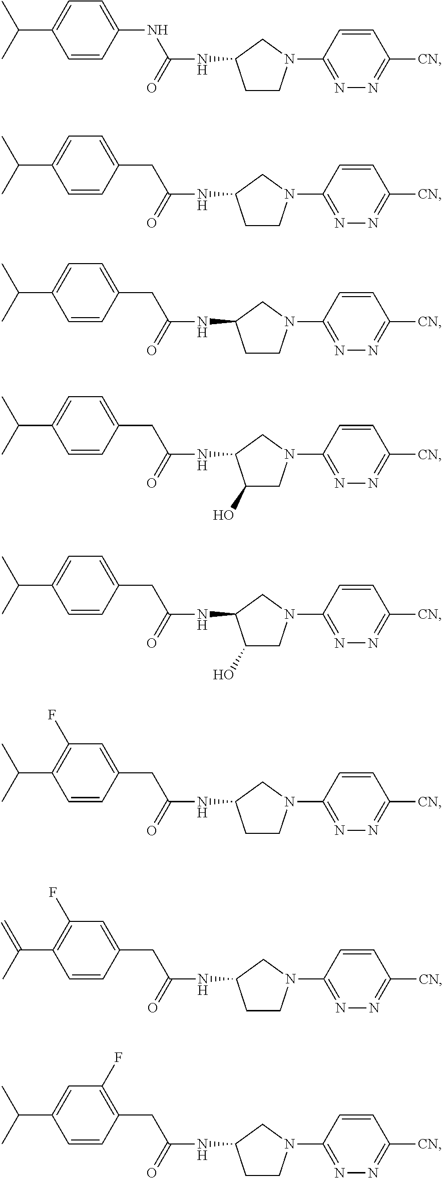

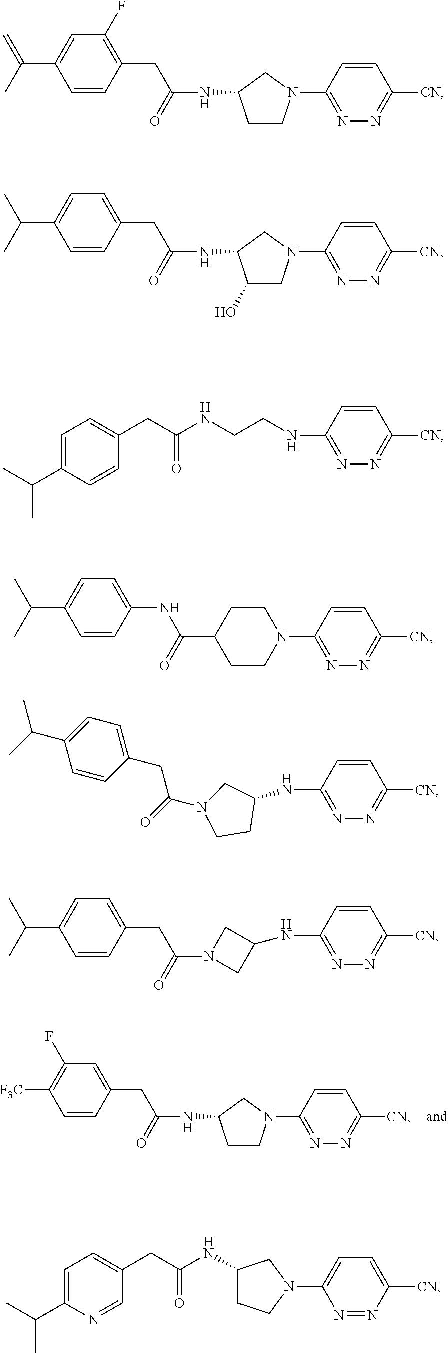

or a pharmaceutically acceptable salt thereof.

In a further aspect, the compound has a structure represented by a formula:

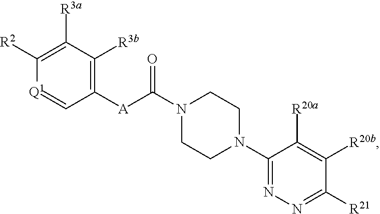

or a pharmaceutically acceptable salt thereof.

In a still further aspect, the compound has a structure represented by a formula:

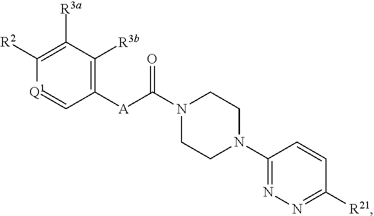

or a pharmaceutically acceptable salt thereof.

In yet a further aspect, the compound has a structure represented by a formula selected from:

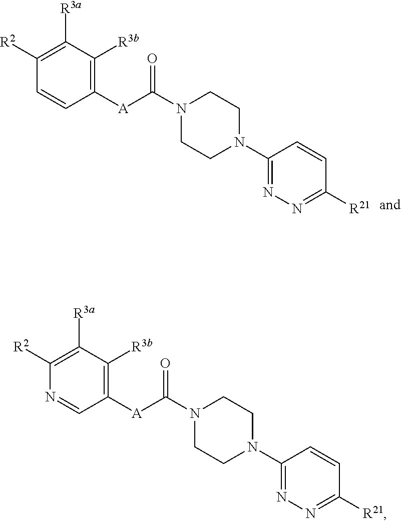

or a pharmaceutically acceptable salt thereof.

In an even further aspect, the compound has a structure represented by a formula:

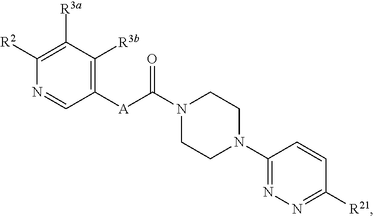

or a pharmaceutically acceptable salt thereof.

In a still further aspect, the compound has a structure represented by a formula:

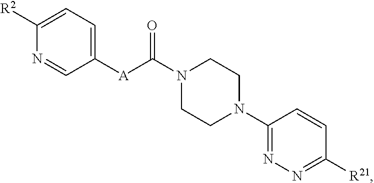

or a pharmaceutically acceptable salt thereof.

In yet a further aspect, the compound is selected from:

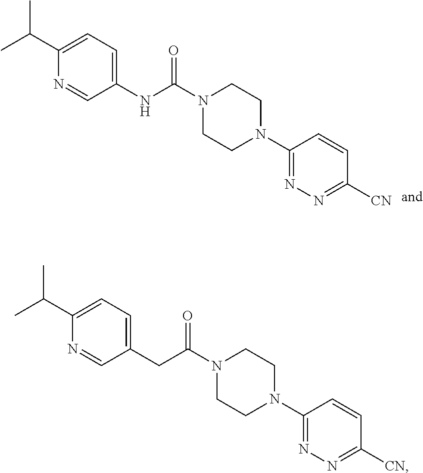 [SMILES: CC(C)c1ccc(NC(=O)N2CCN(c3ccc(C#N)nn3)CC2)cn1 ; CC(C)c1ccc(CC(=O)N2CCN(c3ccc(C#N)nn3)CC2)cn1]

or a pharmaceutically acceptable salt thereof.

In a further aspect, the compound has a structure represented by a formula:

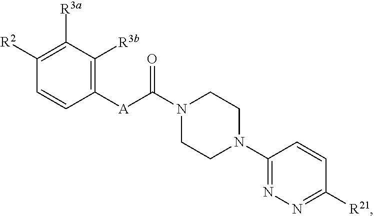

or a pharmaceutically acceptable salt thereof.

In a still further aspect, the compound has a structure represented by a formula:

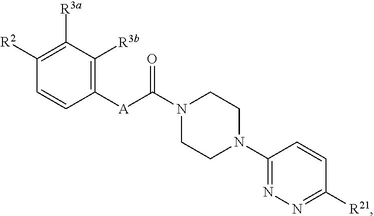

or a pharmaceutically acceptable salt thereof.

In yet a further aspect, the compound has a structure represented by a formula:

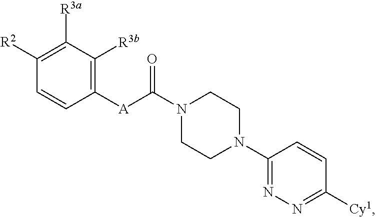

or a pharmaceutically acceptable salt thereof.

In an even further aspect, the compound is:

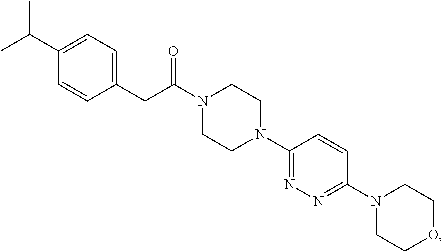 [SMILES: CC(C)c1ccc(CC(=O)N2CCN(c3ccc(N4CCOCC4)nn3)CC2)cc1]

or a pharmaceutically acceptable salt thereof.

In a further aspect, the compound has a structure represented by a formula:

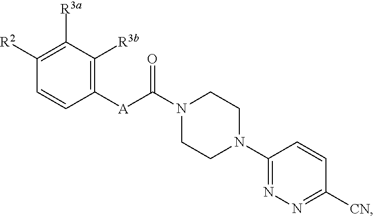

or a pharmaceutically acceptable salt thereof.

In a still further aspect, the compound has a structure represented by a formula:

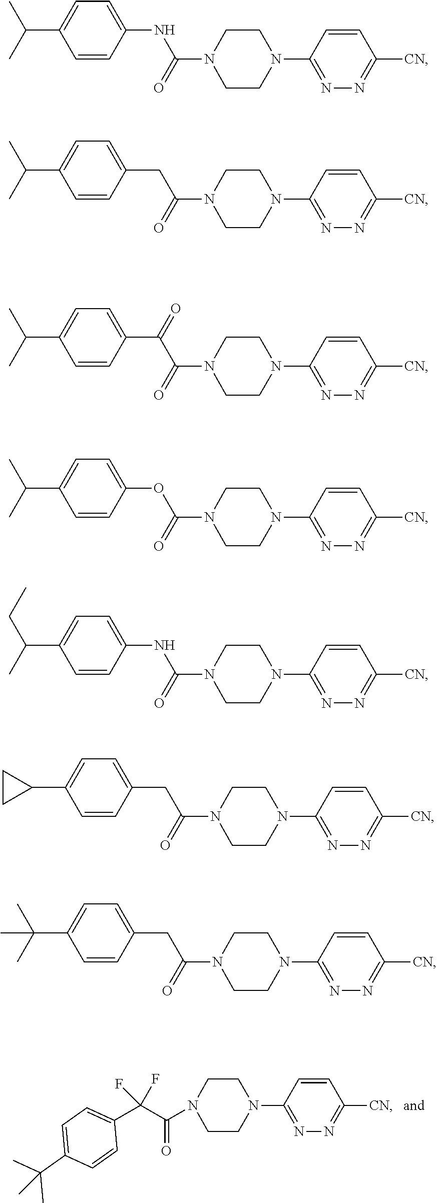

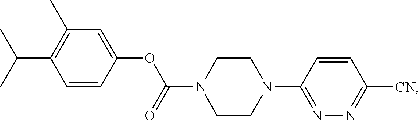 [SMILES: Cc1cc(OC(=O)N2CCN(c3ccc(C#N)nn3)CC2)ccc1C(C)C]

or a pharmaceutically acceptable salt thereof.

In a further aspect, the compound has a structure represented by a formula:

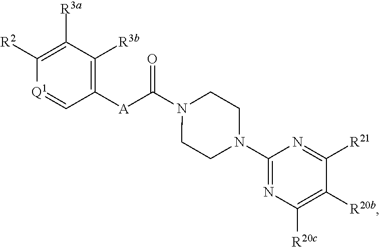

or a pharmaceutically acceptable salt thereof.

In a still further aspect, the compound has a structure represented by a formula:

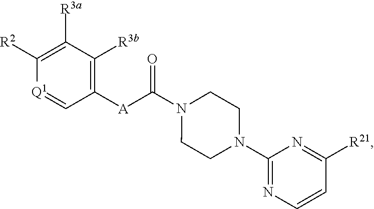

or a pharmaceutically acceptable salt thereof.

In yet a further aspect, the compound has a structure represented by a formula:

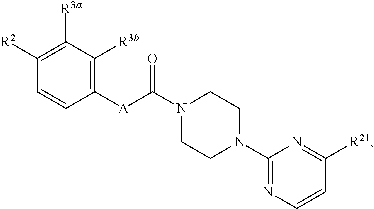

or a pharmaceutically acceptable salt thereof.

In an even further aspect, the compound is selected from:

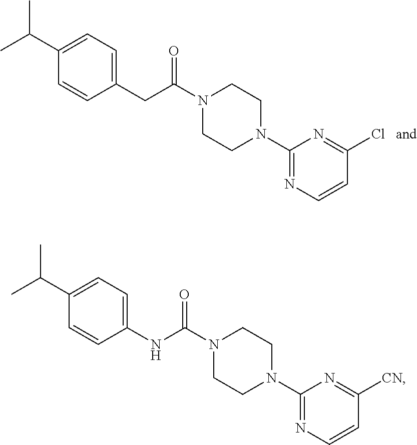 [SMILES: CC(C)c1ccc(CC(=O)N2CCN(c3nccc(Cl)n3)CC2)cc1 ; "and" ; CC(C)c1ccc(NC(=O)N2CCN(c3nccc(C#N)n3)CC2)cc1]

or a pharmaceutically acceptable salt thereof.

In a further aspect, the compound has a structure represented by a formula:

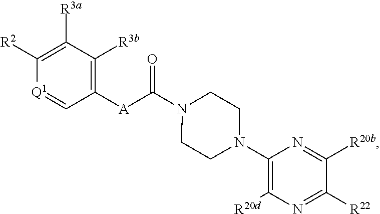

or a pharmaceutically acceptable salt thereof.

In a still further aspect, the compound has a structure represented by a formula:

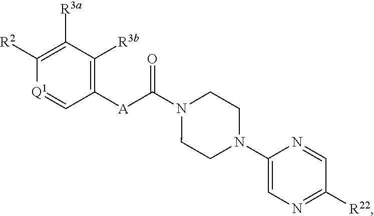

or a pharmaceutically acceptable salt thereof.

In yet a further aspect, the compound has a structure represented by a formula:

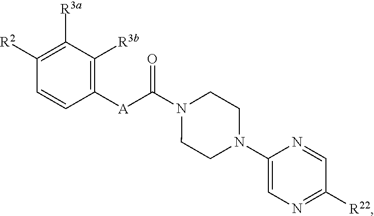

or a pharmaceutically acceptable salt thereof.

In an even further aspect, the compound is selected from:

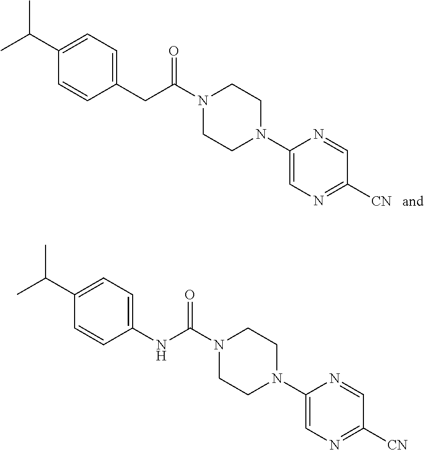 [SMILES: CC(C)c1ccc(CC(=O)N2CCN(c3cnc(C#N)cn3)CC2)cc1 ; "and" ; CC(C)c1ccc(NC(=O)N2CCN(c3cnc(C#N)cn3)CC2)cc1]

or a pharmaceutically acceptable salt thereof.

In a further aspect, the compound has a structure represented by a formula:

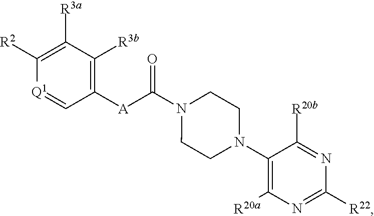

or a pharmaceutically acceptable salt thereof.

In a still further aspect, the compound has a structure represented by a formula:

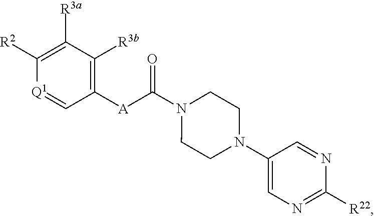

or a pharmaceutically acceptable salt thereof.

In yet a further aspect, the compound has a structure represented by a formula:

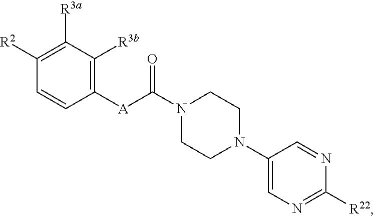

or a pharmaceutically acceptable salt thereof.

In an even further aspect, the compound has a structure represented by a formula:

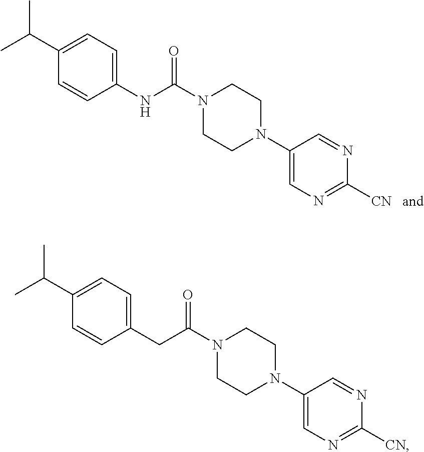 [SMILES: CC(C)c1ccc(NC(=O)N2CCN(c3cnc(C#N)nc3)CC2)cc1 ; "and" ; CC(C)c1ccc(CC(=O)N2CCN(c3cnc(C#N)nc3)CC2)cc1]

or a pharmaceutically acceptable salt thereof.

In a further aspect, the compound is selected from:

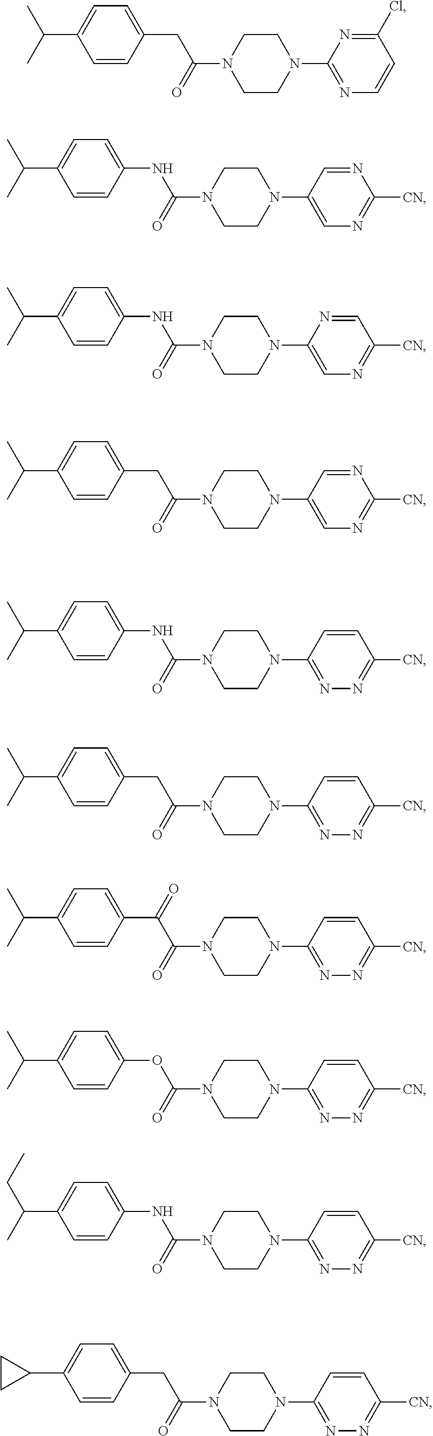

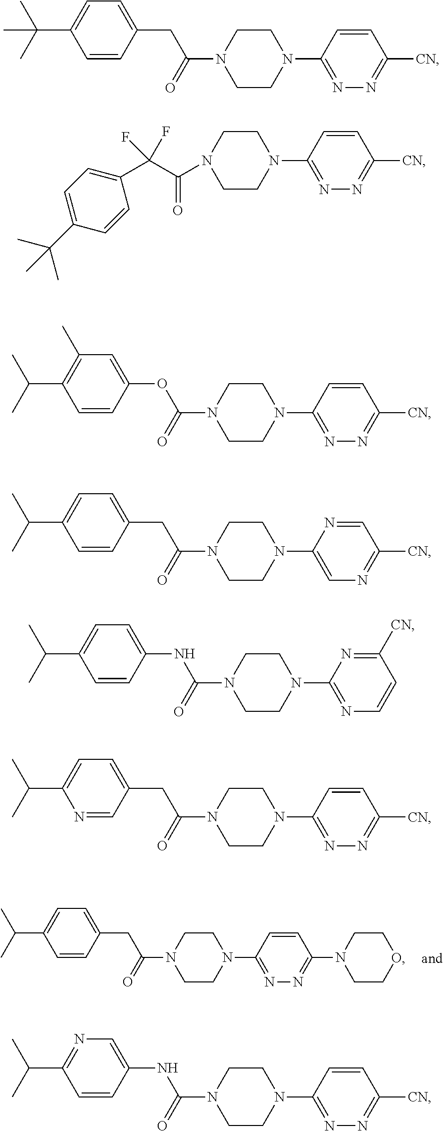

or a pharmaceutically acceptable salt thereof.

In a further aspect, the compound has a structure represented by a formula selected from:

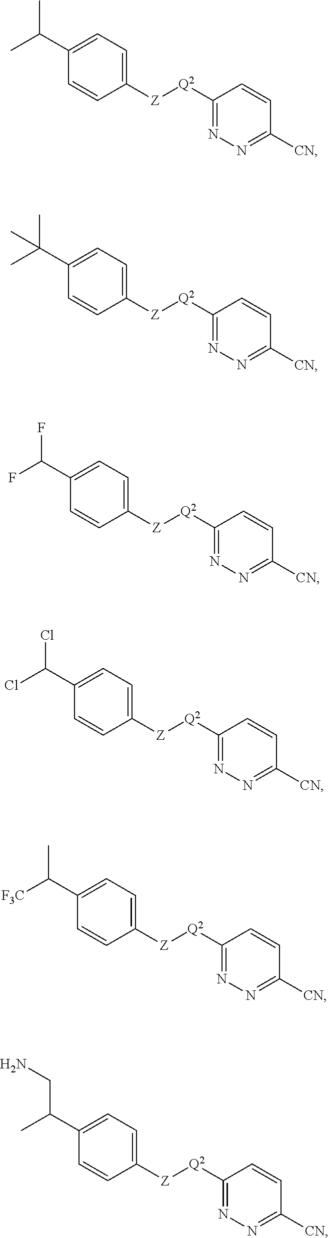

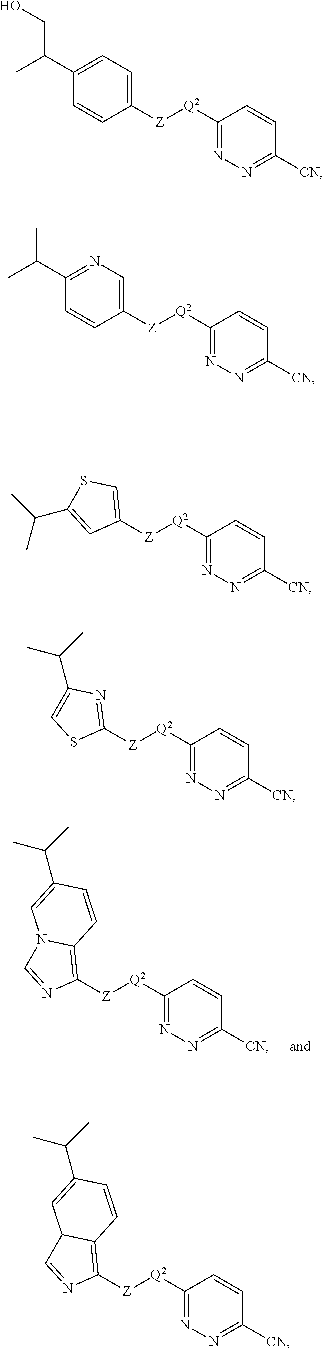

or a pharmaceutically acceptable salt thereof.

In a still further aspect, the compound has a structure represented by a formula selected from:

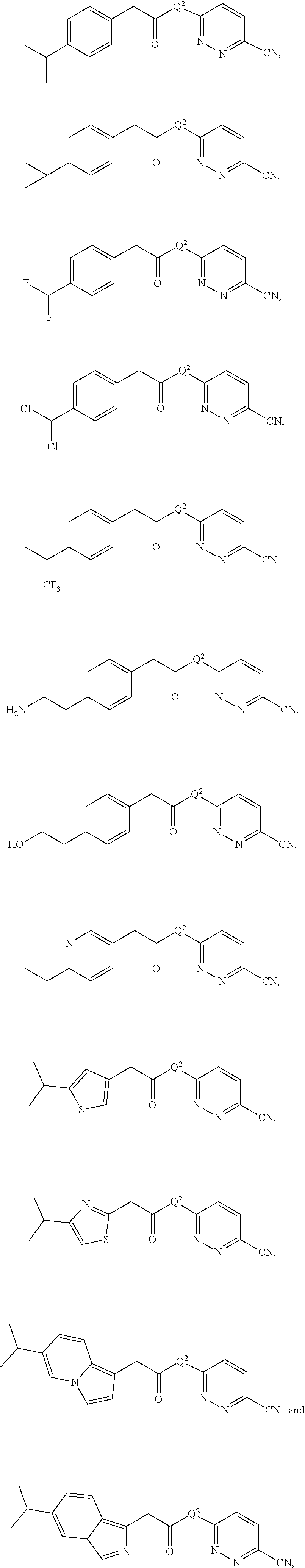

or a pharmaceutically acceptable salt thereof.

In yet a further aspect, the compound has a structure represented by a formula selected from:

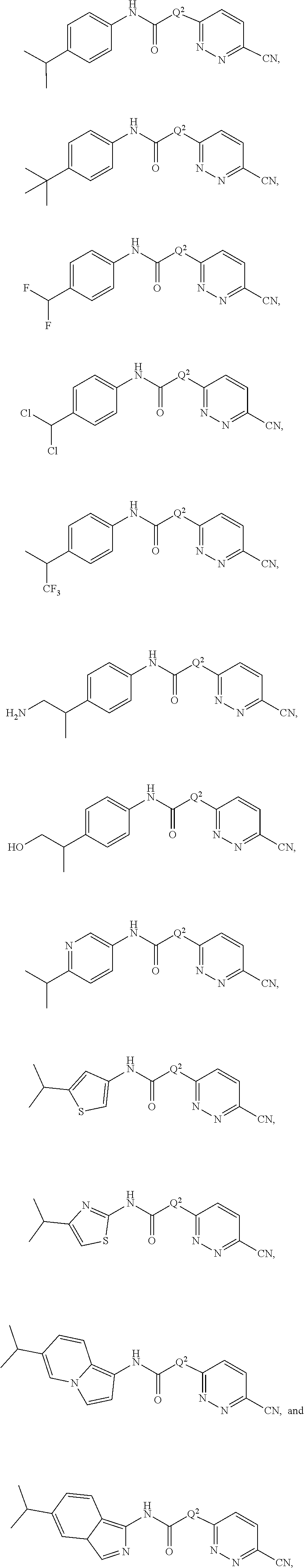

or a pharmaceutically acceptable salt thereof.

In an even further aspect, the compound has a structure represented by a formula selected from:

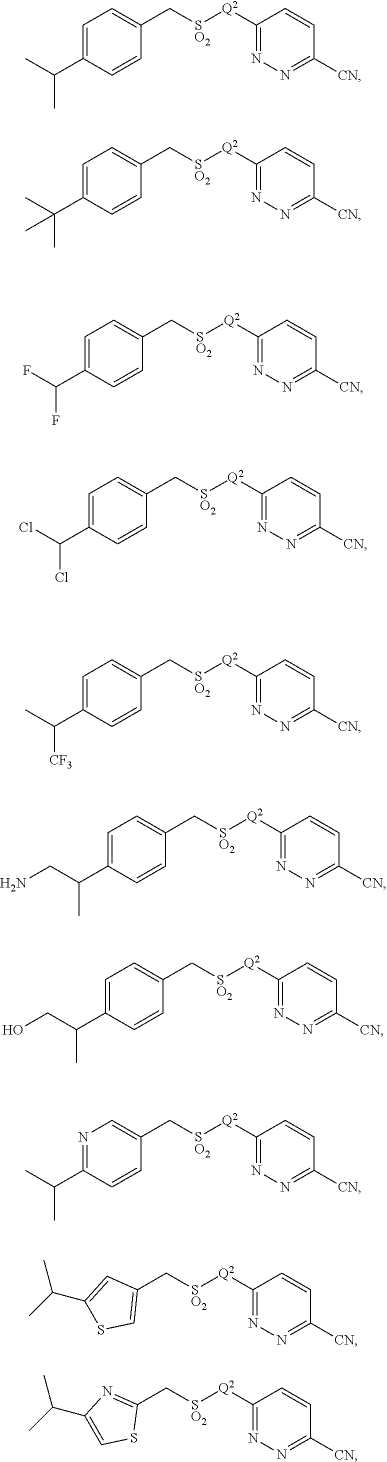

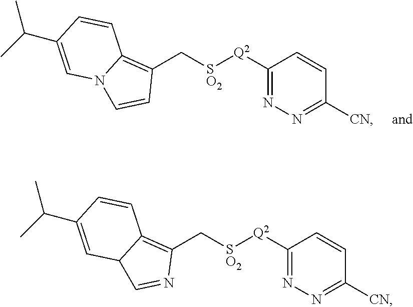

or a pharmaceutically acceptable salt thereof.

In a still further aspect, the compound has a structure represented by a formula selected from:

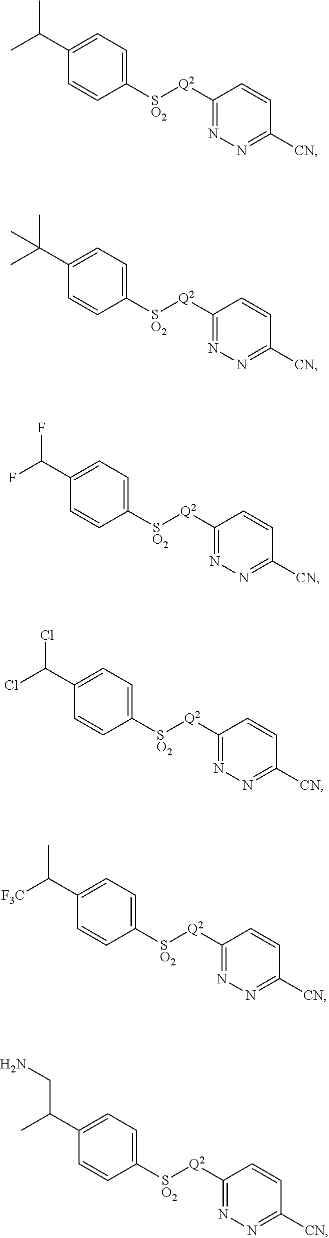

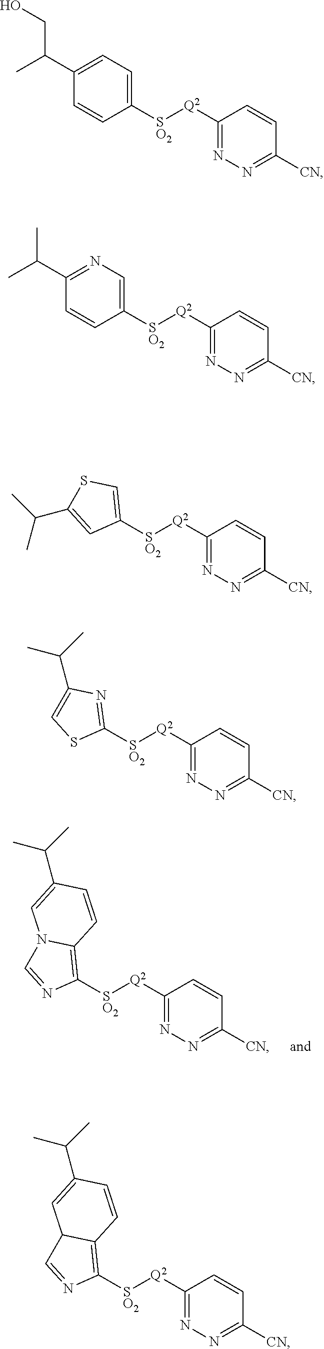

or a pharmaceutically acceptable salt thereof.

In yet a further aspect, the compound has a structure represented by a formula selected from:

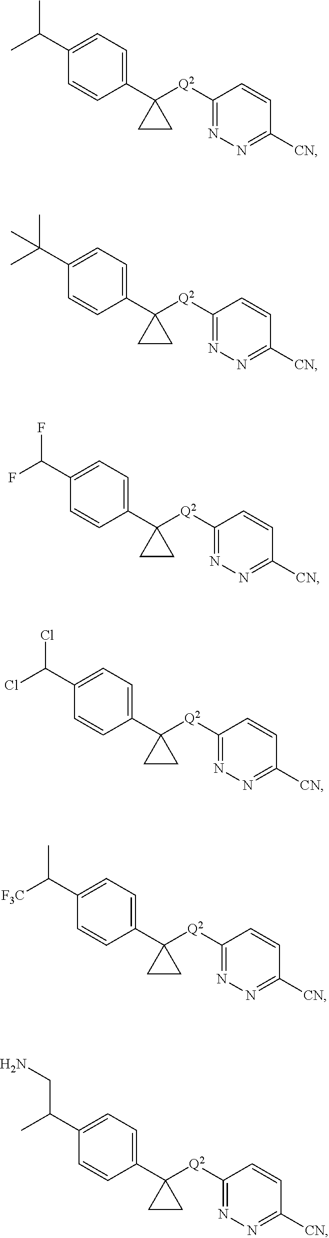

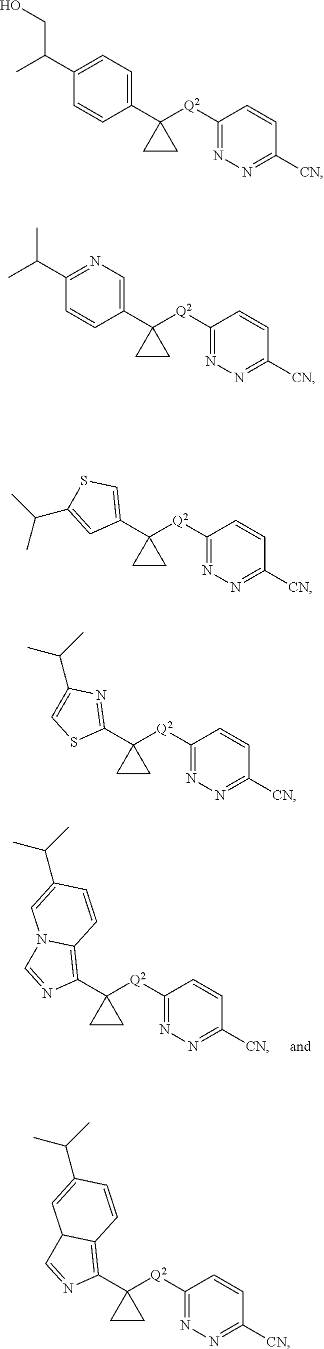

or a pharmaceutically acceptable salt thereof.

In an even further aspect, the compound has a structure represented by a formula selected from:

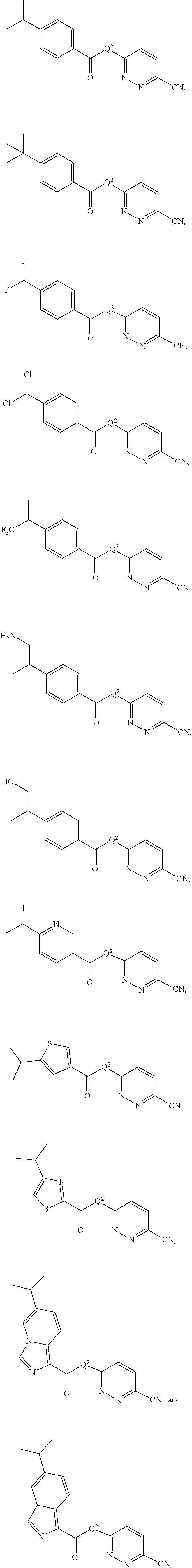

or a pharmaceutically acceptable salt thereof.

In a further aspect, the compound has a structure represented by a formula:

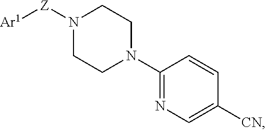

or a pharmaceutically acceptable salt thereof.

In a still further aspect, the compound has a structure represented by a formula selected from:

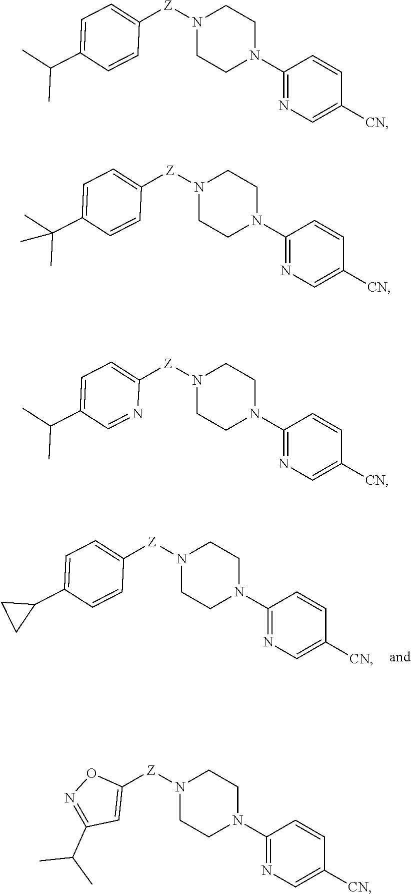

or a pharmaceutically acceptable salt thereof.

In yet a further aspect, the compound is selected from:

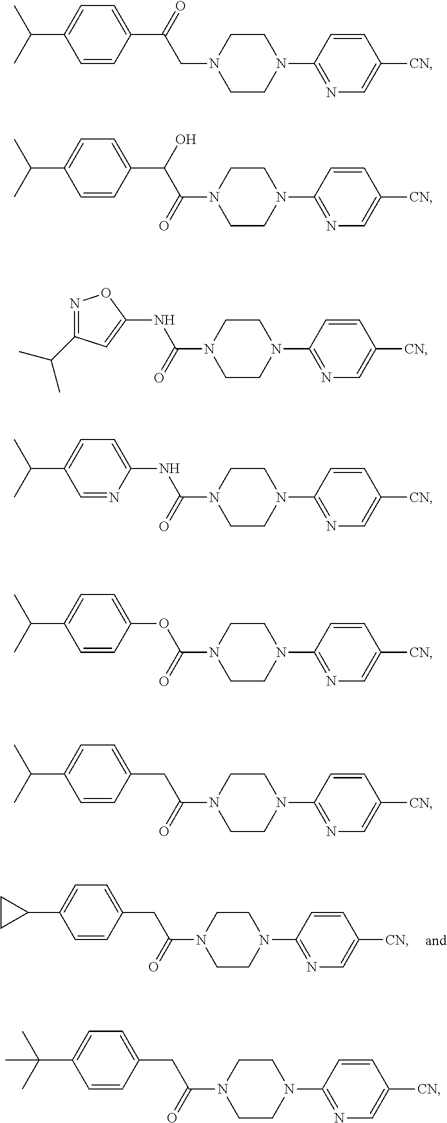

or a pharmaceutically acceptable salt thereof.

In a further aspect, the compound has a structure represented by a formula selected from:

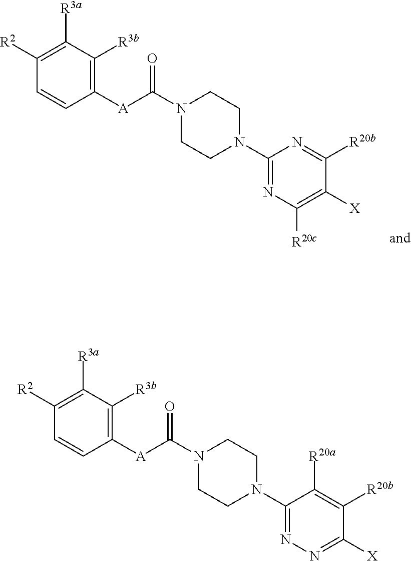

or a pharmaceutically acceptable salt thereof.

In a still further aspect, the compound has a structure represented by a formula selected from:

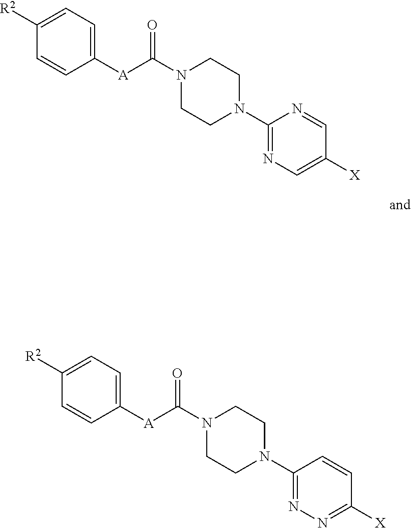

or a pharmaceutically acceptable salt thereof.

In yet a further aspect, the compound is selected from:

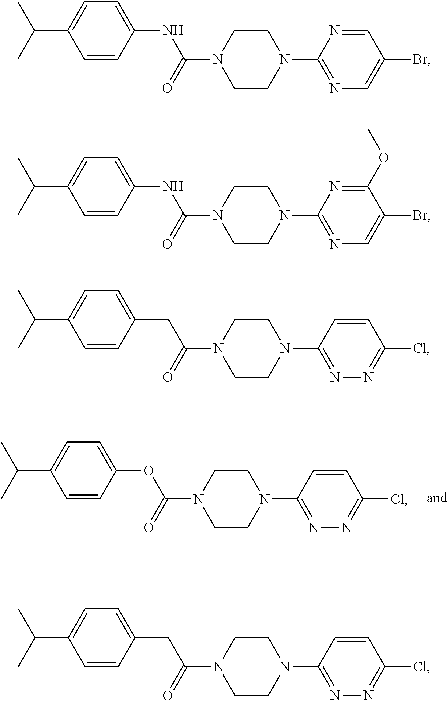

or a pharmaceutically acceptable salt thereof.

In a further aspect, the compound is selected from:

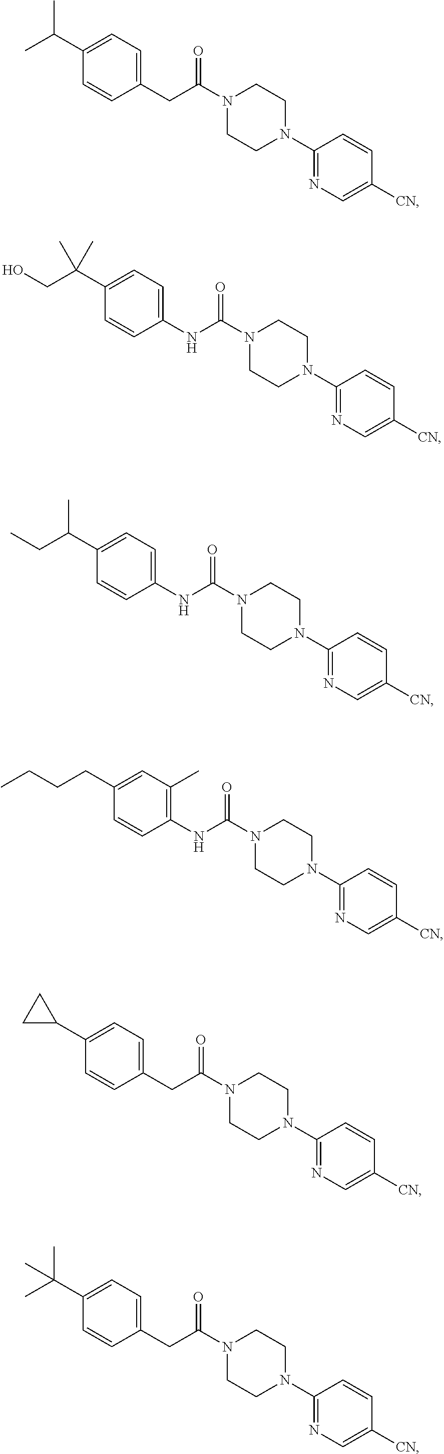 [SMILES: CC(C)c1ccc(CC(=O)N2CCN(c3ccc(C#N)cn3)CC2)cc1 ; CC(C)(CO)c1ccc(NC(=O)N2CCN(c3ccc(C#N)cn3)CC2)cc1 ; CCC(C)c1ccc(NC(=O)N2CCN(c3ccc(C#N)cn3)CC2)cc1 ; CCCCc1ccc(NC(=O)N2CCN(c3ccc(C#N)cn3)CC2)c(C)c1 ; N#Cc1ccc(N2CCN(C(=O)Cc3ccc(C4CC4)cc3)CC2)nc1 ; CC(C)(C)c1ccc(CC(=O)N2CCN(c3ccc(C#N)cn3)CC2)cc1]

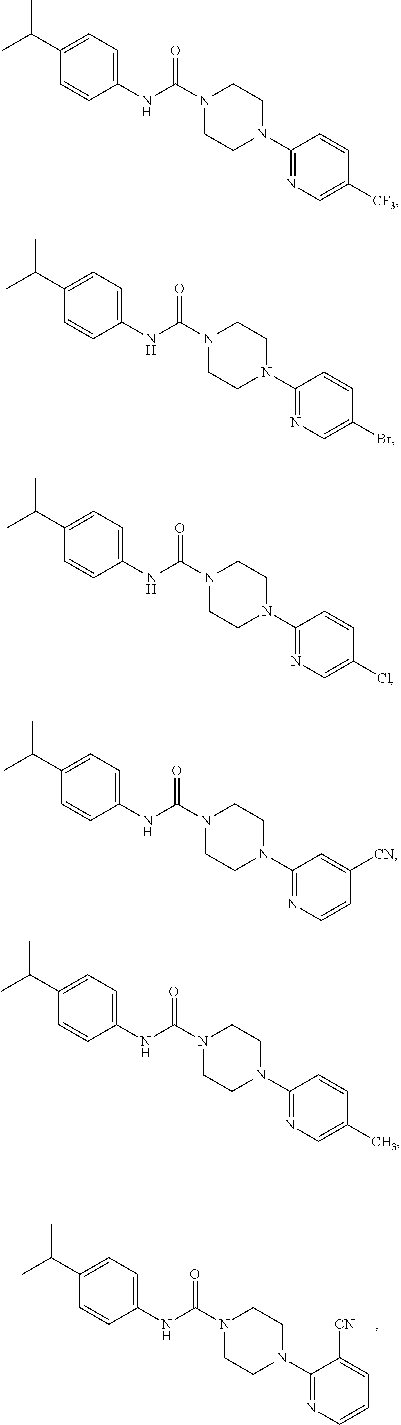 [SMILES: CC(C)c1ccc(NC(=O)N2CCN(c3ccc(C(F)(F)F)cn3)CC2)cc1 ; CC(C)c1ccc(NC(=O)N2CCN(c3ccc(Br)cn3)CC2)cc1 ; CC(C)c1ccc(NC(=O)N2CCN(c3ccc(Cl)cn3)CC2)cc1 ; CC(C)c1ccc(NC(=O)N2CCN(c3cc(C#N)ccn3)CC2)cc1 ; Cc1ccc(N2CCN(C(=O)Nc3ccc(C(C)C)cc3)CC2)nc1 ; CC(C)c1ccc(NC(=O)N2CCN(c3ncccc3C#N)CC2)cc1]

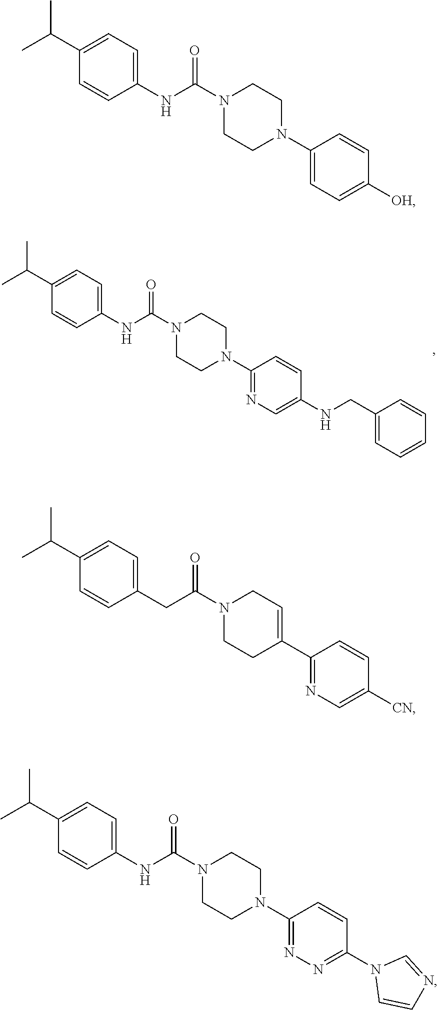 [SMILES: CC(C)c1ccc(NC(=O)N2CCN(c3ccc(O)cc3)CC2)cc1 ; CC(C)c1ccc(NC(=O)N2CCN(c3ccc(NCc4ccccc4)cn3)CC2)cc1 ; CC(C)c1ccc(CC(=O)N2CC=C(c3ccc(C#N)cn3)CC2)cc1 ; CC(C)c1ccc(NC(=O)N2CCN(c3ccc(-n4ccnc4)nn3)CC2)cc1]

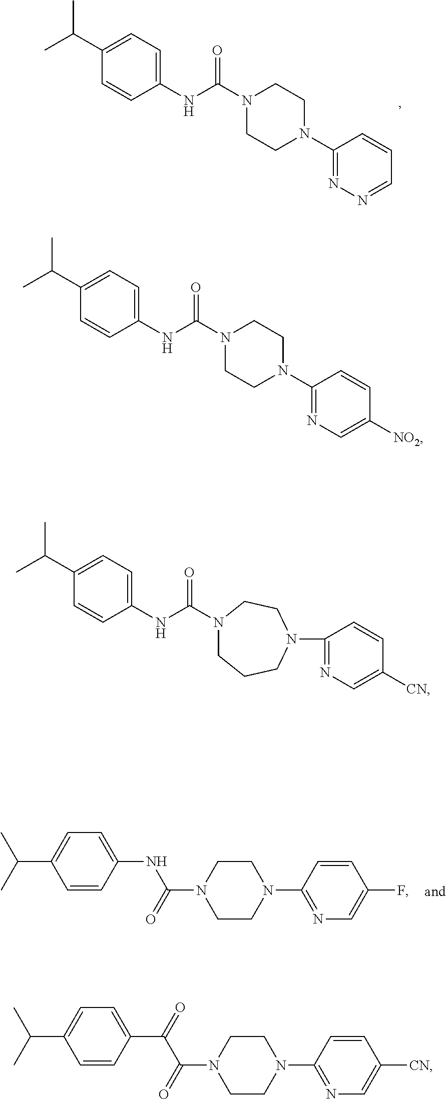 [SMILES: CC(C)c1ccc(NC(=O)N2CCN(c3cccnn3)CC2)cc1 ; CC(C)c1ccc(NC(=O)N2CCN(c3ccc([N+](=O)[O-])cn3)CC2)cc1 ; CC(C)c1ccc(NC(=O)N2CCCN(c3ccc(C#N)cn3)CC2)cc1 ; CC(C)c1ccc(NC(=O)N2CCN(c3ccc(F)cn3)CC2)cc1 ; CC(C)c1ccc(C(=O)C(=O)N2CCN(c3ccc(C#N)cn3)CC2)cc1]

or a pharmaceutically acceptable salt thereof.

In a further aspect, the compound is selected from:

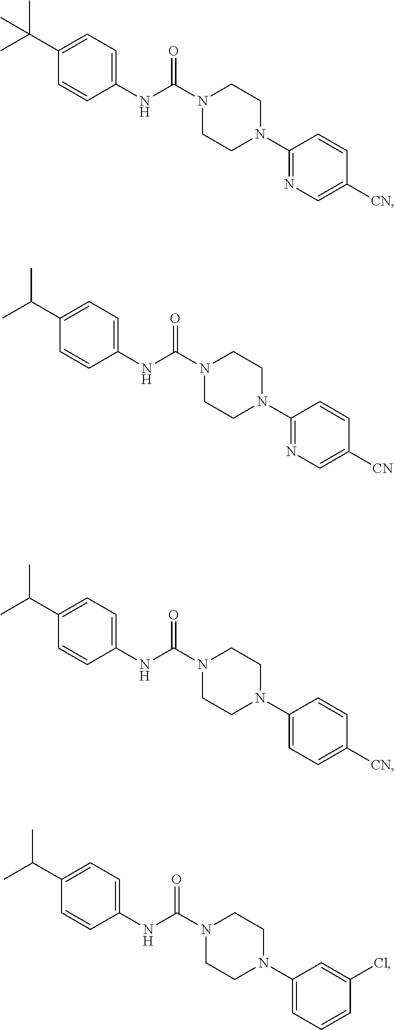 [SMILES: CC(C)(C)c1ccc(NC(=O)N2CCN(c3ccc(C#N)cn3)CC2)cc1 ; CC(C)c1ccc(NC(=O)N2CCN(c3ccc(C#N)cn3)CC2)cc1 ; CC(C)c1ccc(NC(=O)N2CCN(c3ccc(C#N)cc3)CC2)cc1 ; CC(C)c1ccc(NC(=O)N2CCN(c3cccc(Cl)c3)CC2)cc1]

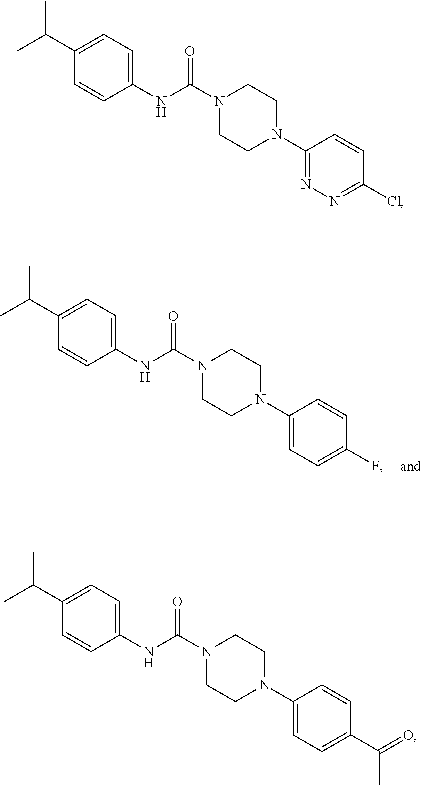 [SMILES: CC(C)c1ccc(NC(=O)N2CCN(c3ccc(Cl)nn3)CC2)cc1 ; CC(C)c1ccc(NC(=O)N2CCN(c3ccc(F)cc3)CC2)cc1 ; CC(=O)c1ccc(N2CCN(C(=O)Nc3ccc(C(C)C)cc3)CC2)cc1]

or a pharmaceutically acceptable salt thereof.

In a further aspect, the compound has a structure represented by a formula selected from:

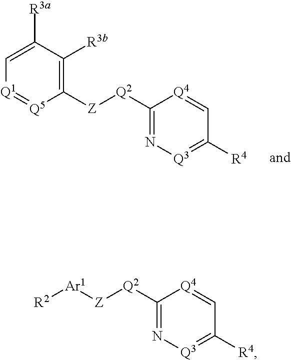

wherein Z is selected from A(C═O), C(O)CH₂, C(O), CH₂SO₂, and SO₂; wherein A is selected from O, CH₂, CF₂, NH, N(CH₃), and CH(OH); wherein each of Q¹ and Q⁵, when present, is independently selected from N and CH; wherein Q³ is N and Q⁴ is CH or wherein Q⁴ is N and Q³ is CH; wherein Q² is a structure selected from:

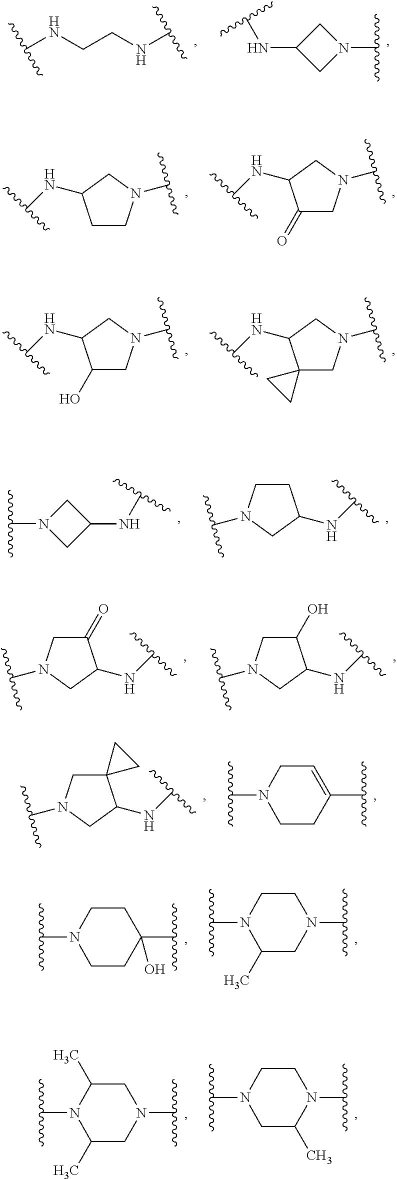

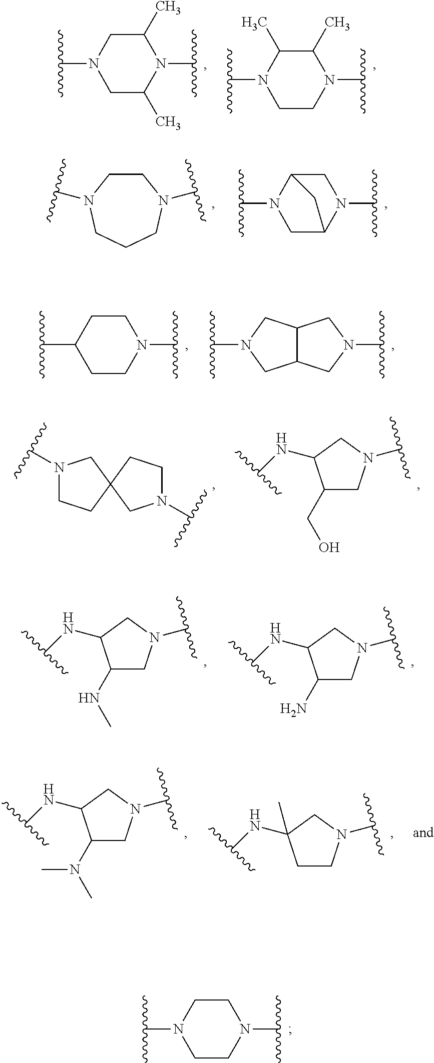

wherein R², when present, is selected from C1-C8 hydroxyalkyl, C1-C8 alkoxy, and cyclopropyl substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl), provided that cyclopropyl, when present, is substituted with at least one halogen group; wherein each of R^{3a} and R^{3b}, when present, is independently selected from hydrogen, halogen, —OH, C1-C4 alkyl, C1-C4 thioalkyl, and C1-C4 alkoxy; wherein R⁴ is selected from hydrogen, halogen, —CN, —NO₂, —SO₂NH₂, and —SO₂CH₃; and wherein Ar¹, when present, is selected from aryl and heteroaryl and is substituted with 0, 1, 2, or 3 groups independently selected from halogen, —NO₂, —CN, —OH, —SH, —NH₂, C1-C8 acyclic alkyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, and —CO(C1-C8 acyclic alkyl), provided that when R³ is C1-C8 hydroxy or C1-C8 alkoxy then R² is not hydrogen, or a pharmaceutically acceptable salt thereof.

In a further aspect, each of Q¹ and Q⁵ is CH.

In a further aspect, Q³ is N and Q⁴ is CH.

In a further aspect, Q² is a structure:

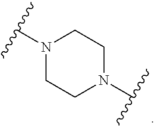 [SMILES: *N1CCN(*)CC1]

In a further aspect, R² is cyclopropyl substituted with 1, 2, or 3 groups independently selected from halogen and C1-C4 acyclic alkyl, provided that cyclopropyl is substituted with at least one halogen group. In a still further aspect, R² is a structure selected from:

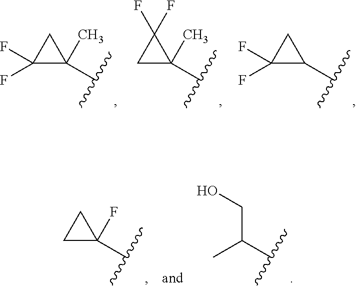

In a further aspect, each of R^{3a} and R^{3b} is hydrogen.

In a further aspect, R⁴ is CN.

In a further aspect, Ar¹ is a structure:

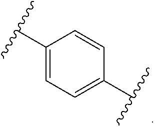 [SMILES: *c1ccc(*)cc1]

In a further aspect, the compound has a structure represented by a formula selected from:

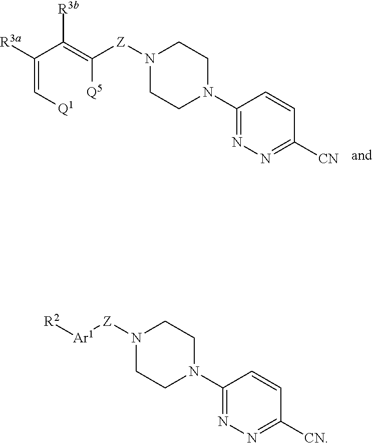

In a further aspect, the compound is selected from:

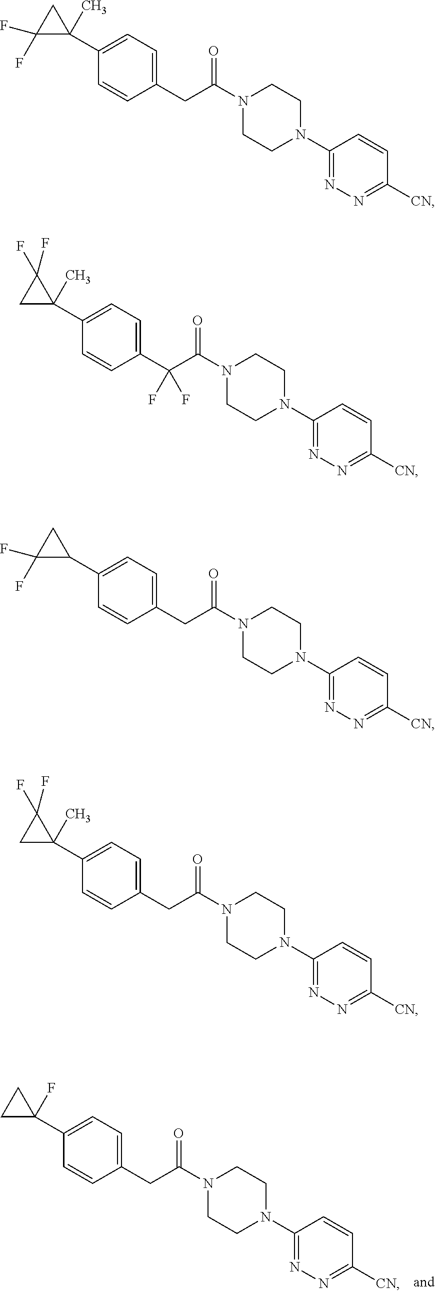 [SMILES: CC1(c2ccc(CC(=O)N3CCN(c4ccc(C#N)nn4)CC3)cc2)CC1(F)F ; CC1(c2ccc(C(F)(F)C(=O)N3CCN(c4ccc(C#N)nn4)CC3)cc2)CC1(F)F ; N#Cc1ccc(N2CCN(C(=O)Cc3ccc(C4CC4(F)F)cc3)CC2)nn1 ; CC1(c2ccc(CC(=O)N3CCN(c4ccc(C#N)nn4)CC3)cc2)CC1(F)F ; N#Cc1ccc(N2CCN(C(=O)Cc3ccc(C4(F)CC4)cc3)CC2)nn1]

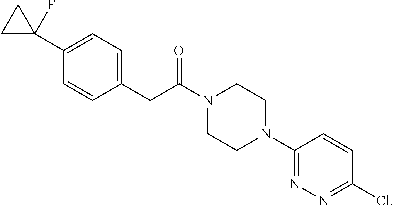 [SMILES: O=C(Cc1ccc(C2(F)CC2)cc1)N1CCN(c2ccc(Cl)nn2)CC1]

In a further aspect, the compound is.

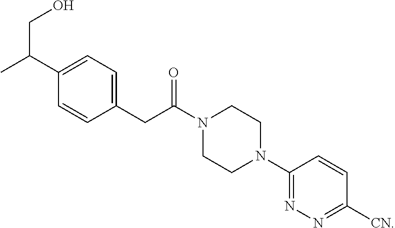 [SMILES: CC(CO)c1ccc(CC(=O)N2CCN(c3ccc(C#N)nn3)CC2)cc1]

a. A Groups

In one aspect, A is selected from O, CO, CH₂, CF₂, NH, N(CH₃), and CH(OH). In one aspect, A is selected from O, CO, CH₂, CF₂, NH, and CH(OH). In one aspect, O, CO, CH₂, CF₂, N(CH₃), and CH(OH). In one aspect, A is selected from O, CO, CH₂, CF₂, and CH(OH).

In a further aspect, A is selected from O, CO, CH₂, and CF₂. In a still further aspect, A is selected from O, CO, and CH₂. In yet a further aspect, A is selected from O and CO. In an even further aspect, A is O. In a still further aspect, A is CO. In yet a further aspect, A is CH₂.

In an even further aspect, A is CF₂.

In a further aspect, A is selected from NH and N(CH₃). In a still further aspect, A is NH. In yet a further aspect, A is N(CH₃).

In a further aspect, A is selected from NH and CH₂.

In a further aspect, A is CH(OH).

b. Q¹ and Q⁵ Groups

In one aspect, Q¹ is selected from N and CH. In one aspect, Q¹ is N. In one aspect, Q¹ is CH.

In one aspect, each of Q¹ and Q⁵, when present, is independently selected from N and CH. In a further aspect, each of Q¹ and Q⁵, when present, is N. In a still further aspect, each of Q¹ and Q⁵, when present, is CH. In yet a further aspect, Q¹, when present, is N and Q⁵, when present, is CH. In an even further aspect, Q¹, when present, is CH and Q⁵, when present, is N.

c. Q² Groups

In one aspect, Q² is a structure selected from:

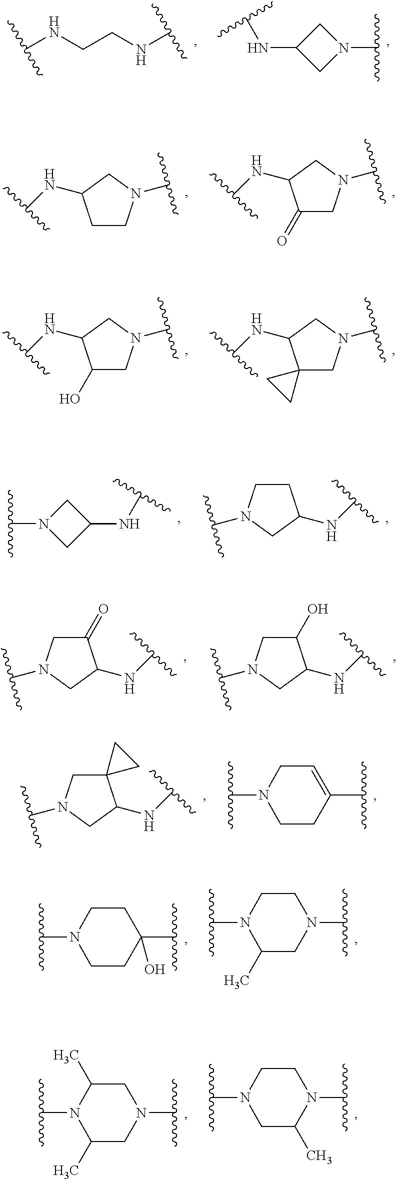

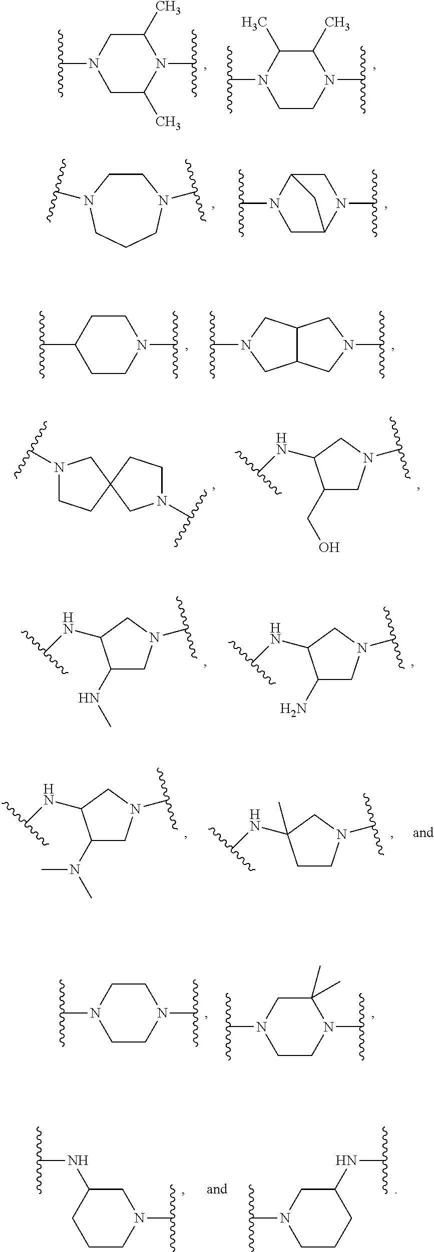

In one aspect, Q² is a structure selected from:

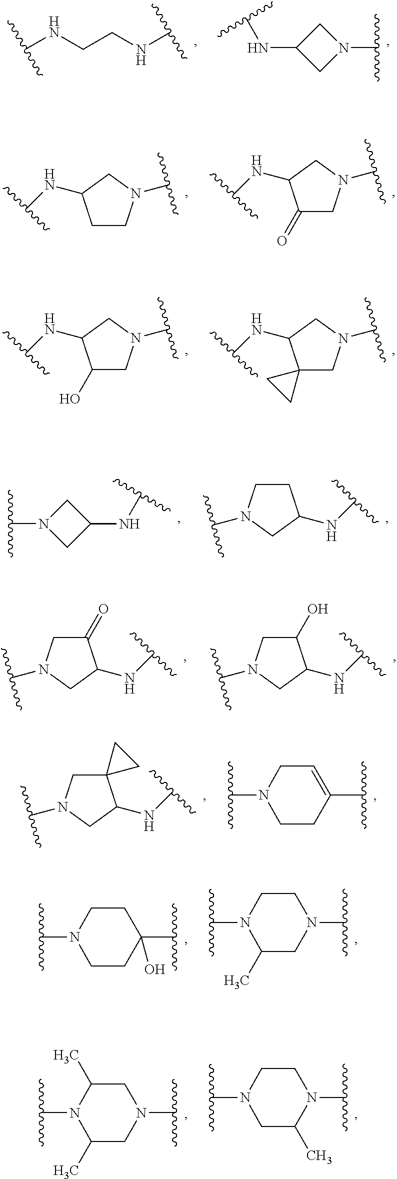

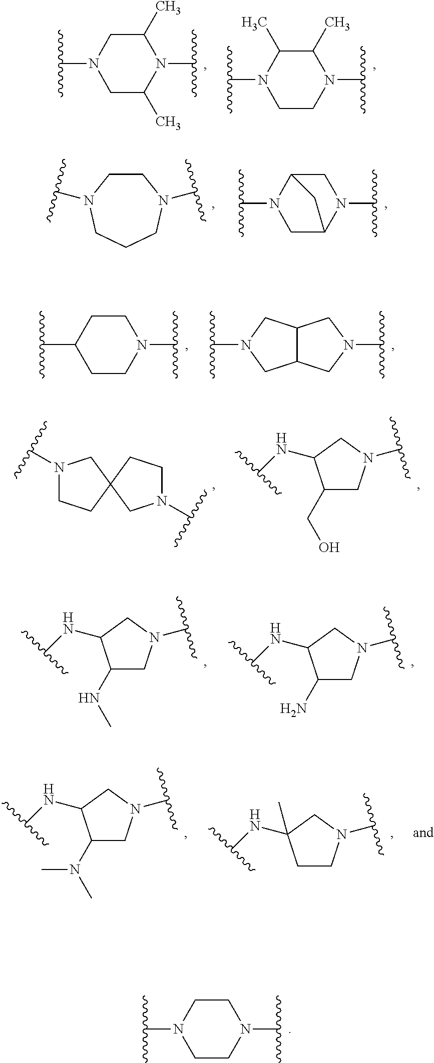

In one aspect, Q² is a structure selected from:

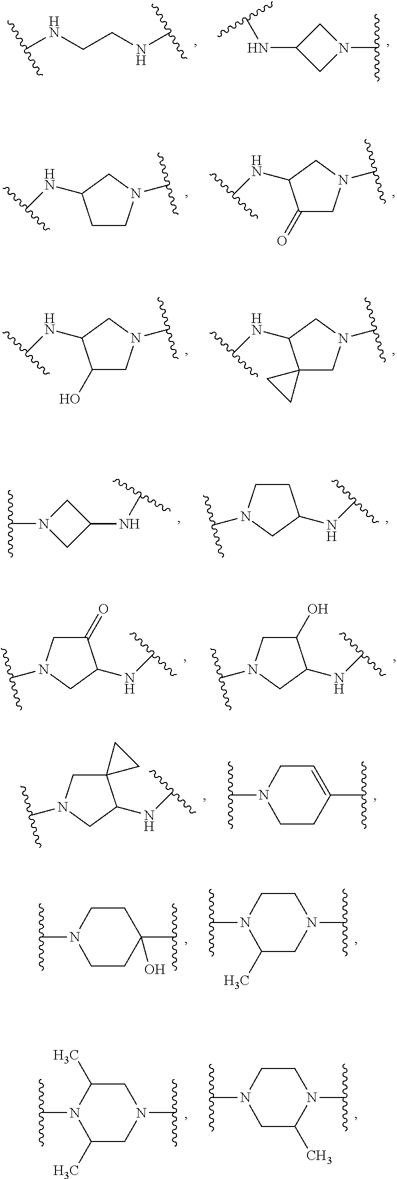

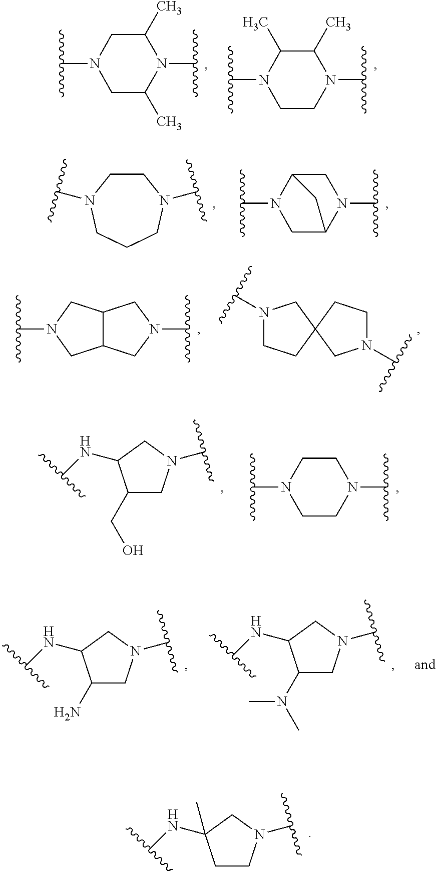

In one aspect, Q² is a structure selected from:

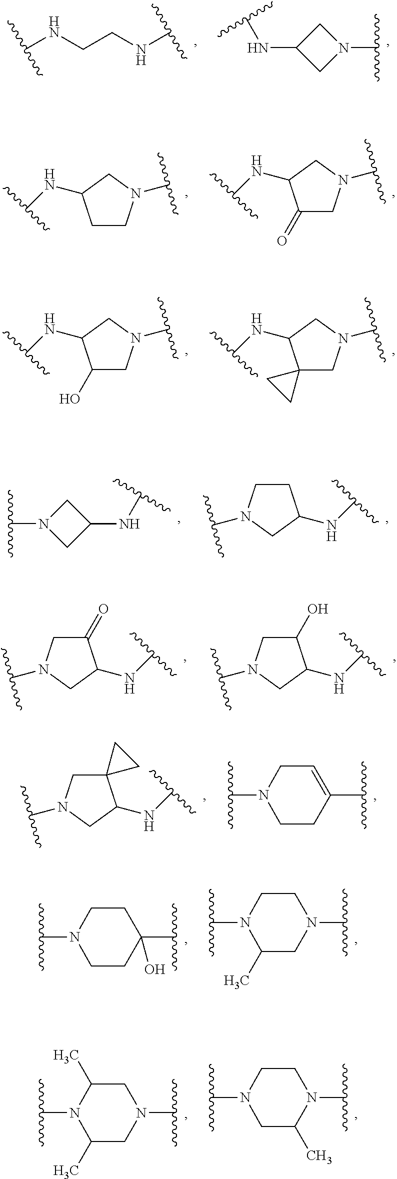

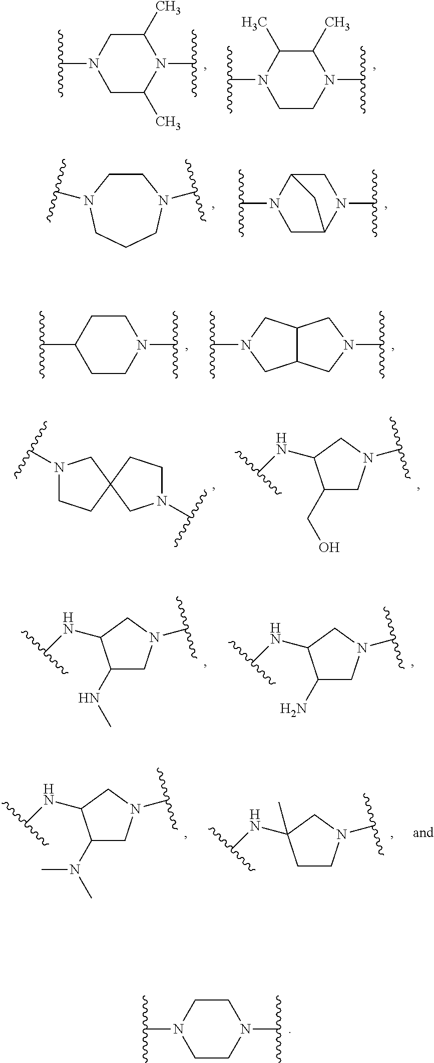

In a further aspect, Q² is a structure selected from:

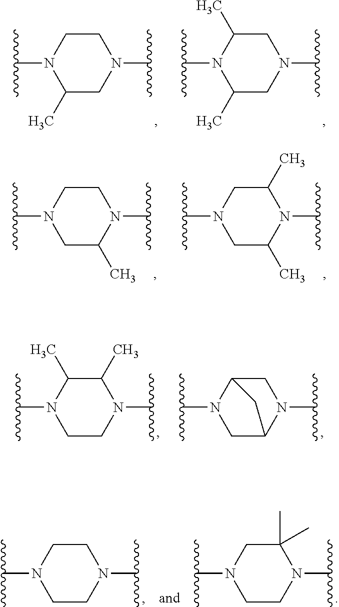

In a further aspect, Q² is a structure selected from:

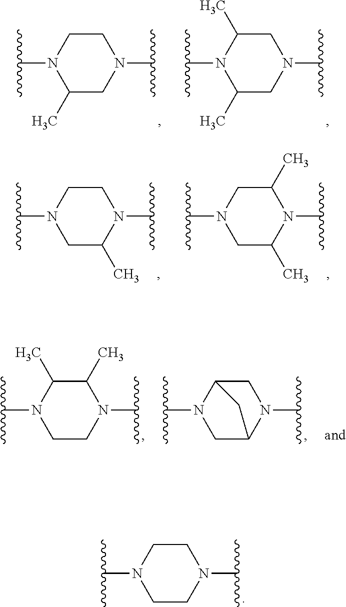

In a further aspect, Q² is a structure selected from:

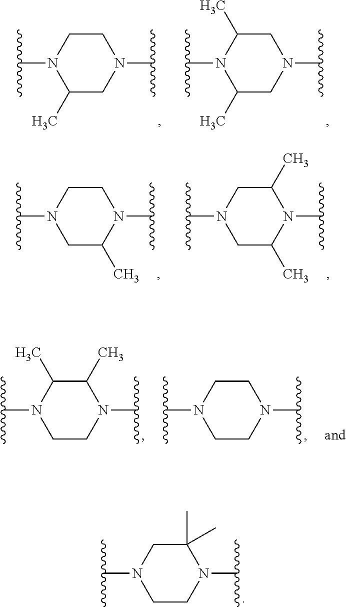

In a further aspect, Q² is a structure selected from:

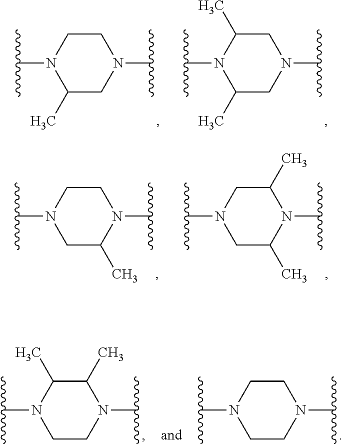

In a further aspect, Q² is a structure:

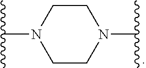 [SMILES: *N1CCN(*)CC1]

In a further aspect, Q² is a structure selected from:

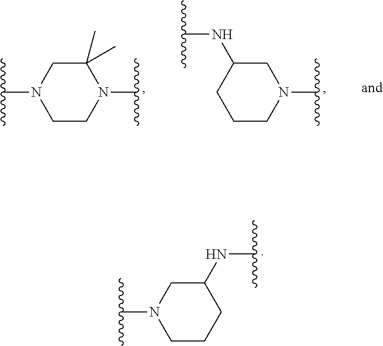

In a further aspect, Q² is a structure selected from:

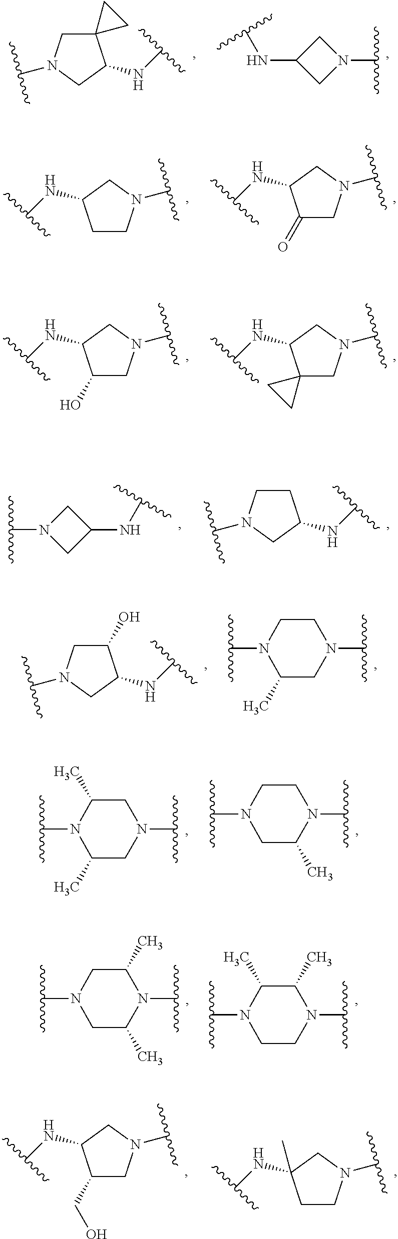

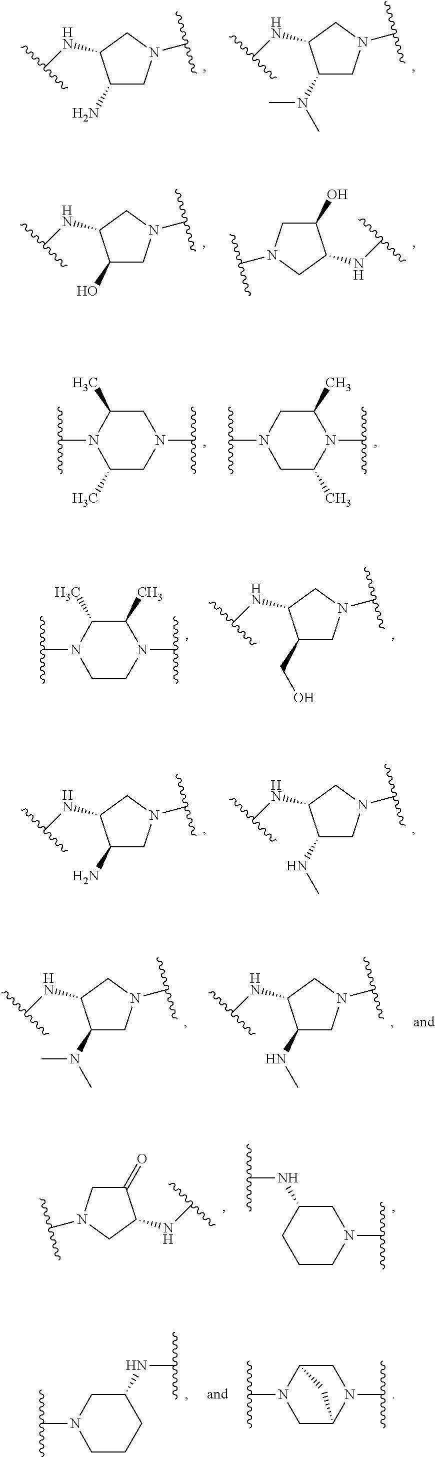

In a further aspect, wherein Q² is a structure selected from:

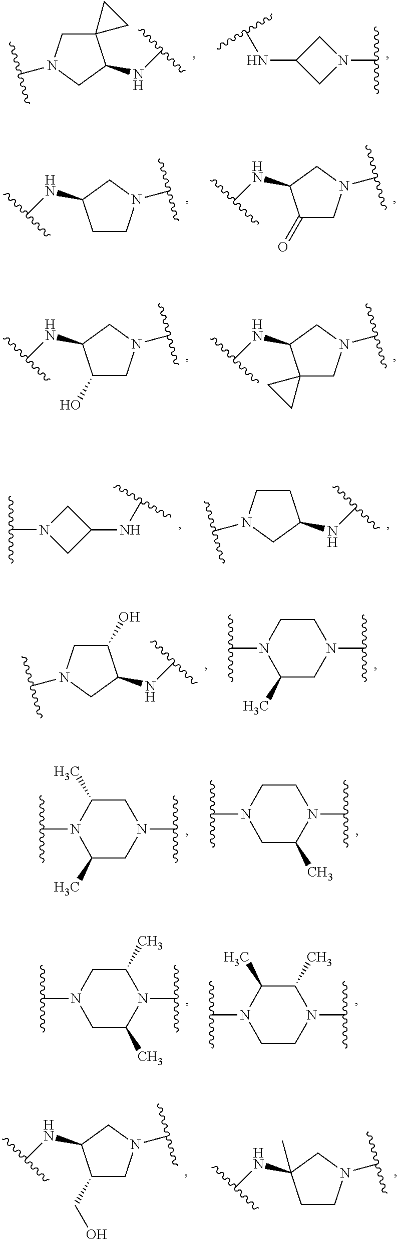

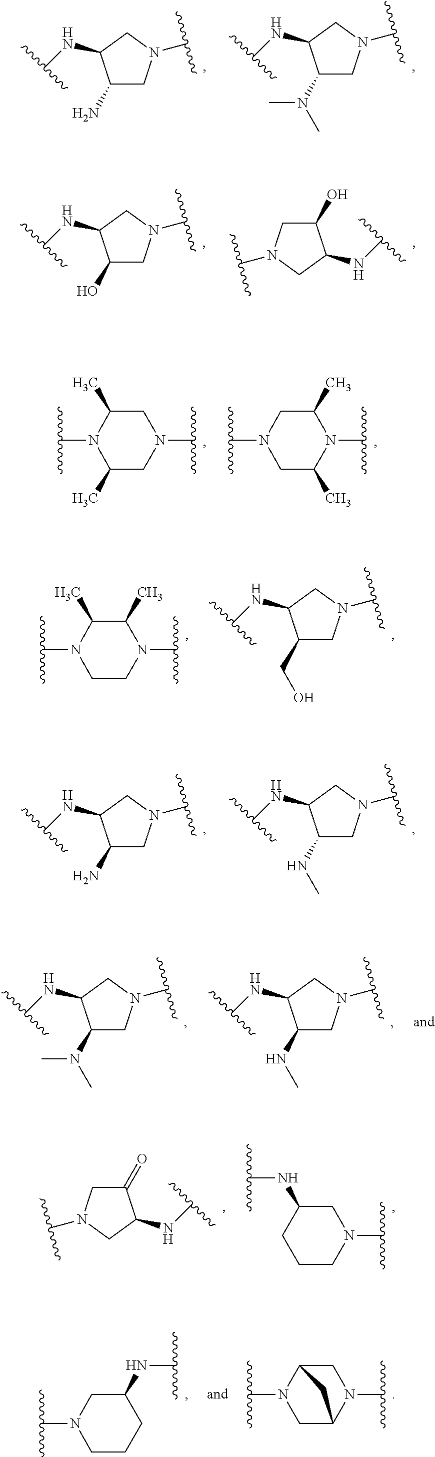

In a further aspect, Q² is a structure selected from:

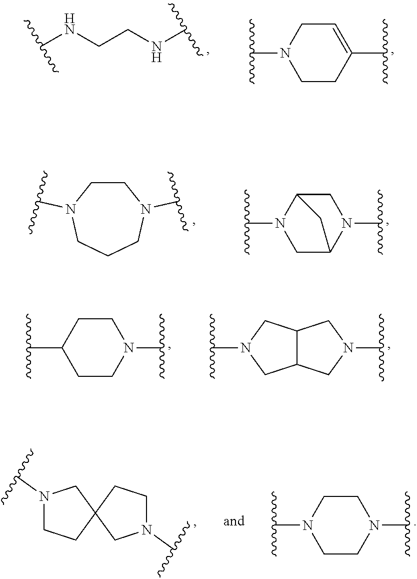

In a still further aspect, Q² is:

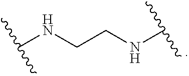 [SMILES: *NCCN*]

In yet a further aspect, Q² is a structure selected from:

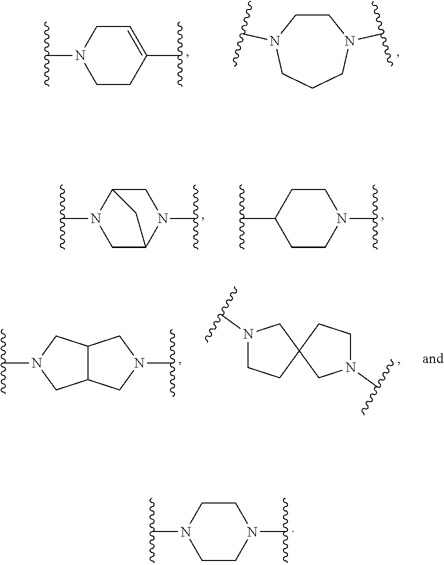

In an even further aspect, Q² is a structure selected from:

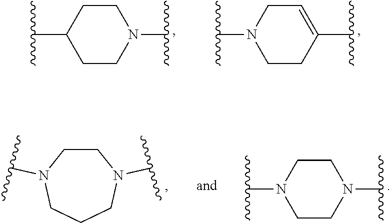 [SMILES: *C1CCN(*)CC1 ; *C1=CCN(*)CC1 ; *N1CCCN(*)CC1 ; *N1CCN(*)CC1]

In a still further aspect, Q² is a structure selected from:

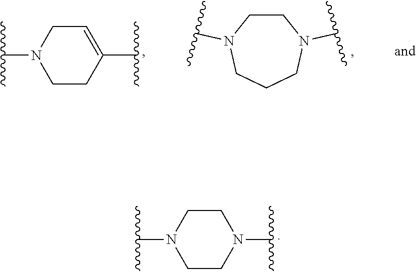 [SMILES: *C1=CCN(*)CC1 ; *N1CCCN(*)CC1 ; *N1CCN(*)CC1]

In yet a further aspect, Q² is a structure selected from:

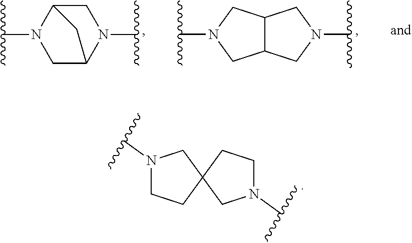

In a further aspect, Q² is a structure selected from:

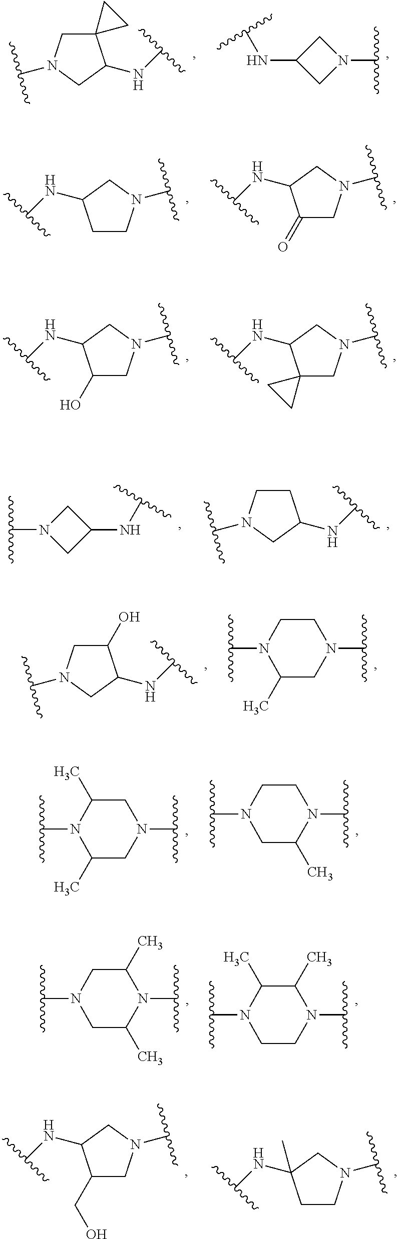

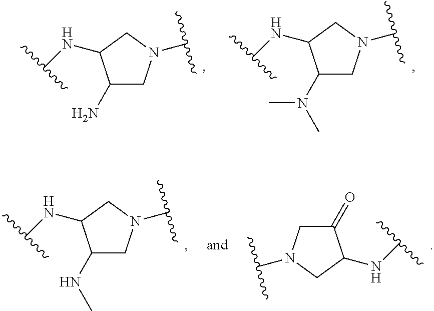

In a still further aspect, Q² is a structure selected from:

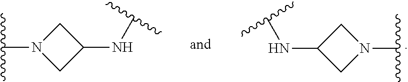 [SMILES: *NC1CN(*)C1 ; *NC1CN(*)C1]

In yet a further aspect, Q² is a structure selected from:

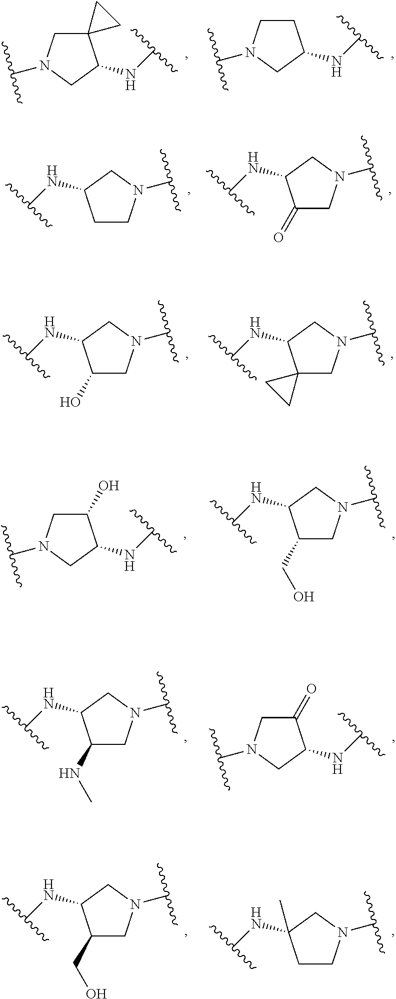

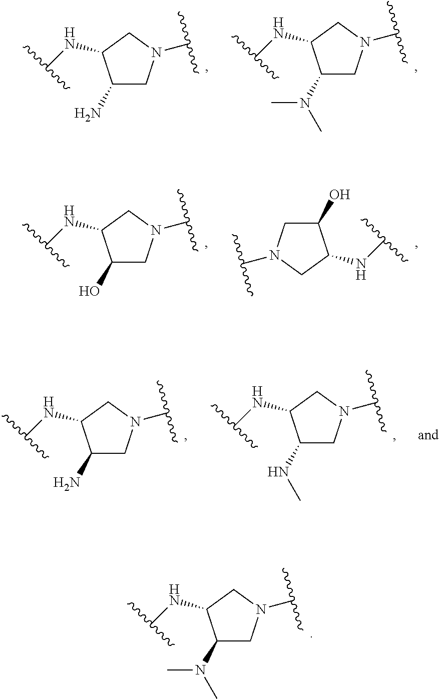

In an even further aspect, Q² is a structure selected from:

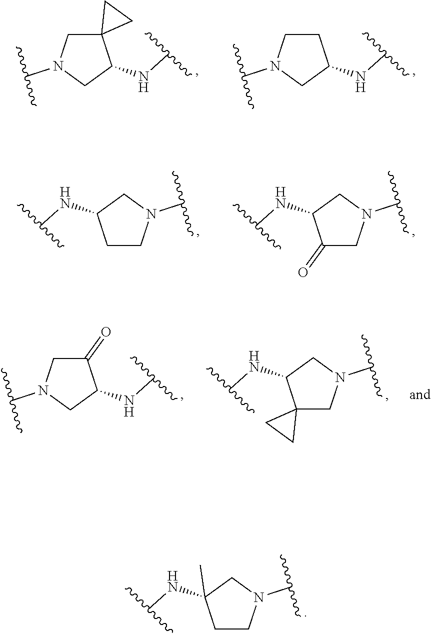

In a still further aspect, Q² is a structure selected from:

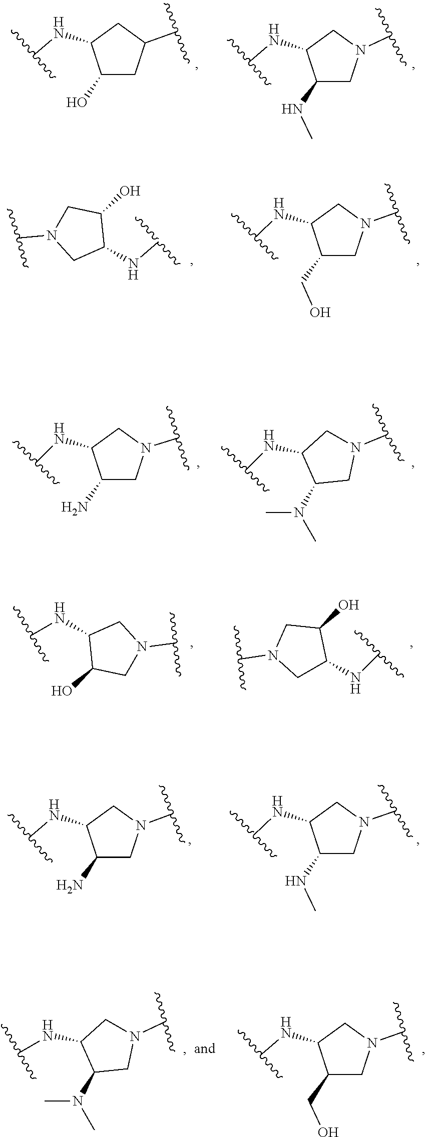

In yet further aspect, Q² is a structure selected from:

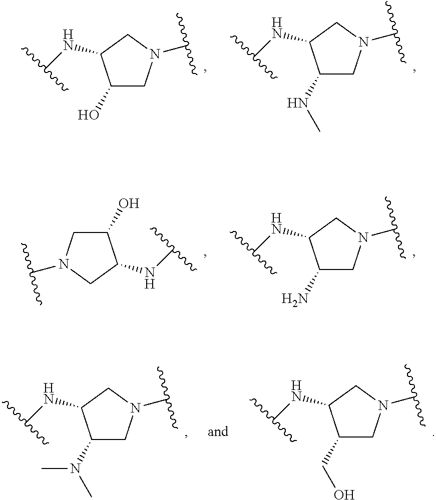

In an even further aspect, Q² is a structure selected from:

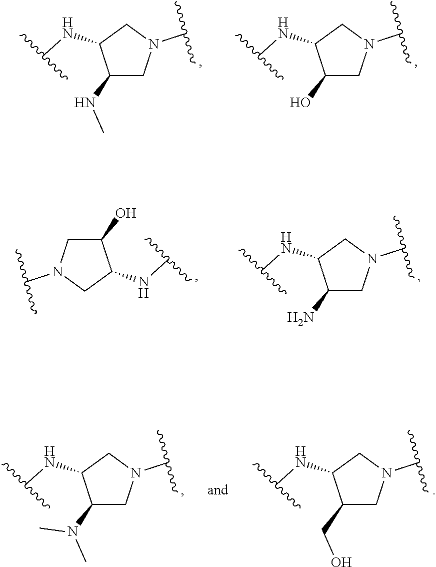

In a still further aspect, Q² is a structure selected from:

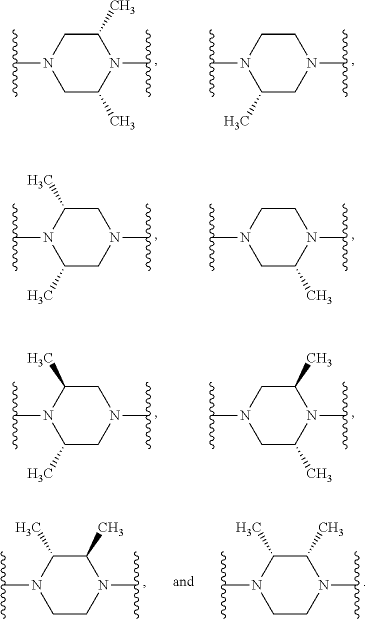

In yet a further aspect, Q² is a structure selected from:

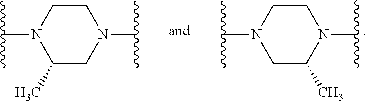

In an even further aspect, Q² is a structure selected from:

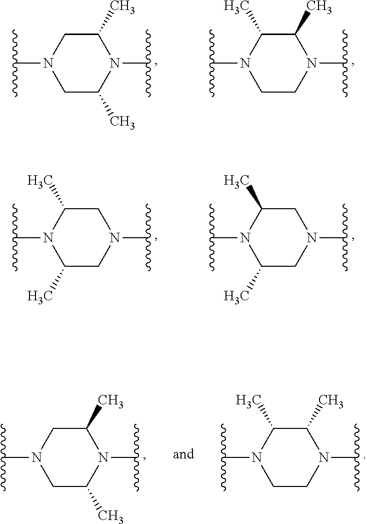

In a still further aspect, Q² is a structure selected from:

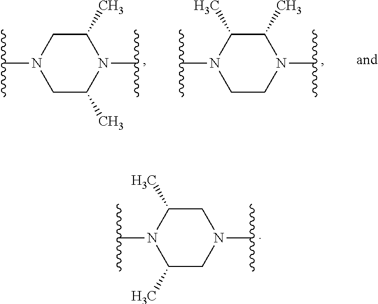

In yet a further aspect, Q² is a structure selected from:

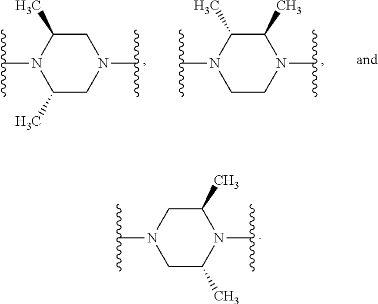

In a further aspect, Q² is a structure selected from:

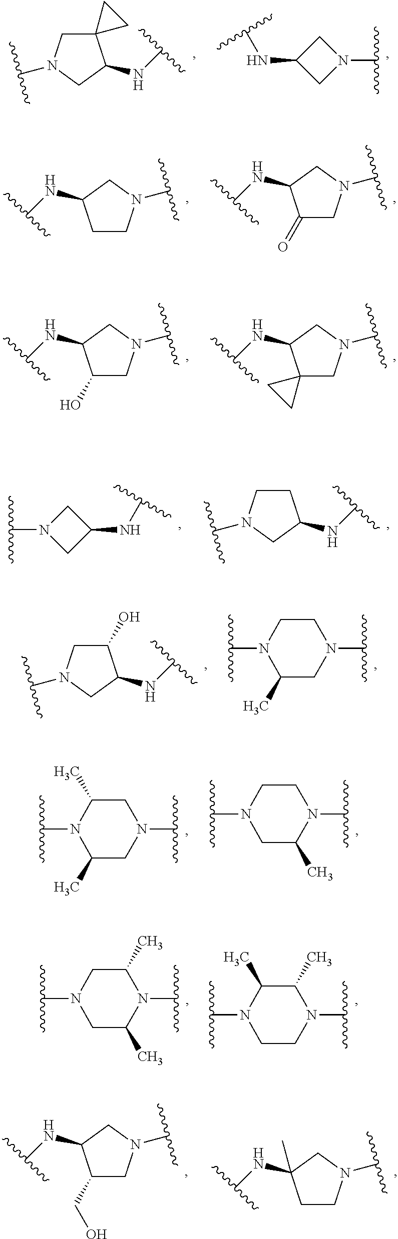

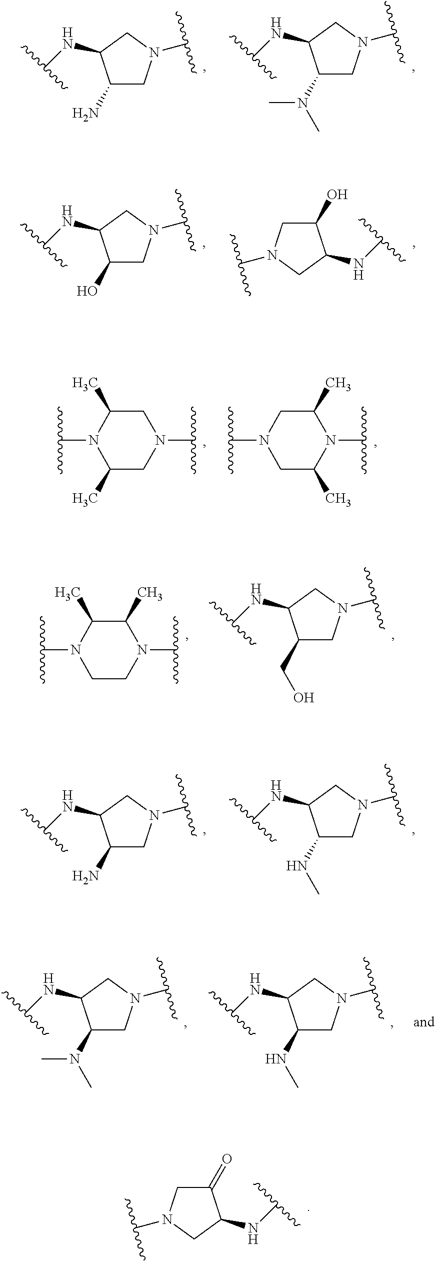

In a still further aspect, Q² is a structure selected from:

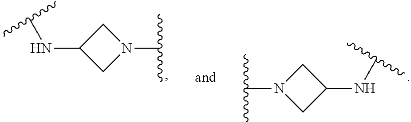 [SMILES: *NC1CN(*)C1 ; *NC1CN(*)C1]

In yet a further aspect, Q² is a structure selected from:

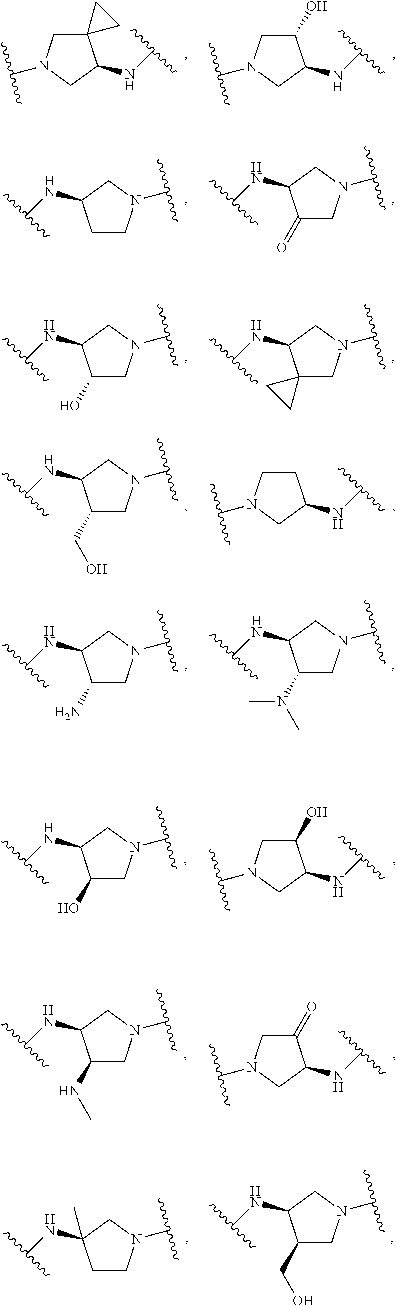

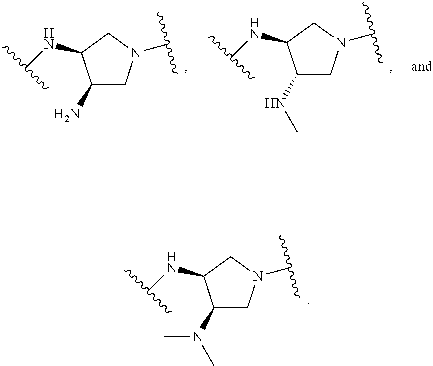

In an even further aspect, Q² is a structure selected from:

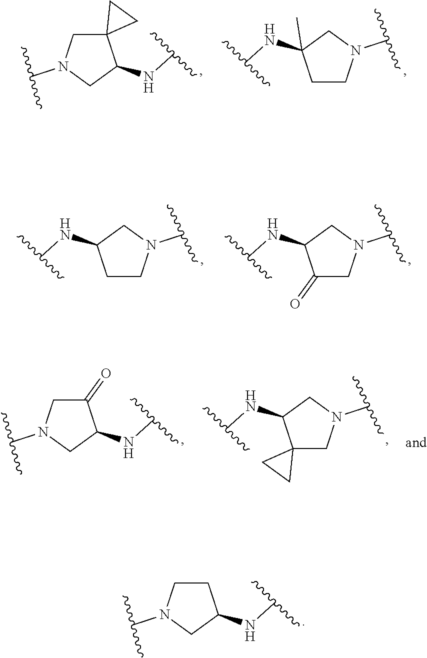

In a still further aspect, Q² is a structure selected from:

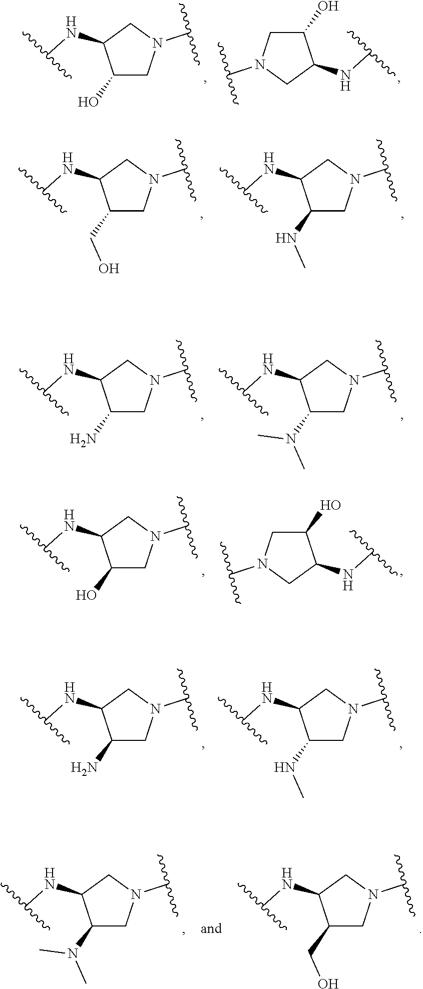

In yet a further aspect, Q² is a structure selected from:

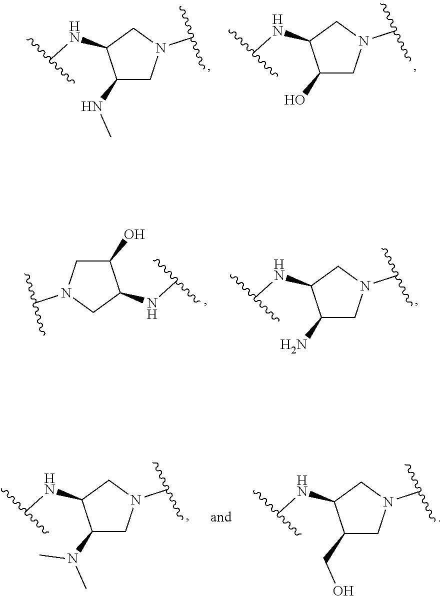

In an even further aspect, Q² is a structure selected from:

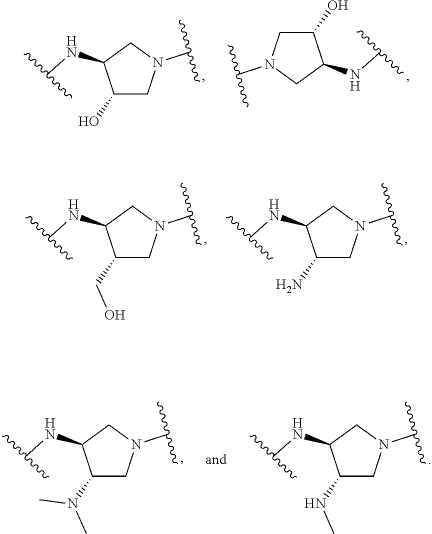

In a still further aspect, Q² is a structure selected from:

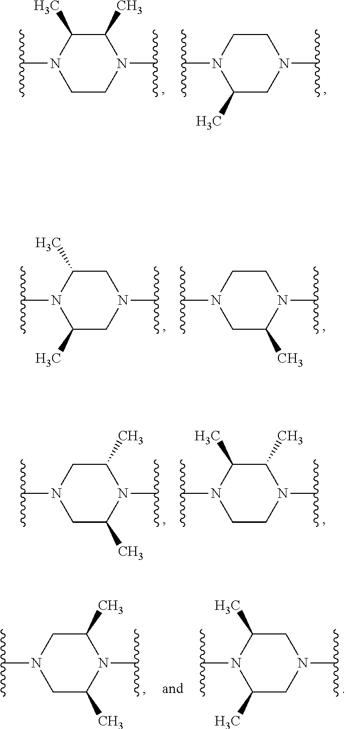

In yet a further aspect, Q² is a structure selected from:

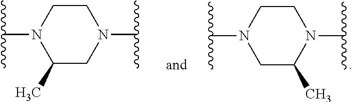

In an even further aspect, Q² is a structure selected from:

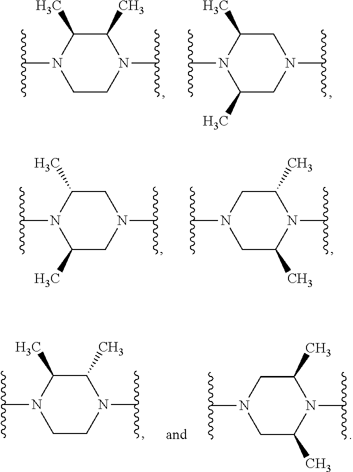

In a still further aspect, Q² is a structure selected from:

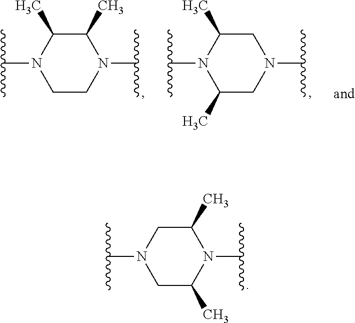

In yet a further aspect, Q² is a structure selected from:

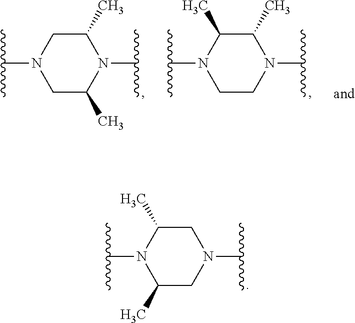

d. Q³ and Q⁴ Groups

In one aspect, Q³ is N and Q⁴ is CH or Q⁴ is N and Q³ is CH. In a further aspect, Q³ is N and Q⁴ is CH. In a still further aspect, Q⁴ is N and Q³ is CH.

e. Q⁵ Groups

In one aspect, each of Q¹ and Q⁵, when present, is independently selected from N and CH. In a further aspect, each of Q¹ and Q⁵, when present, is N. In a still further aspect, each of Q¹ and Q⁵, when present, is CH. In yet a further aspect, Q¹, when present, is N and Q⁵, when present, is CH. In an even further aspect, Q¹, when present, is CH and Q⁵, when present, is N.

f. Z Groups

In one aspect, Z is selected from A(C═O), COCH₂,

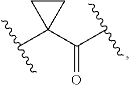 [SMILES: *C(=O)C1(*)CC1]

CO, NHCO, NHCS, CH₂SO₂, and SO₂. In a further aspect, Z is A(C═O).

In a further aspect, Z is selected from O(C═O), CF₂CO, COCH₂, CH₂CO,

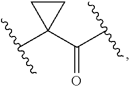 [SMILES: *C(=O)C1(*)CC1]

CO, CH₂SO₂, SO₂, NHCO, and CH(OH)CO.

In a further aspect, Z is selected from O(C═O), CF₂CO, COCH₂, CH₂CO,

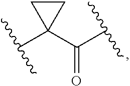 [SMILES: *C(=O)C1(*)CC1]

CO, CH₂SO₂, SO₂, and NHCO. In a still further aspect, Z is selected from O(C═O), CF₂CO, COCH₂, CH₂CO,

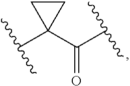 [SMILES: *C(=O)C1(*)CC1]

CO, CH₂SO₂, and SO₂. In yet a further aspect, Z is selected from O(C═O), CF₂CO, COCH₂, CH₂CO,

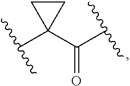 [SMILES: *C(=O)C1(*)CC1]

CO, and CH₂SO₂. In an even further aspect, Z is selected from O(C═O), CF₂CO, COCH₂, CH₂CO,

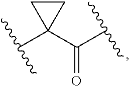 [SMILES: *C(=O)C1(*)CC1]

and CO. In a still further aspect, Z is selected from O(C═O), CF₂CO, COCH₂, and CH₂CO. In yet a further aspect, Z is selected from O(C═O), CF₂CO, and COCH₂. In an even further aspect, Z is selected from O(C═O) and CF₂CO. In a still further aspect, Z is O(C═O). In yet a further aspect, Z is CF₂CO. In an even further aspect, Z is COCH₂. In a still further aspect, Z is CH₂CO. In yet a further aspect, Z is CO. In an even further aspect, Z is

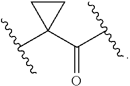 [SMILES: *C(=O)C1(*)CC1]

In a still further aspect, Z is CH₂SO₂. In yet a further aspect, Z is SO₂. In an even further aspect, Z is NHCO. In a still further aspect, Z is CH(OH)CO.

In one aspect, Z is selected from COCH₂, O(C═O), CF₂CO, and CH(OH)CO. In a further aspect, Z is selected from COCH₂, O(C═O), and CF₂CO. In a still further aspect, Z is selected from COCH₂ and O(C═O).

In one aspect, Z is selected from CO,

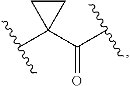 [SMILES: *C(=O)C1(*)CC1]

CH₂CO, COCH₂, NHCO, and NHCS.

In a further aspect, Z is selected from CO,

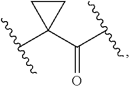 [SMILES: *C(=O)C1(*)CC1]

CH₂CO, COCH₂, and NHCO.

In a still further aspect, Z is selected from CO,

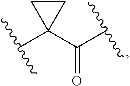 [SMILES: *C(=O)C1(*)CC1]

CH₂CO, and COCH₂. In yet a further aspect, Z is selected from CO,

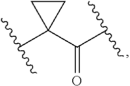 [SMILES: *C(=O)C1(*)CC1]

and CH₂CO. In an even further aspect, Z is selected from CO and

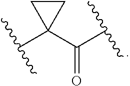 [SMILES: *C(=O)C1(*)CC1]

In a still further aspect, Z is NHCS.

In a further aspect, Z is A(C═O). In some embodiments, Z is CH₂(C═O).

g. R^{1A}, R^{1B}, and R^{1C} Groups

In one aspect, each of R^{1a}, R^{1b}, and R^{1c} is independently selected from hydrogen, halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a further aspect, each of R^{1a}, R^{1b}, and R^{1c} is hydrogen.

In a further aspect, each of R^{1a}, R^{1b}, and R^{1c} is independently selected from hydrogen, —F, —Cl, —Br, —NO₂, —CN, —OH, —SH, —NH₂, C1-C4 alkyl, C1-C4 monohaloalkyl, C1-C4 polyhaloalkyl, C1-C4 alkylamino, and (C1-C4)(C1-C4) dialkylamino. In a still further aspect, each of R^{1a}, R^{1b}, and R^{1c} is independently selected from hydrogen, —F, —Cl, —Br, —NO₂, —CN, —OH, —SH, —NH₂, methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —NHCH₃, —NHCH₂CH₃, —NH(CH₂)₂CH₃, —NHCH(CH₃)₂, —N(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, and —N(CH₃)CH(CH₃)₂. In yet a further aspect, each of R^{1a}, R^{1b}, and R^{1c} is independently selected from hydrogen, —F, —Cl, —Br, —NO₂, —CN, —OH, —SH, —NH₂, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —NHCH₃, —NHCH₂CH₃, —N(CH₃)₂, —N(CH₂CH₃)₂, and —N(CH₃)CH₂CH₃. In an even further aspect, each of R^{1a}, R^{1b}, and R^{1c} is independently selected from hydrogen, —F, —Cl, —Br, —NO₂, —CN, —OH, —SH, —NH₂, methyl, —CH₂F, —CH₂Cl, —CH₂Br, —CHF₂, —CF₃, —CHCl₂, —CCl₃, —CHBr₂, —CBr₃, —NHCH₃, and —N(CH₃)₂.

In a further aspect, each of R^{1a}, R^{1b}, and R^{1c} is independently selected from hydrogen, —F, —Cl, —Br, —NO₂, —CN, —OH, —SH, —NH₂, C1-C4 alkyl, C1-C4 alkoxy, C1-C4 alkylamino, and (C1-C4)(C1-C4) dialkylamino. In a still further aspect, each of R^{1a}, R^{1b}, and R^{1c} is independently selected from hydrogen, —F, —Cl, —Br, —NO₂, —CN, —OH, —SH, —NH₂, methyl, ethyl, n-propyl, i-propyl, —OCH₃, —OCH₂CH₃, —O(CH₂)₂CH₃, —OCH(CH₃)₂, —NHCH₃, —NHCH₂CH₃, —NH(CH₂)₂CH₃, —NHCH(CH₃)₂, —N(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, and —N(CH₃)CH(CH₃)₂. In yet a further aspect, each of R^{1a}, R^{1b}, and R^{1c} is independently selected from hydrogen, —F, —Cl, —Br, —NO₂, —CN, —OH, —SH, —NH₂, methyl, ethyl, —OCH₃, —OCH₂CH₃, —NHCH₃, —NHCH₂CH₃, —N(CH₃)₂, —N(CH₂CH₃)₂, and —N(CH₃)CH₂CH₃. In an even further aspect, each of R^{1a}, R^{1b}, and R^{1c} is independently selected from hydrogen, —F, —Cl, —Br, —NO₂, —CN, —OH, —SH, —NH₂, methyl, —OCH₃, —NHCH₃, —N(CH₃)₂.

In a further aspect, each of R^{1a}, R^{1b}, and R^{1c} is independently selected from hydrogen and C1-C4 alkyl. In a still further aspect, each of R^{1a}, R^{1b}, and R^{1c} is independently selected from hydrogen, methyl, ethyl, n-propyl, and i-propyl. In yet a further aspect, each of R^{1a}, R^{1b}, and R^{1c} is independently selected from hydrogen, methyl, and ethyl. In an even further aspect, each of R^{1a}, R^{1b}, and R^{1c} is independently selected from hydrogen and ethyl. In a still further aspect, each of R^{1a}, R^{1b}, and R^{1c} is independently selected from hydrogen and methyl.

In a further aspect, each of R^{1a}, R^{1b}, and R^{1c} is independently selected from hydrogen, C1-C4 monohaloalkyl, and C1-C4 polyhaloalkyl. In a still further aspect, each of R^{1a}, R^{1b}, and R^{1c} is independently selected from hydrogen, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃. In yet a further aspect, each of R^{1a}, R^{1b}, and R^{1c} is independently selected from hydrogen, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, and —CH₂CBr₃.

In an even further aspect, each of R^{1a}, R^{1b}, and R^{1c} is independently selected from hydrogen, —CH₂F, —CH₂Cl, —CH₂Br, —CHF₂, —CF₃, —CHCl₂, —CCl₃, —CHBr₂, and —CBr₃.

In a further aspect, each of R^{1a}, R^{1b}, and R^{1c} is independently selected from hydrogen and halogen. In a still further aspect, each of R^{1a}, R^{1b}, and R^{1c} is independently selected from hydrogen, —F, —Cl, and —Br. In yet a further aspect, each of R^{1a}, R^{1b}, and R^{1c} is independently selected from hydrogen, —F, and —Cl. In an even further aspect, each of R^{1a}, R^{1b}, and R^{1c} is independently selected from hydrogen and —Br. In a still further aspect, each of R^{1a}, R^{1b}, and R^{1c} is independently selected from hydrogen and —Cl. In yet a further aspect, each of R^{1a}, R^{1b}, and R^{1c} is independently selected from hydrogen and —F.

h. R² Groups

In one aspect, R² is selected from —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cuclobutyl, and oxetane, wherein the cyclopropyl, cyclobutyl, and oxetane are optionally substituted with 1, 2, or 3 groups independently selected from —OH, C1-C4 alkyl, and C1-C4 alkoxy. In a further aspect, R² is selected from —SCH₃, C1-C4 acyclic alkyl, C1-C4 acyclic alkenyl, C1-C4 monohaloalkyl, C1-C4 polyhaloalkyl, C1-C4 alkoxyhaloalkyl, cyclopropyl, cuclobutyl, and oxetane.

In one aspect, R² is selected from halogen, —SCH₃, C1-C8 acyclic alkyl, C1-C8 acyclic alkenyl, C1-C8 monohaloalkyl, C1-C8 polyhaloalkyl, C1-C8 alkoxyhaloalkyl, cyclopropyl, cuclobutyl, and oxetane, wherein the cyclopropyl, cyclobutyl, and oxetane are optionally substituted with 1, 2, or 3 groups independently selected from —OH, C1-C4 alkyl, and C1-C4 alkoxy. In a further aspect, R² is selected from halogen, —SCH₃, C1-C4 acyclic alkyl, C1-C4 acyclic alkenyl, C1-C4 monohaloalkyl, C1-C4 polyhaloalkyl, C1-C4 alkoxyhaloalkyl, cyclopropyl, cuclobutyl, and oxetane.

In one aspect, R² is selected from C1-C8 acyclic alkyl, C1-C8 acyclic alkenyl, C1-C8 monohaloalkyl, C1-C8 polyhaloalkyl, and cyclopropyl. In a further aspect, R² is selected from C1-C4 acyclic alkyl, C1-C4 acyclic alkenyl, C1-C4 monohaloalkyl, C1-C4 polyhaloalkyl, and cyclopropyl.

In one aspect, R² is selected from halogen, C1-C8 acyclic alkyl, C1-C8 acyclic alkenyl, C1-C8 monohaloalkyl, C1-C8 polyhaloalkyl, and cyclopropyl. In a further aspect, R² is selected from halogen, C1-C4 acyclic alkyl, C1-C4 acyclic alkenyl, C1-C4 monohaloalkyl, C1-C4 polyhaloalkyl, and cyclopropyl.

In one aspect, R² is selected from C1-C8 acyclic alkyl, C1-C8 acyclic alkenyl, C1-C8 monohaloalkyl, C1-C8 polyhaloalkyl, (C1-C8)(C1-C8) dialkylamino, and cyclopropyl. In a further aspect, R² is selected from C1-C4 acyclic alkyl, C1-C4 acyclic alkenyl, C1-C4 monohaloalkyl, C1-C4 polyhaloalkyl, (C1-C4)(C1-C4) dialkylamino, and cyclopropyl.

In one aspect, R² is selected from isopropyl and cyclopropyl. In a further aspect, R² is isopropyl. In a further aspect, R² is cyclopropyl.

In a further aspect, R² is selected from C1-C4 acyclic alkyl, C1-C4 acyclic alkenyl, and cyclopropyl. In a still further aspect, R² is selected from methyl, ethyl, n-propyl, i-propyl, ethenyl, 1-propenyl, 2-propenyl, and cyclopropyl. In yet a further aspect, R² is selected from ethyl, n-propyl, i-propyl, ethenyl, 1-propenyl, 2-propenyl, and cyclopropyl. In an even further aspect, R² is selected from n-propyl, i-propyl, 1-propenyl, 2-propenyl, and cyclopropyl.

In a further aspect, R² is selected from cyclopropyl, cyclobutyl, and oxetane and substituted with 1, 2, or 3 groups independently selected from —OH, C1-C4 alkyl, and C1-C4 alkoxy. In a still further aspect, R² is selected from cyclopropyl, cyclobutyl, and oxetane and substituted with 1 or 2 groups independently selected from —OH, C1-C4 alkyl, and C1-C4 alkoxy. In yet a further aspect, R² is selected from cyclopropyl, cyclobutyl, and oxetane and substituted with a group selected from —OH, C1-C4 alkyl, and C1-C4 alkoxy. In an even further aspect, R² is selected from cyclopropyl, cyclobutyl, and oxetane and substituted with a —OH group. In a still further aspect, R² is selected from cyclopropyl, cyclobutyl, and oxetane and substituted with a C1-C4 alkyl group. In yet a further aspect, R² is selected from cyclopropyl, cyclobutyl, and oxetane and substituted with a methyl group. In an even further aspect, R² is selected from cyclopropyl, cyclobutyl, and oxetane and is unsubstituted.

In a further aspect, R² is selected from C1-C4 acyclic alkyl, C1-C4 monohaloalkyl, C1-C4 polyhaloalkyl, C1-C4 alkoxyhaloalkyl, cyclopropyl, cyclobutyl, and oxetane. In a still further aspect, R² is selected from methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —OCH₂F, —OCHF₂, —OCF₃, cyclopropyl, cyclobutyl, and oxetane. In yet a further aspect, R² is selected from ethyl, n-propyl, i-propyl, —CH₂CH₂F, —CH₂CH₂Cl, —CH₂CH₂Br, —(CH₂)₂CH₂F, —(CH₂)₂CH₂Cl, —(CH₂)₂CH₂Br, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —OCH₂F, —OCHF₂, —OCF₃, cyclopropyl, cyclobutyl, and oxetane.

In an even further aspect, R² is selected from n-propyl, i-propyl, —(CH₂)₂CH₂F, —(CH₂)₂CH₂Cl, —(CH₂)₂CH₂Br, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —OCH₂F, —OCHF₂, —OCF₃, cyclopropyl, cyclobutyl, and oxetane.

In a further aspect, R² is selected from —SCH₃, halogen, C1-C4 acyclic alkyl, C1-C4 acyclic alkenyl, cyclopropyl, cyclobutyl, and oxetane. In a still further aspect, R² is selected from —SCH₃, —F, —Cl, —Br, methyl, ethyl, n-propyl, i-propyl, ethenyl, 1-propenyl, 2-propenyl, cyclopropyl, cyclobutyl, and oxetane. In yet a further aspect, R² is selected from —SCH₃, —F, —Cl, —Br, ethyl, n-propyl, i-propyl, 1-propenyl, 2-propenyl, cyclopropyl, cyclobutyl, and oxetane. In an even further aspect, R² is selected from —SCH₃, —F, —Cl, —Br, n-propyl, i-propyl, 1-propenyl, 2-propenyl, cyclopropyl, cyclobutyl, and oxetane.

In a further aspect, R² is selected from C1-C4 acyclic alkyl, C1-C4 acyclic alkenyl, (C1-C4)(C1-C4) dialkylamino, cyclopropyl, cyclobutyl, and oxetane. In a still further aspect, R² is selected from methyl, ethyl, n-propyl, i-propyl, ethenyl, 1-propenyl, 2-propenyl, —NHCH₃, —NHCH₂CH₃, —NH(CH₂)₂CH₃, —NHCH(CH₃)₂, —N(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, —N(CH₃)CH(CH₃)₂, cyclopropyl, cyclobutyl, and oxetane. In yet a further aspect, R² is selected from ethyl, n-propyl, i-propyl, ethenyl, 1-propenyl, 2-propenyl, —NHCH₂CH₃, —NH(CH₂)₂CH₃, —NHCH(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, —N(CH₃)CH(CH₃)₂, cyclopropyl, cyclobutyl, and oxetane. In an even further aspect, R² is selected from n-propyl, i-propyl, 1-propenyl, 2-propenyl, —NH(CH₂)₂CH₃, —NHCH(CH₃)₂, —N((CH₂)₂CH₃)₂, —N(CH(CH₃)₂)₂, —N(CH₃)(CH₂)₂CH₃, —N(CH₃)CH(CH₃)₂, cyclopropyl, cyclobutyl, and oxetane.

i. R^{3A} and R^{3B} Groups

In one aspect, each of R^{3a} and R^{3b} is independently selected from hydrogen, halogen, —OH, C1-C4 alkoxy, and C1-C4 alkyl. In a further aspect, each of R^{3a} and R^{3b} is independently selected from hydrogen, halogen, C1-C4 alkoxy, and C1-C4 alkyl. In a still further aspect, one of R^{3a} and R^{3b} is hydrogen and one of R^{3a} and R^{3b} is —OH.

In one aspect, each of R^{3a} and R^{3b} is independently selected from hydrogen, halogen, and C1-C4 alkyl. In a further aspect, each of R^{3a} and R^{3b} is hydrogen.

In one aspect, each of R^{3a} and R^{3b} is independently selected from hydrogen, halogen, C1-C4 alkyl, and C1-C4 alkoxy.

In a further aspect, each of R^{3a} and R^{3b} is independently selected from hydrogen, —F, —Cl, —Br, methyl, ethyl, n-propyl, i-propyl, n-butyl, i-butyl, t-butyl, and s-butyl. In a still further aspect, each of R^{3a} and R^{3b} is independently selected from hydrogen, —F, —Cl, —Br, methyl, ethyl, n-propyl, and i-propyl. In yet a further aspect, each of R^{3a} and R^{3b} is independently selected from hydrogen, —F, —Cl, —Br, methyl, and ethyl. In an even further aspect, each of R^{3a} and R^{3b} is independently selected from hydrogen, —F, —Cl, —Br, and methyl.

In a further aspect, each of R^{3a} and R^{3b} is independently selected from hydrogen, —F, —Cl, —Br, methyl, ethyl, n-propyl, i-propyl, —OCH₃, —OCH₂CH₃, —O(CH₂)₂CH₃, and —OCH(CH₃)₂. In a still further aspect, each of R^{3a} and R^{3b} is independently selected from hydrogen, —F, —Cl, —Br, methyl, ethyl, —OCH₃, and —OCH₂CH₃. In yet a further aspect, each of R^{3a} and R^{3b} is independently selected from hydrogen, —F, —Cl, —Br, methyl, and —OCH₃.

In a further aspect, each of R^{3a} and R^{3b} is independently selected from hydrogen and C1-C4 alkyl. In a still further aspect, each of R^{3a} and R^{3b} is independently selected from hydrogen, methyl, ethyl, n-propyl, and i-propyl. In yet a further aspect, each of R^{3a} and R^{3b} is independently selected from hydrogen, methyl, and ethyl. In an even further aspect, each of R^{3a} and R^{3b} is independently selected from hydrogen and ethyl. In a still further aspect, each of R^{3a} and R^{3b} is independently selected from hydrogen and methyl.

In a further aspect, each of R^{3a} and R^{3b} is independently selected from hydrogen and halogen. In a further aspect, each of R^{3a} and R^{3b} is independently selected from hydrogen, —F, —Cl, and —Br. In a still further aspect, each of R^{3a} and R^{3b} is independently selected from hydrogen, —F, and —Cl. In yet a further aspect, each of R^{3a} and R^{3b} is independently selected from hydrogen and —I. In an even further aspect, each of R^{3a} and R^{3b} is independently selected from hydrogen and —Br. In a still further aspect, each of R^{3a} and R^{3b} is independently selected from hydrogen and —Cl. In yet a further aspect, each of R^{3a} and R^{3b} is independently selected from hydrogen and —F. In some embodiments, R^{3a} is halogen and R^{3b} is hydrogen. In some embodiments, R^{3a} is —F and R^{3b} is hydrogen.

j. R⁵ Groups

In one aspect, R⁵, when present, is selected from CN, halogen, —NO₂, SO₂NH₂, and SO₂CH₃, provided that if R⁵ is CN and Z is CO then Ar¹ is not substituted with C1-C8 monohaloalkyl or C1-C8 polyhaloalkyl; and provided that if R⁵ is halogen then Ar¹ is selected from 5- and 6-membered heteroaryl and Z cannot be CO. In a further aspect, R⁵, when present, is CN.

In a further aspect, R⁵, when present, is selected from —NO₂, SO₂NH₂, and SO₂CH₃. In a still further aspect, R⁵, when present, is selected from SO₂NH₂ and SO₂CH₃. In yet a further aspect, R⁵, when present, is —NO₂. In an even further aspect, R⁵, when present, is SO₂NH₂. In a still further aspect, R⁵, when present, is SO₂CH₃.

In a further aspect, R⁵ is selected from halogen, —NO₂, SO₂NH₂, and SO₂CH₃. In a still further aspect, R⁵ is selected from —Cl, —F, —NO₂, SO₂NH₂, and SO₂CH₃.

In a further aspect, R⁵, when present, is selected from CN and halogen. In a still further aspect, R⁵, when present, is selected from CN, —Cl, and —F. In yet a further aspect, R⁵, when present, is selected from CN and —F. In an even further aspect, R⁵, when present, is selected from CN and —Cl.

In a further aspect, R⁵, when present, is selected from —I, —Br, —Cl, and —F. In a still further aspect, R⁵, when present, is —I. In yet a further aspect, R⁵, when present, is —Br. In an even further aspect, R⁵, when present, is —Cl. In a still further aspect, R⁵, when present, is —F.

k. R⁶ Groups

In one aspect, R⁶ is selected from —NHCH₂C₆H₅ and Ar². In a further aspect, R⁶ is —NHCH₂C₆H₅. In a still further aspect, R⁶ is Ar².

l. R^{20A}, R^{20B}, R^{20C}, and R^{20d} Groups

In one aspect, each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl.

In one aspect, each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl.

In one aspect, each of R^{20a}, R^{20b}, R^{20c}, and R^{20d}, when present, is independently selected from hydrogen, halogen, —CN, —NO₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl. In a further aspect, each of R^{20a}, R^{20b}, R^{20c}, and R^{20d}, when present, is hydrogen.

In one aspect, each of R^{20a}, R^{20b}, R^{20c}, and R^{20d}, when present, is independently selected from hydrogen, halogen, —CN, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl.

In a further aspect, each of R^{20a}, R^{20b}, R^{20c}, and R^{20d}, when present, is independently selected from hydrogen, —F, —Cl, —Br, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl. In a still further aspect, each of R^{20a}, R^{20b}, R^{20c}, and R^{20d}, when present, is independently selected from hydrogen, —F, —Cl, —Br, —CN, —NO₂, —NH₂, methyl, ethyl, n-propyl, i-propyl, —OCH₃, —OCH₂CH₃, —O(CH₂)₂CH₃, —OCH(CH₃)₂, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —NHCH₃, —NHCH₂CH₃, —NH(CH₂)₂CH₃, —NHCH(CH₃)₂, —N(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, —N(CH₃)CH(CH₃)₂. and cyclopropyl. In yet a further aspect, each of R^{20a}, R^{20b}, R^{20c}, and R^{20d}, when present, is independently selected from hydrogen, —F, —Cl, —Br, —CN, —NO₂, —NH₂, methyl, ethyl, —OCH₃, —OCH₂CH₃, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —NHCH₃, —NHCH₂CH₃, —N(CH₃)₂, —N(CH₂CH₃)₂, —N(CH₃)CH₂CH₃, and cyclopropyl. In an even further aspect, each of R^{20a}, R^{20b}, R^{20c}, and R^{20d}, when present, is independently selected from hydrogen, —F, —Cl, —Br, —CN, —NO₂, —NH₂, methyl, —OCH₃, —OCH₂CH₃, —CH₂F, —CH₂Cl, —CH₂Br, —CHF₂, —CF₃, —CHCl₂, —CCl₃, —CHBr₂, —CBr₃, —NHCH₃, —N(CH₃)₂, and cyclopropyl.

In a further aspect, each of R^{20a}, R^{20b}, R^{20c}, and R^{20d}, when present, is independently selected from hydrogen, —F, —Cl, —Br, —CN, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl. In a still further aspect, each of R^{20a}, R^{20b}, R^{20c}, and R^{20d}, when present, is independently selected from hydrogen, —F, —Cl, —Br, —CN, methyl, ethyl, n-propyl, i-propyl, —OCH₃, —OCH₂CH₃, —O(CH₂)₂CH₃, —OCH(CH₃)₂, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —NHCH₃, —NHCH₂CH₃, —NH(CH₂)₂CH₃, —NHCH(CH₃)₂, —N(CH₃)₂, —N(CH₂CH₃)₂, —N((CH₂)₂CH₃)₂, —N(CH(CH₃)₂)₂, —N(CH₃)CH₂CH₃, —N(CH₃)(CH₂)₂CH₃, —N(CH₃)CH(CH₃)₂. and cyclopropyl. In yet a further aspect, each of R^{20a}, R^{20b}, R^{20c}, and R^{20d}, when present, is independently selected from hydrogen, —F, —Cl, —Br, —CN, methyl, ethyl, —OCH₃, —OCH₂CH₃, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —NHCH₃, —NHCH₂CH₃, —N(CH₃)₂, —N(CH₂CH₃)₂, —N(CH₃)CH₂CH₃, and cyclopropyl. In an even further aspect, each of R^{20a}, R^{20b}, R^{20c}, and R^{20d}, when present, is independently selected from hydrogen, —F, —Cl, —Br, —CN, methyl, —OCH₃, —OCH₂CH₃, —CH₂F, —CH₂Cl, —CH₂Br, —CHF₂, —CF₃, —CHCl₂, —CCl₃, —CHBr₂, —CBr₃, —NHCH₃, —N(CH₃)₂, and cyclopropyl.

In a further aspect, each of R^{20a}, R^{20b}, R^{20c}, and R^{20d}, when present, is independently selected from hydrogen and C1-C4 alkyl. In a still further aspect, each of R^{20a}, R^{20b}, R^{20c}, and R^{20d}, when present, is independently selected from hydrogen, methyl, ethyl, n-propyl, and i-propyl. In yet a further aspect, each of R^{20a}, R^{20b}, R^{20c}, and R^{20d}, when present, is independently selected from hydrogen, methyl, and ethyl. In an even further aspect, each of R^{20a}, R^{20b}, R^{20c}, and R^{20d}, when present, is independently selected from hydrogen and ethyl. In a still further aspect, each of R^{20a}, R^{20b}, R^{20c}, and R^{20d}, when present, is independently selected from hydrogen and methyl.

In a further aspect, each of R^{20a}, R^{20b}, R^{20c}, and R^{20d}, when present, is independently selected from hydrogen and halogen. In a still further aspect, each of R^{20a}, R^{20b}, R^{20c}, and R^{20d}, when present, is independently selected from hydrogen, —F, —Cl, and —Br. In yet a further aspect, each of R^{20a}, R^{20b}, R^{20c}, and R^{20d}, when present, is independently selected from hydrogen, —F, and —Cl. In an even further aspect, each of R^{20a}, R^{20b}, R^{20c}, and R^{20d}, when present, is independently selected from hydrogen and —I. In a still further aspect, each of R^{20a}, R^{20b}, R^{20c}, and R^{20d}, when present, is independently selected from hydrogen and —Br. In yet a further aspect, each of R^{20a}, R^{20b}, R^{20c}, and R^{20d}, when present, is independently selected from hydrogen and —Cl. In an even further aspect, each of R^{20a}, R^{20b}, R^{20c}, and R^{20d}, when present, is independently selected from hydrogen and —F.

m. R²¹ Groups

In one aspect, R²¹, when present, is selected from hydrogen, halogen, —CN, —NO₂, —SO₂NH₂, —SO₂CH₃, —SO₂CF₃, and Cy¹.

In one aspect, R²¹, when present, is selected from —CN, —NO₂, SO₂NH₂, SO₂CH₃, SO₂CF₃, and Cy¹. In a further aspect, R²¹, when present, is selected from —CN, —NO₂, SO₂NH₂, SO₂CH₃, and SO₂CF₃. In a still further aspect, R²¹, when present, is selected from —CN, —NO₂, SO₂NH₂, and SO₂CH₃. In yet a further aspect, R²¹, when present, is selected from —CN, —NO₂, and SO₂NH₂. In an even further aspect, R²¹, when present, is selected from —CN, and —NO₂.

In a further aspect, R²¹, when present, is —CN. In a still further aspect, R²¹, when present, is —NO₂. In yet a further aspect, R²¹, when present, is SO₂NH₂. In an even further aspect, R²¹, when present, is SO₂CH₃. In a still further aspect, R²¹, when present, is SO₂CF₃. In yet a further aspect, R²¹, when present, is Cy¹.

In a further aspect, R²¹, when present, is —CN and R²², when present, is selected from —CN and halogen. In a still further aspect, R²¹, when present, is —CN and R²², when present, is selected from —CN, —F, —Cl, and —Br. In yet a further aspect, R²¹, when present, is —CN and R²² when present, is selected from —CN, —F, and —Cl. In an even further aspect, R²¹, when present, is —CN and R²², when present, is —CN. In a still further aspect, R²¹, when present, is —CN and R²², when present, is —I. In yet a further aspect, R²¹, when present, is —CN and R²², when present, is —Br. In an even further aspect, R²¹, when present, is —CN and R²², when present, is —Cl. In a still further aspect, R²¹, when present, is —CN and R²², when present, is —F.

n. R²²Groups

In one aspect, R²², when present, is selected from —CN, halogen, —NO₂, SO₂NH₂, SO₂CH₃, and SO₂CF₃.

In one aspect, R²², when present, is selected from —CN, halogen, —NO₂, SO₂NH₂, SO₂CH₃, and SO₂CF₃. In a further aspect, R²², when present, is selected from —CN, halogen, —NO₂, SO₂NH₂, and SO₂CH₃. In yet a further aspect, R²², when present, is selected from —CN, halogen, —NO₂, and SO₂NH₂. In an even further aspect, R²², when present, is selected from —CN, halogen, and —NO₂. In a still further aspect, R²², when present, is selected from —CN and halogen.

In a further aspect, R²², when present, is —CN. In a still further aspect, R²², when present, is —NO₂. In yet a further aspect, R²², when present, is SO₂NH₂. In an even further aspect, R²², when present, is SO₂CH₃. In a still further aspect, R²², when present, is SO₂CF₃.

In a further aspect, R²², when present, is halogen. In a still further aspect, R²², when present, is selected from —F, —Cl, and —Br. In yet a further aspect, R²², when present, is selected from —F and —Br. In an even further aspect, R²², when present, is selected from —F and —Cl. In a still further aspect, R²², when present, is —I. In yet a further aspect, R²², when present, is —Br. In an even further aspect, R²², when present, is —Cl. In a still further aspect, R²², when present, is —F.

o. R²³ Groups

In one aspect, R²³, when present, is selected from hydrogen, halogen, —CN, —NO₂, —SO₂NH₂, —SO₂CH₃, —SO₂CF₃, cyclohexyl,

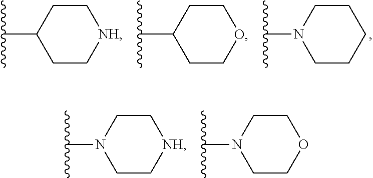

### and Cy¹.

In one aspect, R²³, when present, is selected from —CN, —NO₂, SO₂NH₂, SO₂CH₃, SO₂CF₃, cyclohexyl,

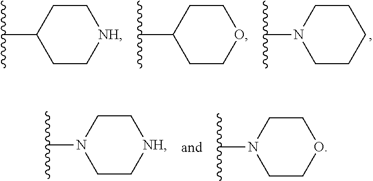

further aspect, R²³, when present, is selected from —CN, —NO₂, SO₂NH₂, and SO₂CH₃. In yet a further aspect, R²³, when present, is selected from —CN, —NO₂, and SO₂NH₂. In an even further aspect, R²³, when present, is selected from —CN, and —NO₂.

In a further aspect, R²³, when present, is —CN. In a still further aspect, R²³, when present, is —NO₂. In yet a further aspect, R²³, when present, is SO₂NH₂. In an even further aspect, R²³, when present, is SO₂CH₃. In a still further aspect, R²³, when present, is SO₂CF₃.

In a further aspect, R²³, when present, is selected from cyclohexyl,

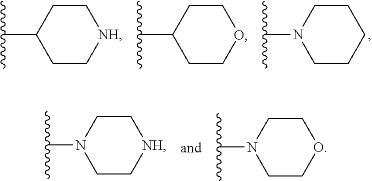

In a still further aspect, R²³, when present, is selected from

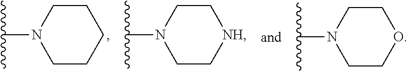

In yet a further aspect, R²³, when present, is selected from cyclohexyl,

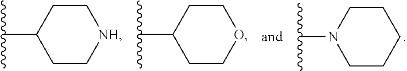

In an even further aspect, R²³, when present, is selected from

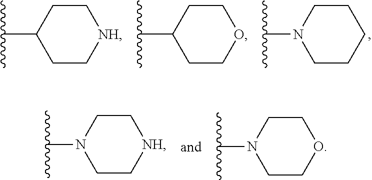

In a still further aspect, R²³, when present, is cyclohexyl.

In a further aspect, R²³ is selected from hydrogen, halogen, —CN, SO₂NH₂, SO₂CH₃, SO₂CF₃, and NO₂. In a further aspect, R²³ is hydrogen.

In a further aspect, R²³ is selected from —CN, SO₂NH₂, SO₂CH₃, SO₂CF₃, and NO₂. In a still further aspect, R²³ is selected from —CN, SO₂NH₂, SO₂CH₃, and SO₂CF₃. In yet a further aspect, R²³ is selected from —CN, SO₂NH₂, and SO₂CH₃. In an even further aspect, R²³ is selected from —CN and SO₂NH₂. In a still further aspect, R²³ is NO₂. In yet a further aspect, R²³ is SO₂CF₃. In an even further aspect, R²³ is SO₂CH₃. In a still further aspect, R²³ is SO₂NH₂. In yet a further aspect, R²³ is —CN.

In a further aspect, R²³ is selected from hydrogen and halogen. In a still further aspect, R²³ is selected from hydrogen, —F, —Cl, and —Br. In yet a further aspect, R²³ is selected from hydrogen, —F, and —Cl. In an even further aspect, R²³ is selected from hydrogen and —I. In a still further aspect, R²³ is selected from hydrogen and —Br. In yet a further aspect, R²³ is selected from hydrogen and —Cl. In an even further aspect, R²³ is selected from hydrogen and —F. In some embodiments, R²³ is halogen. In some embodiments, R²³ is —Cl.

p. R²⁴ Groups

In one aspect, R²⁴, when present, is selected from —CN, halogen, —NO₂, SO₂NH₂, SO₂CH₃, and SO₂CF₃.

In one aspect, R²⁴, when present, is selected from —CN, halogen, —NO₂, SO₂NH₂, SO₂CH₃, and SO₂CF₃, provided that if A is NH or N(CH₃), then R²⁴ is not —NO₂. In a further aspect, R²⁴, when present, is selected from —CN, halogen, —NO₂, SO₂NH₂, and SO₂CH₃. In yet a further aspect, R²⁴, when present, is selected from —CN, halogen, —NO₂, and SO₂NH₂. In an even further aspect, R²⁴, when present, is selected from —CN, halogen, and —NO₂. In a still further aspect, R²⁴, when present, is selected from —CN and halogen.

In a further aspect, R²⁴, when present, is —CN. In a still further aspect, R²⁴, when present, is —NO₂. In yet a further aspect, R²⁴, when present, is SO₂NH₂. In an even further aspect, R²⁴, when present, is SO₂CH₃. In a still further aspect, R²⁴, when present, is SO₂CF₃.

In a further aspect, R²⁴, when present, is halogen. In a still further aspect, R²⁴, when present, is selected from —F, —Cl, and —Br. In yet a further aspect, R²⁴, when present, is selected from —F and —Br. In an even further aspect, R²⁴, when present, is selected from —F and —Cl. In a still further aspect, R²⁴, when present, is —I. In yet a further aspect, R²⁴, when present, is —Br. In an even further aspect, R²⁴, when present, is —Cl. In a still further aspect, R²⁴, when present, is —F.

q. R²⁵ Groups

In one aspect, R²⁵, when present, is selected from —CN, —NO₂, SO₂NH₂, SO₂CH₃, and SO₂CF₃.

In one aspect, R²⁵, when present, is selected from —CN, —NO₂, SO₂NH₂, SO₂CH₃, and SO₂CF₃. In a further aspect, R²⁵, when present, is selected from —CN, —NO₂, SO₂NH₂, and SO₂CH₃. In yet a further aspect, R²⁵, when present, is selected from —CN, —NO₂, and SO₂NH₂. In an even further aspect, R²⁵, when present, is selected from —CN and —NO₂.

In a further aspect, R²⁵, when present, is —CN. In a still further aspect, R²⁵, when present, is —NO₂. In yet a further aspect, R²⁵, when present, is SO₂NH₂. In an even further aspect, R²⁵, when present, is SO₂CH₃. In a still further aspect, R²⁵, when present, is SO₂CF₃.

r. R²⁶ Groups

In one aspect, R²⁶, when present, is selected from —Br, —Cl, —F, —CN, —NO₂, —CF₃, and methyl.

s. AR¹ Groups

In one aspect, Ar¹ is selected from aryl and heteroaryl and substituted with 0, 1, 2, or 3 groups independently selected from halogen, —NO₂, —CN, —OH, —SH, —NH₂, C1-C8 thioalkyl, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), C1-C8 alkoxyhaloalkyl, and cyclopropyl, cyclobutyl, and oxetane, wherein the cyclopropyl, cyclobutyl, and oxetane are optionally substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl).

In one aspect, Ar¹ is selected from aryl and heteroaryl and substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy.

In one aspect, Ar¹ is selected from aryl and heteroaryl and substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl.

In one aspect, Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino.

In a further aspect, Ar¹ is selected from aryl and heteroaryl and substituted with 1 or 2 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In a still further aspect, Ar¹ is selected from aryl and heteroaryl and monosubstituted with a group selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In yet a further aspect, Ar¹ is selected from aryl and heteroaryl and unsubstituted.

In a further aspect, Ar¹ is aryl substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In a still further aspect, Ar¹ is aryl substituted with 1 or 2 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In yet a further aspect, Ar¹ is aryl monosubstituted with a group selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In an even further aspect, Ar¹ is unsubstituted aryl.

In a further aspect, Ar¹ is phenyl substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In a still further aspect, Ar¹ is phenyl substituted with 1 or 2 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In yet a further aspect, Ar¹ is phenyl monosubstituted with a group selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In an even further aspect, Ar¹ is unsubstituted phenyl.

In a further aspect, Ar¹ is heteroaryl substituted with 1, 2, or 3 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In a still further aspect, Ar¹ is heteroaryl substituted with 1 or 2 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In yet a further aspect, Ar¹ is heteroaryl monosubstituted with a group selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In an even further aspect, Ar¹ is unsubstituted heteroaryl.

In a further aspect, Ar¹ is selected from phenyl and monocyclic heteroaryl and substituted with 1 or 2 groups independently selected from halogen, —NO₂, —CN, —OH, —SH, —NH₂, C1-C8 acyclic alkyl, C1-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl. In some embodiments, Ar¹ is selected from phenyl and monocyclic heteroaryl and substituted with 1 or 2 groups independently selected from halogen and cyclopropyl.

In a further aspect, Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is selected from furanyl, 3-isopropylisoxazole, 6-isopropylpyridin-2-yl, 5-isopropylpyridin-2-yl, 5-tertbutylpyridin-2-yl, 5-bromopyridin-2-yl, 5-(prop-1-en-2-yl)pyridin-2-yl, 3-pyridinyl, 4-pyridinyl, and pyrimidinyl, and unsubstituted.

In a further aspect, Ar¹ is furanyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is furanyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is furanyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted furanyl.

In a further aspect, Ar¹ is 3-isopropylisoxazole substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 3-isopropylisoxazole substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 3-isopropylisoxazole monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 3-isopropylisoxazole.

In a further aspect, Ar¹ is 6-isopropylpyridin-2-yl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 6-isopropylpyridin-2-yl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 6-isopropylpyridin-2-yl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 6-isopropylpyridin-2-yl.

In a further aspect, Ar¹ is 5-isopropylpyridin-2-yl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 5-isopropylpyridin-2-yl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 5-isopropylpyridin-2-yl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 5-isopropylpyridin-2-yl.

In a further aspect, Ar¹ is 5-tertbutylpyridin-2-yl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 5-tertbutylpyridin-2-yl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 5-tertbutylpyridin-2-yl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 5-tertbutylpyridin-2-yl.

In a further aspect, Ar¹ is 5-bromopyridin-2-yl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 5-bromopyridin-2-yl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 5-bromopyridin-2-yl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 5-bromopyridin-2-yl.

In a further aspect, Ar¹ is 5-(prop-1-en-2-yl)pyridin-2-yl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 5-(prop-1-en-2-yl)pyridin-2-yl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 5-(prop-1-en-2-yl)pyridin-2-yl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 5-(prop-1-en-2-yl)pyridin-2-yl.

In a further aspect, Ar¹ is 3-pyridinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is 3-pyridinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is 3-pyridinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted 3-pyridinyl.

In a further aspect, Ar¹ is pyridinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is pyridinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is pyridinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted pyridinyl. In a further aspect, Ar¹ is pyrimidinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Ar¹ is pyrimidinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Ar¹ is pyrimidinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Ar¹ is unsubstituted pyrimidinyl.

t. AR² Groups

In one aspect, Ar² is a structure represented by a formula selected from:

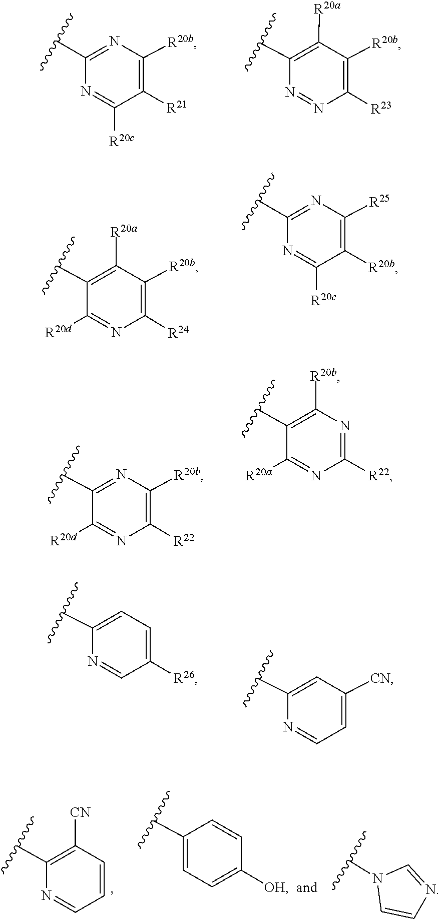

In one aspect, Ar² is a structure represented by a formula selected from:

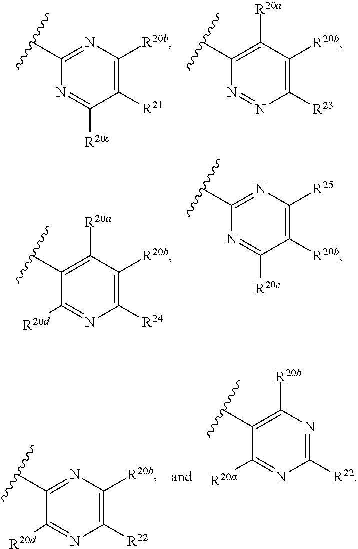

In one aspect, Ar² is a structure represented by a formula selected from:

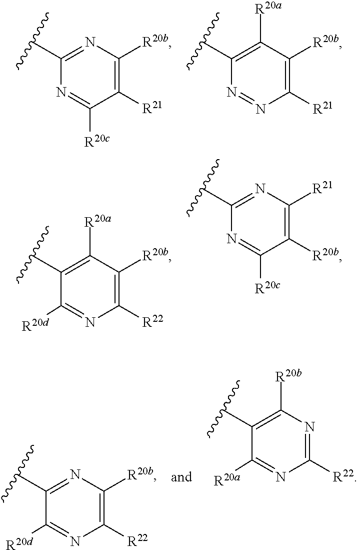

In one aspect, Ar² is a structure represented by a formula selected from:

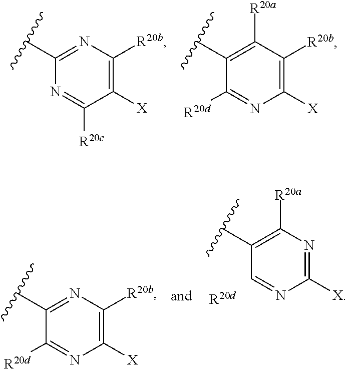

In one aspect, Ar² is a structure represented by a formula:

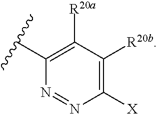

In a further aspect, Ar² is a structure represented by a formula:

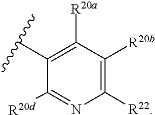

In a still further aspect, Ar² is a structure represented by a formula:

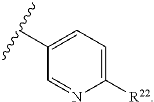

In yet a further aspect, Ar² is a structure represented by a formula selected from:

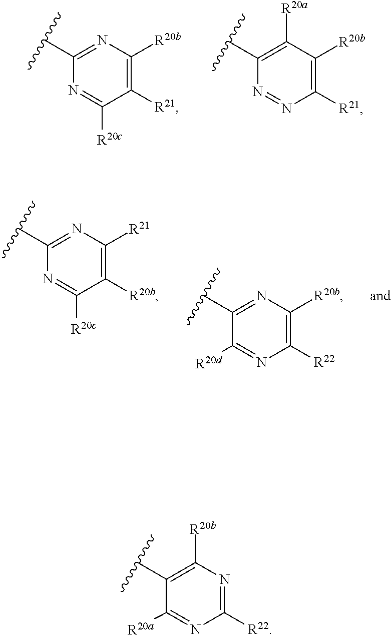

In an even further aspect, Ar² is a structure represented by a formula selected from:

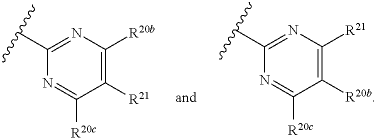

In a still further aspect, Ar² is a structure represented by a formula selected from:

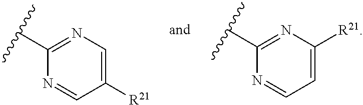

In yet a further aspect, Ar² is a structure represented by a formula selected from:

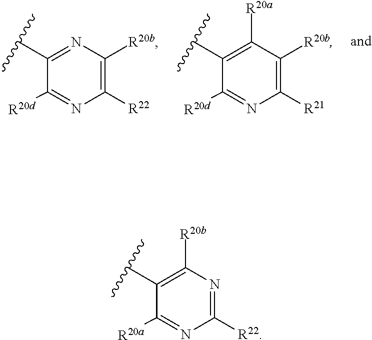

In an even further aspect, Ar² is a structure represented by a formula selected from:

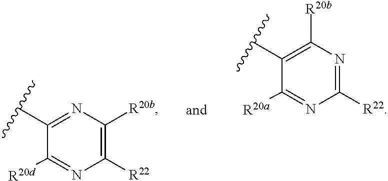

In a still further aspect, Ar² is a structure represented by a formula selected from:

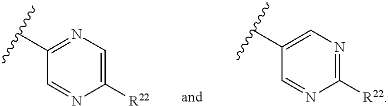

In yet a further aspect, Ar² is a structure represented by a formula:

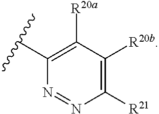

In a further aspect, Ar² is a structure represented by a formula:

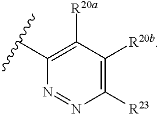

In an even further aspect, Ar² is a structure represented by a formula:

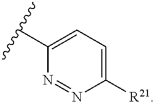

In a further aspect, Ar² is a structure represented by a formula:

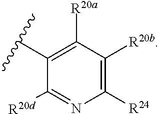

In a further aspect, Ar² is a structure represented by a formula:

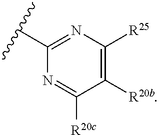

In a further aspect, Ar² is a structure represented by a formula selected from:

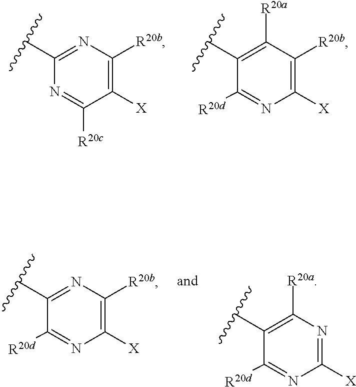

In a still further aspect, Ar² is a structure represented by a formula:

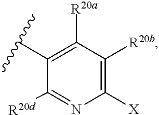

In yet a further aspect, Ar² is a structure represented by a formula:

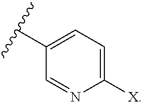

In an even further aspect, Ar² is a structure represented by a formula selected from:

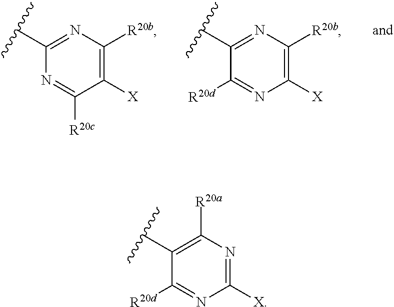

In a still further aspect, Ar² is a structure represented by a formula selected from:

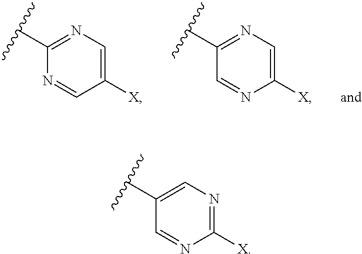

In a further aspect, Ar² is a structure represented by a formula:

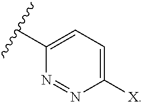

u. AR³ Groups

In one aspect, Ar³ is a structure selected from:

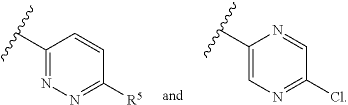

In a further aspect, Ar³ is:

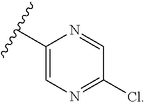 [SMILES: *c1cnc(Cl)cn1]

In a further aspect, Ar³ is:

In a further aspect, Ar³ is:

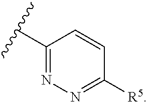

In a further aspect, Ar³ is:

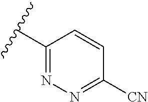 [SMILES: *c1ccc(C#N)nn1]

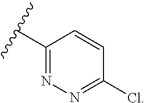 [SMILES: *c1ccc(Cl)nn1]

v. CY¹ Groups

In one aspect, Cy¹, when present, is selected from cycle, heterocycle, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino.

In one aspect, Cy¹, when present, is selected from cycloalkyl, heterocycloalkyl, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino.

In a further aspect, Cy¹, when present, is selected from cycloalkyl, heterocycloalkyl, aryl, and heteroaryl and substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is selected from cycloalkyl, heterocycloalkyl, aryl, and heteroaryl and substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is selected from cycloalkyl, heterocycloalkyl, aryl, and heteroaryl and monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is selected from cycloalkyl, heterocycloalkyl, aryl, and heteroaryl and unsubstituted.

In a further aspect, Cy¹, when present, is selected from cyclopropyl, imidazolyl, pyrazolyl, pyrrolyl, piperidinyl, morpholinyl, and piperazinyl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is selected from cyclopropyl, imidazolyl, pyrazolyl, pyrrolyl, piperidinyl, morpholinyl, and piperazinyl and substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is selected from cyclopropyl, imidazolyl, pyrazolyl, pyrrolyl, piperidinyl, morpholinyl, and piperazinyl and substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is selected from cyclopropyl, imidazolyl, pyrazolyl, pyrrolyl, piperidinyl, morpholinyl, and piperazinyl and monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is selected from cyclopropyl, imidazolyl, pyrazolyl, pyrrolyl, piperidinyl, morpholinyl, and piperazinyl and unsubstituted.

In a further aspect, Cy¹, when present, is selected from cycloalkyl and heterocycloalkyl and substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is selected from cycloalkyl and heterocycloalkyl and substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is selected from cycloalkyl and heterocycloalkyl and monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is selected from cycloalkyl and heterocycloalkyl and unsubstituted.

In a further aspect, Cy¹, when present, is cycloalkyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is cycloalkyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is cycloalkyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted cycloalkyl.

In a further aspect, Cy¹, when present, is cyclopropyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is cyclopropyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is cyclopropyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted cyclopropyl.

In a further aspect, Cy¹, when present, is heterocycloalkyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is heterocycloalkyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is heterocycloalkyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted heterocycloalkyl.

In a further aspect, Cy¹, when present, is morpholinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is morpholinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is morpholinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted morpholinyl.

In a further aspect, Cy¹, when present, is piperidinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is piperidinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is piperidinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted piperidinyl.

In a further aspect, Cy¹, when present, is piperazinyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is piperazinyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is piperazinyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted piperazinyl.

In a further aspect, Cy¹, when present, is selected from aryl and heteroaryl and substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is selected from aryl and heteroaryl and substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is selected from aryl and heteroaryl and monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is selected from aryl and heteroaryl and unsubstituted.

In a further aspect, Cy¹, when present, is aryl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is aryl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is aryl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted aryl.

In a further aspect, Cy¹, when present, is heteroaryl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is heteroaryl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is heteroaryl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted heteroaryl.

In a further aspect, Cy¹, when present, is imidazolyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is imidazolyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is imidazolyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted imidazolyl.

In a further aspect, Cy¹, when present, is pyrazolyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is pyrazolyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is pyrazolyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted pyrazolyl.

In a further aspect, Cy¹, when present, is pyrrolyl substituted with 0, 1, or 2 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In a still further aspect, Cy¹, when present, is pyrrolyl substituted with 0 or 1 group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In yet a further aspect, Cy¹, when present, is pyrrolyl monosubstituted with a group selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino. In an even further aspect, Cy¹, when present, is unsubstituted pyrrolyl.

### 2. Example Compounds

In one aspect, a compound can be present as one or more of the following structures:

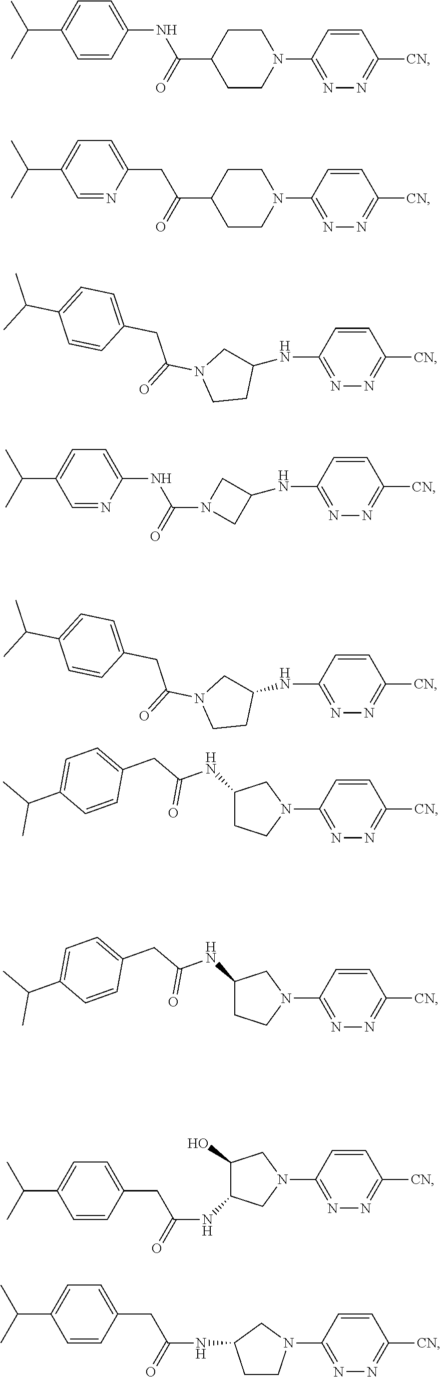

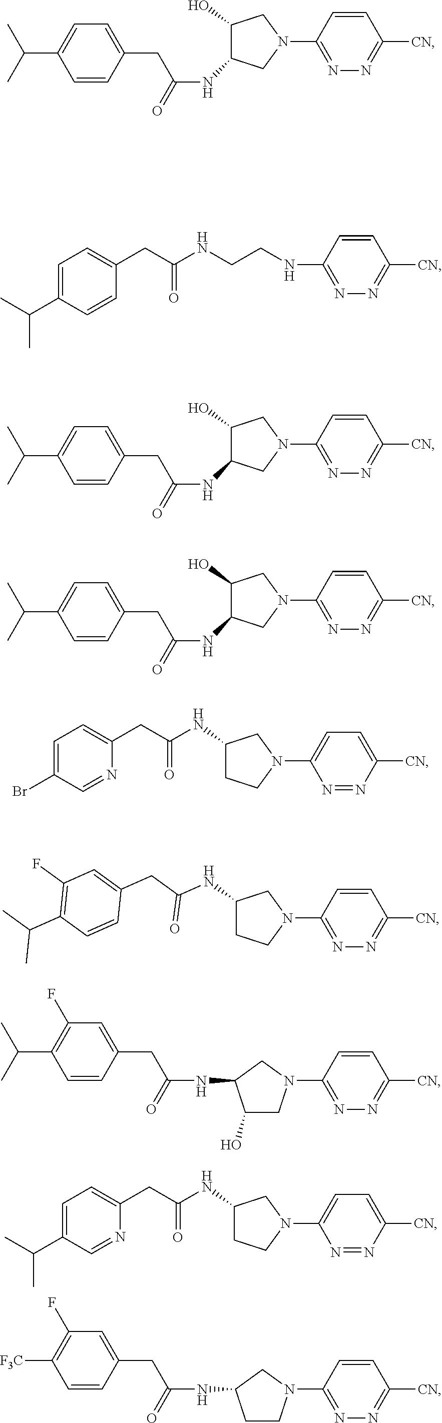

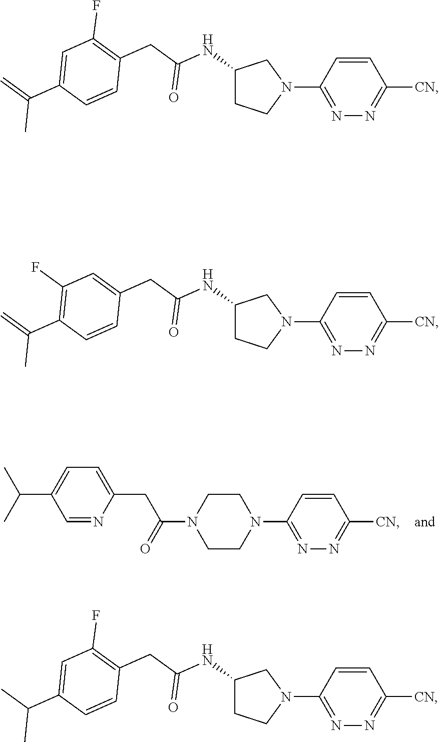 [SMILES: C=C(C)c1ccc(CC(=O)N[C@H]2CCN(c3ccc(C#N)nn3)C2)c(F)c1 ; C=C(C)c1ccc(CC(=O)N[C@H]2CCN(c3ccc(C#N)nn3)C2)cc1F ; CC(C)c1ccc(CC(=O)N2CCN(c3ccc(C#N)nn3)CC2)nc1 ; CC(C)c1ccc(CC(=O)N[C@H]2CCN(c3ccc(C#N)nn3)C2)c(F)c1]

or a pharmaceutically acceptable salt thereof.

In one aspect, a compound can be present as one or more of the following structures:

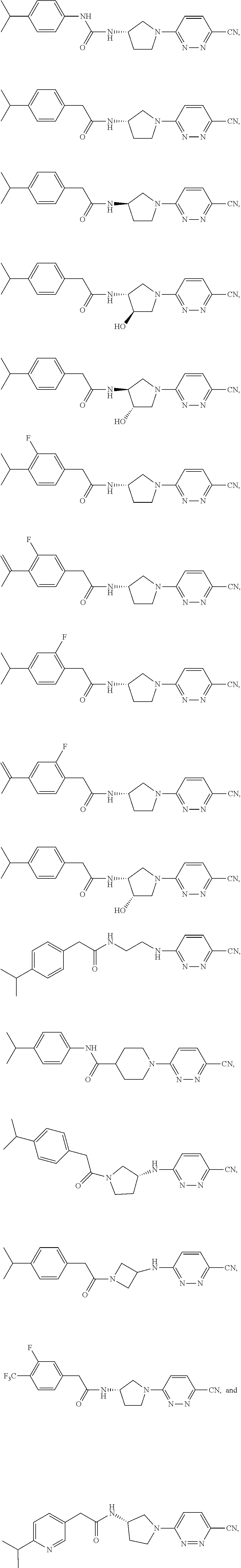

or a pharmaceutically acceptable salt thereof.

In one aspect, a compound can be present as one or more of the following structures:

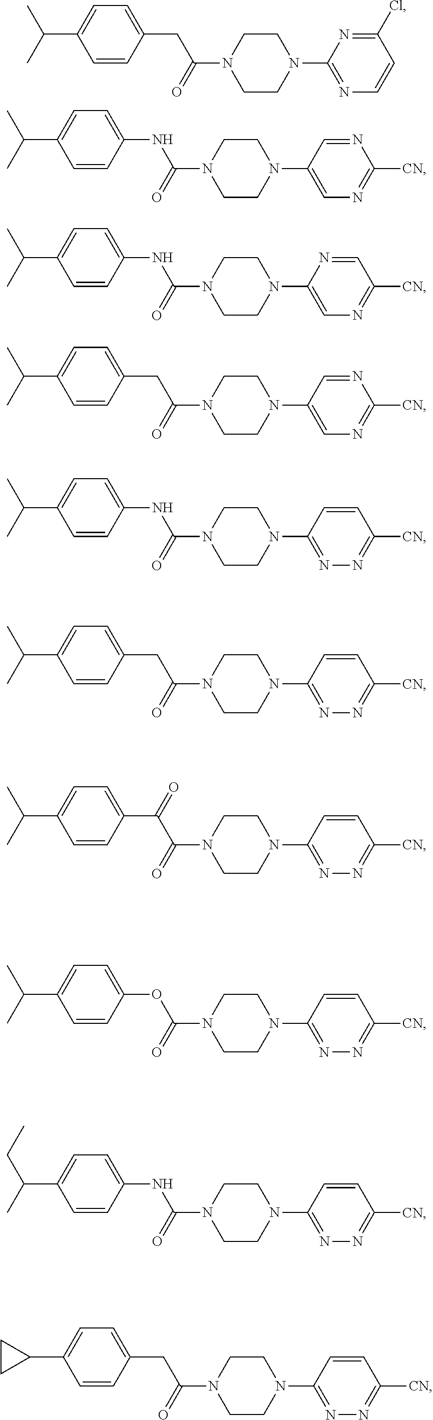

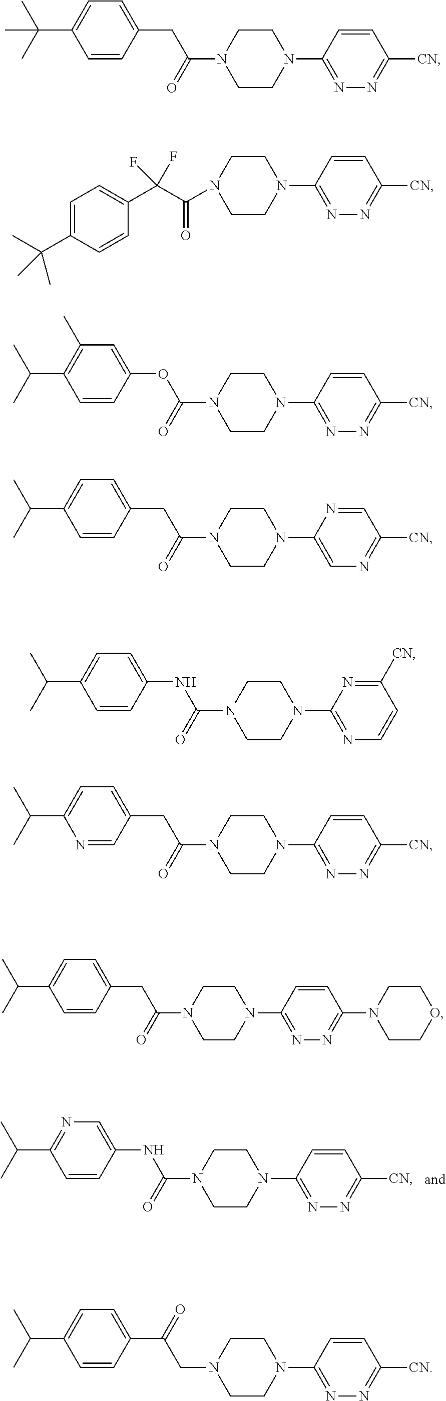

or a pharmaceutically acceptable derivative thereof.

In one aspect, a compound can be present as one or more of the following structures:

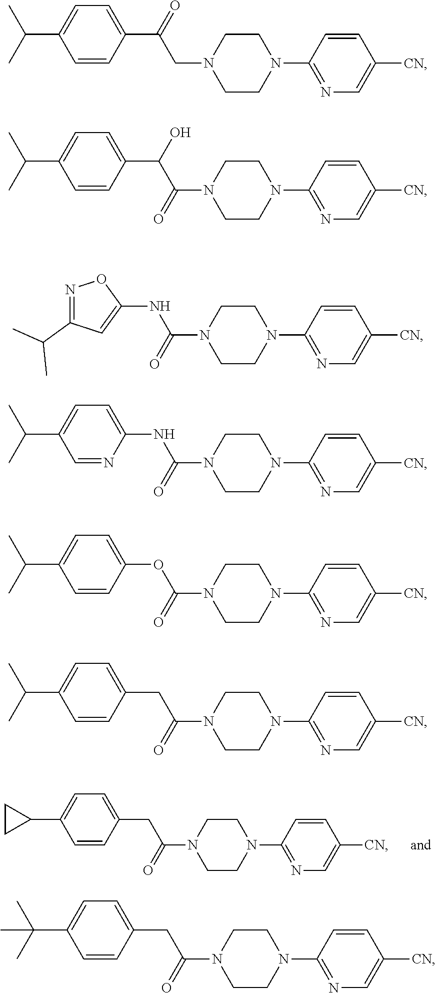

or a pharmaceutically acceptable derivative thereof.

In one aspect, a compound can be present as one or more of the following structures:

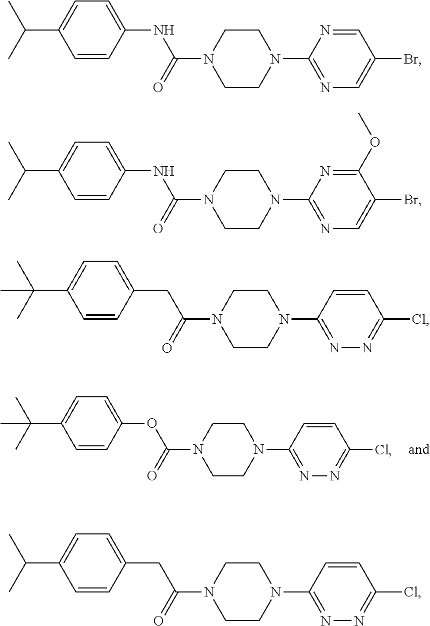

or a pharmaceutically acceptable salt thereof.

In one aspect, a compound can be present as one or more of the following structures:

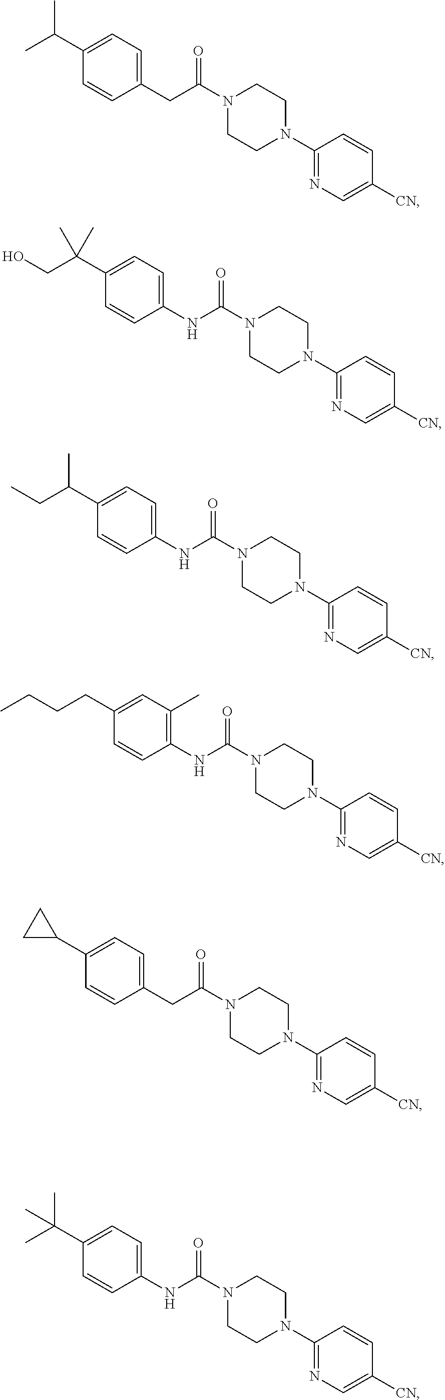 [SMILES: CC(C)c1ccc(CC(=O)N2CCN(c3ccc(C#N)cn3)CC2)cc1 ; CC(C)(CO)c1ccc(NC(=O)N2CCN(c3ccc(C#N)cn3)CC2)cc1 ; CCC(C)c1ccc(NC(=O)N2CCN(c3ccc(C#N)cn3)CC2)cc1 ; CCCCc1ccc(NC(=O)N2CCN(c3ccc(C#N)cn3)CC2)c(C)c1 ; N#Cc1ccc(N2CCN(C(=O)Cc3ccc(C4CC4)cc3)CC2)nc1 ; CC(C)(C)c1ccc(NC(=O)N2CCN(c3ccc(C#N)cn3)CC2)cc1]

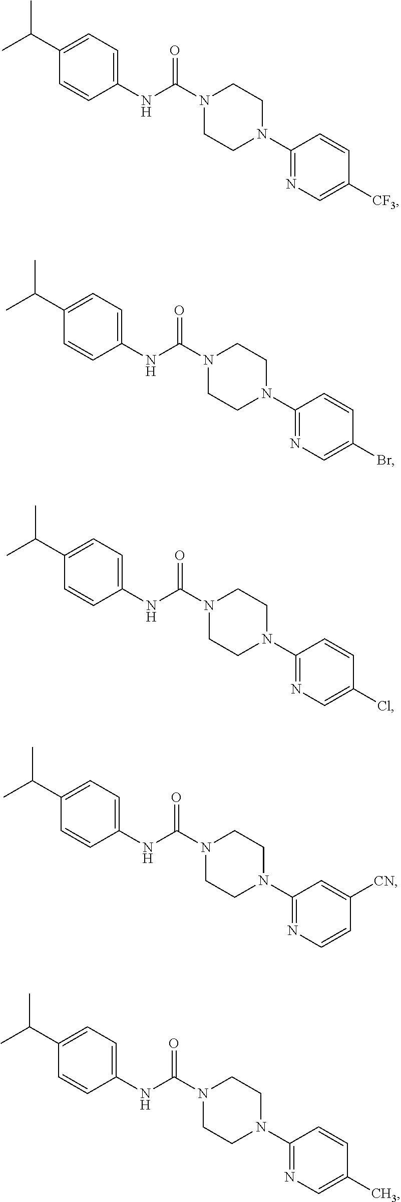 [SMILES: CC(C)c1ccc(NC(=O)N2CCN(c3ccc(C(F)(F)F)cn3)CC2)cc1 ; CC(C)c1ccc(NC(=O)N2CCN(c3ccc(Br)cn3)CC2)cc1 ; CC(C)c1ccc(NC(=O)N2CCN(c3ccc(Cl)cn3)CC2)cc1 ; CC(C)c1ccc(NC(=O)N2CCN(c3cc(C#N)ccn3)CC2)cc1 ; Cc1ccc(N2CCN(C(=O)Nc3ccc(C(C)C)cc3)CC2)nc1]

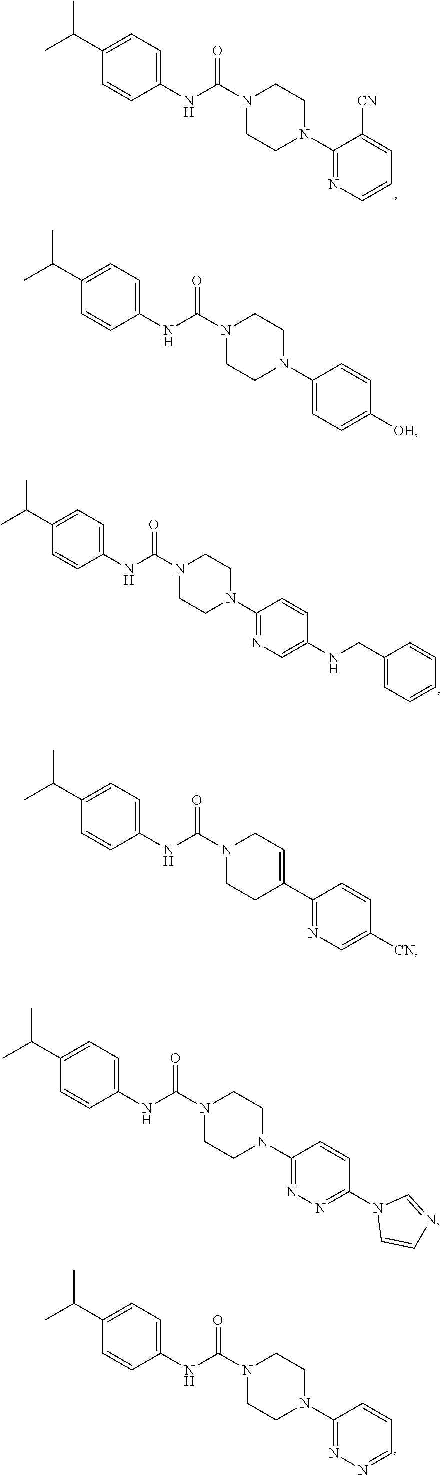 [SMILES: CC(C)c1ccc(NC(=O)N2CCN(c3ncccc3C#N)CC2)cc1 ; CC(C)c1ccc(NC(=O)N2CCN(c3ccc(O)cc3)CC2)cc1 ; CC(C)c1ccc(NC(=O)N2CCN(c3ccc(NCc4ccccc4)cn3)CC2)cc1 ; CC(C)c1ccc(NC(=O)N2CC=C(c3ccc(C#N)cn3)CC2)cc1 ; CC(C)c1ccc(NC(=O)N2CCN(c3ccc(-n4ccnc4)nn3)CC2)cc1 ; CC(C)c1ccc(NC(=O)N2CCN(c3cccnn3)CC2)cc1]

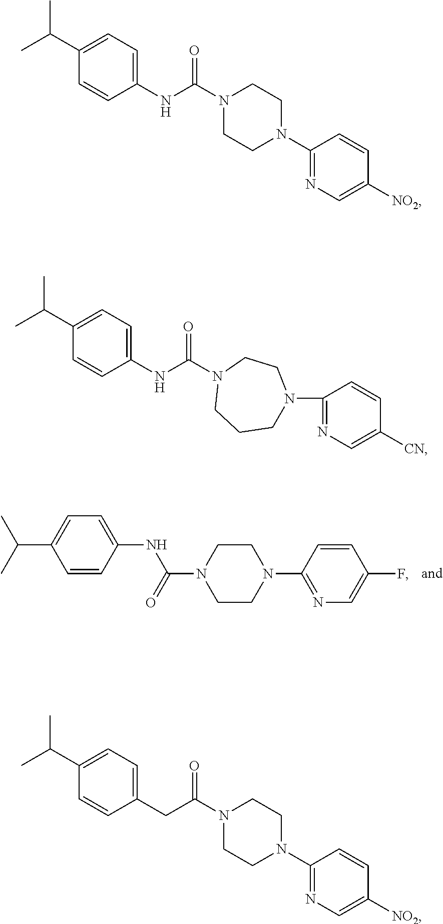 [SMILES: CC(C)c1ccc(NC(=O)N2CCN(c3ccc([N+](=O)[O-])cn3)CC2)cc1 ; CC(C)c1ccc(NC(=O)N2CCCN(c3ccc(C#N)cn3)CC2)cc1 ; CC(C)c1ccc(NC(=O)N2CCN(c3ccc(F)cn3)CC2)cc1 ; CC(C)c1ccc(CC(=O)N2CCN(c3ccc([N+](=O)[O-])cn3)CC2)cc1]

or a pharmaceutically acceptable salt thereof.

In one aspect, a compound can be present as one or more of the following structures:

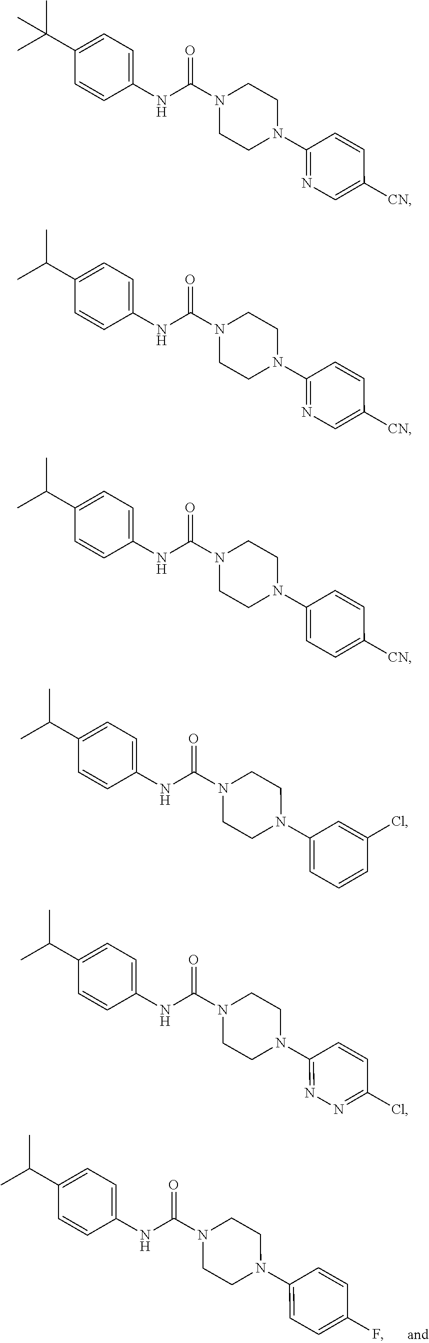 [SMILES: CC(C)(C)c1ccc(NC(=O)N2CCN(c3ccc(C#N)cn3)CC2)cc1 ; CC(C)c1ccc(NC(=O)N2CCN(c3ccc(C#N)cn3)CC2)cc1 ; CC(C)c1ccc(NC(=O)N2CCN(c3ccc(C#N)cc3)CC2)cc1 ; CC(C)c1ccc(NC(=O)N2CCN(c3cccc(Cl)c3)CC2)cc1 ; CC(C)c1ccc(NC(=O)N2CCN(c3ccc(Cl)nn3)CC2)cc1 ; CC(C)c1ccc(NC(=O)N2CCN(c3ccc(F)cc3)CC2)cc1]

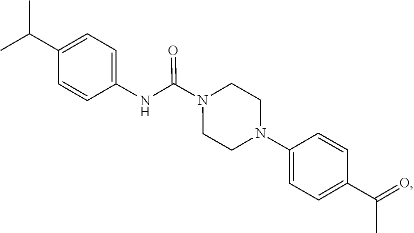 [SMILES: CC(=O)c1ccc(N2CCN(C(=O)Nc3ccc(C(C)C)cc3)CC2)cc1]

or a pharmaceutically acceptable salt thereof.

In one aspect, a compound can be present as one or more of the following structures:

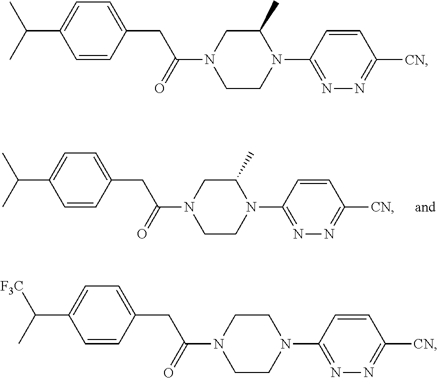 [SMILES: CC(C)c1ccc(CC(=O)N2CCN(c3ccc(C#N)nn3)[C@H](C)C2)cc1 ; CC(C)c1ccc(CC(=O)N2CCN(c3ccc(C#N)nn3)[C@@H](C)C2)cc1 ; CC(c1ccc(CC(=O)N2CCN(c3ccc(C#N)nn3)CC2)cc1)C(F)(F)F]

or a pharmaceutically acceptable salt thereof.

In one aspect, a compound can be present as one or more of the following structures:

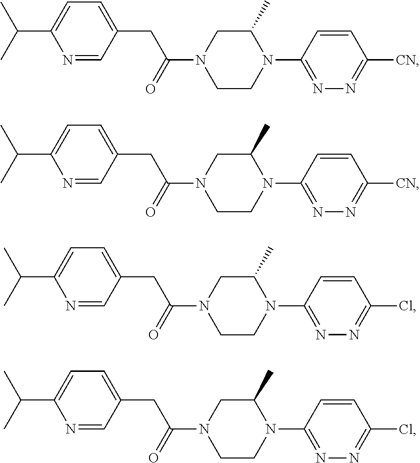

or a pharmaceutically acceptable salt thereof

In one aspect, a compound can be present as one or more of the following structures:

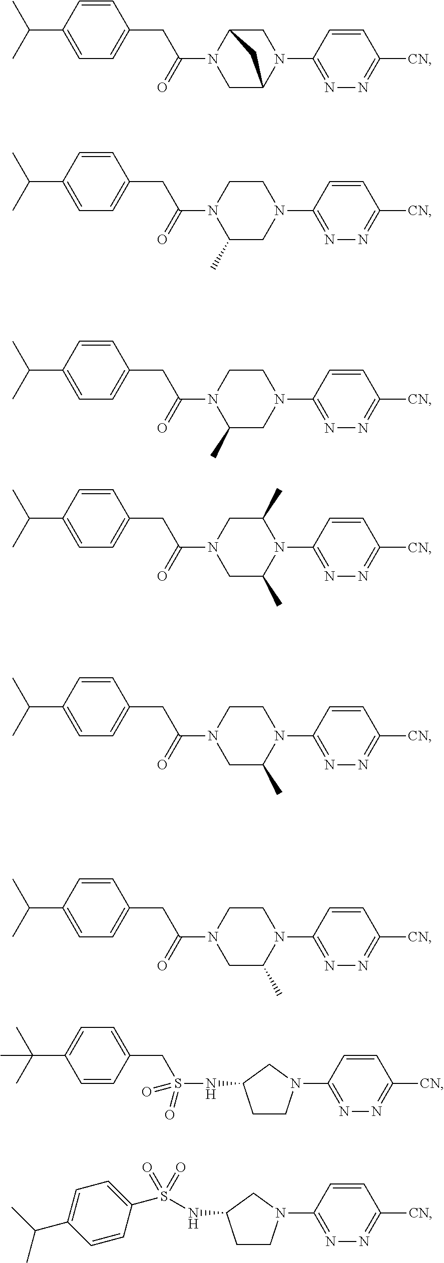

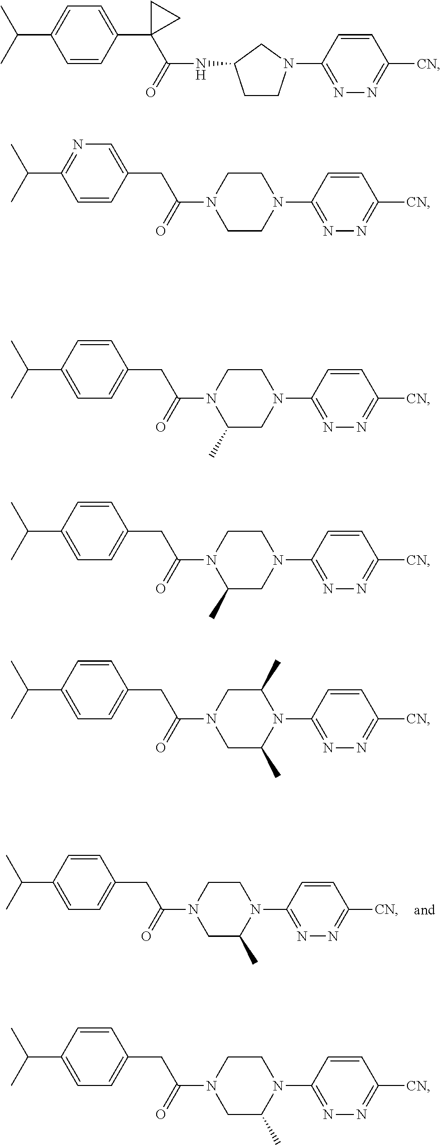

or a pharmaceutically acceptable salt thereof.

In one aspect, a compound can be present as one or more of the following structures:

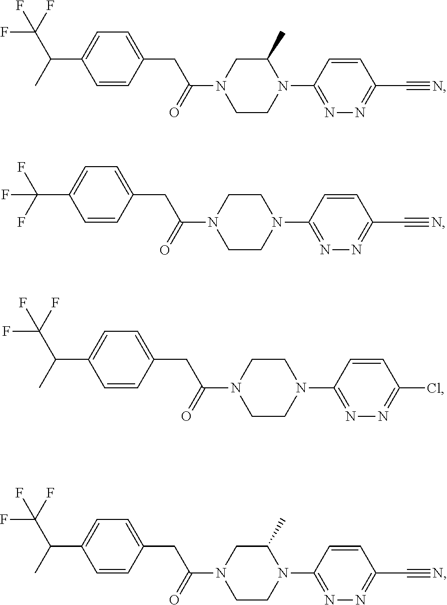 [SMILES: CC(c1ccc(CC(=O)N2CCN(c3ccc(C#N)nn3)[C@H](C)C2)cc1)C(F)(F)F ; N#Cc1ccc(N2CCN(C(=O)Cc3ccc(C(F)(F)F)cc3)CC2)nn1 ; CC(c1ccc(CC(=O)N2CCN(c3ccc(Cl)nn3)CC2)cc1)C(F)(F)F ; CC(c1ccc(CC(=O)N2CCN(c3ccc(C#N)nn3)[C@@H](C)C2)cc1)C(F)(F)F]

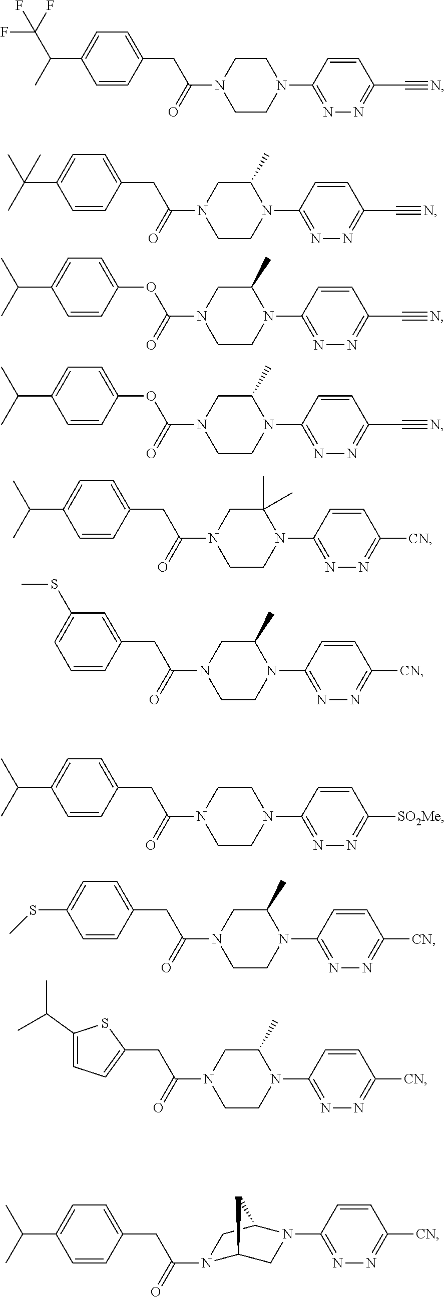

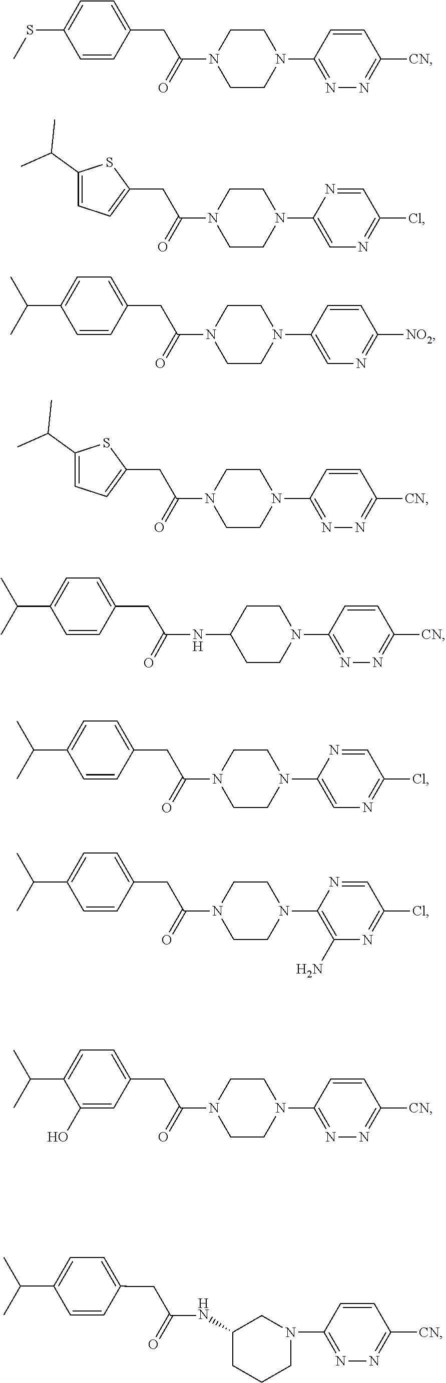

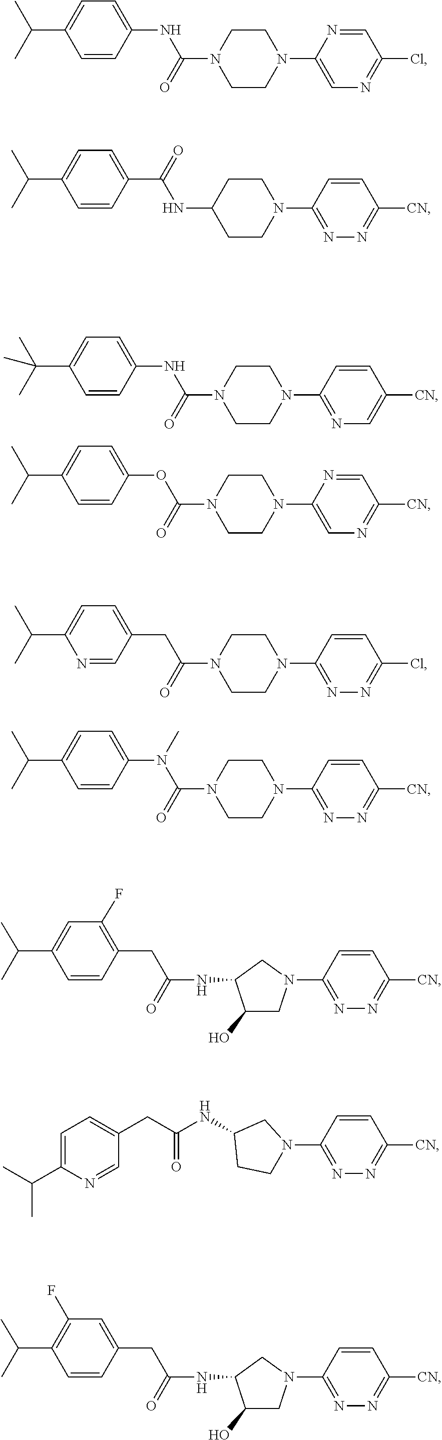

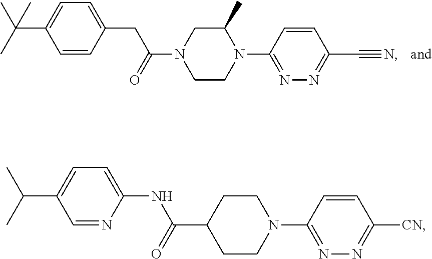 [SMILES: C[C@@H]1CN(C(=O)Cc2ccc(C(C)(C)C)cc2)CCN1c1ccc(C#N)nn1 ; CC(C)c1ccc(NC(=O)C2CCN(c3ccc(C#N)nn3)CC2)nc1]

or a pharmaceutically acceptable salt thereof.

In one aspect, a compound can be present as one or more of the following structures:

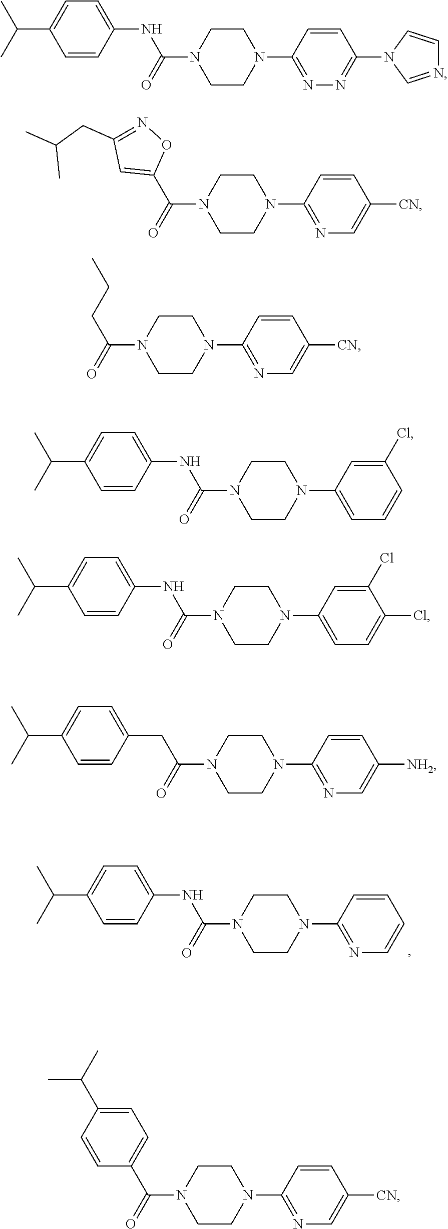

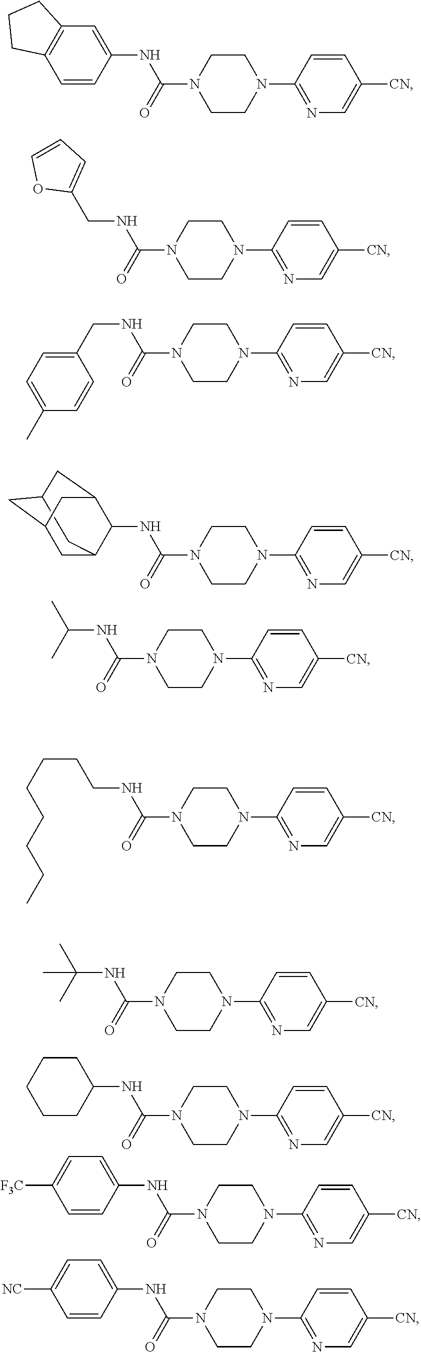

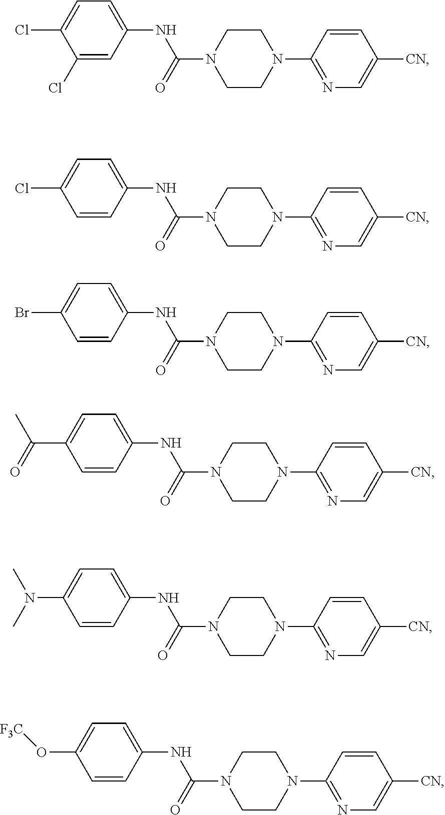 [SMILES: N#Cc1ccc(N2CCN(C(=O)Nc3ccc(Cl)c(Cl)c3)CC2)nc1 ; N#Cc1ccc(N2CCN(C(=O)Nc3ccc(Cl)cc3)CC2)nc1 ; N#Cc1ccc(N2CCN(C(=O)Nc3ccc(Br)cc3)CC2)nc1 ; CC(=O)c1ccc(NC(=O)N2CCN(c3ccc(C#N)cn3)CC2)cc1 ; CN(C)c1ccc(NC(=O)N2CCN(c3ccc(C#N)cn3)CC2)cc1 ; N#Cc1ccc(N2CCN(C(=O)Nc3ccc(OC(F)(F)F)cc3)CC2)nc1]

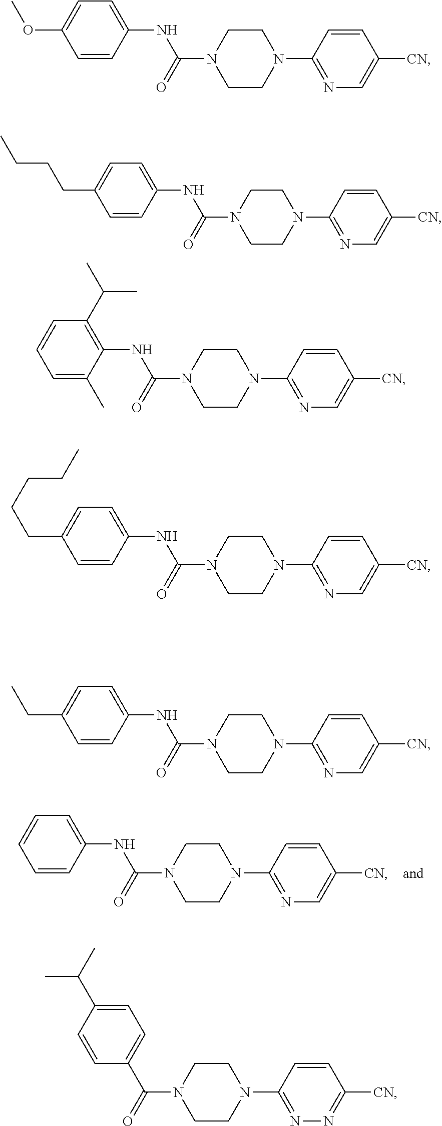

or a pharmaceutically acceptable salt thereof.

In one aspect, a compound can be:

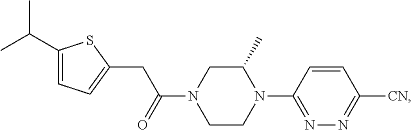 [SMILES: CC(C)c1ccc(CC(=O)N2CCN(c3ccc(C#N)nn3)[C@@H](C)C2)s1]

or a pharmaceutically acceptable salt thereof.

In one aspect, a compound can be:

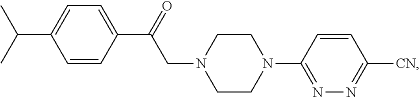 [SMILES: CC(C)c1ccc(C(=O)CN2CCN(c3ccc(C#N)nn3)CC2)cc1]

or a pharmaceutically acceptable salt thereof.

In one aspect, a compound can be:

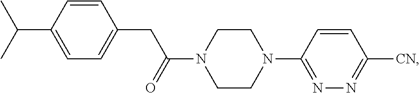 [SMILES: CC(C)c1ccc(CC(=O)N2CCN(c3ccc(C#N)nn3)CC2)cc1]

or a pharmaceutically acceptable salt thereof.

In one aspect, a compound can be:

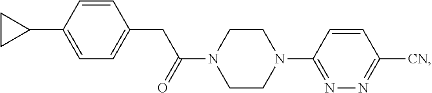 [SMILES: N#Cc1ccc(N2CCN(C(=O)Cc3ccc(C4CC4)cc3)CC2)nn1]

or a pharmaceutically acceptable salt thereof.

In one aspect, a compound can be present as one or more of the following structures:

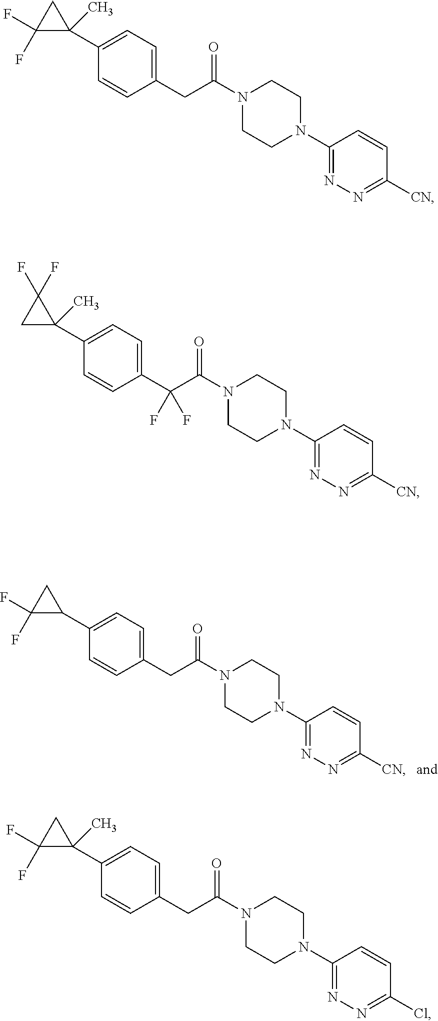 [SMILES: CC1(c2ccc(CC(=O)N3CCN(c4ccc(C#N)nn4)CC3)cc2)CC1(F)F ; CC1(c2ccc(C(F)(F)C(=O)N3CCN(c4ccc(C#N)nn4)CC3)cc2)CC1(F)F ; N#Cc1ccc(N2CCN(C(=O)Cc3ccc(C4CC4(F)F)cc3)CC2)nn1 ; "and" ; CC1(c2ccc(CC(=O)N3CCN(c4ccc(Cl)nn4)CC3)cc2)CC1(F)F]

or a pharmaceutically acceptable salt thereof.

In one aspect, a compound can be present as the following structure:

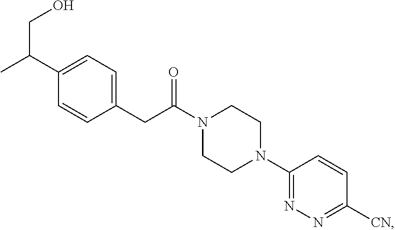 [SMILES: CC(CO)c1ccc(CC(=O)N2CCN(c3ccc(C#N)nn3)CC2)cc1]

or a pharmaceutically acceptable salt thereof.

In one aspect, a compound can be present as one or more of the following structures:

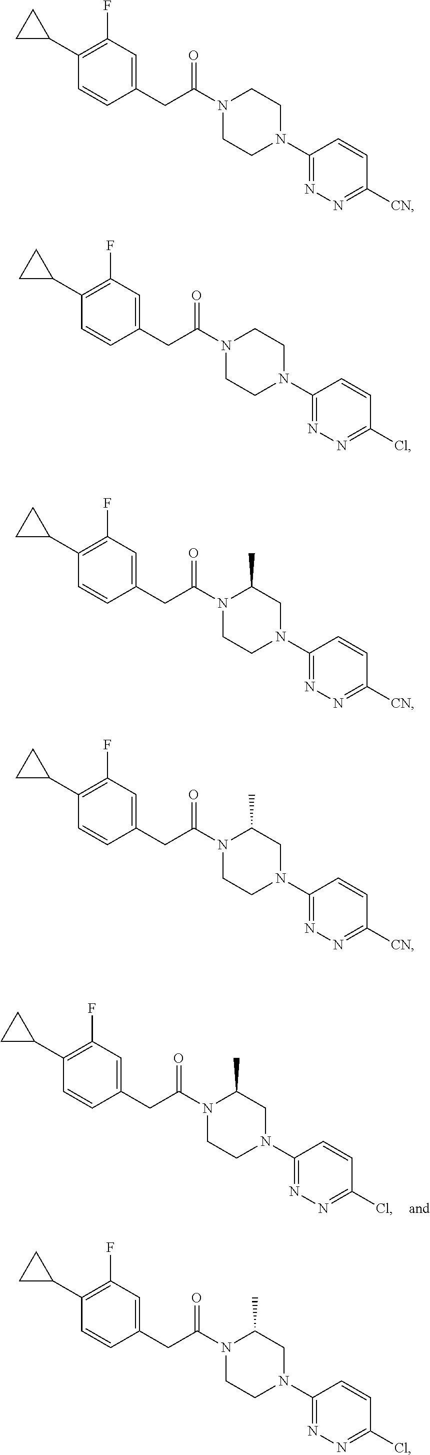

or a pharmaceutically acceptable salt thereof.

In a further aspect, a compound can be present as one or more of the following structures:

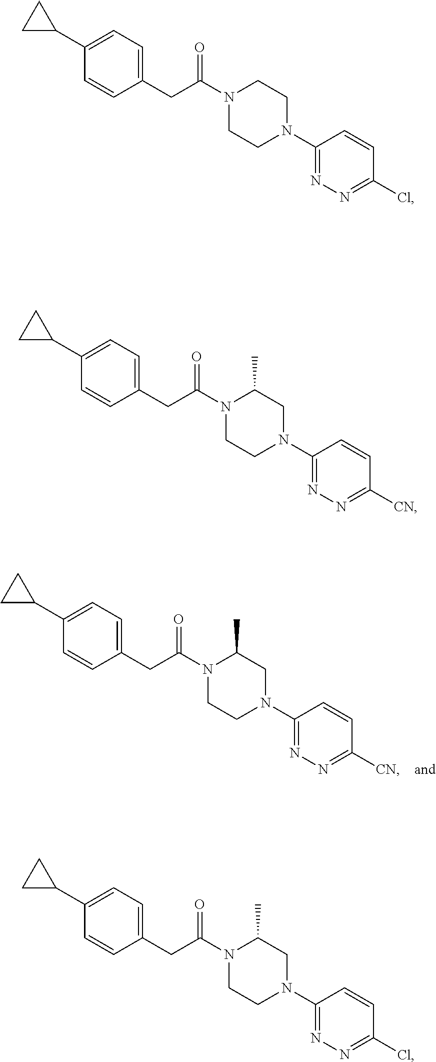 [SMILES: O=C(Cc1ccc(C2CC2)cc1)N1CCN(c2ccc(Cl)nn2)CC1 ; C[C@@H]1CN(c2ccc(C#N)nn2)CCN1C(=O)Cc1ccc(C2CC2)cc1 ; C[C@H]1CN(c2ccc(C#N)nn2)CCN1C(=O)Cc1ccc(C2CC2)cc1 ; C[C@@H]1CN(c2ccc(Cl)nn2)CCN1C(=O)Cc1ccc(C2CC2)cc1]

or a pharmaceutically acceptable salt thereof.

The compounds described herein can be prepared using various schemes, including those shown in International Patent Publications Nos. WO2019133632, WO2019133635, and WO2017223474, which are herein incorporated by reference in their entireties. Details of the compounds described herein, including characterization and synthetic details, are also included in these publications.

Without wishing to be bound by theory, the compounds described herein may be capable of allosterically activating alternate PANK isoforms and may therefore be useful as a potential therapeutic for PKAN and other disorders described herein. Administration of such compounds may yield physiological improvements including weight gain, improved locomoter activity, and life span.

### V. COMPOSITION

In some embodiments, a compound provided herein may be included in a pharmaceutical composition comprising the compound, or a pharmaceutically acceptable salt thereof, and a pharmaceutically acceptable carrier.

In various aspects, the compounds and compositions of the present disclosure can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined. In various aspects, the pharmaceutical composition is sterile or sterilizable. The therapeutic compositions featured in the present disclosure can contain carriers or excipients, many of which are known to skilled artisans.

Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the present disclosure can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, P A 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this disclosure can include a pharmaceutically acceptable carrier and a compound or a pharmaceutically acceptable salt of the compounds of the present disclosure. The compounds of the present disclosure, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this disclosure can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present disclosure comprise a compound described herein (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Compositions containing a compound of the present disclosure, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with pantothenate kinase activity such as, for example, PKAN and diabetes.

It is understood that the disclosed compositions can be prepared from the disclosed compounds. It is also understood that the disclosed compositions can be employed in the disclosed methods of using.

### VI. DOSING

A dose may provide a therapeutic benefit while balancing safety considerations. Data obtained from cell culture assays and further animal studies can be used in formulating a range of dosage for use in humans. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

### VII. ROUTE OF ADMINISTRATION

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In some embodiments of any of the methods provided herein, administration of a compound provided herein, or a form thereof, or a pharmaceutical composition comprising the same, is oral administration.

In various aspects, the modes of administration described above may be combined in any order.

### VIII. EMBODIMENTS

Embodiment B1. A method for identifying a subject in need of a treatment with a therapeutic agent, comprising:
(a) using a magnetic resonance technique to measure a substance in the brain of the subject; and
(b) based at least in part on the amount of the substance, identifying the subject as being in need of the treatment with the therapeutic agent, wherein the therapeutic agent comprises a compound has a structure represented by the formula:

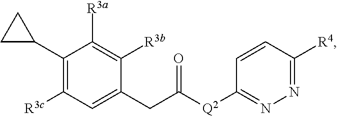

wherein Q² is a structure selected from

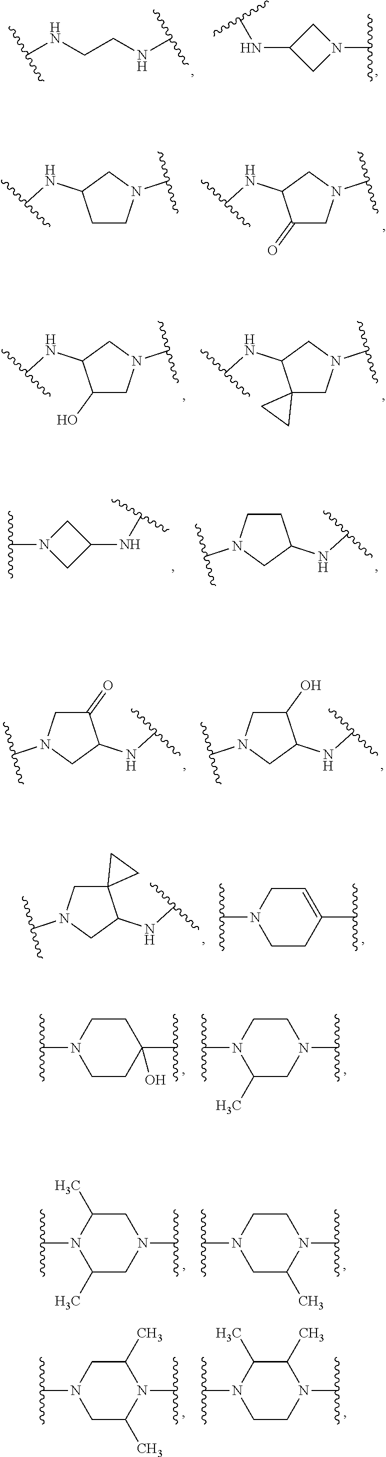

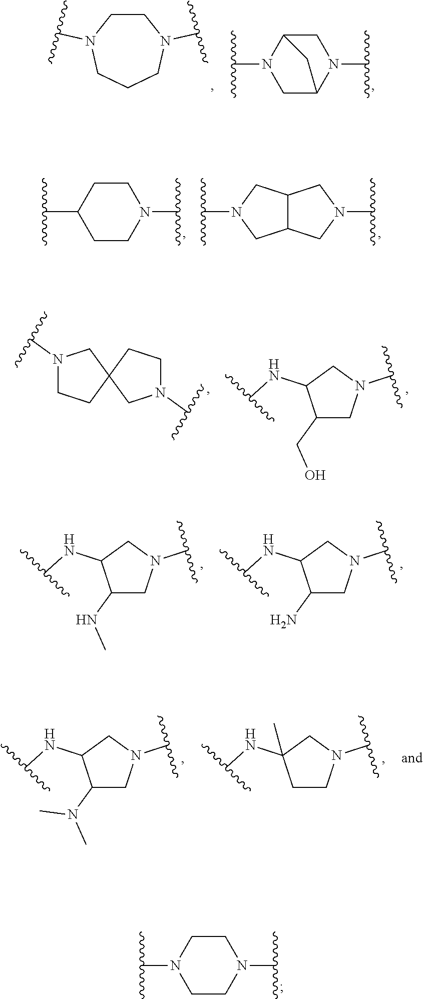

wherein each of R^{3a}, R^{3b}, and R^{3c} is independently selected from hydrogen, halogen, C1-C4 alkoxy and C1-C4 alkyl, provided at least one of R^{3a}, R^{3b}, and R^{3c} is halogen; and
wherein R⁴ is selected form hydrogen, halogen, —CN, SO₂NH₂, SO₂CH₃, SO₂CF₃, and NO₂, or a pharmaceutically acceptable salt thereof.

Embodiment B2. The method of embodiment B1, wherein Q² is a structure:

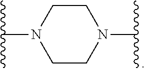 [SMILES: *N1CCN(*)CC1]

Embodiment B3. The method of embodiment B1 or B2, wherein R^{3a} is halogen.

Embodiment B4. The method of embodiment B3, wherein each of R^{3b} and R^{3c} is hydrogen.

Embodiment B5. The method of embodiment B1 or B2, wherein R^{3c} is halogen.

Embodiment B6. The method of any one of embodiments B1 to B5, wherein R⁴ is —CN or —Cl.

Embodiment B7. The method of embodiment B1, wherein the compound has a structure:

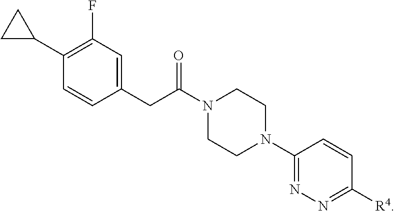

Embodiment B8. The method of embodiment B1, wherein the compound is selected from:

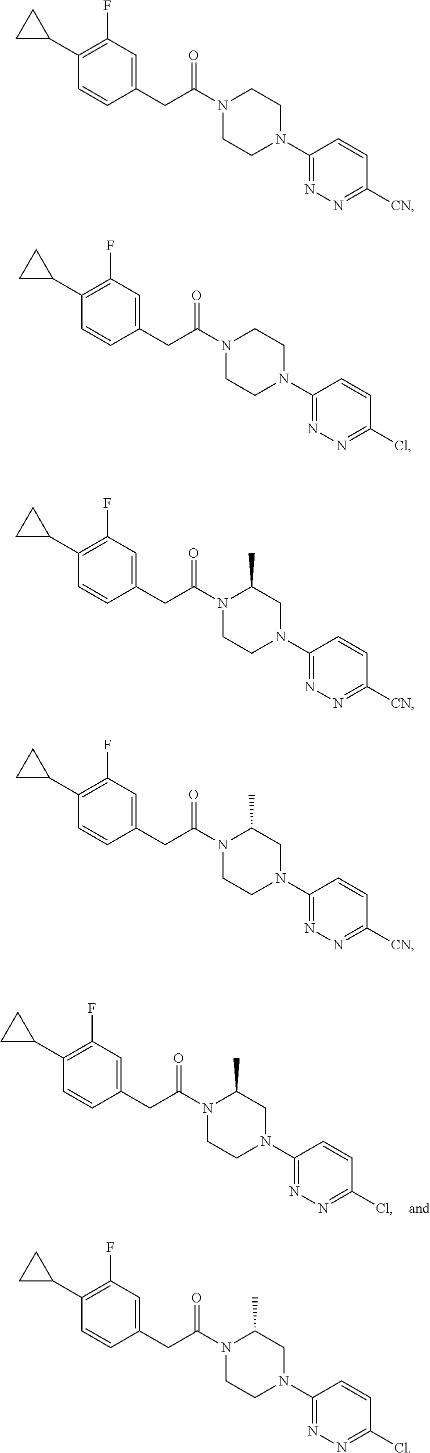

Embodiment B9. The method of embodiment B1, wherein the compound is selected from:

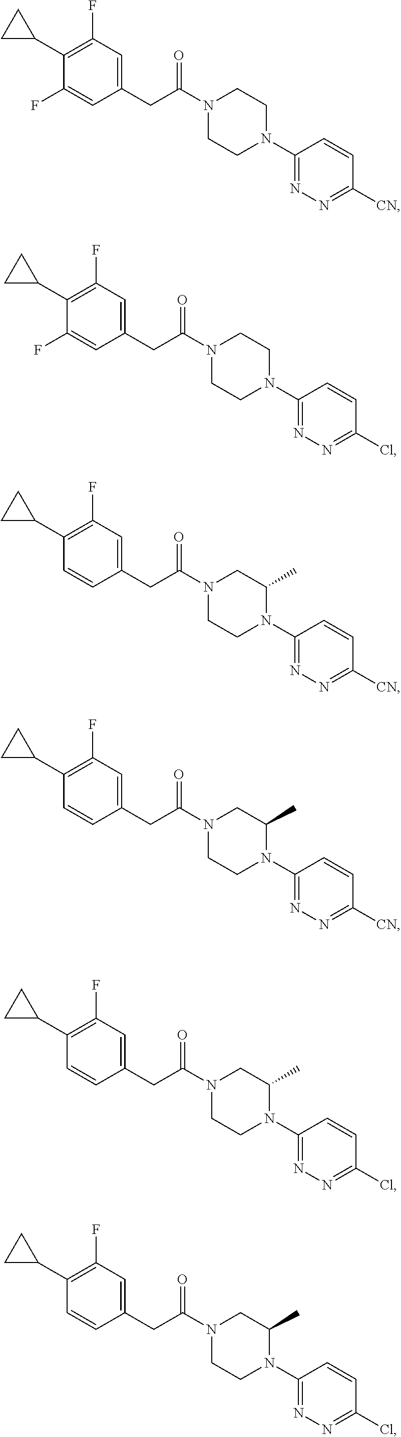 [SMILES: N#Cc1ccc(N2CCN(C(=O)Cc3cc(F)c(C4CC4)c(F)c3)CC2)nn1 ; O=C(Cc1cc(F)c(C2CC2)c(F)c1)N1CCN(c2ccc(Cl)nn2)CC1 ; C[C@H]1CN(C(=O)Cc2ccc(C3CC3)c(F)c2)CCN1c1ccc(C#N)nn1 ; C[C@@H]1CN(C(=O)Cc2ccc(C3CC3)c(F)c2)CCN1c1ccc(C#N)nn1 ; C[C@H]1CN(C(=O)Cc2ccc(C3CC3)c(F)c2)CCN1c1ccc(Cl)nn1 ; C[C@@H]1CN(C(=O)Cc2ccc(C3CC3)c(F)c2)CCN1c1ccc(Cl)nn1]

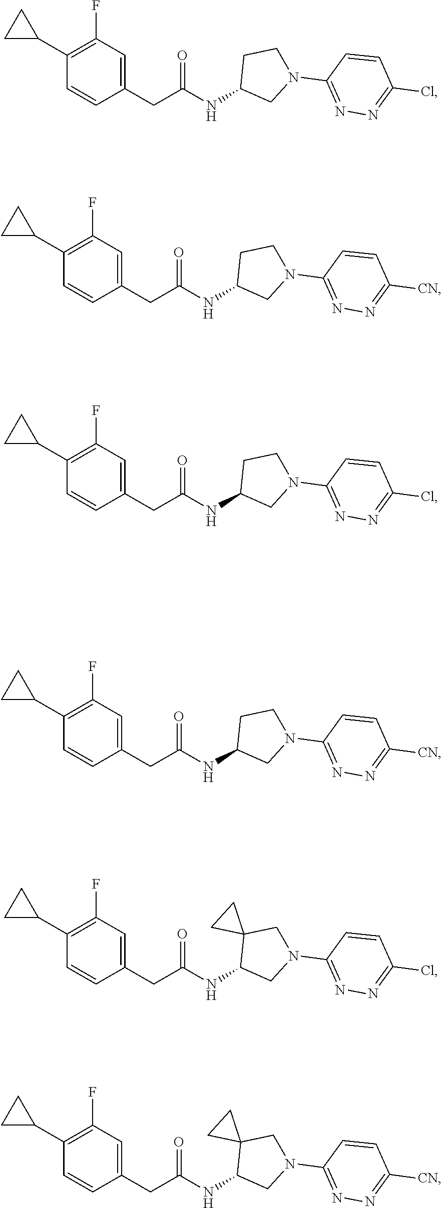 [SMILES: O=C(Cc1ccc(C2CC2)c(F)c1)N[C@@H]1CCN(c2ccc(Cl)nn2)C1 ; N#Cc1ccc(N2CC[C@@H](NC(=O)Cc3ccc(C4CC4)c(F)c3)C2)nn1 ; O=C(Cc1ccc(C2CC2)c(F)c1)N[C@H]1CCN(c2ccc(Cl)nn2)C1 ; N#Cc1ccc(N2CC[C@H](NC(=O)Cc3ccc(C4CC4)c(F)c3)C2)nn1 ; O=C(Cc1ccc(C2CC2)c(F)c1)N[C@H]1CN(c2ccc(Cl)nn2)CC12CC2 ; N#Cc1ccc(N2C[C@H](NC(=O)Cc3ccc(C4CC4)c(F)c3)C3(CC3)C2)nn1]

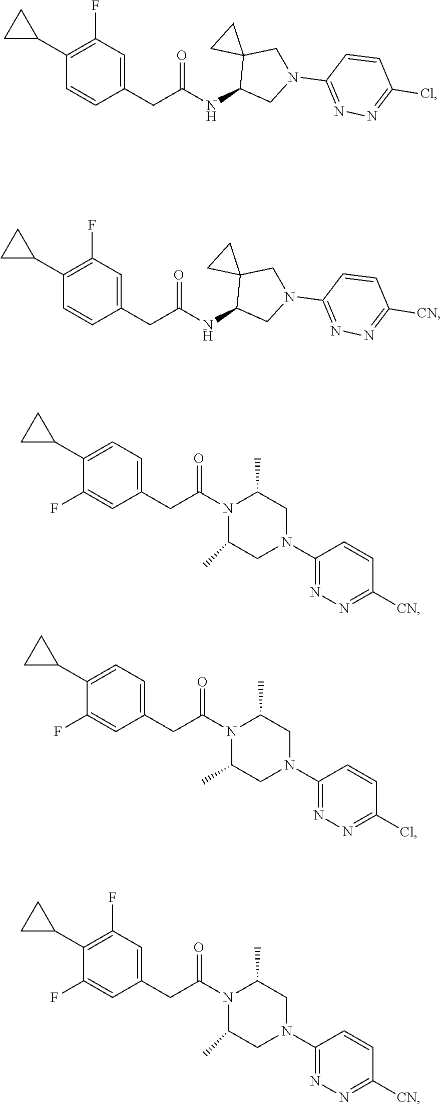 [SMILES: O=C(Cc1ccc(C2CC2)c(F)c1)N[C@@H]1CN(c2ccc(Cl)nn2)CC12CC2 ; N#Cc1ccc(N2C[C@@H](NC(=O)Cc3ccc(C4CC4)c(F)c3)C3(CC3)C2)nn1 ; C[C@@H]1CN(c2ccc(C#N)nn2)C[C@H](C)N1C(=O)Cc1ccc(C2CC2)c(F)c1 ; C[C@@H]1CN(c2ccc(Cl)nn2)C[C@H](C)N1C(=O)Cc1ccc(C2CC2)c(F)c1 ; C[C@@H]1CN(c2ccc(C#N)nn2)C[C@H](C)N1C(=O)Cc1cc(F)c(C2CC2)c(F)c1]

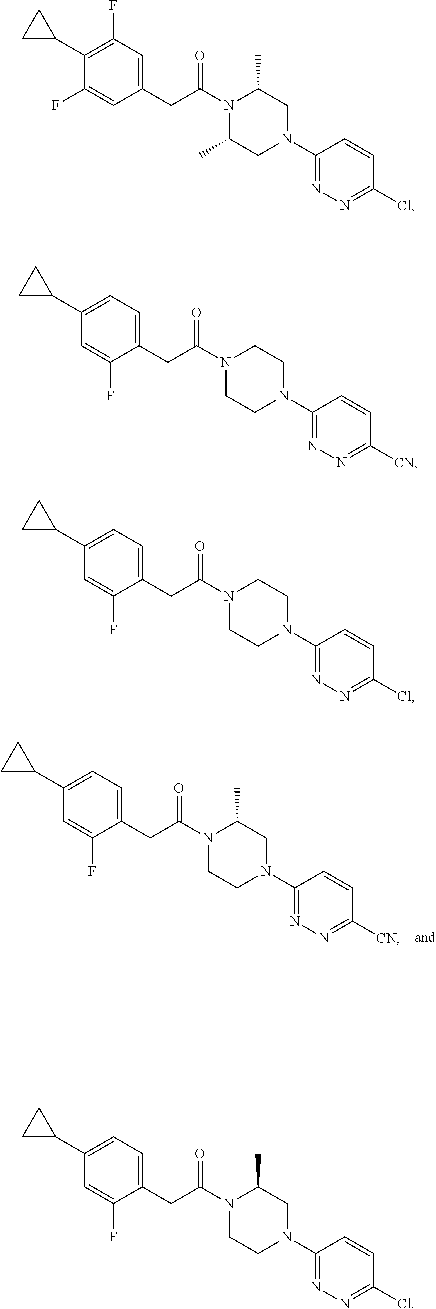 [SMILES: C[C@@H]1CN(c2ccc(Cl)nn2)C[C@H](C)N1C(=O)Cc1cc(F)c(C2CC2)c(F)c1 ; N#Cc1ccc(N2CCN(C(=O)Cc3ccc(C4CC4)cc3F)CC2)nn1 ; O=C(Cc1ccc(C2CC2)cc1F)N1CCN(c2ccc(Cl)nn2)CC1 ; C[C@@H]1CN(c2ccc(C#N)nn2)CCN1C(=O)Cc1ccc(C2CC2)cc1F ; C[C@H]1CN(c2ccc(Cl)nn2)CCN1C(=O)Cc1ccc(C2CC2)cc1F]

Embodiment B10. The method of any one of embodiments B1 to B9, wherein the subject is a human.

Embodiment B11. The method of any one of embodiments B1 to B10, wherein the magnetic resonance technique comprises magnetic resonance imaging.

Embodiment B12. The method of any one of embodiments B1 to B11, wherein the substance is glutamate/glutamine (Glx), γ-aminobutyric acid (GABA), lactate, inositol, choline, taurine, or N-acetyl aspartate.

Embodiment B13. The method of any one of embodiments B1 to B12, wherein the substance is Glx, GABA, lactate, or N-acetyl aspartate.

Embodiment B14. The method of any one of embodiments B1 to B13, wherein the subject is diagnosed with a metabolic disease, neurologic disorder, or a coenzyme A reduction, elevation, sequestration, toxicity, or redistribution (CASTOR) disorder, a metabolic disease, or a neurologic disorder.

Embodiment B15. The method of embodiment B14, wherein the subject is diagnosed with pantothenate kinase-associated neurodegeneration (PKAN).

Embodiment B16. The method of embodiment B14, wherein the subject is diagnosed with a CASTOR disorder.

Embodiment B17. The method of embodiment B16, wherein the subject is diagnosed with defects in fatty acid oxidation enzymes, methylmalonic acidemia, glutaric acidemia, propionic acidemia, or HMG-CoA lyase deficiency.

Embodiment B18. A method for assessing a therapeutic regimen comprising administration of a first amount of a therapeutic agent at a first frequency, comprising:
(a) using a magnetic resonance technique to measure a substance in the brain of the subject at a first time;
(b) using the magnetic resonance technique to measure the substance in the brain of the subject at a second time after the first time;
(c) assessing the difference between the amount of the substance at the first time and the second time; and
(d) based at least in part on (c), changing the first amount of the therapeutic regimen to a second amount and/or changing the first frequency of the therapeutic regimen to a second frequency if the amount in (b) or the difference in (c) exceeds or does not meet a threshold level or not changing the first amount and first frequency of the therapeutic regimen if the amount in (b) or the difference in (c) does not exceed the threshold level, wherein the therapeutic agent comprises a compound having a structure represented by the formula:

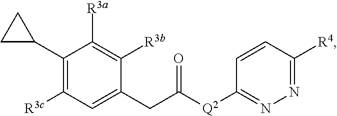

wherein Q² is a structure selected from

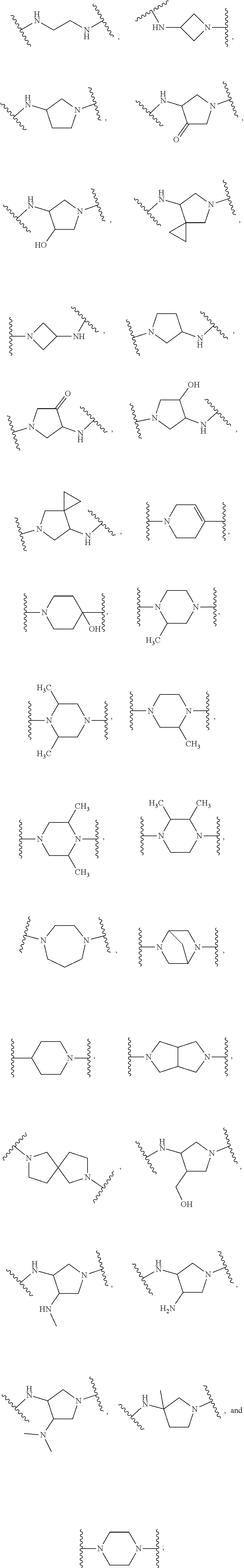

wherein each of R^{3a}, R^{3b}, and R^{3c} is independently selected from hydrogen, halogen, C1-C4 alkoxy and C1-C4 alkyl, provided at least one of R^{3a}, R^{3b}, and R^{3c} is halogen; and
wherein R⁴ is selected form hydrogen, halogen, —CN, SO₂NH₂, SO₂CH₃, SO₂CF₃, and NO₂, or a pharmaceutically acceptable salt thereof.

Embodiment B19. The method of embodiment B18, wherein Q² is a structure:

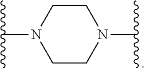 [SMILES: *N1CCN(*)CC1]

Embodiment B20. The method of embodiment B18 or B19, wherein R^{3a} is halogen.

Embodiment B21. The method of embodiment B20, wherein each of R^{3b} and R^{3c} is hydrogen.

Embodiment B22. The method of any one of embodiments B18 to B21, wherein R⁴ is —CN or —Cl.

Embodiment B23. The method of embodiment B18, wherein the compound has a structure:

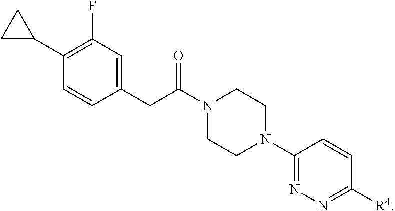

Embodiment B24. The method of embodiment B18, wherein the compound is selected from:

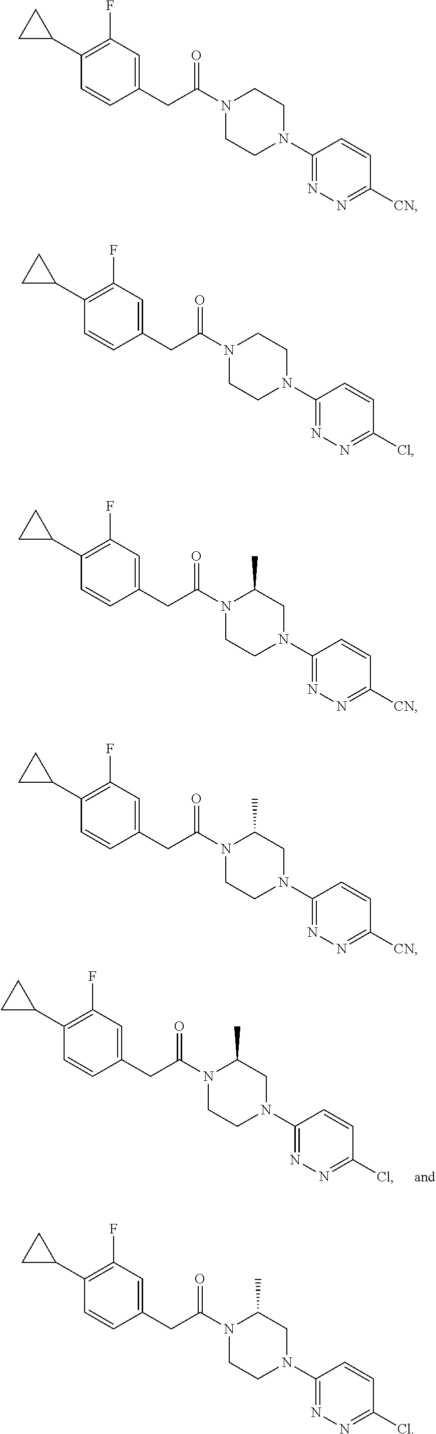 [SMILES: N#Cc1ccc(N2CCN(C(=O)Cc3ccc(C4CC4)c(F)c3)CC2)nn1 ; O=C(Cc1ccc(C2CC2)c(F)c1)N1CCN(c2ccc(Cl)nn2)CC1 ; C[C@H]1CN(c2ccc(C#N)nn2)CCN1C(=O)Cc1ccc(C2CC2)c(F)c1 ; C[C@@H]1CN(c2ccc(C#N)nn2)CCN1C(=O)Cc1ccc(C2CC2)c(F)c1 ; C[C@H]1CN(c2ccc(Cl)nn2)CCN1C(=O)Cc1ccc(C2CC2)c(F)c1 ; C[C@@H]1CN(c2ccc(Cl)nn2)CCN1C(=O)Cc1ccc(C2CC2)c(F)c1]

Embodiment B25. The method of embodiment B18, wherein the compound is selected from:

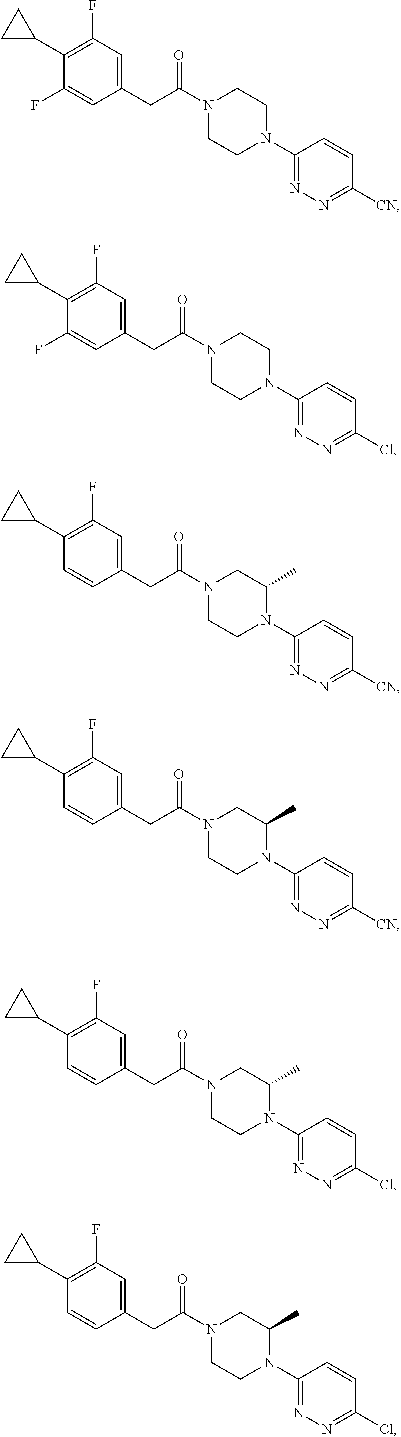 [SMILES: N#Cc1ccc(N2CCN(C(=O)Cc3cc(F)c(C4CC4)c(F)c3)CC2)nn1 ; O=C(Cc1cc(F)c(C2CC2)c(F)c1)N1CCN(c2ccc(Cl)nn2)CC1 ; C[C@H]1CN(C(=O)Cc2ccc(C3CC3)c(F)c2)CCN1c1ccc(C#N)nn1 ; C[C@@H]1CN(C(=O)Cc2ccc(C3CC3)c(F)c2)CCN1c1ccc(C#N)nn1 ; C[C@H]1CN(C(=O)Cc2ccc(C3CC3)c(F)c2)CCN1c1ccc(Cl)nn1 ; C[C@@H]1CN(C(=O)Cc2ccc(C3CC3)c(F)c2)CCN1c1ccc(Cl)nn1]

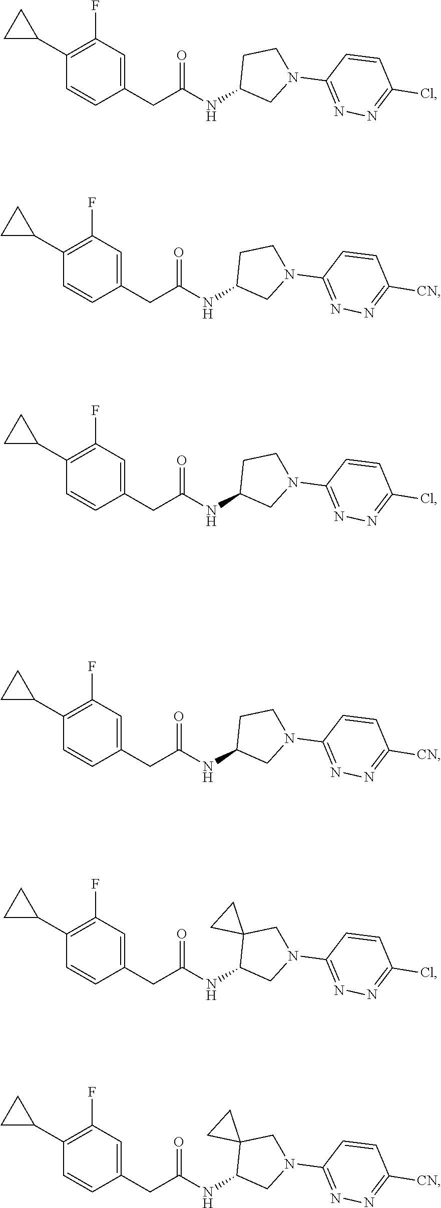 [SMILES: O=C(Cc1ccc(C2CC2)c(F)c1)N[C@@H]1CCN(c2ccc(Cl)nn2)C1 ; N#Cc1ccc(N2CC[C@@H](NC(=O)Cc3ccc(C4CC4)c(F)c3)C2)nn1 ; O=C(Cc1ccc(C2CC2)c(F)c1)N[C@H]1CCN(c2ccc(Cl)nn2)C1 ; N#Cc1ccc(N2CC[C@H](NC(=O)Cc3ccc(C4CC4)c(F)c3)C2)nn1 ; O=C(Cc1ccc(C2CC2)c(F)c1)N[C@H]1CN(c2ccc(Cl)nn2)CC12CC2 ; N#Cc1ccc(N2C[C@H](NC(=O)Cc3ccc(C4CC4)c(F)c3)C3(CC3)C2)nn1]

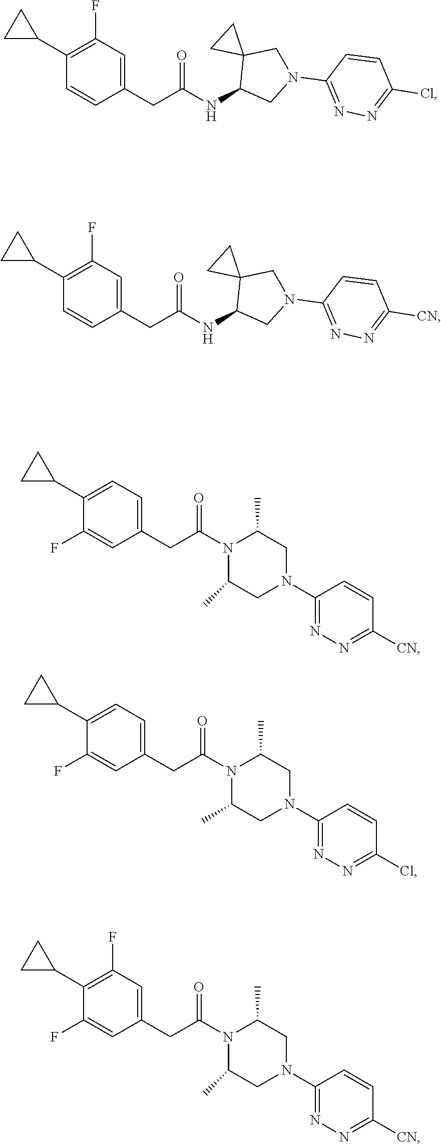 [SMILES: O=C(Cc1ccc(C2CC2)c(F)c1)N[C@@H]1CN(c2ccc(Cl)nn2)CC12CC2 ; N#Cc1ccc(N2C[C@@H](NC(=O)Cc3ccc(C4CC4)c(F)c3)C3(CC3)C2)nn1 ; C[C@@H]1CN(c2ccc(C#N)nn2)C[C@H](C)N1C(=O)Cc1ccc(C2CC2)c(F)c1 ; C[C@@H]1CN(c2ccc(Cl)nn2)C[C@H](C)N1C(=O)Cc1ccc(C2CC2)c(F)c1 ; C[C@@H]1CN(c2ccc(C#N)nn2)C[C@H](C)N1C(=O)Cc1cc(F)c(C2CC2)c(F)c1]

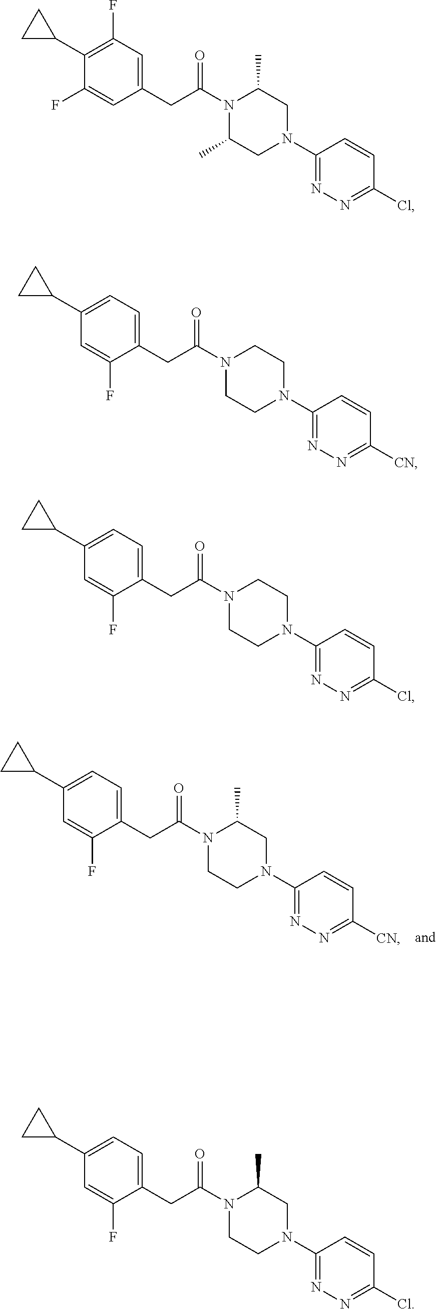 [SMILES: C[C@@H]1CN(c2ccc(Cl)nn2)C[C@H](C)N1C(=O)Cc1cc(F)c(C2CC2)c(F)c1 ; N#Cc1ccc(N2CCN(C(=O)Cc3ccc(C4CC4)cc3F)CC2)nn1 ; O=C(Cc1ccc(C2CC2)cc1F)N1CCN(c2ccc(Cl)nn2)CC1 ; C[C@@H]1CN(c2ccc(C#N)nn2)CCN1C(=O)Cc1ccc(C2CC2)cc1F ; C[C@H]1CN(c2ccc(Cl)nn2)CCN1C(=O)Cc1ccc(C2CC2)cc1F]

Embodiment B26. The method of any one of embodiments B18-B25, wherein the subject is a human.

Embodiment B27. The method of any one of embodiments B18 to B26, wherein the magnetic resonance technique comprises magnetic resonance imaging.

Embodiment B28. The method of any one of embodiments B18 to B27, wherein the substance is glutamate/glutamine (Glx), γ-aminobutyric acid (GABA), lactate, inositol, choline, taurine, and/or N-acetyl aspartate.

Embodiment B29. The method of any one of embodiments B18 to B28, wherein the substance is Glx, GABA, lactate, or N-acetyl aspartate.

Embodiment B30. The method of any one of embodiments B18 to B29, wherein the subject is diagnosed with a disorder selected from metabolic disease, neurological disorder, or a coenzyme A reduction, elevation, sequestration, toxicity, or redistribution (CASTOR) disorder.

Embodiment 31. The method of embodiment B30, wherein the subject is diagnosed with pantothenate kinase-associated neurodegeneration (PKAN).

Embodiment 32. The method of embodiment B30, wherein the subject is diagnosed with a CASTOR disorder.

Embodiment 33. The method of embodiment B32, wherein the subject is diagnosed with defects in fatty acid oxidation enzymes, methylmalonic acidemia, glutaric acidemia, propionic acidemia, or HMG-CoA lyase deficiency.

Embodiment B34. The method of any one of embodiments B30 to B33, wherein the subject is diagnosed with the disorder (i) before performance of (a); (ii) after performance of (a) but before performance of (b); or (iii) after performance of (a) and (b).

Embodiment B35. The method of any one of embodiments B18 to B34, wherein the subject is undergoing the therapy regimen prior to performance of (a).

Embodiment B36. The method of any one of embodiments B18 to B35, wherein the subject is undergoing the therapy regimen prior to performance of (b).

Embodiment B37. The method of any one of embodiments B18 to B36, wherein (d) comprises decreasing a dosage of the compound if the difference in (c) exceeds a first threshold level.

Embodiment B38. The method of any one of embodiments B18 to B36, wherein (d) comprises increasing a dosage of the compound if the difference in (c) does not meet a second threshold level.

Embodiment B39. The method of any one of embodiments B18 to B36, wherein (d) comprises decreasing or increasing a dosage of the compound if the amount in (b) exceeds or does not meet a threshold level.

Embodiment B40. The method of any one of embodiments B18 to B36, wherein (d) comprises decreasing a dosage of the compound if the amount in (b) exceeds a first threshold level.

Embodiment B41. The method of any one of embodiments B18 to B36, wherein (d) comprises increasing a dosage of the compound if the amount in (b) does not meet a second threshold level.

Embodiment B42. The method of any one of embodiments B18 to B36, wherein (d) comprises not changing the therapy regimen if the amount in (b) does not exceed a threshold level.

Embodiment B43. The method of any one of embodiments B18 to B36, wherein (d) comprises not changing the therapy regimen if the difference in (c) does not exceed a threshold level.

### IX. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the inventions and are not intended to limit the scope of what the inventors regard as their inventions in any way. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Activation of Pantothenate Kinase Relieves Coenzyme a Sequestration and Improves Mitochondrial Function in a Mouse Model of Propionic Academia

1. Propionic Acidemia

Propionic acidemia (PA) is a rare autosomal-recessive metabolic disease that arises from mutations in propionyl-CoA (C3-CoA) carboxylase. Reduced enzyme activity blocks C3-CoA metabolism leading to an elevated plasma C3:C2-carnitine ratio, the hallmark biomarker of PA. The metabolic imbalances experienced in PA are poorly defined, and here we use a hypomorphic PA mouse model to demonstrate that C3-CoA accumulation results in a significant reduction in liver non-esterified CoA (CoASH) and acetyl-CoA (C2-CoA). Tricarboxylic acid (TCA) cycle intermediates that are normally metabolized accumulate in the urine providing direct evidence for compromised mitochondrial function in PA. Pantothenate kinase (PanK) catalyzes the rate-controlling step in CoA biosynthesis and its inhibition by C3-CoA prevents a compensatory increase in CoA biosynthesis to alleviate CoASH sequestration. PZ-3022 is an allosteric PanK activator that counteracts C3-CoA inhibition. PZ-3022 therapy increases hepatic CoASH and C2-CoA, and decreases C3-CoA, leading to significant improvement in the intracellular C3:C2-CoA ratio and the clinically relevant biomarker, the plasma C3:C2-carnitine ratio. Elevated urinary malate is a major component of the metabolic signature for TCA cycle dysfunction in the PA mouse and the 80% reduction in urine malate by PZ-3022 therapy indicates the restoration of mitochondrial function. Thus, CoASH sequestration in PA leads to reduced TCA cycle activity that is relieved following PZ-3022 providing preclinical proof of concept for PanK activators as a therapy to attenuate the underlying mitochondrial defect in PA.

Here, we show that CoASH sequestration occurs in the Pcca^{-/-}PCCA(A138T)^{tg/0} (PA) mouse model. CoASH sequestration leads to a reduction in liver total CoA, CoASH, C2-CoA and C2-carnitine, along with compromised TCA cycle activity as revealed by a large elevation in urinary TCA cycle intermediates. Pantazines are drug-like small molecules that elevate CoASH in cells and tissues by binding to PanK and rendering the enzyme refractory to feedback inhibition by CoA thioesters. Treatment of PA mice with an optimized pantazine, PZ-3022, leads to the normalization of hepatic total CoA, CoASH, C2-CoA and C2-carnitine levels. PZ-3022 therapy lowers the plasma C3:C2-carnitine ratio and significantly reduces the excretion of TCA cycle intermediates into the urine, which collectively represent a biomarker for mitochondrial function in PA. These data show that PanK activation by PZ-3022 relieves the metabolic stress on the TCA cycle that arises from trapping CoASH as C3-CoA in PA.

2. PCCA^{-/-}PCCA(A138T)^{TG/0} (PA) Mice

Pcca^{-/-} mice die shortly after birth and exhibit massive elevation in serum C3-carnitine and methylcitrate. These animals are not suitable as PA models. Instead, a hypomorphic mouse was created by complementing the Pcca^{-/-} mice with a transgene expressing the mutant human PCCA(A138T) allele. The transgene expresses a protein with ˜9.4% of the wild-type PCC activity. Although the mutant PCCA allele is not expressed in its native genetic context, this mouse has been used extensively to model PA disease. Human PA arises from many different mutations in PCC and the metabolic phenotypes vary from mild to severe depending on the nature of the mutation, meaning any knock-in or transgenic mouse would model only a subset of human PA. We used a breeding program to generate Pcca^{-/-}PCCA(A138T)^{tg/0} mice that harbor a single copy of the transgene; however, control over the transgene copy number is not specified in many studies suggesting the mice may be a mixture. A mouse model with two copies of the transgene may be less affected than an animal with a single transgene copy. Our first goal was to establish a baseline for the metabolic markers in the Pcca^{-/-}PCCA(A138T)^{tg/0} mouse and validate the model, and we established the PA mouse liver as representative of human disease to study the metabolic imbalances caused by mutations that reduce liver PCC activity.

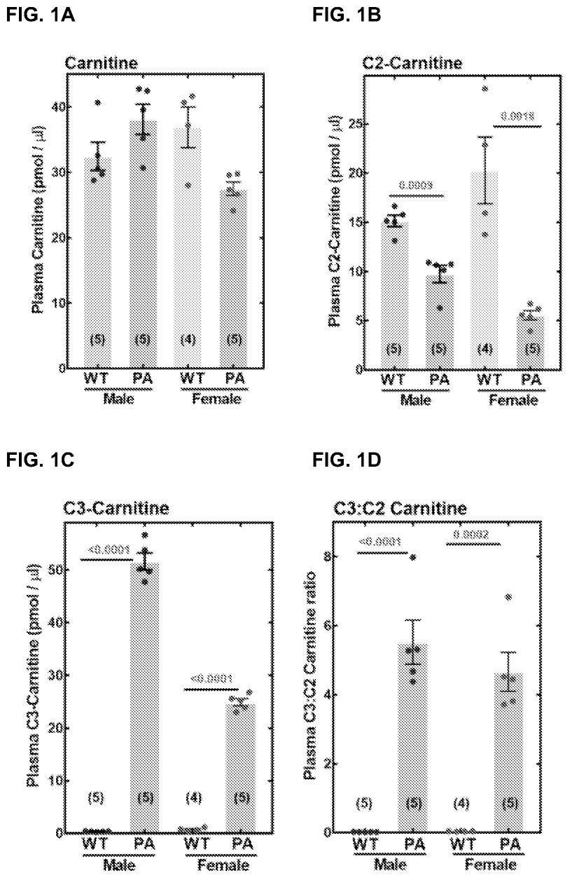

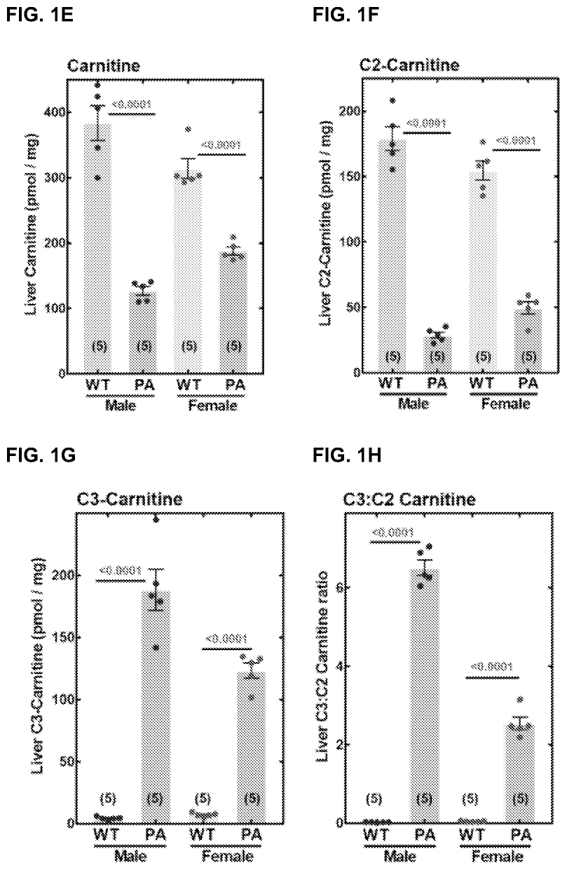

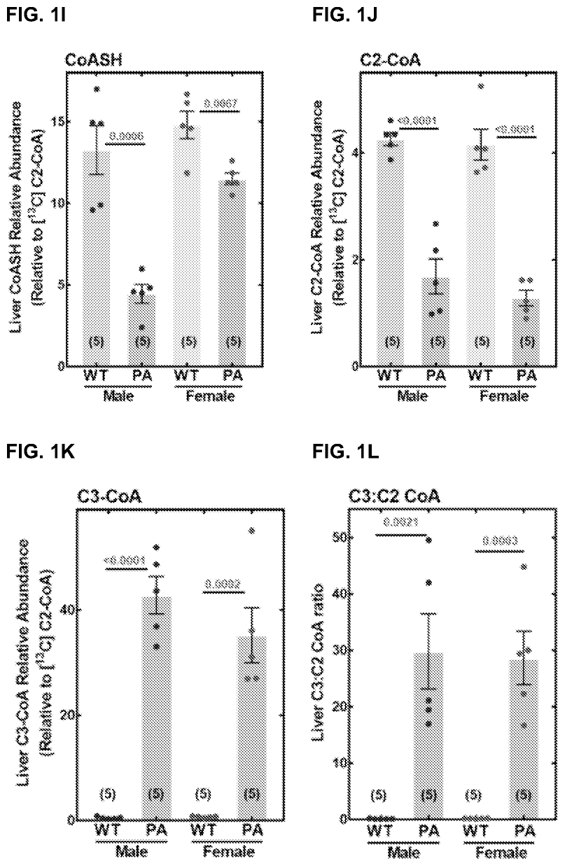

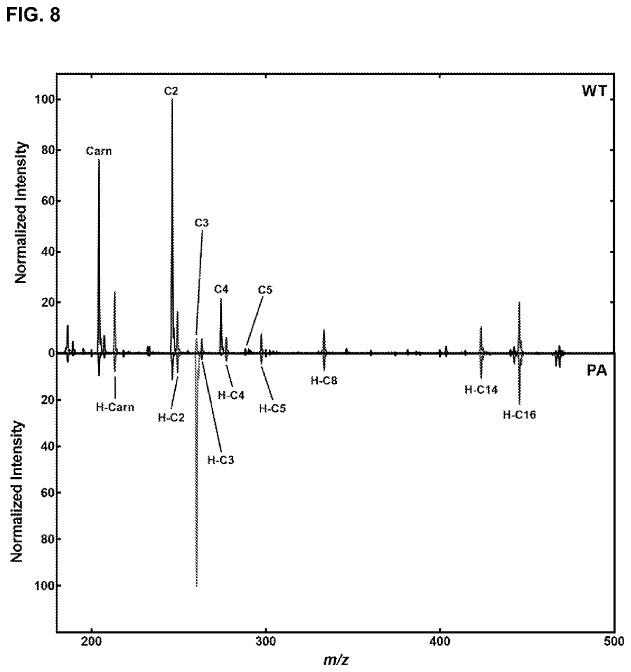

The acyl-carnitines were quantified by mass spectrometry using [d₉]carnitine, [d₃]C2-carnitine and [d₃]C3-carnitine as internal standards. Other carnitine species were present in low levels, did not change in PA mice and were not analyzed further (FIG. 8). Free carnitine (FIG. 1A) and C2-carnitine (FIG. 1B) were the major species in plasma of wild-type animals, whereas C3-carnitine (FIG. 1C) was in low abundance. The PA mice exhibited a massive elevation in plasma C3-carnitine that is the hallmark biomarker of human PA (FIG. 1C). Male PA mice had higher levels of plasma C2- and C3-carnitines compared to the female PA mice (FIGS. 1B and 1C), but the plasma C3:C2-carnitine ratios in both the male and female PA mice were similar (FIG. 1D). The clinical assay for the C3:C2 carnitine ratio is performed using MALDI mass spectrometry, which evaluates the relative ratios of the two metabolites. Our quantitative results show that a significant decrease in circulating C2-carnitine (FIG. 1B) in the PA mice contributes to the elevated C3:C2-carnitine ratio (FIG. 1D) in addition to increased C3-carnitine (FIG. 1C). In liver, carnitine (FIG. 1E) and C2-carnitine (FIG. 1F) were the major species in wild-type mice, and while C3-carnitine was detected, it was a minor component (FIG. 1G). The PA mice had significantly higher hepatic C3-carnitine coupled with decreased carnitine and C2-carnitine levels. The C3-carnitine levels were lower, and the C2-carnitine and carnitine levels were higher in female compared to male PA livers. As result, the liver C3:C2-carnitine ratio was higher in males compared to females (FIG. 1H). Sexual dimorphism in acyl-carnitines is recognized in rodents and is evident in PA mice. Together, these data show that PA mice have the hallmark metabolic signature of PA.

3. Coash Sequestration in PA

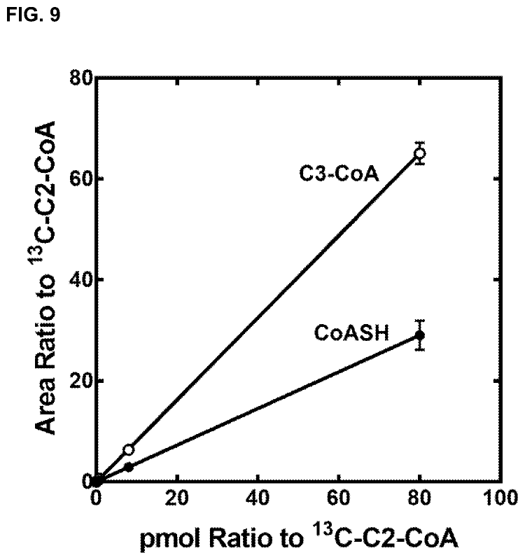

The CoASH sequestration hypothesis posits that the accumulation of C3-CoA leads to reduced levels of CoASH and C2-CoA. The levels of liver CoASH, C2-CoA and C3-CoA were determined by mass spectrometry relative to a [¹³C]acetyl-CoA internal standard in wild-type and PA liver. The detector response to C3-CoA is similar to C2-CoA, but CoASH is detected at about 2.4-fold lower efficiency (FIG. 9) meaning that CoASH is about 2.4-fold more abundant than it appears in this analysis. There was no difference in the liver levels of CoASH (FIG. 1I) or C2-CoA (FIG. 1J) in male and female wild-type mice, and C3-CoA was detected at low levels (FIG. 1K). In PA mice, C3-CoA rose significantly (FIG. 1K) and was associated with significant decrease in CoASH (FIG. 1I) and C2-CoA (FIG. 1J). The C3:C2-CoA ratio was ˜30 (FIG. 1L) which was 5-10 times higher than the C3:C2-carnitine ratios in either the liver or the plasma of affected animals. These data show that CoA is trapped as C3-CoA in PA liver leading to the depression of CoASH and C2-CoA levels.

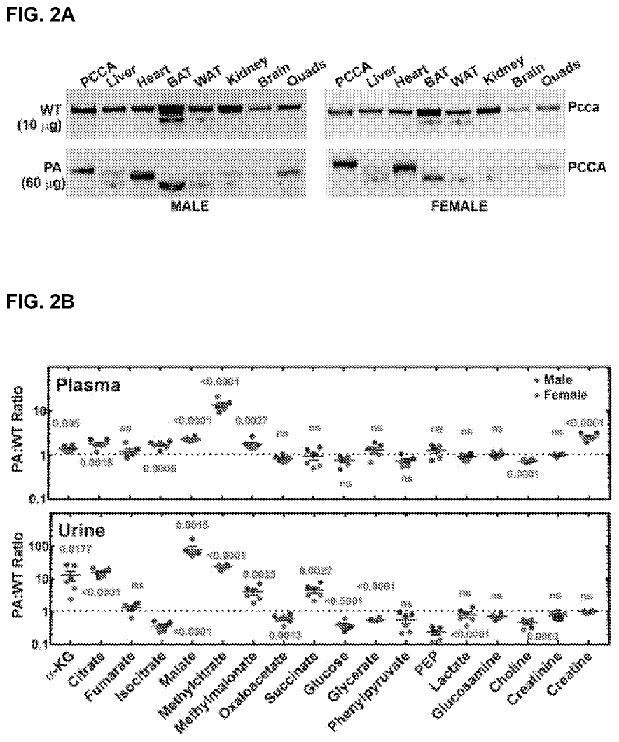

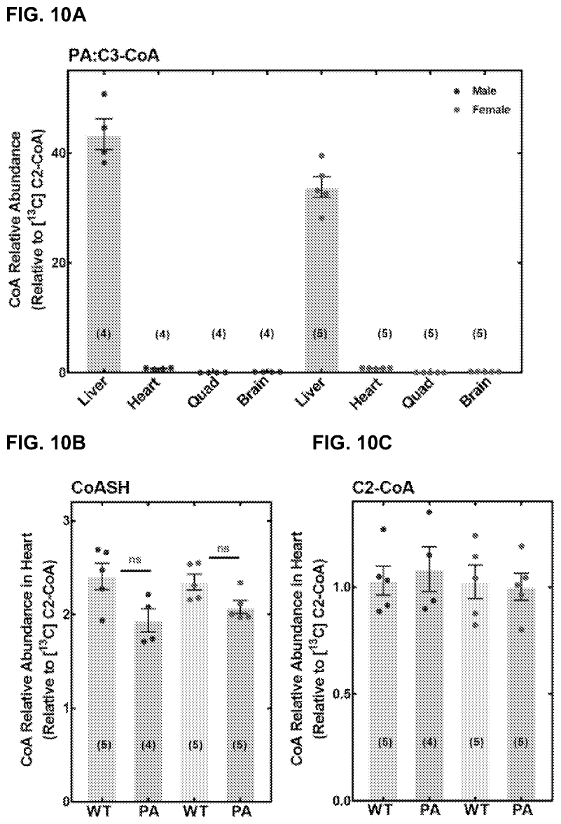

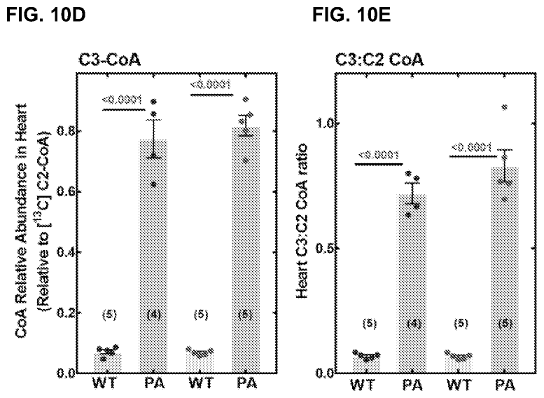

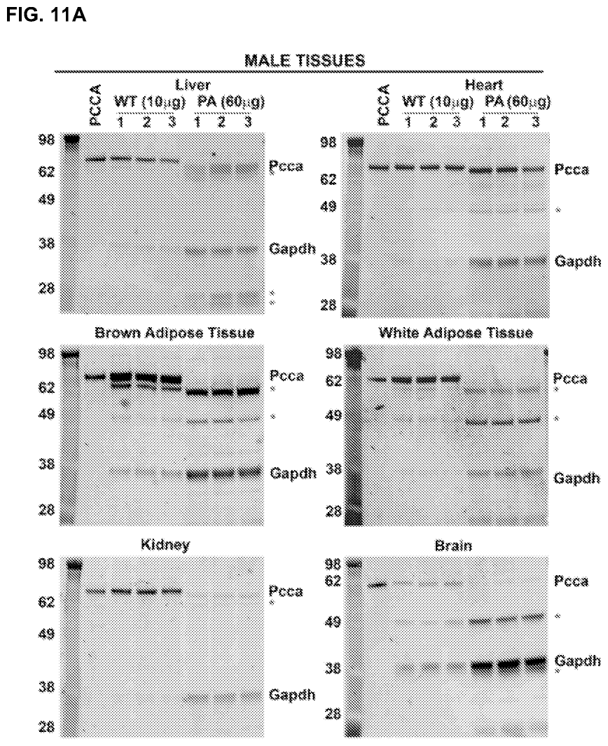

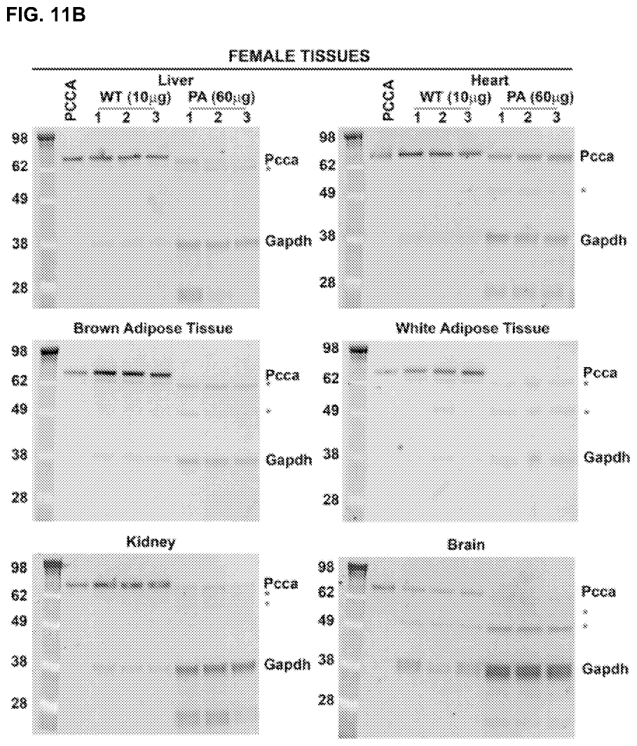

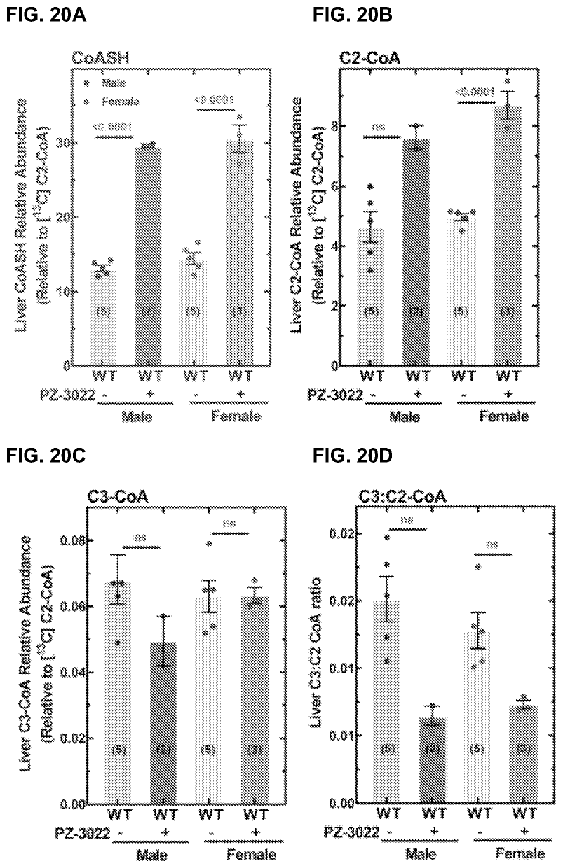

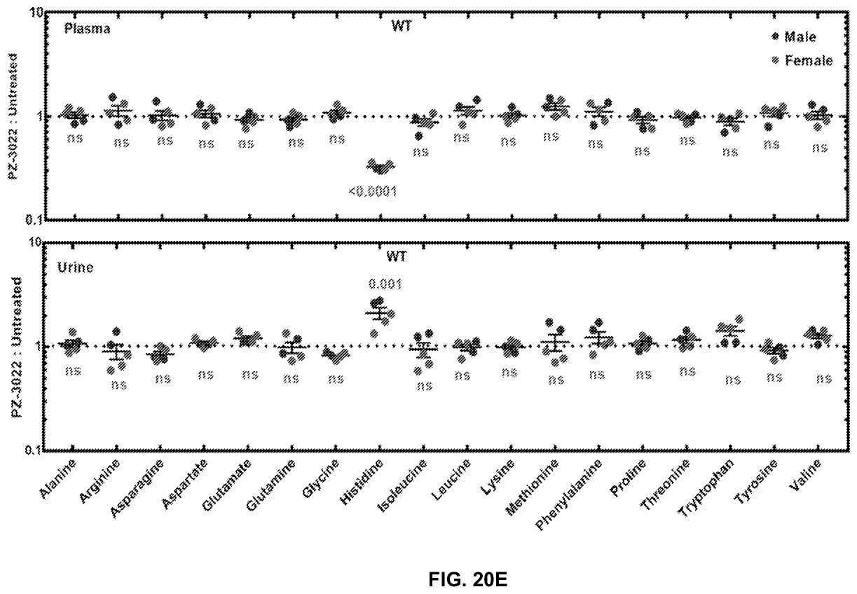

Liver had the highest levels of C3-CoA compared to heart, quadriceps muscle and brain (FIG. 10A). Heart complications arise in human PA and cardiac dysfunction was noted in 58% of 8-month old PA mice in a previous study. Therefore, heart CoA pool composition were examined in more detail. The trend in CoASH reduction was not significant at the P<0.01 level (FIG. 10B). Heart C2-CoA levels were not altered in the PA mouse (FIG. 10C), and although the C3-CoA was elevated in heart (FIG. 10D), the magnitude of heart C3-CoA accumulation was 40-fold lower than in liver. In contrast with liver, C2-CoA does not notably decrease in the heart, illustrating that CoA sequestration does not occur in the heart in this PA mouse model. There were no significant alterations in the mouse electrocardiograms between 70-day old wild-type and PA mice (Table 2). These data indicate that the 160 reduction in ejection fraction noted in the 8-month PA mice is not present in the 70-day old animals. We did observe an increase in the heart:body weight ratio as reported in the PA mouse (FIG. 20B). The high level of PCCA transgene expression in heart compared to other tissues (FIG. 2A; FIG. 11) likely accounts for the modest impact on heart C3-CoA levels and CoA sequestration (FIGS. 10A-10E) and weakens the utility of this PA mouse in modeling the cardiac complications in PA disease.

TABLE 2
Echocardiogram data for 70-day old wild type and PA mice

| | | WT (M) | PA-M | WT (F) | PA (F) |
|---|---|---|---|---|---|
| Parameters^{a} | Units | (n = 6) | (n = 5) | (n = 5) | (n = 6) |
| Heart Rate | beats/min | 354.3 ± 14.7 | 360.9 ± 7.4 | 336.1 ± 8.6 | 339.3 ± 16.4 |
| LVID; s | mm | 2.4 ± 0.3 | 2.5 ± 0.2 | 2.7 ± 0.2 | 2.5 ± 0.1 |
| LVID; d | mm | 3.4 ± 0.3 | 3.6 ± 0.2 | 3.6 ± 0.2 | 3.6 ± 0.1 |
| LV; s | μL | 22.3 ± 5.6 | 22.7 ± 4.7 | 27.8 ± 5.7 | 21.8 ± 2.1 |
| LV; d | μL | 51.1 ± 7.5 | 53.7 ± 6 | 56.6 ± 6.3 | 53.3 ± 3 |
| SV | μL | 28.8 ± 3.2 | 31.0 ± 1.7 | 28.8 ± 0.7 | 31.5 ± 2.8 |
| EF | % | 61 ± 6.7 | 59.4 ± 4.4 | 53 ± 4.8 | 58.9 ± 3.8 |
| FS | % | 33.2 ± 5.1 | 31.2 ± 3 | 27 ± 3 | 30.8 ± 2.5 |
| CO | ml/min | 10.4 ± 1.3 | 11.2 ± 0.8 | 9.7 ± 0.4 | 10.9 ± 1.4 |
| LV Mass | mg | 119.4 ± 9.5 | 102.0 ± 8.3 | 114.1 ± 4.7 | 122.7 ± 13.5 |
| LV Mass Cor | mg | 95.6 ± 7.6 | 81.6 ± 6.7 | 91.2 ± 3.8 | 98.2 ± 10.8 |
| LVAW; s | mm | 1.3 ± 0.1 | 1.1 ± 0.1 | 1.2 ± 0.1 | 1.4 ± 0.1 |
| LVAW; d | mm | 1.1 ± 0.1 | 0.9 ± 0.1 | 1 ± 0.04 | 1.1 ± 0.09 |
| LVPW; s | mm | 1.2 ± 0.1 | 1.1 ± 0.03 | 1 ± 0.2 | 1.1 ± 0.07 |
| LVPW; d | mm | 0.8 ± 0.1 | 0.8 ± 0.03 | 0.77 ± 0.2 | 0.8 ± 0.08 |

^{a}Values are expressed as mean ± SD. LVID; s, Left ventricular end-systolic diameter; LVID; d, Left ventricular end-diastolic diameter; LV; s, Left ventricular end-systolic volume; LV; d, Left ventricular end-diastolic volume; SV, Stroke volume; EF, Ejection fraction; FS, Fractional shortening; CO, Cardiac Output; LV Mass, Left Ventricular mass; LV Mass Corr, Left ventricular mass corrected for body surface area; LVAW; s, Left ventricular end-systolic anterior wall thickness; LVAW; d, Left ventricular end-diastolic anterior wall thickness; LVPW; s, Left ventricular end-systolic posterior wall thickness; LVPW; d, Left ventricular end-diastolic posterior wall thickness. Mouse groups were: WT (M), male wild-type mice; PA (M): male PA mice; WT (F), female wild-type mice; PA (F), female PA mice. The number of mice in each analysis is given in parenthesis.

4. PCCA(A138T) Expression

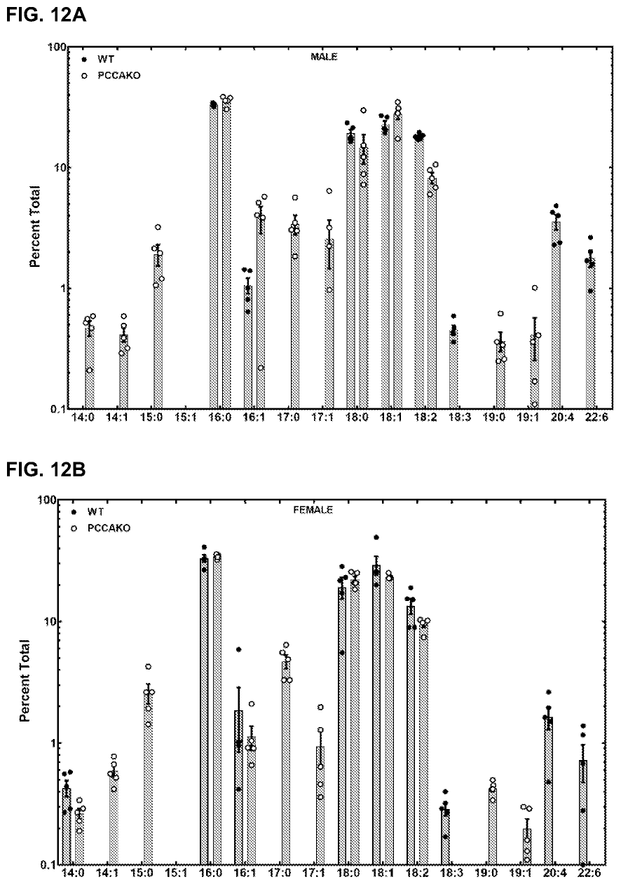

The hybrid cytomegalovirus early enhancer/chicken β-actin/rabbit β-globin (CAG) promoter is used to drive expression of the PCCA(A138T) transgene. The expression levels of the transgene-derived human PCCA protein levels are compared to the mouse Pcca protein levels normally found in a series of tissues (FIG. 2A; FIG. 11). Pcca protein exhibits a tissue-specific abundance pattern in mice that is highest in adipose tissue followed by liver, kidney and heart. Human PCCA protein expressed by the PCCA(A138T) transgene was higher in skeletal muscle and heart compared to other tissues, which is similar to that reported in transgenic rodents expressing green fluorescent protein, human ATP7A or PANK2 driven by the same CAG promoter. The transgene-encoded PCCA protein did not have the same tissue-specific abundance as mouse Pcca. For example, liver contained far less of the transgene-expressed human protein than was present in wild-type mice, and mouse adipose tissue, which expresses the highest levels of endogenous mouse Pcca, was devoid of human PCCA protein expression. In contrast, heart and quadriceps muscles express about the same level of transgene-derived PCCA as murine Pcca. Thus, heart is not as compromised in PCCA protein levels as the liver in PA mice. PA mice also had an altered composition of liver total fatty acids (FIGS. 12A-12B). Odd-chain fatty acids are below detection in wild-type mice but collectively rise to ˜8.8% of the total in male and female PA mice. These odd-chain fatty acids arise from the initiation of fatty acid synthesis with C3-CoA. Thus, the tissue distribution of PCCA(A138T) transgene expression identifies liver as a tissue representative of human disease to study the impact of reduced PCC activity because it has significantly reduced, but not absent, PCCA protein levels.

5. TCA Metabolite Levels

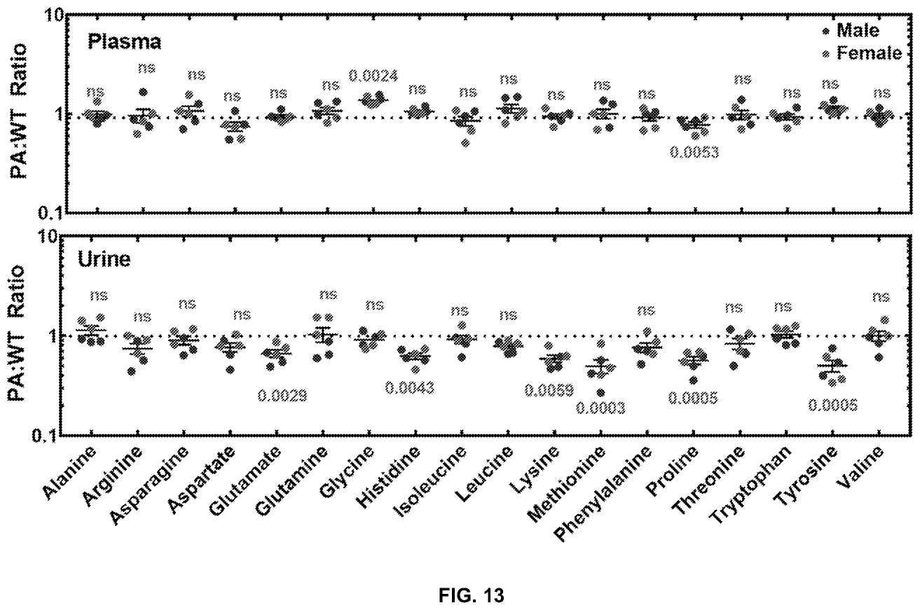

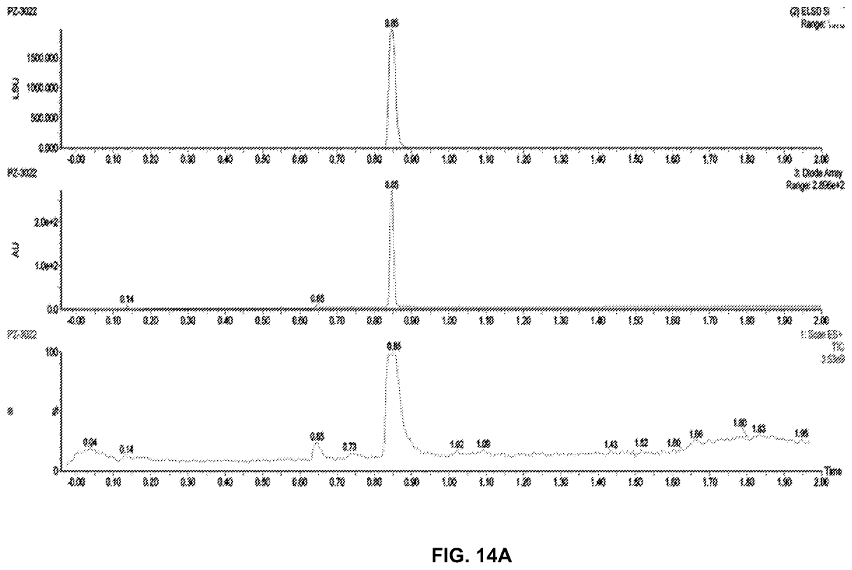

Dysfunctional mitochondrial metabolism is a characteristic of PA, and the release of TCA cycle intermediates from cells and tissues signals mitochondrial malfunction. Our hypothesis is that reduced mitochondrial production of C2-CoA via PDH is a consequence of CoA sequestration and low CoASH leading to alternate fates for pyruvate and TCA cycle intermediates. The profiling of liver metabolites following propionate stress shows that when C3-CoA becomes the dominant CoA species, malate increases 6.6-fold along with other TCA cycle metabolites. Urinary TCA intermediate levels are not routinely used as biomarkers of human PA, but there is evidence for elevated levels of TCA cycle intermediates in the urine of PA patients. A metabolomics screen was used to identify changes in TCA cycle metabolites, selected amino acids and other metabolites in the PA mouse. Levels of individual amino acids in plasma were not greatly altered in the PA mouse (FIG. 13). There were modest decreases in some urinary amino acids, like tyrosine, proline and methionine, but these changes were not large enough to be sensitive biomarkers (FIG. 13). Glycine levels are elevated 2-10 fold in human PA depending on the patient, but there was only a modest elevation (˜50%) in plasma glycine in the PA mouse. Glycine was measured as its benzoyl derivative to increase detection sensitivity; plasma glycine was elevated 40% in plasma and was unchanged in urine of PA mice (FIG. 13). The major alterations observed were in the levels of TCA cycle intermediates (FIG. 2B). Methylcitrate, formed from C3-CoA and oxaloacetate by citrate synthase, is an established marker of PA disease and was significantly elevated in plasma and urine. Plasma levels of malate, methylmalonate, isocitrate, citrate and α-ketoglutarate were all elevated, but TCA metabolite levels in urine were the clearest markers of mitochondrial dysfunction in PA mice. Malate (85-fold), succinate (5-fold), α-ketoglutarate (9-fold), methylmalonate (4-fold) and citrate (20-fold) were all significantly elevated in urine (FIG. 2B). Malate was the most elevated biomarker in urine, and the smaller increase in malate in the plasma compared to urine was consistent with the saturation of malate resorption by kidney. These data suggest that pyruvate-derived TCA cycle intermediates normally used for energy production or intermediary metabolism are eliminated in the urine, providing a collective indicator of mitochondrial dysfunction in PA mice.

6. PZ-3022 Relieves CoASH Sequestration

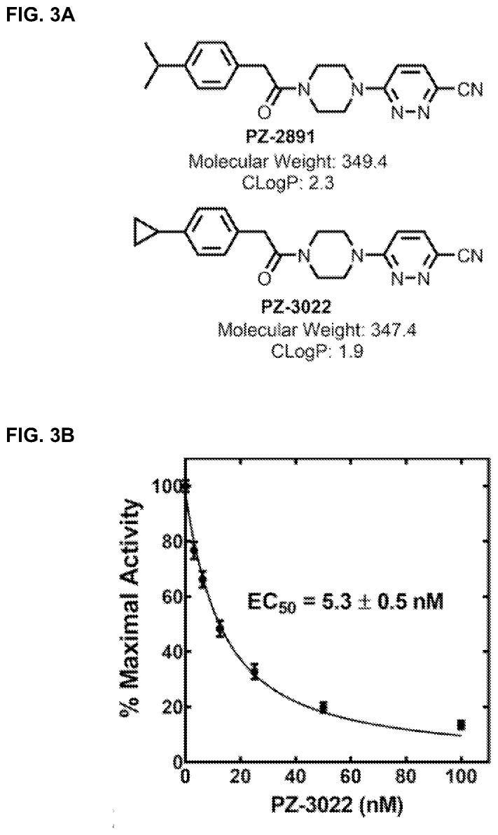

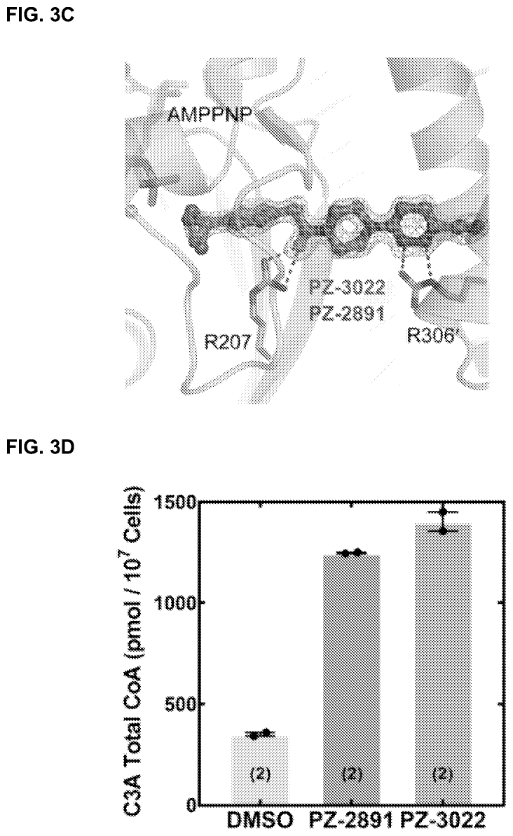

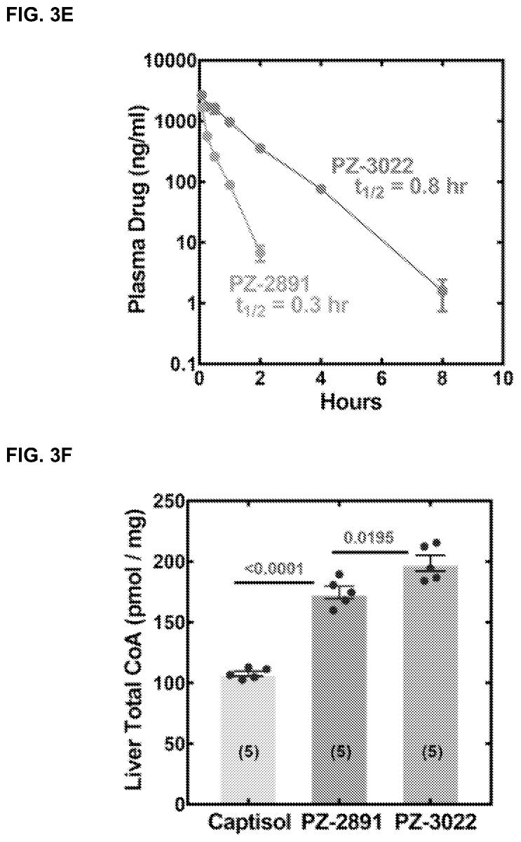

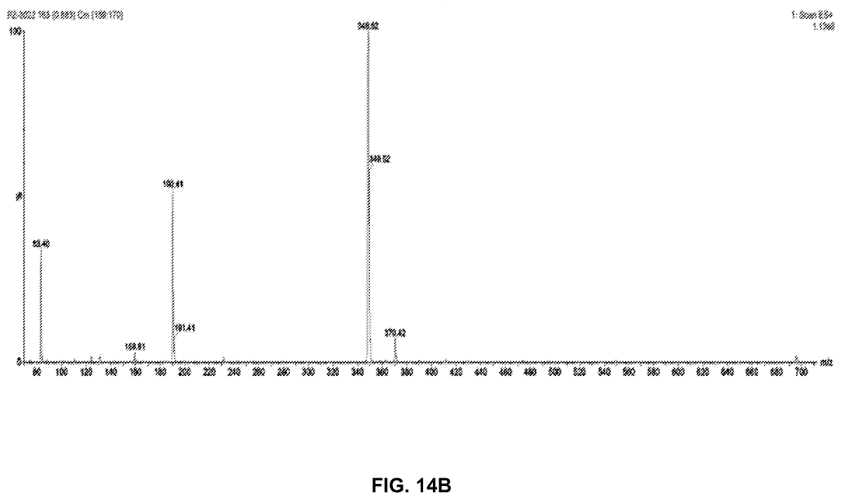

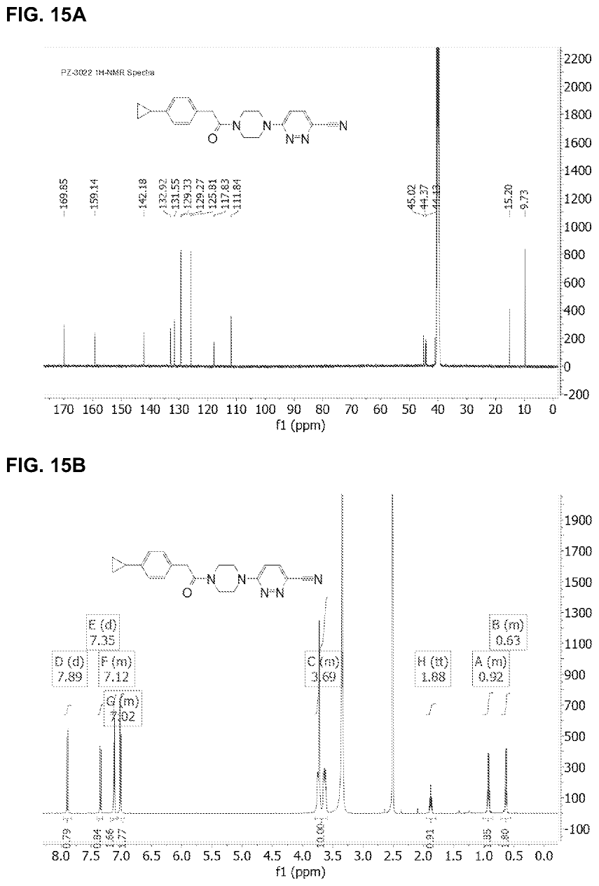

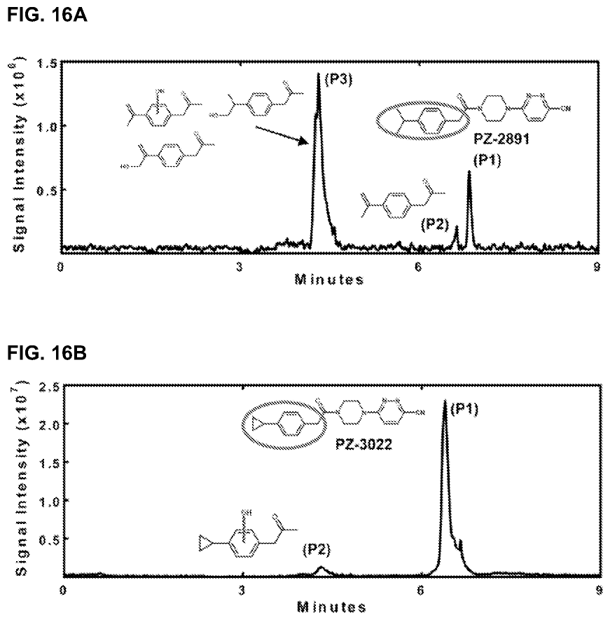

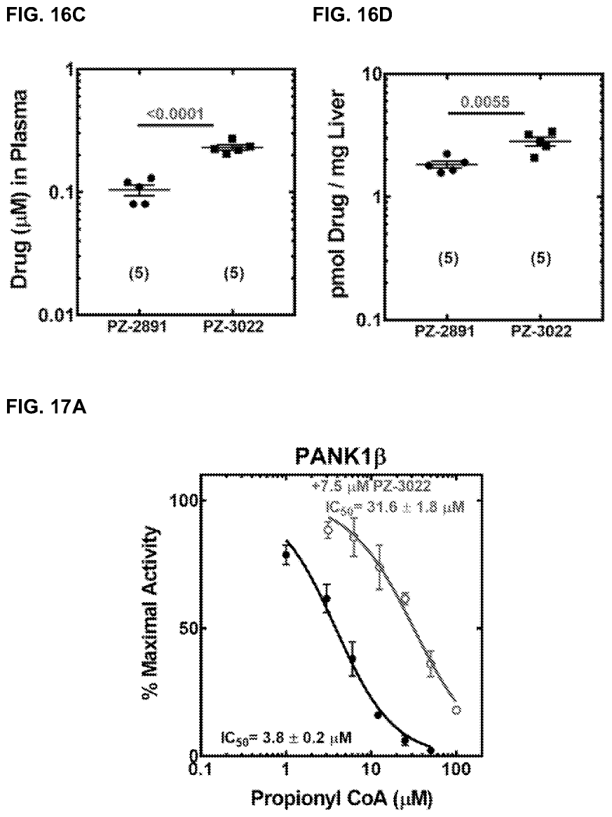

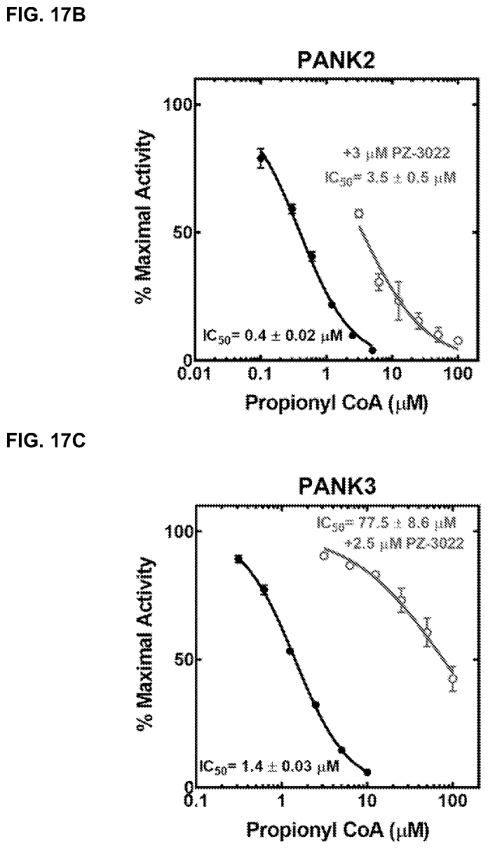

We developed a series of drug-like, small molecule allosteric activators of PanK called pantazines that render the enzyme resistant to feedback inhibition by CoA thioesters. The first generation pantazine, PZ-2891, effectively elevated intracellular CoA in cell cultures, and in the tissues of treated mice. Another pantazine, PZ-3022, replaces the isopropyl group of PZ-2891 with a cyclopropyl moiety (FIGS. 3A, 14A-14B, and 15A-15B). PZ-3022 has an EC₅₀ of 5.3 nM against PanK3 (FIG. 3B) and has a lower affinity for each of the PanK isoforms compared to PZ-2891 (Table 3). The crystal structure shows PZ-3022 bound to the PanK dimer in the same location and orientation as PZ-2891 (FIG. 3C; Table 4). The cyclopropyl group of PZ-3022 binds where the isopropyl group of PZ-2891 resides and PZ-3022 extends across the dimer interface to affect the active site on the opposite protomer. PZ-3022 was just as effective at raising total CoA levels in cultured cells as PZ-2891 (FIG. 3D). The major impact of introducing the cyclopropyl group was the significantly decreased rate of PZ-3022 clearance compared to PZ-2891 (Table 5) due to a significantly longer half-life (FIG. 3E). The key metabolite arises from hydroxylation of the isopropyl sidechain of PZ-2891 that, in turn, triggers further metabolism to a variety of hydroxylated products (FIG. 16A). PZ-3022 is refractory to this hydroxylation event leading to reduced drug metabolism and higher levels of circulating PZ-3022 (FIG. 16B). These properties combined with the high oral bioavailability of PZ-3022 resulted in higher levels of circulating (FIG. 16C) and liver (FIG. 16D) PZ-3022 compared to PZ-2891 following the same oral drug dosage. Like PZ-2891, PZ-3022 rendered all three PanK isoforms refractory to inhibition by C3-CoA (FIGS. 17A-17C). A direct comparison of the ability of PZ-3022 and PZ-2891 to elevate hepatic total CoA in C57BL/6J mice showed that PZ-3022 was slightly more potent in elevating CoA than PZ-2891 (FIG. 3F). Thus, the enhanced metabolic stability and bioavailability of PZ-3022 improves the overall efficacy of pantazine therapy in mice.

The recombinant human PANK proteins were purified and 100 ng of PANK1β 100 ng of PANK2 and 50 ng of PANK3 were used in radiochemical assays measuring the conversion of [¹⁴C]pantothenic acid to phospho[¹⁴C]pantothenate. The PZ-2891 and PZ-3022; EC₅₀'s were calculated using the Morrison equation. Assays were performed three times in duplicate and the data complied to estimate the EC₅₀ for each enzyme.

TABLE 3
EC₅₀ of PZ-2891 and PZ-3022 with pantothenate kinases

| Pantothenate | PZ-2891 | PZ-3022 |
|---|---|---|
| Kinase | EC₅₀ (nM) | EC₅₀ (nM) |
| PANK1β | 40 ± 4.4 | 789 ± 48 |
| PANK2m | 0.7 ± 0.08 | 15 ± 1.2 |
| PANK3 | 1 ± 0.1 | 5.3 ± 0.5 |

^{a}PANK2m corresponds to the mature, processed PANK2 protein (amino acids 141-570).

TABLE 4
X-ray data collection and refinement statistics
PANK3•AMPPNP•Mg²⁺•PZ-3022 Complex (PDB ID: 6PE6)

| Data Collection^{a} | | |
|---|---|---|
| Wavelength (Å) | 1.0 | |
| Space group | P3₁21 | |
| Cell dimensions | | |
| a, b, c (Å) | 97.8, 97.8, 69.3 | |
| α, β, γ (°) | 90.0, 90.0, 120.0 | |
| Resolution (Å) | 50.0-1.60 | (1.63-1.60) |
| R_{merge} | 0.068 | (0.807) |
| CC_{1/2} | 0.997 | (0.786) |
| Completeness (%) | 98.9 | (97.5) |
| Redundancy | 9.2 | (7.1) |
| I/σ(I) | 30.7 | (2.2) |
| Unique reflections | 50,252 | (2,440) |

| | |
|---|---|
| Refinement | |
| Resolution (Å) | 36.1-1.6 |
| No. of reflections | 49,320 |
| No. of atoms | |
| Protein | 2,735 |
| Ligand/ion | 58 |
| Water | 175 |
| R_{work} | 0.190 |
| R_{free} | 0.224 |
| B factors (Å²) | |
| Protein | 19.9 |
| Ligand/ion | 13.6 |
| Water | 29.1 |
| R.m.s. deviations | |
| Bond lengths (Å) | 0.007 |
| Bond angles (°) | 0.995 |
| Ramachandran plot | |
| Favored (%) | 98.56 |
| Allowed (%) | 1.44 |
| Outliers (%) | 0.00 |
| PDB ID | 6PE6 |

^{a}Values in parentheses are for the highest-resolution shell.

Mean pharmacokinetic parameters of PZ-2891 and PZ-3022 in plasma following single intravenous (Dose=2 mg/kg) or oral (Dose=10 mg/kg) administration to male BALB/c mice^{a}.

TABLE 5
Pharmacokinetics of PZ-2891 and PZ-3022.

| | | Dose | Tₘₐₓ | C₀/Cₘₐₓ^{c} | AUCₗₐₛₜ | AUC_{inf} | T_{1/2} | CL | Vss | |
|---|---|---|---|---|---|---|---|---|---|---|
| Drug | Route^{b} | (mg/kg) | (h) | (ng/ml) | (h*ng/ml) | (h*ng/ml) | (h) | (ml/min/kg) | (L/kg) | % F^{d} |
| PZ- | i.v. | 2 | — | 2808.3 | 608.6 | 611.5 | 0.3 | 54.5 | 0.9 | 46 |
| 2891 | p.o. | 10 | 0.3 | 1886.5 | 1397.8 | 1424.0 | — | — | — | |
| PZ- | i.v. | 2 | — | 3285.1 | 2957.3 | 2960.3 | 0.8 | 11.3 | 0.7 | 100 |
| 3022 | p.o. | 10 | 0.5 | 5223.9 | 16884 | 17283.1 | — | — | — | |

^{a}Pharmacokinetics measurements performed by Sai Life Sciences Limited. Full reports available on request.
^{b}30% Captisol was used to formulate PZ-2891 and PZ-3022.
^{c}Back extrapolated concentration for i.v. group.
^{d}Bioavailability was calculated based on AUCₗₐₛₜ.

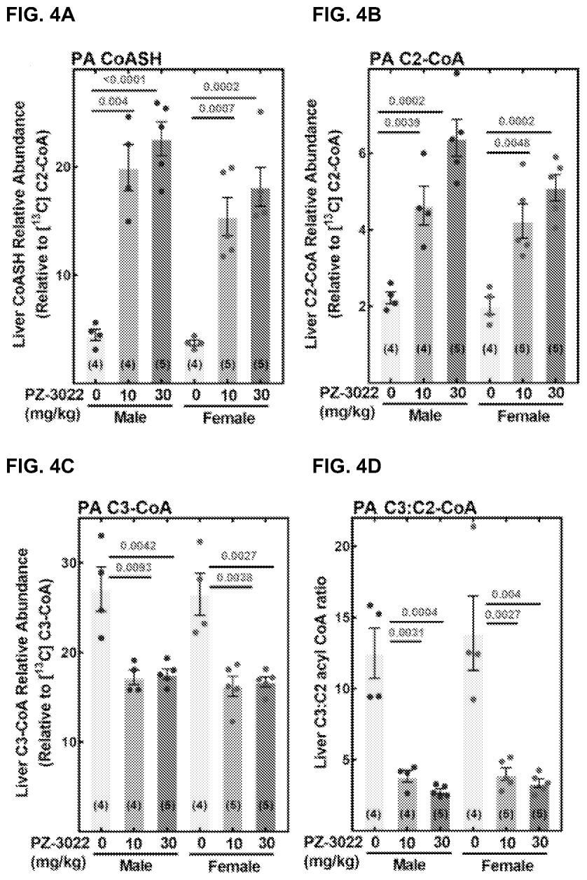

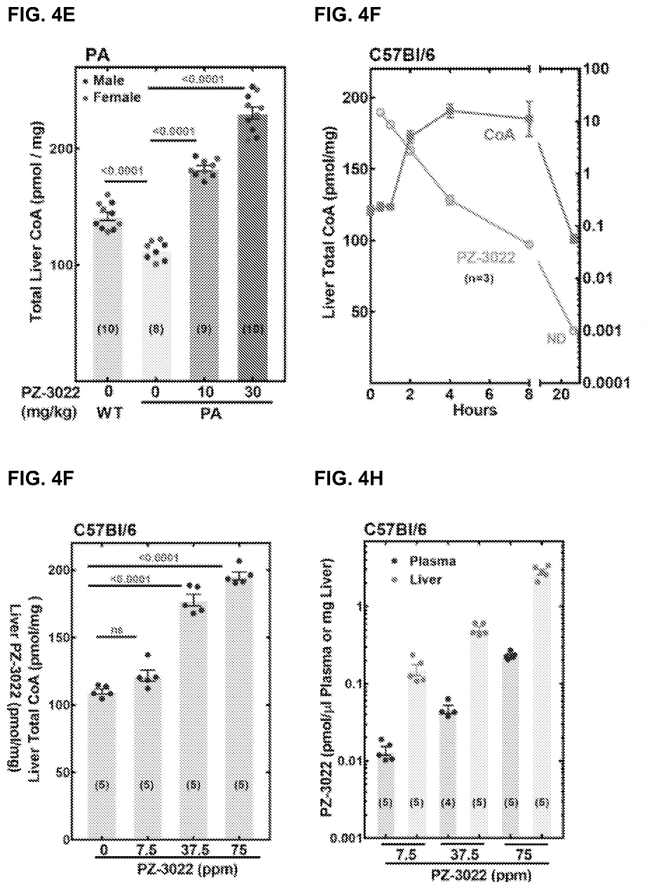

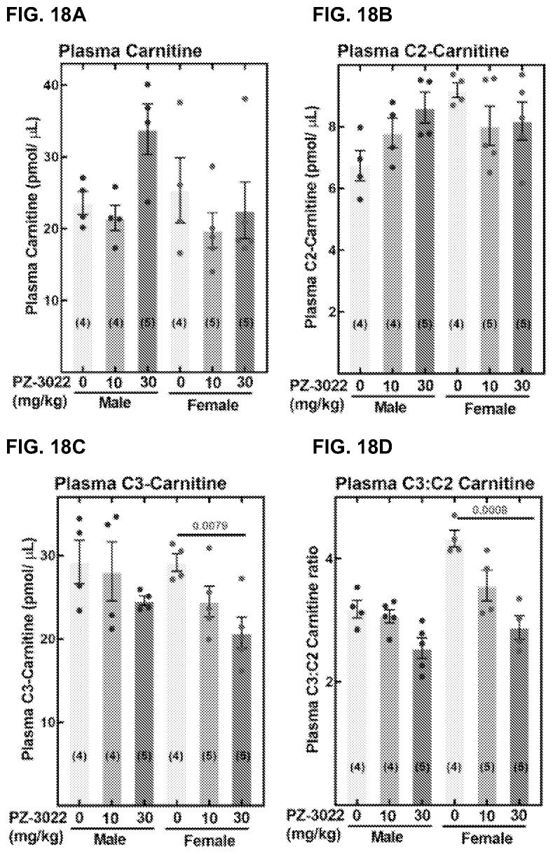

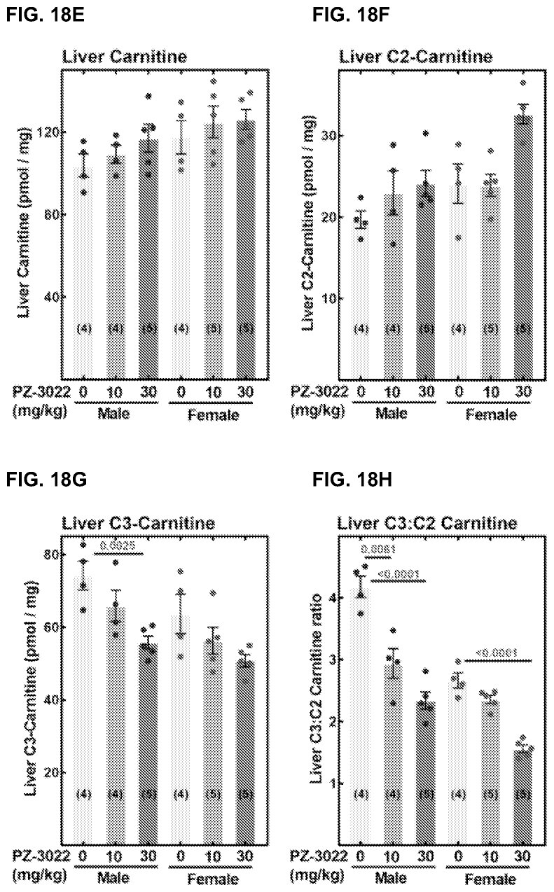

The impact of treating the PA mice by oral gavage with 10 or 30 mg/kg of PZ-3022 on hepatic CoA species distribution and plasma acyl-carnitines was evaluated. Pantothenate potentiates the effect of pantazines, therefore a 50 mg/kg dose of pantothenate was included with PZ-3022, and a dose of 50 mg/kg of pantothenate served as the control. The animals were orally gavaged with 3 doses delivered at 24 h intervals, and samples were collected four hours after the last dose. Both 10 and 30 mg/kg doses of PZ-3022 had a significant impact on the levels of liver CoA thioesters. Along with significantly elevated CoASH (FIG. 4A), C2-CoA increased (FIG. 4B) and C3-CoA decreased but was not eliminated (FIG. 4C). These metabolic changes led to a marked improvement in the C3:C2-CoA ratio (FIG. 4D). The levels of plasma and liver carnitine species were modestly altered by short-term PZ-3022 therapy resulting in a significant reduction at the 30 mg/kg dose in the C3:C2-carnitine ratio (FIGS. 18A-18H). These data show that PZ-3022 treatment relieved CoASH sequestration in liver by increasing hepatic CoASH and C2-CoA and decreasing C3-CoA, consistent with the stimulation of TCA cycle flux. The total amount of CoA was measured using a fluorescent derivatization and liquid chromatography method that quantified CoASH together with acyl-CoA species. Total CoA measurements revealed a reduced total CoA in PA liver and a significant, dose-dependent elevation in total CoA in the livers of PZ-3022-treated PA mice (FIG. 4E). Male and female total CoA levels responded similarly to PZ-3022.

We next determined the relationship between liver CoA elevation and drug exposure. These data show that in C57BL/6J mice the elevation of liver CoA begins at 2 h after PZ-3022 administration and is maintained up to 8 h (FIG. 4F). However, circulating PZ-3022 was not detected at 24 h and the hepatic CoA levels had returned to the pre-dose level, meaning that more frequent dosing of PZ-3022 would be needed to maintain elevated liver CoA (FIG. 4F). This was accomplished by formulating PZ-3022 in the diet. We maintained wild-type C57BL/6J mice for one week on three concentrations of PZ-3022 in the chow and determined the impact on hepatic CoA (FIG. 4G). These data show a dose response relationship between total liver CoA and dose of PZ-3022 in chow. Measurement of the plasma and liver PZ-3022 levels showed a dose-dependent increase in drug (FIG. 4H). Based on the amount of CoA elevation exhibited by liver, the 75-ppm dose of PZ-3022 was selected to evaluate PZ-3022 therapy in the PA mouse model.

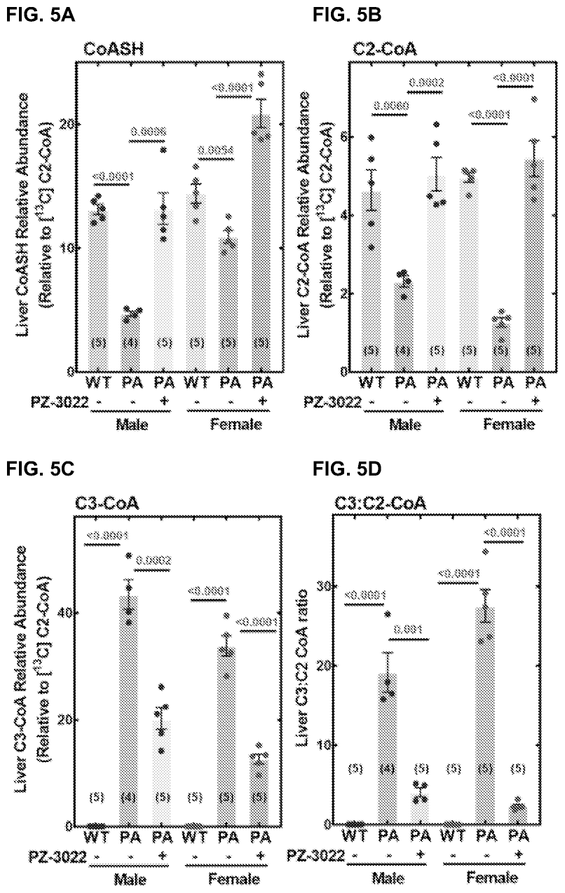

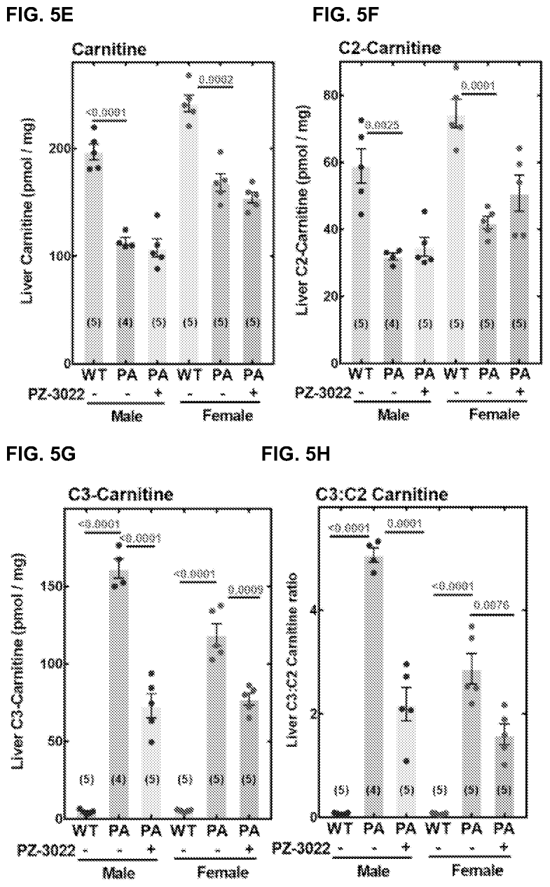

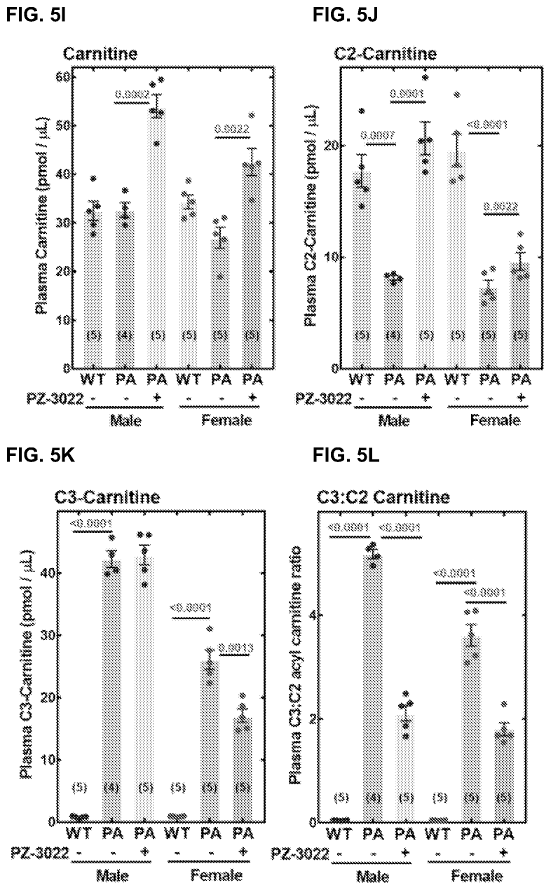

PZ-3022 was tested as a PA therapeutic by introducing the compound into the mouse chow to provide a daily dose of the compound over the course of weeks. Mice were maintained on a mouse chow formulated with 75 ppm PZ-3022 and 1000 ppm pantothenate to determine if the alterations in liver CoA thioesters induced by short-term PZ-3022 therapy were durable. Animals were placed on the diets at weaning (21 days) and when the mice reached 70 days of age, tissue and plasma samples were analyzed (FIGS. 5A-5L). PZ-3022 therapy restored liver CoASH levels to wild-type in both males and females (FIG. 5A). The depressed liver levels of C2-CoA were restored to wild-type levels by PZ-3022 therapy (FIG. 5B). There was also a significant drop in hepatic C3-CoA (FIG. 5C) leading to a substantial decrease in the C3:C2-CoA ratio (FIG. 5D). These data show that PZ-3022 therapy alleviates the impact of C3-CoA accumulation on CoASH and C2-CoA levels in liver.

Liver carnitine profiles were also improved by PZ-3022 therapy. Carnitine (FIG. 5E) and C2-carnitine (FIG. 5F) did not exhibit significant increases following PZ-3022 treatment; however, liver C3-carnitine formation was significantly decreased in both male and female mice (FIG. 5G) leading to a corresponding decrease in the C3:C2-carnitine ratio in liver (FIG. 5H). An increase in plasma carnitine was observed in both male and female mice following PZ-3022 therapy (FIG. 5I). A key result was that PZ-3022 therapy significantly increased C2-carnitine levels in the plasma of both male and female PA mice (FIG. 5J). The C3-carnitine levels in plasma were the same in the treated and untreated male mice but were significantly lower in the female mice where treatment reduced C3-carnitine (FIG. 5K). We do not have a mechanistic explanation for the differences in the male and female responses to PA and therapy, but sexual dimorphism is also observed in the PA mouse response to gene therapy. PZ-3022 therapy substantially reduced the plasma C3:C2-carnitine ratio in both sexes (FIG. 5L) indicating a substantial improvement in the treated mice. These data show that PZ-3022 therapy relieves hepatic CoA sequestration leading to elevated CoASH and C2-CoA. These alterations in hepatic CoA metabolism resulted in lower C3:C2-CoA levels in liver and significantly reduced C3:C2-carnitine ratios in circulation, indicating an improvement in the metabolic state in PZ-3022-treated PA mice.

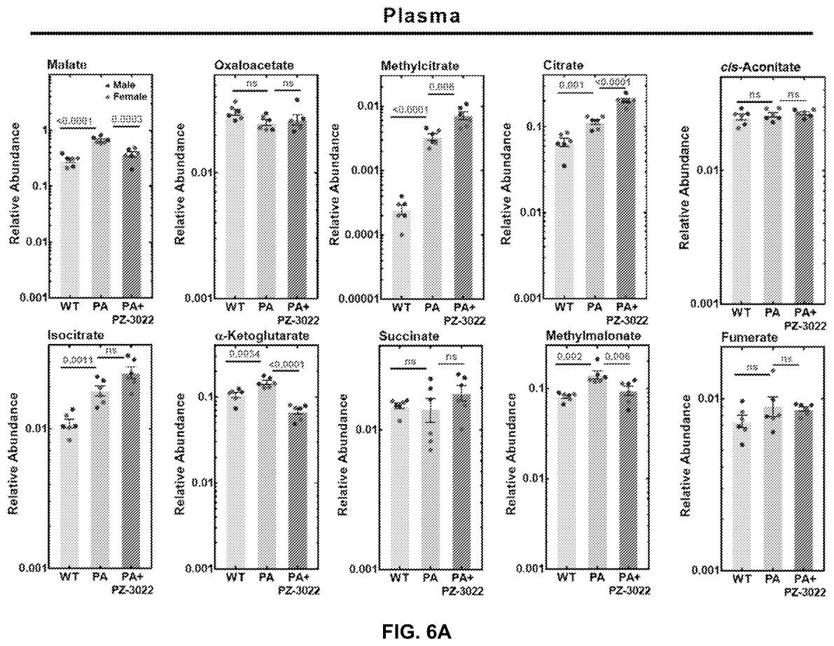

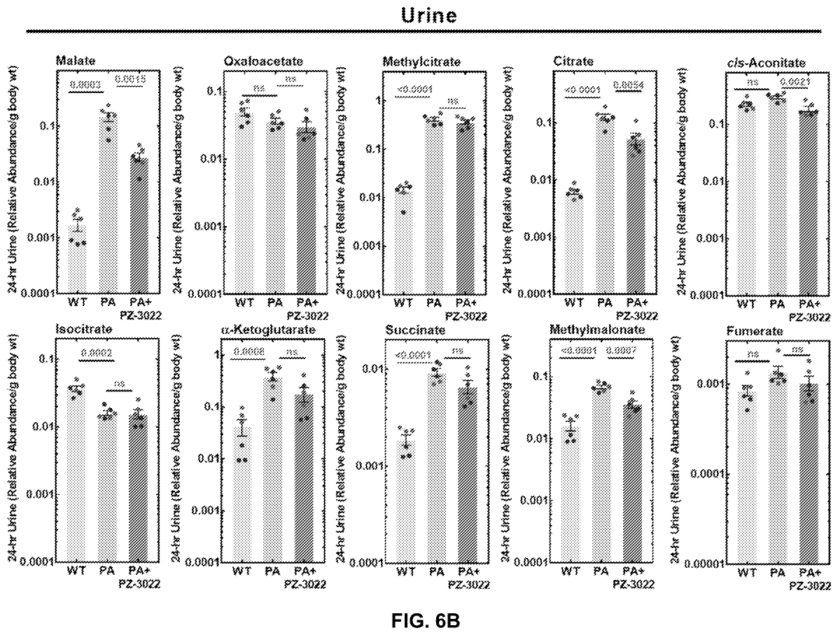

We examined a spectrum of urinary TCA cycle intermediates and related metabolites in plasma and urine to determine if mitochondrial utilization of these metabolites was improved by PZ-3022 therapy. Plasma levels of malate, citrate, isocitrate, α-ketoglutarate, and methylmalonate were significantly elevated in plasma from PA mice indicating the leakage of TCA cycle intermediates out of the tissues (FIG. 6A). However, plasma metabolite levels are capped by the saturation of the kidney resorption mechanisms, and the most sensitive indicator of the amount of TCA cycle dysfunction was the accumulation of intermediates in urine (FIG. 6B). The levels of malate, citrate, α-ketoglutarate and methylmalonate were significantly reduced by PZ-3022 therapy in urine. Malate was the most perturbed indicator of TCA cycle dysfunction, and PZ-3022 therapy reduced the excretion of malate by 80%. Urinary TCA cycle intermediate levels are a robust indicator of mitochondrial function with the potential to be a clinical indicator of the severity of TCA cycle dysfunction in PA disease. These data show that the relief of CoA sequestration by PZ-3022 therapy substantially improves compromised mitochondrial function in PA based on the reduction of malate and other TCA cycle intermediates excreted in urine.

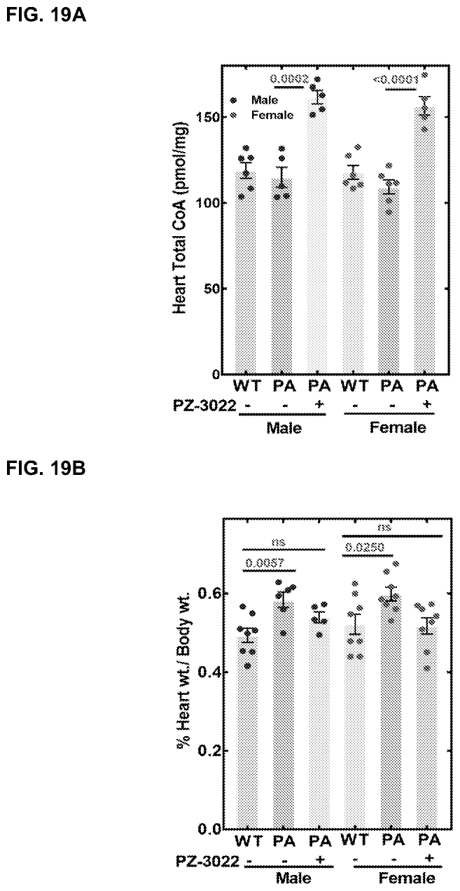

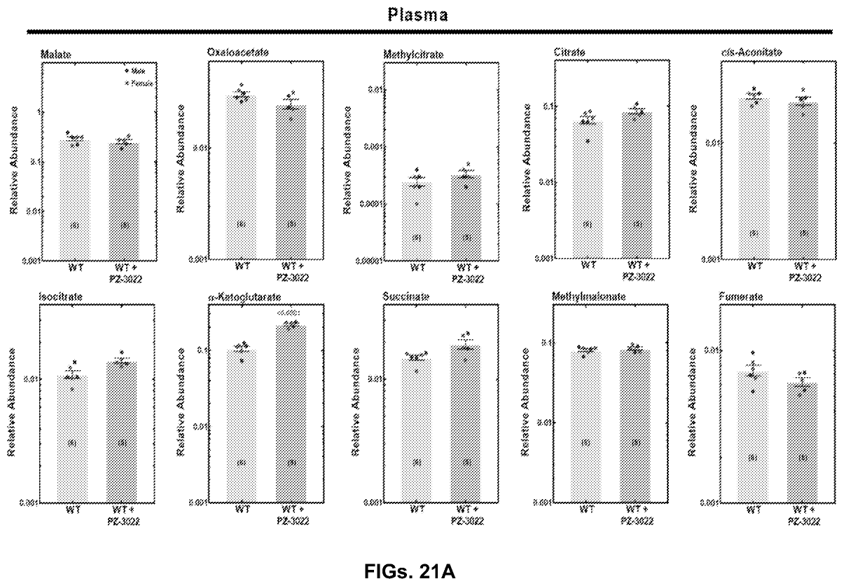

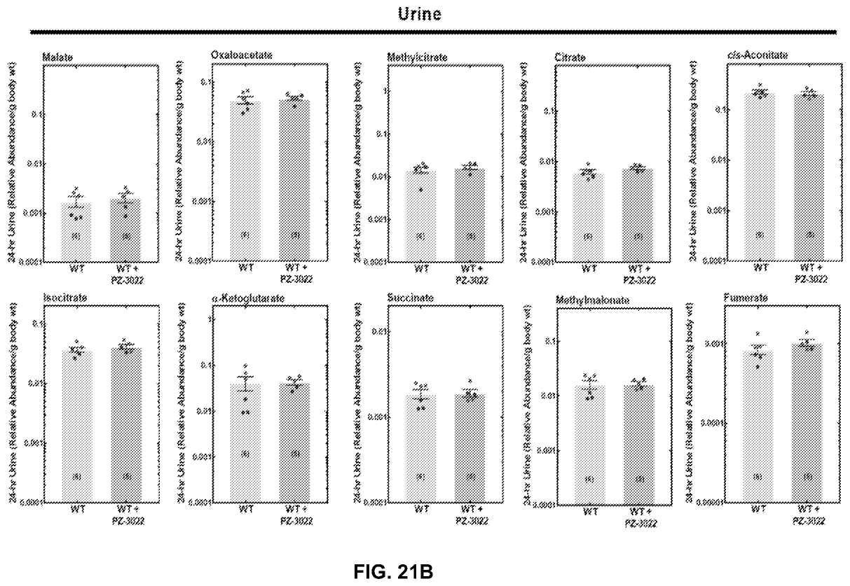

Heart total CoA increased following PZ-3022 therapy (FIG. 19A), but there was no deficiency in C2-CoA to correct (FIG. 10C). There was a statistically significant, but modest increase in the heart:body weight ratio in the PA mouse (FIG. 19B), whereas there was not a statistically significant difference in the heart:body weight ratio comparing the wild-type and PZ-3022-treated groups (FIG. 19B) suggesting a beneficial effect of therapy. Liver CoASH and acetyl-CoA levels increased as expected in PZ-3022-treated wide-type mice (FIG. 20A-20B). C3-CoA levels (FIG. 20C) and the C3:C2 ratios (FIG. 20D) were low and were not altered by PZ-3022. The plasma and urine amino acids of treated wild-type mice were not changed with the exception of histidine being lower in plasma and higher in urine of PZ-3022-treated mice (FIG. 20E). There were no differences in plasma (FIG. 21A) and urine (FIG. 21B) TCA cycle-related metabolite levels in PZ-3022 treated and untreated wild-type mice.

### Discussion

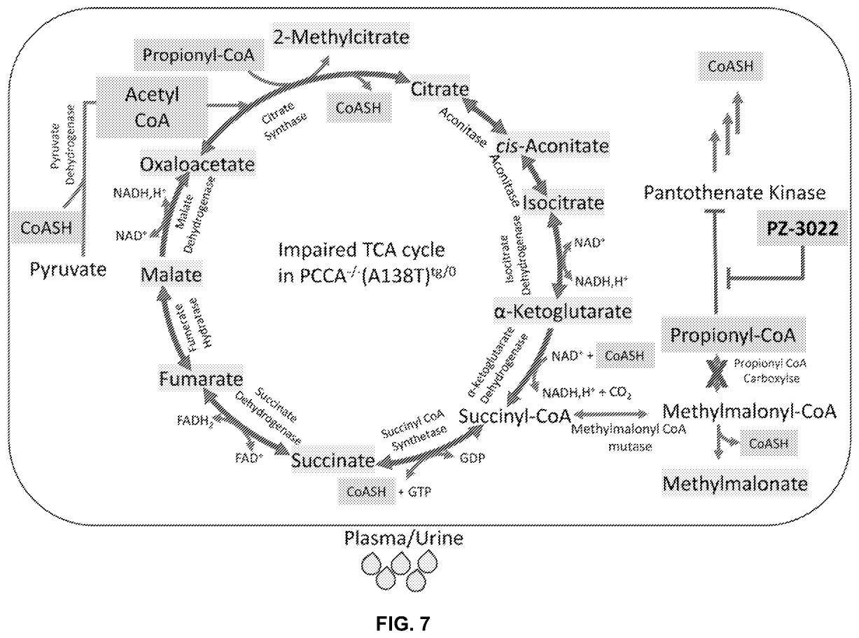

Our study demonstrates that CoASH sequestration contributes to the metabolic imbalance in PA disease. Normally, C3-CoA arising from catabolism enters the TCA cycle for disposition following its conversion to succinyl-CoA by the reversible methylmalonyl-CoA mutase (FIG. 7). In PA, this pathway is constricted at the PCC step leading to the accumulation of C3-CoA. C2-CoA is usually the most abundant feedback regulator of PanK, but in PA, C3-CoA levels are 10-fold higher than C2-CoA. PanK inhibition by the massive accumulation of C3-CoA blocks CoASH biosynthesis thereby exacerbating the CoASH deficiency (FIG. 7). Reduced hepatic CoASH impairs mitochondrial function at two key steps (pyruvate and α-ketoglutarate dehydrogenases) leading to reduced C2-CoA in PA liver and the accumulation of TCA cycle intermediates in plasma and urine. Metabolism responds using several pathways to convert C3-CoA to CoASH and eliminate propionate from the body. Major pathways are the conversion of C3-CoA to either C3-carnitine or methylcitrate, which both liberate CoASH. C3-carnitine and methylcitrate exit tissues into the plasma and are eliminated from the body in the urine. This report identifies methylmalonyl-CoA hydrolysis as another pathway to liberate CoASH and excess propionate is removed from tissues by eliminating methylmalonate in the urine. These measures work together to counteract CoASH sequestration, but in the PA mouse liver they are insufficient to maintain CoASH and C2-CoA levels. PZ-3022 allosterically activates PanK to counteract its inhibition by C3-CoA and normalizes the intracellular levels of total CoA, CoASH, and C2-CoA, and modestly reduces C3-CoA in PA liver. Together, these changes result in a significant improvement in the plasma C3:C2-carnitine ratio, the hallmark biomarker of human PA.

Metabolomics profiling establishes a dysfunctional TCA cycle as an underlying metabolic consequence of PA that arises from CoASH sequestration (FIG. 7). Animal models of CoA deficiency indicate that compromised mitochondrial metabolism is a major consequence of insufficient cellular CoASH suggesting that reduced CoASH is at the root of mitochondrial dysfunction in PA. CoASH is a key substrate for two irreversible steps in the TCA cycle, pyruvate and α-ketoglutarate dehydrogenases (FIG. 7). CoASH deficiency slows these steps, reducing the ability of mitochondria to metabolize pyruvate and TCA cycle intermediates. Instead, these intermediates diffuse out of the tissues into the plasma and accumulate in urine. Elevated TCA cycle intermediates in PA mouse urine provide direct evidence for a dysfunctional TCA cycle and corroborate the similar observations in human PA. TCA cycle intermediates are not routinely assessed in plasma or urine of PA patients but may serve as biomarkers for the severity of mitochondrial impairment and to assess treatment efficacy. Urinary malate is a sensitive indicator of TCA cycle function in the PA model that has not been previously appreciated. Plasma malate increases 3-fold, but malate is 85-fold higher in urine. This relationship between plasma and urinary malate levels is consistent with the established limit on malate reabsorption by the kidney, a process that may explain why many TCA intermediates are more significantly elevated in urine than in plasma. PZ-3022 therapy results in an 80% reduction in urinary malate providing direct evidence that PZ-3022 therapy enhances mitochondrial function that is compromised by PA. TCA cycle dysfunction is so compromised by CoASH sequestration in PA that the methylmalonyl-CoA produced by the limited amount of PCC activity present in PA liver cannot be metabolized by the TCA cycle. Instead, methylmalonyl-CoA is cleaved to methylmalonate to release CoASH and is eliminated in the urine. Following PZ-3022 therapy, succinyl-CoA arising from C3-CoA is utilized by the TCA cycle leading to a 50% reduction in C3-CoA in liver and a 47% reduction in urinary methylmalonate. Gene or RNA therapy to correct PCC activity in PA liver is another current focus for therapeutics development. These therapies restore liver PCC activity and reduce circulating C3-carnitine and methylcitrate biomarkers. However, CoASH biosynthesis is cell autonomous and correcting liver metabolism would not alleviate CoASH sequestration and TCA cycle malfunction if these occur in other tissues such as heart and brain. PZ-3022 stabilizes intermediary metabolism and TCA cycle function in the PA mouse model providing translational proof-of-concept that pantazine therapy may improve mitochondrial function in human PA disease.

Pantazines are first in class allosteric PanK activators with the potential to be disease modifying drugs in other conditions involving CoASH sequestration or mitochondrial dysfunction. CoASH is the major organic acid carrier in biology and several organic acidemias and β-oxidation inborn errors of metabolism are predicted to trigger the accumulation of a particular CoA thioester that sequesters CoASH and inhibits CoASH biosynthesis. Pantazines may treat toxicities arising from exposure to xenobiotic carboxylic. Valproate is a widely used antiepileptic drug, but hepatotoxicity remains a dose-limiting side effect. Impaired mitochondrial function in valproate toxicity may arise from CoASH sequestered as thioesters of valproate and its degradation products. The ability of PZ-3022 to rapidly (2 h) alleviate liver CoASH sequestration suggests that pantazines would be useful in the treatment of valproate toxicity. Many age-related and degenerative diseases are associated with mitochondrial dysfunction and the pantazines may benefit these diseases by increasing CoASH availability to support TCA cycle function. Depressed CoASH and C2-CoA coupled with elevated TCA cycle intermediates may disrupt intermediary metabolism at several levels in light of their multiple roles in metabolism, metabolic regulation, epigenetic control and protein interaction networks. Examining the impact of all these disruptions is beyond the scope of this study, but the normalization of metabolite levels by PZ-3022 therapy would dampen any negative effect of their accumulation on intermediary metabolism.

### Study Design

In this study, an established PA mouse model was used to interrogate the metabolic imbalance as related to CoA sequestration. Pcca^{-/-}PCCA(A138T)^{tg/0} (PA) mice were obtained from the Mayo Clinic, re-animated and generated at St. Jude Children's Research Hospital. PA mice were treated with PZ-3022, a member of a novel class of allosteric modulators of PanK, the controlling activity in CoA biosynthesis. PZ-3022 was synthesized and developed at St. Jude Children's Research Hospital. For all experiments, we used numbers of biological replicates consistent with previously published studies following similar experimental protocols. These numbers are sufficient to produce statistically meaningful results (p<0.01). The number of biological replicates for each experiment is specified in the figures or legends. Experimental techniques were developed to measure CoA sequestration and established methods were used to evaluate other biochemical disease markers in wild type and PA mice. Pharmacokinetic analysis of PZ-3022 in plasma was performed at SAI LifeSciences Ltd., India. Multiple experimenters performed biochemical analyses and were blinded during data acquisition. Male and female PA mice were randomized into treatment groups without exclusions.

Materials. Sources of supplies were: d-[1-¹⁴C]pantothenate (specific activity, 55 mCi/mmol), American Radiolabeled Chemicals; Ni-NTA resin, Qiagen corporation; d-pantothenic acid, hemicalcium salt, Sigma-Aldrich; Dulbecco's Modified Eagle medium (DMEM; BioWhitaker), Thermo Fisher Scientific; CoA and CoA thioesters, Avanti Polar Lipids (Croda International); 1-[methyl-d₉]carnitine, [methyl-d₃]acetyl-carnitine, [methyl-ds]propionyl-carnitine, [methyl-d₃]butyryl-carnitine, [methyl-d₉]isovaleryl-carnitine, and Labeled Carnitine Standards Set B, Cambridge Isotope Labs; [¹³C₂]acetyl-CoA (Sigma-Aldrich). All other materials were reagent grade or better.

PANK activity assays. PANK activity assays in FIGS. 3B and 16A-16D were performed as described earlier using purified recombinant human PANK1β, mitochondrial PANK2, or PANK3 proteins. Briefly, the reaction mixture contained 100 mM Tris-HCl, pH 7.5, 10 mM MgCl₂, 2.5 mM ATP, 45 μM d-[1-¹⁴C]pantothenate (specific activity, 22.5 mCi/mmol) and 1-30 nM of PANK protein±0-0.1 μM PZ-3022. Each assay was incubated at 37° C. and the reaction was stopped after 10 min by the addition of 4 μl 10% (v/v) acetic acid. The mixture was spotted onto a DE81 ion-exchange filter disk and analyzed as described previously. Activity measurements in presence of C3-CoA used 10-100 ng of PANK and 1-5 μg PANK protein for assays+2-10 μM PZ-3022, and 1 mM ATP, with C3-CoA added at 0-0.1 mM. All experiments were done twice with duplicate samples, the data combined and averaged ±SEM.

PANK3 crystallization and structure determination. PANK3 with two amino acids (DD) added to the carboxy-terminus was expressed, purified and crystallized. Crystals of the PANK3⋅AMPPNP⋅Mg²⁺⋅pantothenate complex were soaked with 1 μM PZ-3022 in mother liquor (0.2 M ammonium acetate, 0.1 M citrate, pH 5.6, 50 mM MgCl₂, 32% polyethylene glycol (4K), 4% DMSO, and 10 mM adenosine 5′-(β,γ-imido)triphosphate for two days to replace the bound pantothenate with PZ-3022. The crystal was cryoprotected with 29% ethylene glycol. Diffraction data were collected at the SER-CAT beam line 22-ID at the Advanced Photon Source and processed using HKL2000. The structure was solved by molecular replacement using the PANK3 structure (PDB ID: 6B3V) and the program PHASER. The structure was refined and optimized using PHENIX and COOT, respectively. The refined structure was validated using MolProbity. The atomic coordinates and structure factors have been deposited in the Protein Data Bank as PDB entry 6PE6. The data collection and refinement statistics are presented in Table 4. All structures were rendered with PyMOL (version 2.3, Schrödinger, LLC).

Pharmacokinetic evaluation in mice. Pharmacokinetic parameters of PZ-2891 and PZ-3022 in plasma following a single intravenous dose of 2 mg/kg administration to male Balb/c mice was done at SAI LifeSciences Ltd., India (FIG. 3E). Briefly, eighteen male mice were divided into two groups as Group 1 (IV: 2 mg/kg) and Group 2 (PO: 10 mg/kg). Animals in Group 1 and Group 2 were administered intravenously and orally with PZ-2891 or PZ-3022 formulated in Captisol (30% w/v) at 2 mg/kg and 10 mg/kg dose, respectively. Blood samples (approximately 60 μl) were collected from retro-orbital plexus under light isoflurane anesthesia such that the samples were obtained pre-dose, 0.08, 0.25, 0.5, 1, 2, 4, 8 and 24 h (IV) and pre-dose, 0.25, 0.5, 1, 2, 4, 6, 8 and 24 h (PO). The blood samples were collected from sets of three mice at each time point in labeled microcentrifuge tubes containing EDTA as anticoagulant. Plasma samples were separated by centrifugation of whole blood and stored below −70° C. until analysis. Plasma (50 μl) was processed for analysis by protein precipitation using acetonitrile containing 50 ng/ml Lansoprazole for internal standard and was analyzed with LC/MS/MS (LLOQ: 1.26 ng/ml). Pharmacokinetic parameters were calculated using the non-compartmental analysis tool of Phoenix WinNonlin (Version 6.3). Mean pharmacokinetic parameters are summarized in Table 5.

Cell culture. Human C3A (HepG2/C3A) cells (#CRL-1074) were purchased from ATCC® and maintained in DMEM supplemented with 2 mM glutamine, 10% fetal bovine serum (Atlanta Biologicals), 50 U/ml penicillin and 50 mg/ml streptomycin. HEK293T cells (ATCC, #CRL-3216) were used for expression of human PCCA protein standard and were maintained in DMEM plus supplements as described above. Each cell line was confirmed to be mycoplasma-free. C3A cells in FIG. 3D were grown to a density of 6-8×10⁶ in 100-mm petri dishes at 37° C. in 5% CO₂:95% air for 24 h following addition of PZ-2891 or PZ-3022 (10 μM final) dissolved in DMSO (0.1% final). DMSO alone was added to control cultures. Culture medium was aspirated off the dish, cells were quickly washed with ice-cold phosphate-buffered saline that was quickly aspirated off the dish, and cells were quickly washed again with ice-cold water that was aspirated off to remove residual saline. Ice-cold water (1 ml) was added to the culture dish, cells were scraped into the cold water and the cell suspension was transferred to a glass test tube containing 400 μl of 0.25 μM KOH and 1.5 ml water for analysis of total CoA content. Parallel cultures with or without PZ-2891 or PZ-3022 were prepared and cells were collected after 24-h incubation for determination of final cell numbers and cell viabilities using a Nucleocounter (New Brunswick Scientific) according to the manufacturer's directions.

Animal experiments. C57BL/6J mice (age 8 weeks) were purchased from Jackson Laboratory. The genetic mouse model of propionic acidemia was obtained as frozen sperm from Michael Barry (Mayo Clinic, Rochester Minnesota, USA). Following re-animation, mice were genotyped using the Pcca, PCCA, and GFP primers described in Table 6. Pcca^{-/-}PCCA(A138T)^{tg/0} mice were crossed with Pcca^{+/-} mice to maintain the transgene as single copy in all study animals. Progeny distribution followed a Mendelian pattern of inheritance, and 1 in 8 pups had the desired Pcca^{-/-}PCCA(A138T)^{tg/0} genotype. Matched wild-type mice had a Pcca^{+/+} genotype. Animals were maintained on LabDiet 5013, a soy/grain-based chow, at room temperature 72±2° F., humidity 50%±10% and a 14-hour light/10-hour dark cycle with the dark cycle starting at 20:00 (FIGS. 1A-1L and 12A-12B). Water was supplied ad libitum. Prior to oral administration of PZ-3022, C57BL/6J mice were fed an isocaloric purified diet (Envigo TD.170542) containing 17.9% protein, 62.4% carbohydrate, 6% fat for 2 weeks (FIGS. 4A-4H and 18A-18H). PZ-2891 or PZ-3022 were formulated in 30% Captisol and delivered by gavage (FIG. 3F) or in diet (FIGS. 16A-16D). The Pcca^{-/-}PCCA(A138T)^{tg/0} mice on the therapy studies were fed the control Envigo TD.170542 diet supplemented with 1000 ppm pantothenate or the same diet containing 75 ppm PZ-3022 (FIGS. 2A-2B, 3A-3F, 4A-4H, 5A-5L, 6A-6B, 10A-10E, 11A-11B, 12A-12B, 13, 18A-18H, 19A-19B, 20A-20E, and 21A-21B). Food monitoring showed that males ate an average of 2.5 g/day and females ate 2.0 g/day. Mice were euthanized, tissues collected and flash frozen, and plasma collected in Na₂EDTA then frozen, at 4 hours after the last oral dose, or at 8:00 am after the 50-day study. Urine was collected over a 24-h period from mice singly housed in metabolic cages, following prior acclimatization for 24 hours.

TABLE 6
Primers and PCR conditions for genotyping

| | Primer | | |
|---|---|---|---|
| Gene | Name | Sequence 5¢-3¢ | PCR Conditions |
| Pcca | mPCCA | aggaagccaggaaaggttatttg | 94ºC, 5 min + 45 cycles (94° C., |
| | For2 | | 30 sec + 64° C., 30 sec + 72° C., |
| | mPCCA | CCCTACAGGCAATTTCTCCTC | 30 sec) + 72° C., 2 min + 4° C., ∞ |
| | Rev3 | | WT = 457 bp KO = 688 bp |
| | mPCCA | GGATGATCTGGACGAAGAGC | |
| | NeoRev4 | | |
| PCCA(A158T) | hPCCA For | acagtgcttaatggtgtccc | 94ºC, 5 min + 40 cycles (94° C., |
| | hPCCA Rev | cttctgcatccttgactactcc | 30 sec + 60°° C., 30 sec + 72° C., |
| | oIMR0015 | CAAATGTTGCTTGTCTGGTG | 45 sec) + 72° C., 2 min + 4° C., ∞ |
| | oIMR0016 | GTCAGTCGAGTGCACAGTTT | Positive = 485 bp Int'l |
| | | | Control = 200 bp |
| Gfp | GFP-For1: | CACATGAAGCAGCACGACTT | 94° C., 5 min + 40 cycles (94° C., |
| | GFP-Rev1: | TGCTCAGGTAGTGGTTGTCG | 30 sec + 58)C, 30 sec + 72° C., |
| | oIMR0015: | CAAATGTTGCTTGTCTGGTG | 30 sec) + 72°C, 2 min + 4° C., ∞ |
| | oIMR0016: | GTCAGTCGAGTGCACAGTTT | Positive = 380 bp Int'l |
| | | | Control = 200 bp |

Western blotting. The human PCCA cDNA (Invitrogen) was cloned into the pCDNA3.1(−) vector following restriction digestion with NheI/XhoI (pJW16-1). After confirming the plasmid DNA sequences, HEK293T cells were transfected with either plasmid using FuGENE® 6 reagent (Promega) combined with 12 μg plasmid in a 100-mm dish. Cell lysates were prepared 48 hours after transfection by resuspending washed cells in RIPA buffer (Pierce #89900) and protein content was determined using the Bradford method. Lysates from cells expressing PCCA were used as a Western blotting standard. Tissues from wild-type and PA mice maintained on Envigo TD.190398 (FIGS. 2A and 11A-11B) were homogenized in RIPA buffer, and 10 μg protein of tissue lysate from wild-type mice and 60 μg protein of tissue lysate from PA mouse tissues were used per lane for fractionation by gel electrophoresis using NuPAGE 10% Bis-Tris Gels (Invitrogen). Following transfer to PVDF membrane (iBlot 2 PVDF Regular Stacks; Invitrogen), the blots were blocked for 1 h in 5% milk/TBS-T and then exposed to primary antibody anti-PCCA (Protein tech, 21988-1-AP) that reacts with mouse Pcca and human PCCA at 1:3000 dilution, together with anti-GAPDH (Abcam, ab9485) 1:2500 dilution overnight. Following washing in 3% BSA/TBS-T, blots were incubated with secondary antibody (anti-Rabbit AP conjugated; Sigma Aldrich #3687) in 1% milk/TBS-T for 1 hour at 1:5000 dilution. The blots were washed extensively and exposed to the ECF substrate for 5 minutes, and the bands on the dried membrane were quantified on the Typhoon FLA9500 using ImageQuant TL software (GE Healthcare).

Acyl-Carnitine quantification. Acyl-carnitines were extracted from 20 μl of plasma that was mixed with 180 μl water and 800 μl acetonitrile. To this mixture, 10 μl of labeled Carnitine Standards Set B (Cambridge Isotope Labs) or 25 μl of Heavy Carnitine Standard Mix was added and the mixture was incubated on ice for 10 min. The sample was then centrifuged at 10,000×g for 10 min to remove any precipitated debris. Acyl-carnitines were extracted from freshly thawed liver (30-40 mg) by homogenizing the tissue in 2 ml of 80% acetonitrile, followed by addition of 25 μl of Heavy Carnitine Standard Mix and incubation at −80° C. for 30 minutes. The sample was then centrifuged at 5000×g for 10 minutes. The supernatant obtained from the plasma or liver extractions was cleaned on a solid-phase extraction column, Bond Elut-PRS (Varian) which was activated by washing the column with 3 ml of methanol and 3 ml 100:3.5 (v/v) methanol:HCl followed by two 3 ml washes of water. After loading, the column was washed twice with 3 ml water and once with 3 ml methanol. Elution of the acyl-carnitines from the column was done with 3 ml of 40 mM BaCl₂ in 75% methanol, and the eluant was dried under nitrogen gas. The residue was resuspended in 3 ml acetonitrile and transferred to a new tube to be dried under nitrogen. To the residue, 500 μl of 4:1 (v/v) 2-propanol:acetylchloride was added and incubated a 65° C. for 30 min. The reaction mix was dried under nitrogen. Samples were resuspended in 80% acetonitrile prior to analysis by HPLC/MS/MS.

Acyl-carnitines were fractionated, identified and quantified using a Shimadzu Prominence UFLC attached to a QTrap 4500 equipped with a Turbo V ion source (Sciex). Samples (5 μl) were injected onto an XSelect® HSS C18, 2.5 μm, 3.0×150 mm column (Waters) using a flow rate of 0.4 ml/min. Solvent A was 0.1% formic acid in water, and Solvent B was acetonitrile±0.1% formic acid. The HPLC program was the following: starting solvent mixture of 25% B, 0 to 2 min isocratic with 25% B; 2 to 10 min linear gradient to 100% B; 10 to 20 min isocratic with 100% B; 20 to 22 min linear gradient to 25% B; 22 to 25 min isocratic with 25% B. The QTrap 4500 was operated in the positive mode, and the ion source parameters were: ion spray voltage, 5500 V; curtain gas, 25 psi; temperature, 350° C.; collision gas, medium; ion source gas 1, 20 psi; ion source gas 2, 25 psi; declustering potential, 60 V; and collision energy, 40 V. The following MRMs were used: carnitine, 204.4/85.0; [d₉]carnitine, 213.4/85.0; C2-carnitine, 246.4/85.0; [d₃]acetyl-carnitine, 249.4/85.0; C3-carnitine, 260.4/85.0; and [d₃]propionyl-carnitine; 263.4/85.0. The system was controlled by Analyst® software (Sciex) and analyzed with MultiQuant™ 3.0.2 software (Sciex). Heavy Carnitine Standard Mix contained the following: 150 μM 1-[methyl-d₉]carnitine, 40 μM [methyl-d₃]acetyl-carnitine, 30 μM [methyl-d₃]propionyl-carnitine, 5 μM [methyl-d₃]butyryl-carnitine, and 5 μM [methyl-d₉]isovaleryl-carnitine.

Total CoA determination. CoA was quantified according to published procedures. Briefly, suspensions of cultured C3A cells in KOH were incubated for 2 hours at 55° C. to hydrolyze CoA thioesters, the pH was lowered to 8 by addition of 1 μM Trizma-HCl, and monobromobimane (mBBr, Life technologies) was added in excess to derivatize the CoASH. Similarly, 30-40 mg of freshly thawed liver was homogenized in KOH to hydrolyze all the CoA thioesters, the pH was lowered and CoASH in the mixture was derivatized with mBBr. The CoA-bimane product in the mixture was purified on a SPE 2-(2-pyridyl)ethyl column and fractionated by HPLC with fluorescent detection λex=393 nm, λem=470 nm or absorbance detection at 393 nm. The retention time was determined by running a CoA-bimane standard before each set of samples. Quantification was done using a calibration curve made with the CoA-bimane standard.

Acyl-CoA measurement by mass spectrometry. Acyl-CoAs were quantified as described earlier. Briefly, 30-40 mg of liver was homogenized in 2 ml methanol and 1 ml water and incubated on ice for 30 minutes. Chloroform 1.5 ml and 1.2 ml water were added to remove the lipids. The aqueous top layer was collected and 30 μmol of [¹³C₂]acetyl-CoA (Sigma) was added, and dried overnight in a Savant Speedvac Concentrator SPD 1010 (Thermo-Fisher). The dried sample was resuspended in 300 μl water and 20 μl were injected into a mass spectrometer. Acyl-CoA was analyzed using a Shimadzu Prominence UFLC attached to a QTrap 4500 equipped with a Turbo V ion source (Sciex). Samples were injected onto an Acquity UPLC HSS C18, 2.5 μm, 3.0×150 mm column at 40° C. (Waters) using a flow rate of 0.2 ml/min. Solvent A was 10 mM ammonium acetate pH 6.8, and Solvent B was 95% acetonitrile+10 mM ammonium acetate pH 6.8. The HPLC program was the following: starting solvent mixture of 95% A/5% B, 0 to 2 min isocratic with 5% B; 2 to 20 min linear gradient 100% B; 20 to 25 min isocratic with 100% B; 25 to 27 min linear gradient to 5% B; 27 to 31 min isocratic with 5% B. The QTrap 4500 was operated in the positive mode, and the ion source parameters were: ion spray voltage, 5500 V; curtain gas, 15 psi; temperature, 400° C.; collision gas, medium; ion source gas 1, 15 psi; ion source gas 2, 20 psi; declustering potential, 60 V; and collision energy, 45 V. The MRM transitions are: CoASH, 168.1/261.1; C2-CoA, 810.1/303.1; C3-CoA, 824.1/317.1; and [¹³C]acetyl-CoA, 812.1/305.1. [¹³C]Acetyl-CoA was used to calculate relative abundance of the acyl-CoAs in tissue. The system was controlled by the Analyst® software (Sciex) and analyzed with MultiQuant™ 3.0.2 software (Sciex). Peaks corresponding to individual acyl-CoA species were quantified relative to the internal standard.

PZ-2891 and PZ-3022 quantification by LC-MS/MS. The concentrations of PZ-2891 and PZ-3022 in plasma and liver were determined as described earlier. Briefly, 20 μl freshly thawed plasma was added to 100 μl acetonitrile containing 0.6 μM warfarin. Samples were incubated on ice for 30 min, then centrifuged at 3500×g for 10 min to pellet debris, and the supernatant was transferred to a glass vial. A PZ-2891 or PZ-3022 standard curve was generated by adding known concentrations of PZ-2891 or PZ-3022 to 20 μl of mouse plasma from an untreated animal and following the above procedure. Freshly thawed liver (30-40 mg) was homogenized in 2 ml of 80% methanol containing 0.1 μM warfarin and incubated at −80° C. for 4 hours. Samples were centrifuges at 3500×g for 10 min to pellet debris, supernatant was transferred to a glass tube and dried down using a Savant SPD1010 Speed-Vac (Thermo Scientific) overnight. Samples were resuspended in 400 μl of 80% acetonitrile to a final concentration of 0.5 μM and transferred to a glass vial.

PZ-2891 or PZ-3022 were analyzed using a Shimadzu Prominence UFLC attached to a QTrap 4500 equipped with a Turbo V ion source (Sciex). Samples (5 μl) were injected onto an XSelect® HSS C18, 2.5 μm, 3.0×150 mm column (Waters) using a flow rate of 0.25 ml/min. Solvent A was 0.1% formic acid in water, and Solvent B was acetonitrile+0.1% formic acid. The UFLC program was the following: starting solvent mixture of 50% B, 0 to 0.5 min isocratic with 50% B; 0.5 to 1.5 min linear gradient to 95% B; 1.5 to 20 min isocratic with 95% B; 20 to 21 min linear gradient to 50% B; 21 to 25 min isocratic with 50% B. The QTrap 4500 was operated in the positive mode, and the ion source parameters were: ion spray voltage, 5500 V; curtain gas, 30 psi; temperature, 450° C.; collision gas, medium; ion source gas 1, 30 psi; and ion source gas 2, 40 psi. The MRM transition for PZ-2891 was 350.2/190.0 m/z; PZ-3022 was 348.2/190.0 m/z, and warfarin was 309.1/163.0 m/z with a declustering potential, 65 V and collision energy, 30 V. The system was controlled by the Analyst® software (Sciex) and analyzed with MultiQuant™ 3.0.2 software (Sciex).

Fatty acid analysis. Lipids were extracted from 50 mg liver using a modification of the Bligh and Dyer procedure optimized for lipid quantitation by the LipidMaps group. The amounts of each fatty acid, including free fatty acid and lipid fatty acyl groups, were determined following extraction and methyl ester preparation, followed by hexane extraction. The amount of each fatty acid methyl ester was determined by flame-ionization detection following gas chromatography using a Hewlett Packard 5890A gas chromatograph equipped with a DB-225 column (Agilent). The fatty acids were identified by co-migration with authentic standards and quantified by integration of the signal peaks.

Metabolites in urine and plasma. Water was added to a 24-h urine sample to a final volume of 1 ml for each mouse. To 100 μl of urine or 20 μl of plasma, warfarin standard was added at a final concentration of 0.2 μM and the sample was incubated at −80° C. for 1 hour. The sample was centrifuged at 6000×g for 10 minutes to remove precipitants and the supernatant was analyzed by LC/MS/MS. Analysis was performed using a Shimadzu Prominence UFLC attached to a QTrap 4500 equipped with a Turbo V ion source (Sciex). Samples (5 μl) were injected onto an XSelect® HSS C18, 2.5 μm, 3.0×150 mm column (Waters) using a flow rate of 0.4 ml/min. Solvent A was 0.1% formic acid in water, and Solvent B was acetonitrile+0.1% formic acid. The HPLC program was the following: starting solvent mixture of 25% B, 0 to 2 min isocratic with 25% B; 2 to 10 min linear gradient to 100% B; 10 to 20 min isocratic with 100% B; 20 to 22 min linear gradient to 25% B; 22 to 25 min isocratic with 25% B. The QTrap 4500 was operated in the negative mode using the following ion source parameters: ion spray voltage, −4500 V; curtain gas, 40 psi; temperature, 500° C.; collision gas, medium; ion source gas 1, 50 psi; ion source gas 2, 50 psi. In the positive mode, the ion source parameters were: ion spray voltage, 5500 V; curtain gas, 20 psi; temperature 400° C.; collision gas, medium; ion source gas 1, 25 psi; and ion source gas 2, 40 psi. The MRMs for metabolites in either the positive or negative mode were taken from. The MRMs for the warfarin internal standard were: negative mode, 307.1/161.0; and positive mode, 309.1/163.0. The system was controlled by the Analyst® software (Sciex) and analyzed with MultiQuant™ 3.0.2 software (Sciex). The relative amount of metabolite was normalized to the amount of warfarin. To improve glycine detection, this metabolite was measured after derivatization with benzoyl chloride using the technology and MRMs described.

Echocardiography. Echocardiography was performed using a VEVO-3100 preclinical imaging system equipped with a MX550, 40 MHz transducer (Fujifilm-Visualsonics, Toronto, ON, Canada). Prior to scanning, fur was removed from the chest of each animal using Nair (Church and Dwight, Ewing, NJ). Mice were anesthetized with isoflurane (2%-3% in 100% oxygen) and positioned supine on the heated platform. The chest was coated with ultrasound gel (Aquasonics, Parker Labs, Fairfield, NJ) and scanned in B-mode for positioning with reference to the heart. Parasternal long axis (PLA) and parasternal short axis (PSA) scans were performed in M-mode. AH scans were taken with the heart rate between 300 and 400 beats per minute (bpm). Cardiac function parameters (Table 2) were calculated from M-mode traces using Vevo LAB 3.2.0 software (Fujifilm-Visualsonics).

Statistical analysis: Statistical tests were performed using GraphPad Prism software v8.43 (http://graphpad.com/scientific-software/prism/). Unpaired parametric t test was used when comparing samples. The number of replicates, the mean±SEM and the p values are specified in the figures or legends. Significance means p<0.01 and the actual values are reported in red in the figures. Not significant (ns) means p>0.01.

### Example 2: Synthesis of 6-(4-(2-(4-cyclopropylphenyl)acetyl)piperazin-1-yl)pyridazine-3-carbonitrile

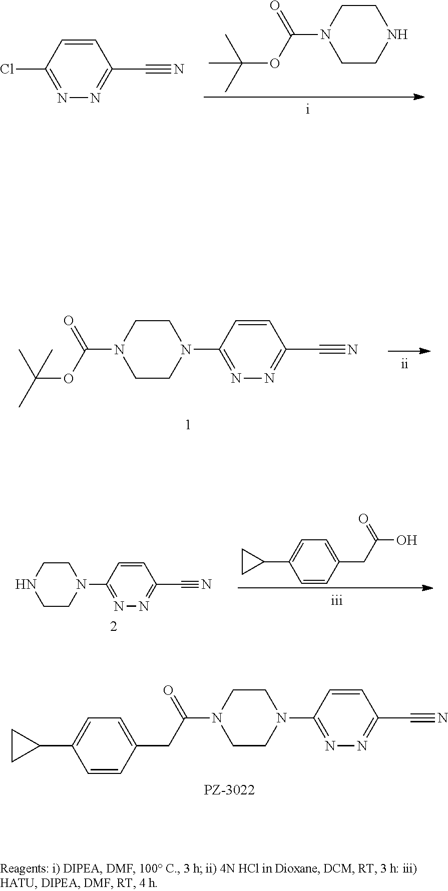

The mixture of 1-boc-piperazine (13.35 g, 71.7 mmol), 6-chloropyridazine-3-carbonitrile (10 g, 71.7 mmol) and diisopropyl ethylamine (DIPEA) (25.03 ml, 143 mmol) in dimethylformamide (DMF) (90 ml) was heated to 100° C. for 3 h. The reaction was followed by LC/MS. The reaction mixture was cooled to room temperature and diluted with cold water (200 ml). The solids separated were collected by filtration, washed with water and followed by cold acetonitrile to get the intermediate 1 (tert-butyl 4-(6-cyanopyridazin-3-yl)piperazine-1-carboxylate) in quantitative yields, which was used in next step without further purification. ESI-MS: 290.50 (M+1).

The intermediate 1 (1.7 g, 5.8 mmol) in dichloromethane (DCM) (10 ml) was treated with 4 N HCl in dioxane (8 ml) and allowed to stir for 3 h at room temperature. The reaction mixture was then evaporated to dryness under reduced pressure to get the intermediate 2 (6-(piperazin-1-yl)pyridazine-3-carbonitrile) as HCl salt (quantitative yields) which was used in next step without further purification. ESI-MS: 190.42 (M+1). To a mixture of 2-(4-cyclopropylphenyl)acetic acid (100 mg, 0.567 mmol), DIPEA (193 μl, 1.13 mmol) in DMF (2 ml) was added (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium) (HATU; 237 mg, 0.624 mmol) and allowed to stir for 15 min, then intermediate 2 (141 mg, 0.624 mmol) was added and allowed to stir at RT for 4 h. The reaction was followed by LC/MS, after completion of reaction the mixture was diluted with cold water (6 ml), collected the solids by filtration, washed with water and dried. The crude product was subjected to flash Silica Gel column chromatography using a linear gradient of ethyl acetate in hexanes from 0%-100%, the product was eluted at 100% ethyl acetate and upon concentration of solvents afforded 110 mg of PZ-3022 (59% yield). ¹H NMR (500 MHz, DMSO-d₆) δ 7.89 (d, J=9.7 Hz, 1H), 7.35 (d, J=9.7 Hz, 1H), 7.18-7.07 (m, 2H), 7.06-6.95 (m, 2H), 3.79-3.59 (m, 1OH), 1.88 (t, J=8.4, 5.1 Hz, 1H), 1.01-0.85 (m, 2H), 0.72-0.56 (m, 2H). ¹³C NMR (126 MHz, DMSO) δ 169.85, 159.14, 142.18, 132.92, 131.55, 129.33, 129.27, 125.81, 117.83, 111.84, 45.02, 44.37, 44.13, 15.20, 9.73. ESI-MS: 348.52 (M+1)

### Example 3: Proton Magnetic Resonance Spectroscopy Detects Cerebral Metabolic Derangement in a Mouse Model of Brain Coenzyme a Deficiency

Proton magnetic resonance spectroscopy (¹H MRS) was used to monitor a PKAN therapeutic in a mouse model of the disease. Neuronal SynCre⁺Pank1,2 dKO mice were treated with Compound 1 and ¹H MRS was used to identify prominent neurochemical metabolites and evaluate treatment response. Overall survivability and physiological markers, as well as neurochemical biomarkers, were investigated. The cerebral metabolite levels, including glutamate+glutamine (Glx), N-acetyl aspartate (NAA), and lactate, were found to be altered in the mid-brain of the animal model. These results suggest that Glx levels, in particular, may be an indicator of restored CoA levels in response to treatment with a compound provided herein, such as Compound 1

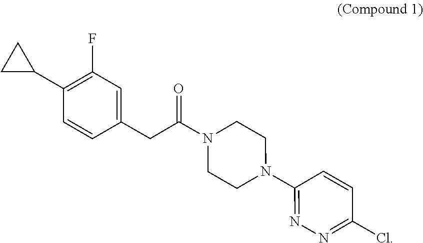 [SMILES: O=C(Cc1ccc(C2CC2)c(F)c1)N1CCN(c2ccc(Cl)nn2)CC1]

### Animal Care and Use

All procedures were reviewed and approved by the St. Jude Children's Research Hospital Institutional Animal Care and Use Committee. The neuronal SynCre⁺Pank1,Pank2 dKO mice were generated as described in Sharma et al. “A therapeutic approach to pantothenate kinase associated neurodegeneration,” Nature communications, 9, 1-15, 2018, which is herein incorporated by reference in its entirety. The mice were maintained at room temperature, humidity 50±10% and a 14/10-hour light/dark cycle. After weaning, animals were maintained on a purified chow diet (Envigo TD.170542)±75 parts per million (ppm) Compound 1. Water was supplied ad libitum. Mice were randomized into the treatment arms at weaning. SynCre⁺Pank1,2 dKO (n=5) received Compound 1 fortified chow for 25 days post weaning. The steady state levels of Compound 1 in plasma (1 micromolar (μM)) and brains (2.5-3 picomoles per milligram (pmol/mg) wet weight) of mice on 75 ppm Compound 1 chow were determined 2-4 hours after cessation of the dark cycle by mass spectrometry analyses as described by Sharma et al. and found to be consistent within the treatment group.

SynCre⁺Pank1,2 dKO (n=6) and SynCre⁻Pank1^{fl/fl},Pank2^{fl/fl} wildtype controls (n=13) received matched control chow without Compound 1 for the same length of time. The untreated SynCre⁺Pank1,Pank2 dKO had a median lifespan of approximately 42 days and survival following treatment was 25% higher. Weight gain was 10% greater in the Compound 1 treatment group at the time of evaluation. MRS was performed on mice at an age of 6-7 weeks. Neuron-specific deletion of Pank1 and Pank2 was confirmed by PCR genotyping of brain and liver postmortem as described by Sharma et al.

### ¹H Magnetic Resonance Spectroscopy in Mice

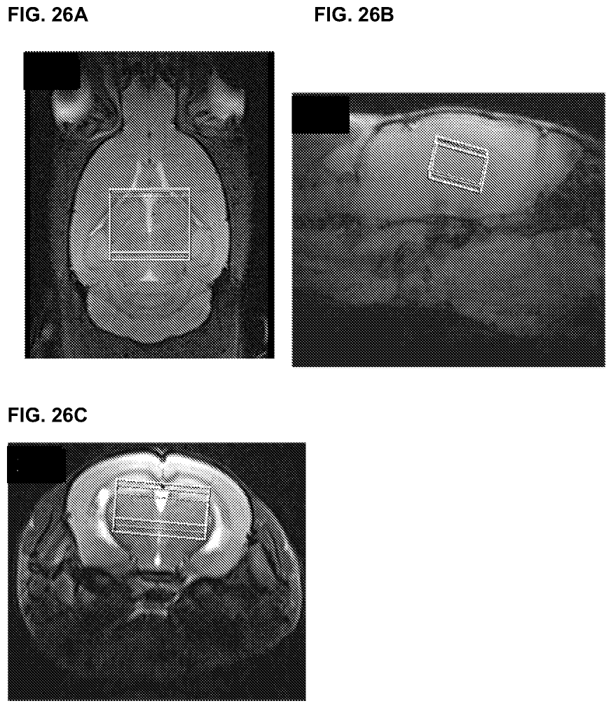

MRI/MRS studies were performed on a Bruker Clinscan 7T magnetic resonance imaging (MRI) scanner (Bruker BioSpin MRI GmbH, Ettlingen, Germany). Mice were anesthetized using isoflurane mixed with oxygen (1-2%) and the respiration rate was monitored. The total scan time was 21 minutes. MRI was acquired with a mouse brain surface receive coil positioned over the mouse head and placed inside a 72 millimeter (mm) transmit/receive coil. After the localizer, a T₂-weighted turbo spin echo sequence was performed in the coronal (TR/TE=2290/41 milliseconds (ms), matrix size=192×256, slice thickness 0.5 millimeters (mm), number of slices=14) and axial (TR/TE=3841/50 ms, matrix size=192×144, slice thickness 0.4 mm, number of slices=42) orientations. The T₂-weighted scans were used to position a 3.5×4.5×2.0 mm³ voxel for spectroscopy in the midbrain to cover thalamus and hippocampus. A ¹H MR spectrum was generated with that voxel using a PRESS sequence (repetition time/echo time=3000/11 ms, averages=128, data length=2048, spectral width=2900 Hertz (Hz)). Voxel positioning is displayed in FIGS. 26A-26C.

### ¹H MRS Data Processing

Metabolite to tCr ratios measured by in vivo ¹H MRS were quantified using LCModel software (v.6.3), a widely applied MRS analysis tool that employs a least-squares-based prior-knowledge fitting program. LCModel applied a 7T spin echo (TE=11 ms) basis set incorporating the following resonances: alanine (Ala), aspartic acid (Asp), creatine (Cre), phosphocreatine, γ-amino butyric acid (GABA), glucose, glutamine (Gln), glutamate (Glu), glycerophosphocholine, phosphocholine, glutathione, myo-inositol (m-Ins), N-acetyl aspartate (NAA), NAA+Glu, sycllo-inositol and taurine, with lipid resonances at 0.9, 1.3 and 2.0 ppm and macromolecule resonances at 0.9, 1.2, 1.4, 1.7 and 2.0 ppm. Metabolite concentrations are reported relative to total creatine (tCr).

### Statistics

Mean and standard deviation were calculated for MRS parameters in each mouse group. Wilcoxon Rank sum tests were used to test whether MRS parameters are different between wild type, SynCre⁺Pank1,Pank2 dKO or SynCre⁺Pank1,Pank2 dKO+ Compound 1 groups. Bonferroni correction was done for the nine comparisons, and thus a p-value <0.05 was deemed to be statistically significant. All the analyses were done using ‘R’ 4.0.2.

### Results and Discussion

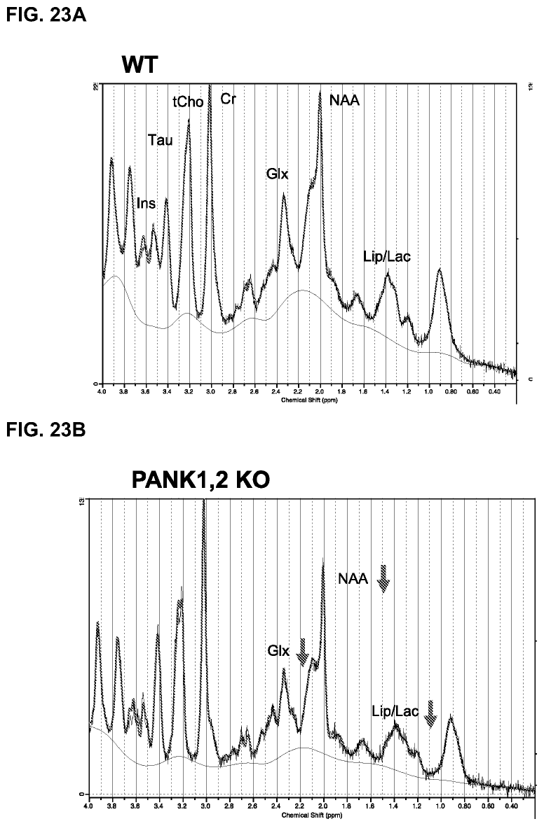

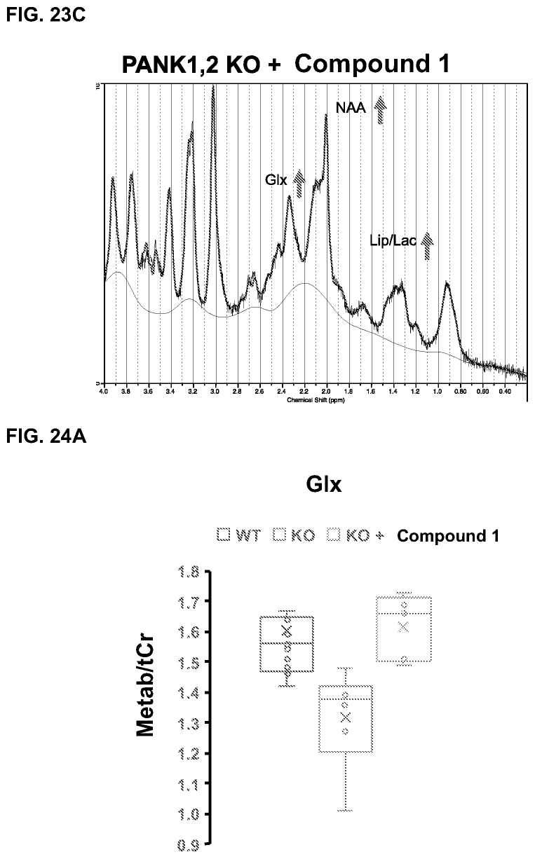

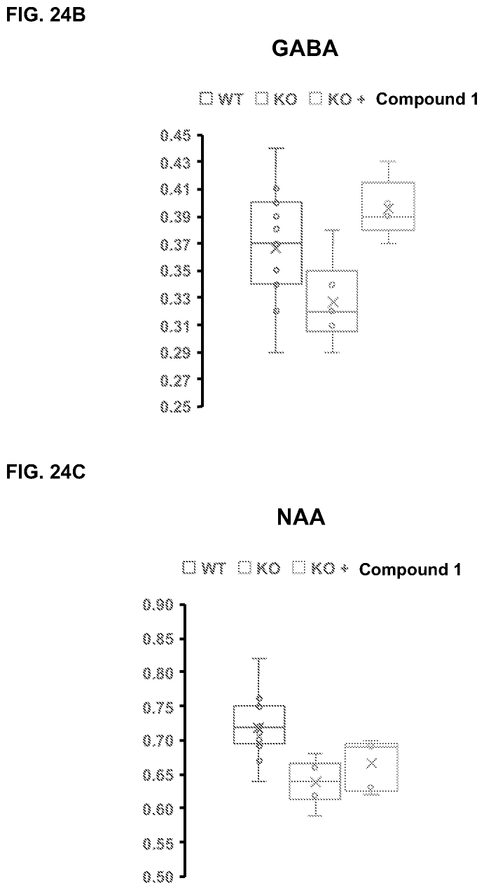

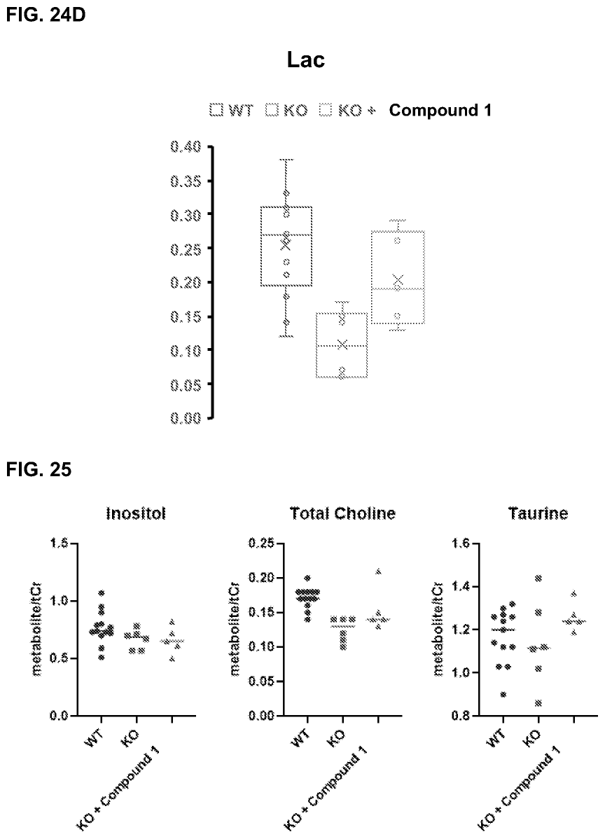

Neuronal SynCre⁺Pank1, 2 dKO mice show clear physiological symptoms of CoA deficiency such as reduced growth rate and impaired locomotor activity. The neuronal SynCre⁺Pank1,2 dKO mice treated with Compound 1 showed improvement of growth rate, lifespan, and movement dysfunction, similar to previous results from Sharma et al. FIGS. 23A-23C represent the ¹H MRS spectra acquired from the midbrain of representative mice in each group. There is a clear reduction in the Glx/tCr, NAA/tCr and lactate/tCr ratios in the midbrains of the neuronal SynCre⁺Pank1,2 dKO mice compared to WT (Wild Type). Glx is the summed group of Glu and Gln, where Glu is the most abundant excitatory neurotransmitter in brain and Gln is the main precursor for Glu. Reduction of Glx points toward impaired excitatory neuronal metabolism in the dKO mice and Glx fully recovered following treatment with Compound 1 (FIGS. 24A-24C, Tables 6 and 7). Reduction of NAA indicated loss of neuronal integrity and function, and the NAA/tCr ratio trended toward improvement but was not significant following Compound 1 treatment. Reduction of NAA in the globus pallidus is consistently reported in patients with PKAN. The lactate/tCr ratio was lower in the dKO mice representing the net balance between lactate production and consumption. Reduced cerebral glycolysis and/or enhanced lactate oxidation to pyruvate to maintain the redox state in the neurons is suggested from the data. The lactate/tCr ratio trended toward improvement with treatment but without statistical significance. The metabolic recovery following therapy, together with a strong signal strength in the ¹H MRS points to Glx as a promising biomarker. NAA and lactate may also be useful biomarkers for study of PKAN. Additional metabolites-inositol, choline, and taurine did not show notable changes but are included in FIG. 25.

TABLE 7
Mean values and standard deviations for FIGs. 24A-24C.

| Metabolite/ | | | KO + |
|---|---|---|---|
| tCr | WT | KO | Compound 1 |
| Glx | 1.60 ± 0.21 | 1.32 ± 0.17 | 1.62 ± 0.11 |
| NAA | 0.72 ± 0.04 | 0.64 ± 0.03 | 0.67 ± 0.04 |
| Lac | 0.25 ± 0.08 | 0.11 ± 0.05 | 0.20 ± 0.07 |

Glx, glutamate/glutamine; KO, untreated Pank1/2 neuronal dKO mice; KO + Compound 1, Compound 1-treated Pank1/2 neuronal dKO mice; Lac, lactate; NAA, N-acetyl aspartate; tCr, total creatine; WT, wild-type.

TABLE 8
p Values for FIGs. 24A-24C.

| Comparison groups | Glx/tCr | NAA/tCr | Lac/tCr |
|---|---|---|---|
| WT vs. Pank1/2 KO | 0.02 | 0.02 | 0.03 |
| WT vs. KO + Compound 1 | 2.70 | 0.20 | 1.95 |
| Pank1/2 KO vs. KO + | 0.04 | 1.49 | 0.60 |
| Compound 1 | | | |

Glx, glutamate/glutamine; KO, untreated Pank1/2 neuronal dKO mice; KO + Compound 1, Compound 1-treated Pank1/2 neuronal dKO mice; Lac, lactate; NAA, N-acetyl aspartate; tCr, total creatine; WT, wild-type.

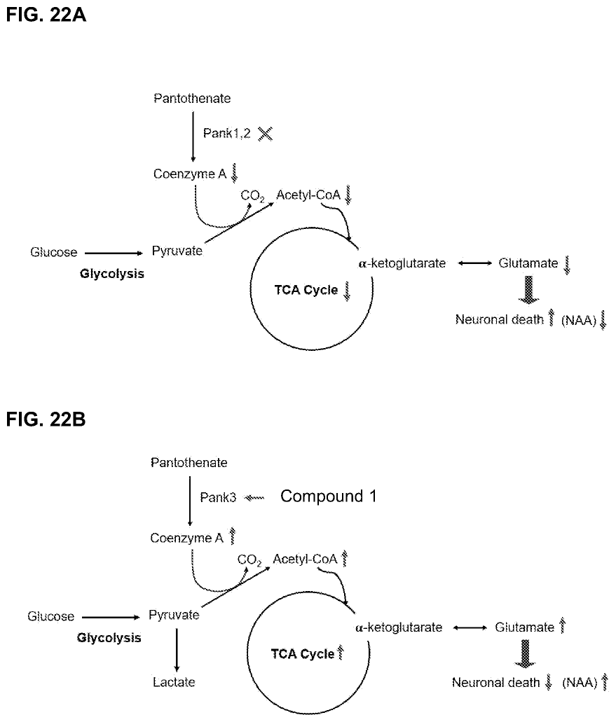

The treatment response to Compound 1 was associated with improved physical behavior of the neuronal Pank1,2 dKO mouse model and recovery of cerebral Glx. The strongest recovery was seen in Glx with Compound 1 treatment that activates the PANK3 isoform to increase CoA production that, in turn, has a direct role on TCA cycle metabolism (FIGS. 22A-22B) and hence Glx production. In contrast to our findings, a previous study performed on 3 human PKAN patients reported an increase of Glx in the white matter (Hájek et al., “MR relaxometry and 1H MR spectroscopy for the determination of iron and metabolite concentrations in PKAN patients,” European radiology, 15, 1060-1068, 2005). However, that clinical study was performed in patients with chronic disease progression and potential involvement of multiple brain cell types while this preclinical study was performed in a mouse model with rapidly progressing disease that is specific to neurons. Although neurons have been identified as a focal target of disease in PKAN, the role of glial cells in disease progression, for example, is not understood. A clinical MRS study reported elevated myo-inositol (m-Ins) levels in white matter due to gliosis and glial proliferation in patients with PKAN (Kitis et al., “Identification of axonal involvement in Hallervorden-Spatz disease with magnetic resonance spectroscopy,” Journal of Neuroradiology, 33, 129-132, 2006). However, we did not find any significant changes in the m-Ins levels in the neuronal Pank1,2 dKO mice.

The neuronal Pank1,2 dKO mouse model represents only selected molecular effects of PKAN, and the study has limitations. Alternative mouse models were not appropriate for evaluation of cerebral MRS either due to the lack of disease-related movement dysfunction and normal lifespan or early postnatal death. Analysis of human PKAN brains identified gene expression signatures that indicated neurons as a focal target of disease and development of the neuronal Pank1,2 dKO mouse model resulted in a longer-lived animal with consistent and measurable movement dysfunction that was tractable for MRS. Previous studies reported white matter pathology in the patients of PKAN. The previous ¹H MRS studies in human patients were often performed in white matter, which is not easily detected in the mouse model since the mouse brain consists of less than 12% white matter, as compared to 43% white matter in humans. Mouse models are recognized as not effective for studying white matter pathologies. In addition, there was no observable iron accumulation in the model, in contrast with the iron accumulation often observed in the basal ganglia of PKAN patients. This study may be limited to detection of metabolic changes in the gray matter in the neuronal Pank1,2 dKO mice.

PKAN is a life-threatening neurological disorder that can be caused by a variety of PANK2 mutations that affect protein expression, enzyme activity and/or stability, resulting in loss or reduction of kinase activity. PKAN diagnosis is based on characteristics of the movement dysfunction, exon sequencing and MRI evidence for iron accumulation in the basal ganglia with a characteristic “eye-of-the-tiger” pattern in T₂-weighted images. Our study showed no differences between the three groups in the T₂-weighted images. There are currently no approved clinical therapies for this genetic disorder. Progress with newly developing PKAN therapeutics has been made by evaluating mouse models with deactivated Pank genes in which brain CoA biosynthesis has been disrupted. One of the current challenges is to reliably identify molecular events associated with PKAN dysfunction in addition to monitoring the therapeutic responses. Thus, a technique which can quantitatively, non-invasively, and reproducibly analyze neurometabolism is needed and has potential value.

In summary, ¹H MRS was successfully applied to investigate neuronal chemical alterations in a mouse model of brain CoA deficiency, thought to be an underlying cause of PKAN. The effects of a potential PKAN therapeutic were evaluated by comparing the cerebral metabolic derangements among three mouse groups, where reductions of important metabolites (Glx, NAA, and Lactate) were observed in the model of brain CoA deficiency and recoveries of the same metabolites were evaluated following treatment with a newly developed Pantazine, Compound 1. The most promising biomarker for this potential PKAN therapeutic was the recovery of the Glx/tCr ratio. This study shows that ¹H MRS can be a powerful tool in evaluating therapeutic metabolic responses of neurological disorders.

Although the foregoing inventions have been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method, comprising:
(a) providing a subject having abnormal levels of one or more tricarboxylic acid (TCA) cycle metabolites; and
(b) based at least in part on (a), identifying the subject as being in need of a treatment with a therapeutic agent useful in the treatment of a disorder associated with Coenzyme A (CoA) reduction, elevation, sequestration, toxicity, or redistribution (CASTOR), a neurological disorder, and/or a metabolic disorder.

2. The method of claim 1, further comprising administering the therapeutic agent to the subject.

3. A method, comprising:
(a) providing a subject having abnormal levels of one or more tricarboxylic acid (TCA) cycle metabolites; and
(b) based at least in part on (a), administering a therapeutically effective amount of a therapeutic agent useful in the treatment of a disorder associated with Coenzyme A (CoA) reduction, elevation, sequestration, toxicity, or redistribution (CASTOR), a neurological disorder, and/or a metabolic disorder to the subject.

4. The method of any one of claims 1 to 3, wherein the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine.

5. The method of claim 4, wherein the one or more TCA cycle metabolites are selected from the group consisting of α-ketoglutarate, malate, methylcitrate, methylmalonate, oxaloacetate, and succinate.

6. The method of claim 5, wherein the one or more TCA cycle metabolites comprise malate.

7. The method of any one of claims 1 to 6, wherein the level at least one of the one or more TCA cycle metabolites is elevated.

8. The method of any one of claims 1 to 6, wherein the level of at least one of the one or more TCA cycle metabolites is depressed.

9. The method of any one of claims 1 to 8, wherein the level of:
(i) α-ketoglutarate in the serum of the subject is between about 0.1-30 micromolar (μM), such as between about 0.1-20 μM, 0.1-10 μM, 0.1-9 μM, 0.1-8 μM, 0.1-1.0 μM, 0.5-30 μM, 0.5-20 μM, 0.5-15 μM, 0.5-10 μM, 0.5-9 μM, 0.5-8 μM, 1-30 μM, 1-20 μM, 1-15 μM, 1-10 μM, 1-9 μM, 1-8 μM, 3-30 μM, 3-20 μM, 3-15 μM, 3-10 μM, 3-9 μM, 3-8 μM, 5-30 μM, 5-20 μM, 5-15 μM, 5-10 μM, 5-9 μM, 5-8 μM, or any range therein;
(ii) α-ketoglutarate in the urine of the subject is between about 0-600 micromoles per millimoles creatine (μmol/mmol Cr), such as between about 0-500 μmol/mmol Cr, 0-400 μmol/mmol Cr, 0-300 μmol/mmol Cr, 0-200 μmol/mmol Cr, 0-150 μmol/mmol Cr, 0-120 μmol/mmol Cr, 0-100 μmol/mmol Cr, 0-80 μmol/mmol Cr, 0-60 μmol/mmol Cr, 0-40 μmol/mmol Cr, 0-20 μmol/mmol Cr, or any range therein;
(iii) methylcitrate in the serum of the subject is between about 0-10 μM, such as between about 0-8 μM, 0-6 μM, 0-4 μM, 0-2 μM, 0.01-10 μM, 0.01-8 μM, 0.01-6 μM, 0.01-4 μM, 0.01-2 μM, 0.05-10 μM, 0.05-8 μM, 0.05-6 μM, 0.05-4 μM, 0.05-2 μM, 0.05-1 μM, or any range therein;
(iv) methylcitrate in the urine of the subject is between about 0-50 μmol/mmol Cr, such as between about 0-40 μmol/mmol Cr, 0-30 μmol/mmol Cr, 0-20 μmol/mmol Cr, 0-10 μmol/mmol Cr, 3-50 μmol/mmol Cr, 3-40 μmol/mmol Cr, 3-30 μmol/mmol Cr, 3-20 μmol/mmol Cr, 3-10 μmol/mmol Cr, 5-50 μmol/mmol Cr, 5-40 μmol/mmol Cr, 5-30 μmol/mmol Cr, 5-20 μmol/mmol Cr, 5-10 μmol/mmol Cr, or any range therein;
(v) malate in the plasma or serum of the subject is between about 0-20 μM, such as between about 0-18 μM, 0-16 μM, 0-14 μM, 0-12 μM, 0-10 μM, 1-20 μM, 1-18 μM, 1-16 μM, 1-14 μM, 1-12 μM, 1-10 μM, 1-8 μM, 1-6 μM, 1-5 μM, 2-20 μM, 2-18 μM, 2-16 μM, 2-14 μM, 2-12 μM, 2-10 μM, 2-8 μM, 3-20 μM, 3-18 μM, 3-16 μM, 3-14 μM, 3-12 μM, 3-10 μM, 3-8 μM, 3-6 μM, 5-20 μM, 5-10 μM, or any range therein;
(vi) malate in the urine of the subject is between about 0-300 μmol/mml Cr, such as between about 0-200 μmol/mml Cr, 0-150 μmol/mml Cr, 0-120 μmol/mml Cr, 0-100 μmol/mml Cr, 0-80 μmol/mml Cr, 0-60 μmol/mml Cr, 0-40 μmol/mml Cr, 0-20 μmol/mml Cr, 0-15 μmol/mml Cr, 0-10 μmol/mml Cr, 0-5 μmol/mml Cr, 0-3 μmol/mml Cr, 0-2 μmol/mml Cr, 5-300 μmol/mml Cr, 5-200 μmol/mml Cr, 5-100 μmol/mml Cr, 5-80 μmol/mml Cr, 5-60 μmol/mml Cr, 5-40 μmol/mml Cr, 5-20 μmol/mml Cr, or any range therein;
(vii) methylmalonate in the serum or plasma of the subject is between about 0-20 μM, such as between about 0-18 μM, 0-16 μM, 0-14 μM, 0-12 μM, 0-10 μM, 0-8 μM, 0-6 μM, 0-4 μM, 0-2 μM, 0-1 μM, 0-0.9 μM, 0-0.8 μM, 0-0.7 μM, 0-0.6 μM, 0-0.5 μM, 0-0.4 μM, 0-0.3 μM, 0-0.2 μM, 0-0.1 μM, 0-0.05 μM, or any range therein;
(viii) methylmalonate in the urine of the subject is between about 0-100 μmol/mmol Cr, such as between about 0-80 μmol/mmol Cr, 0-60 μmol/mmol Cr, 0-50 μmol/mmol Cr, 0-40 μmol/mmol Cr, 0-30 μmol/mmol Cr, 0-20 μmol/mmol Cr, 0-μmol/mmol Cr, 0-8 μmol/mmol Cr, 0-6 μmol/mmol Cr, 0-4 μmol/mmol Cr, 0-2 μmol/mmol Cr, 2-50 μmol/mmol Cr, 2-40 μmol/mmol Cr, 2-30 μmol/mmol Cr, 2-20 μmol/mmol Cr, 2-10 μmol/mmol Cr, 5-50 μmol/mmol Cr, 5-40 μmol/mmol Cr, 5-30 μmol/mmol Cr, 5-20 μmol/mmol Cr, 5-10 μmol/mmol Cr, or any range therein;
(ix) lactate in the plasma of the subject is between about 0-20 mmol/L, such as between about 0-15 mmol/L, 0-10 mmol/L, 0-8 mmol/L, 0-6 mmol/L, 0-5 mmol/L, 0-4 mmol/L, 0-3 mmol/L, 0-2 mmol/L, 0.5-10 mmol/L, 0.5-8 mmol/L, 0.5-6 mmol/L, 0.5-4 mmol/L, 0.5-3 mmol/L, 0.5-2 mmol/L, 0.5-1 mmol/L, 1-10 mmol/L, 1-8 mmol/L, 1-6 mmol/L, 1-4 mmol/L, 1-3 mmol/L, 1-2 mmol/L, or any range therein; and/or
(x) lactate in the urine of the subject is between about 0-10 mmol/mmol Cr, such as between about 0-8 mmol/mmol Cr, 0-6 mmol/mmol Cr, 0-5 mmol/mmol Cr, 0-4 mmol/mmol Cr, 0-3 mmol/mmol Cr, 0-2 mmol/mmol Cr, 0-1 mmol/mmol Cr, 0-0.5 mmol/mmol Cr, 0-0.4 mmol/mmol Cr, 0-0.3 mmol/mmol Cr, 0-0.2 mmol/mmol Cr, 0-0.1 mmol/mmol Cr, 0-0.08 mmol/mmol Cr, 0-0.07 mmol/mmol Cr, 0-0.06 mmol/mmol Cr, 0-0.05 mmol/mmol Cr, or any range therein.

10. The method of any one of claims 1 to 9, wherein the levels of TCA cycle metabolites of the one or more TCA cycle metabolites are urinary levels and/or plasma levels.

11. The method of any one of claims 1 to 10, further comprising assessing a sample from the subject to determine the levels of the one or more TCA cycle metabolites, optionally wherein the sample is a urine sample or a plasma sample.

12. The method of any one of claims 1 to 11, wherein the subject has:
(i) an elevated C3-carnitine level, a depressed C2-carnitine level, a depressed carnitine level, and/or an elevated C3:C2-carnitine ratio in plasma;
(ii) an elevated C3-carnitine level, a depressed C2-carnitine level, a depressed carnitine level, and/or an elevated C3:C2-carnitine ratio in the liver;
(iii) an elevated C3:C2-Coenzyme A (CoA) level in the liver; and/or
(iv) an elevated C3-CoA level in the liver and/or heart.

13. The method of any one of claims 1 to 12, wherein the subject is a human subject.

14. The method of any one of claims 1 to 13, wherein the subject is diagnosed with a disorder associated with CASTOR or Pantothenate kinase-associated neurodegeneration (PKAN).

15. The method of any one of claims 1 to 14, wherein the subject is diagnosed with propionic acidemia, defects in fatty acid oxidation enzymes, methylmalonic acidemia, glutaric acidemia, or HMG-CoA lyase deficiency.

16. The method of any one of claims 1 to 15, wherein the subject is diagnosed with Pantothenate kinase-associated neurodegeneration (PKAN).

17. The method of any one of claims 1 to 16, wherein the subject has previously undergone a therapeutic regimen for treatment of a disorder associated with CASTOR or pantothenate kinase-associated neurodegeneration (PKAN).

18. The method of any one of claims 1 to 17, wherein the subject has previously been treated with pantothenate, carnitine, pantothenic acid, or a combination thereof.

19. The method of any one of claims 1 to 18, further comprising (i) identifying the subject as in need of treatment with pantothenate, carnitine, pantothenic acid, or a combination thereof and/or (ii) administering pantothenate, carnitine, pantothenic acid, antibiotics, sodium benzoate, or a combination thereof to the subject.

20. A method, comprising:
(a) providing a first analysis of a first sample derived from a subject at a first time, wherein the first analysis provides first levels of one or more tricarboxylic acid (TCA) cycle metabolites;
(b) providing a second analysis of a second sample derived from the subject at a second time after the first time, wherein the second analysis provides second levels of the one or more TCA cycle metabolites;
(c) assessing a difference between a second level and a first level of at least one of the one or more TCA cycle metabolites; and
(d) based at least in part on (c), identifying the subject as being in need of a treatment with a therapeutic regimen comprising administration of a therapeutic agent useful in the treatment of a disorder associated with Coenzyme A (CoA) reduction, elevation, sequestration, toxicity, or redistribution (CASTOR), a neurological disorder, and/or a metabolic disorder.

21. The method of claim 20, further comprising administering the therapeutic agent to the subject.

22. A method, comprising:
(a) providing a first analysis of a first sample derived from a subject at a first time, wherein the first analysis provides first levels of one or more tricarboxylic acid (TCA) cycle metabolites;
(b) providing a second analysis of a second sample derived from the subject at a second time, wherein the second analysis provides second levels of the one or more TCA cycle metabolites, wherein the second time is after the first time, and wherein the subject has undergone treatment with a therapeutic regimen comprising administration of a first amount of a therapeutic agent useful in the treatment of a disorder associated with Coenzyme A (CoA) reduction, elevation, sequestration, toxicity, or redistribution (CASTOR), a neurological disorder, and/or a metabolic disorder at a first frequency between the first time and the second time;
(c) assessing a difference between a second level and a first level of at least one of the one or more TCA cycle metabolites; and
(d) based at least in part on (c),
(i) identifying the subject as being in need of a change in the therapeutic regimen if the difference in (c) exceeds or does not meet a threshold level, wherein the change comprises changing the amount of the therapeutic agent administered from the first amount to a second amount and/or changing the frequency of administration of the therapeutic agent to the subject from the first frequency to a second frequency, or
(ii) identifying the subject as not being in need of a change in the therapeutic regimen in the difference in (c) does not exceed the threshold level.

23. The method of claim 22, wherein (d) comprises decreasing a dosage of the therapeutic agent if the difference in (c) exceeds the threshold level.

24. The method of claim 22, wherein (d) comprises increasing a dosage of the the therapeutic agent if the difference in (c) does not meet the threshold level.

25. The method of claim 22, wherein (d) comprises not changing the therapy regimen if the difference in (c) does not exceed the threshold level.

26. The method of claim 22, wherein the threshold level of:
(i) lactate is between about 0.5-3.5 millimoles per liter (mmol/L) in plasma;
(ii) lactate is between about 0-0.07 mmol/mmol creatine (Cr) in urine;
(iii) malate is between about 3-12 micromolar (μM) in plasma;
(iv) malate is between about 0-120 micromoles per millimole Creatine (μmol/mmol Cr) in urine;
(v) methylmalonate is less than about 1 μM in serum or plasma;
(vi) methylmalonate is less than about 10 mmol/mol Cr in urine;
(vii) α-ketoglutarate is between about 5-10 μM in serum or plasma;
(viii) α-ketoglutarate is between about 0-150 μmol/mmol Cr in urine;
(ix) methylcitrate is between about 0-2 μM in serum or plasma; and/or
methylcitrate is between about 0-20 μmol/mmol Cr.

27. The method of any one of claims 20 to 22, wherein the subject is diagnosed with a disorder associated with CASTOR, a neurological disorder, and/or a metabolic disorder (i) after performance of (a) but before performance of (b); (ii) before performance of (a); or (iii) after performance of (b).

28. The method of any one of claims 20 to 22, wherein the subject has undergone treatment with the therapeutic regimen prior to (a).

29. The method of any one of claims 20 to 22, wherein the first time is at least one week before the second time.

30. The method of any one of claims 20 to 22, wherein the first time is at least one month before the second time.

31. The method of any one of claims 20 to 22, wherein the first time is at least six months before the second time.

32. The method of any one of claims 20 to 31, wherein the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine.

33. The method of claim 32, wherein the one or more TCA cycle metabolites are selected from the group consisting of α-ketoglutarate, malate, methylcitrate, methylmalonate, oxaloacetate, and succinate.

34. The method of claim 33, wherein the one or more TCA cycle metabolites comprise malate.

35. The method of any one of claims 20 to 34, wherein the second level at least one of the one or more TCA cycle metabolites is lower than the first level of the at least one of the one or more TCA cycle metabolites.

36. The method of claim 35, wherein the second level of malate level is lower than the first level of malate.

37. The method of any one of claims 20 to 34, wherein the second level at least one of the one or more TCA cycle metabolites is higher than the first level of the at least one of the one or more TCA cycle metabolites.

38. The method of any one of claims 20 to 37, wherein the first sample and the second sample are urine samples.

39. The method of any one of claims 20 to 37, wherein the first sample and the second sample are plasma samples.

40. The method of any one of claims 20 to 39, wherein, prior to (b), the subject has:
(i) an elevated C3-carnitine level, a depressed C2-carnitine level, a depressed carnitine level, and/or an elevated C3:C2-carnitine ratio in plasma;
(ii) an elevated C3-carnitine level, a depressed C2-carnitine level, a depressed carnitine level, and/or an elevated C3:C2-carnitine ratio in the liver;
(iii) an elevated C3:C2-Coenzyme A (CoA) level in the liver; and/or
(iv) an elevated C3-CoA level in the liver and/or heart.

41. The method of any one of claims 20 to 40, wherein, subsequent to treatment with the therapeutic regimen, (i) the C3-carnitine level and/or the C3:C2-carnitine ratio in the plasma and/or liver of the subject decreases; and/or (ii) the C3:C2-Coenzyme A (CoA) level in the liver of the subject decreases.

42. The method of any one of claims 20 to 41, wherein the subject is a human subject.

43. The method of any one of claims 20 to 42, wherein, prior to (a), the subject is diagnosed with a disorder associated with CASTOR or Pantothenate kinase-associated neurodegeneration (PKAN).

44. The method of any one of claims 20 to 43, wherein, prior to (a), the subject is diagnosed with propionic acidemia, defects in fatty acid oxidation enzymes, methylmalonic acidemia, glutaric acidemia, or HMG-CoA lyase deficiency.

45. The method of any one of 20 to 44, wherein, prior to (a), the subject has previously undergone a therapeutic regimen for treatment of a disorder associated with CASTOR or Pantothenate kinase-associated neurodegeneration (PKAN).

46. The method of any one of claims 20 to 45, wherein, prior to (a), the subject was treated with pantothenate, carnitine, pantothenic acid, or a combination thereof.

47. The method of any one of claims 20 to 46, wherein further comprising (i) identifying the subject as in need of treatment with pantothenate, carnitine, pantothenic acid, or a combination thereof; and/or administering pantothenate, carnitine, pantothenic acid, antibiotics, sodium benzoate, or a combination thereof to the subject.

48. The method of any one of claims 1 to 47, wherein the subject has a protein restricted diet.

49. The method of any one of claims 1 to 48, wherein the therapeutic agent is a compound having a structure represented by a formula: 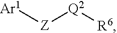
wherein Z is selected from A(C═O), COCH₂, 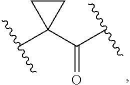 [SMILES: *C(=O)C1(*)CC1]
CO, NHCO, NHCS, CH₂SO₂, and SO₂;
wherein A is selected from O, CO, CH₂, CF₂, NH, N(CH₃), and CH(OH);
wherein Q² is a structure selected from: 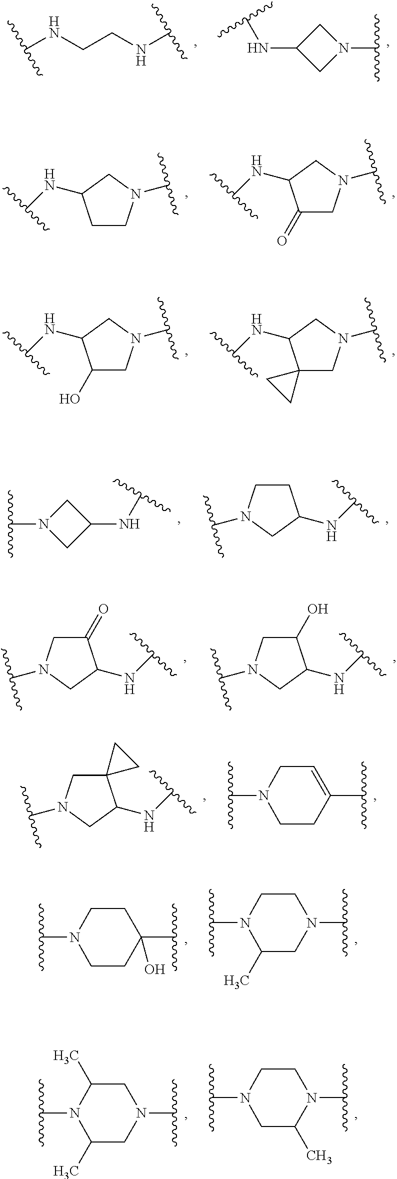 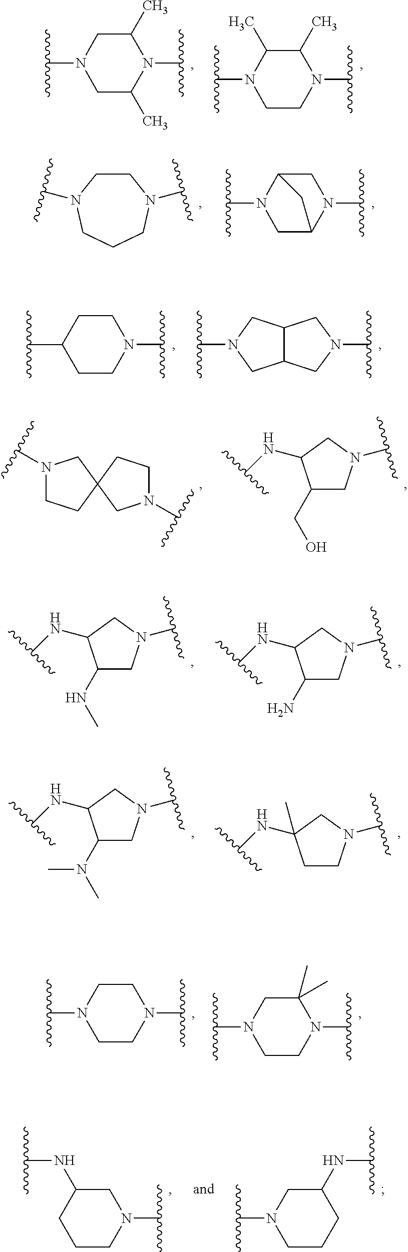
wherein Ar¹ is selected from aryl and heteroaryl and substituted with 0, 1, 2, or 3 groups independently selected from halogen, —NO₂, —CN, —OH, —SH, —NH₂, C1-C8 thioalkyl, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), C1-C8 alkoxyhaloalkyl, and cyclopropyl, cyclobutyl, and oxetane, wherein the cyclopropyl, cyclobutyl, and oxetane are optionally substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl);
wherein R⁶ is selected from —NHCH₂C₆H₅ and Ar²;
wherein Ar² is a structure represented by a formula selected from: 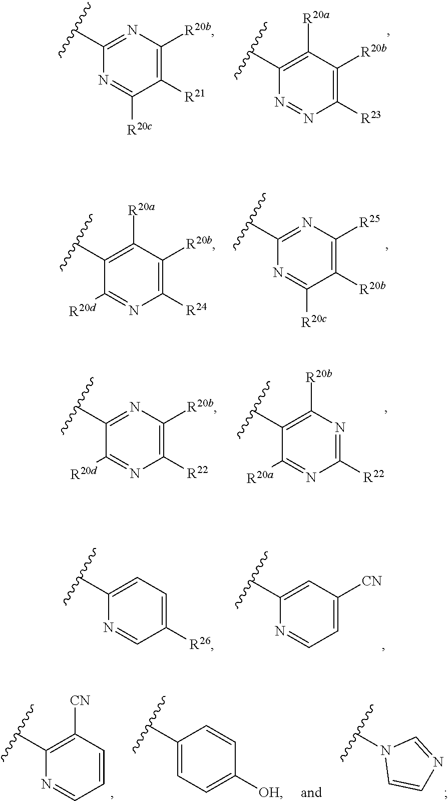
wherein each of R^{20a}, R^{20b}, R^{20c}, and R^{20d}, when present, is independently selected from hydrogen, halogen, —CN, —NO₂, —NH₂, C1-C4 alkyl, C1-C4 monohaloalkyl, C1-C4 polyhaloalkyl, C1-C4 alkoxy, C1-C4 monohaloalkoxy, C1-C4 polyhaloalkoxy, C1-C4 alkylamino, (C1-C4)(C1-C4) dialkylamino, and cyclopropyl;
wherein R²¹, when present, is selected from hydrogen, halogen, —CN, —NO₂, —SO₂NH₂, —SO₂CH₃, —SO₂CF₃, and Cy¹;
wherein Cy¹, when present, is selected from cycle, heterocycle, aryl, and heteroaryl and substituted with 0, 1, 2, or 3 groups independently selected from halogen, —NO₂, —CN, —OH, —SH, —NH₂, C1-C4 alkyl, C1-C4 monohaloalkyl, C1-C4 polyhaloalkyl, C1-C4 alkoxy, C1-C4 alkylamino, and (C1-C4)(C1-C4) dialkylamino;
wherein R²², when present, is selected from —CN, halogen, —NO₂, SO₂NH₂, SO₂CH₃, and SO₂CF₃;
wherein R²³, when present, is selected from hydrogen, halogen, —CN, —NO₂, —SO₂NH₂, —SO₂CH₃, —SO₂CF₃, cyclohexyl, 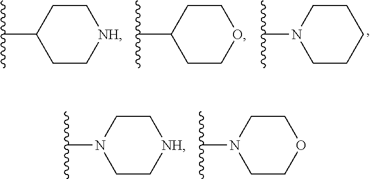
and Cy¹;
wherein R²⁴, when present, is selected from —CN, halogen, —NO₂, SO₂NH₂, SO₂CH₃, and SO₂CF₃;
wherein R²⁵, when present, is selected from —CN, —NO₂, SO₂NH₂, SO₂CH₃, and SO₂CF₃;
wherein R²⁶, when present, is selected from —Br, —Cl, —F, —CN, —NO₂, —CF₃, and methyl;
or a pharmaceutically acceptable salt thereof.

50. The method of claim 49, wherein the compound has a structure represented by a formula: 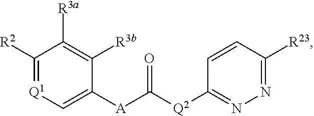
wherein Q¹ is CH; and wherein R² is selected from —SCH₃, C1-C8 acyclic alkyl, C2-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; or
wherein Q¹ is N; and R² is selected from halogen, —SCH₃, C1-C8 acyclic alkyl, C2-C8 acyclic alkenyl, C1-C8 monohaloalkyl, C1-C8 polyhaloalkyl, C1-C8 alkoxyhaloalkyl, cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy;
wherein Q² is a structure selected from: 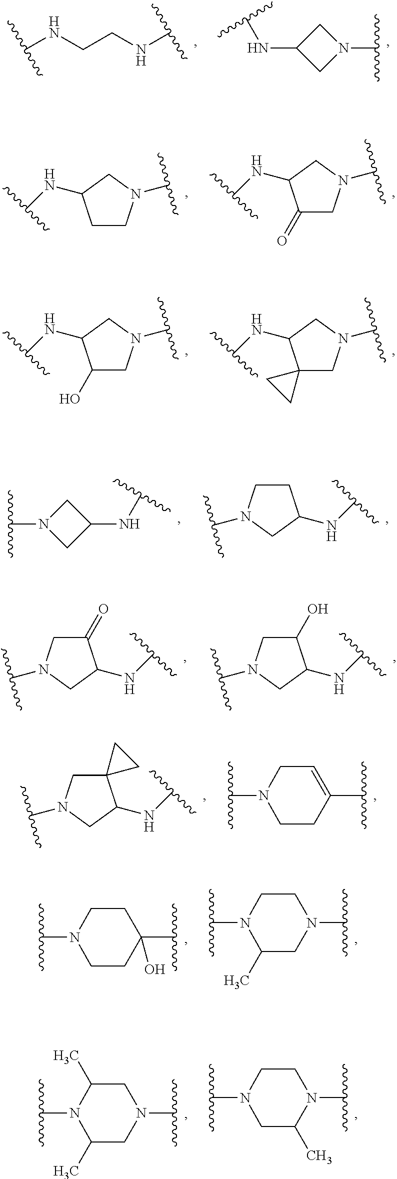 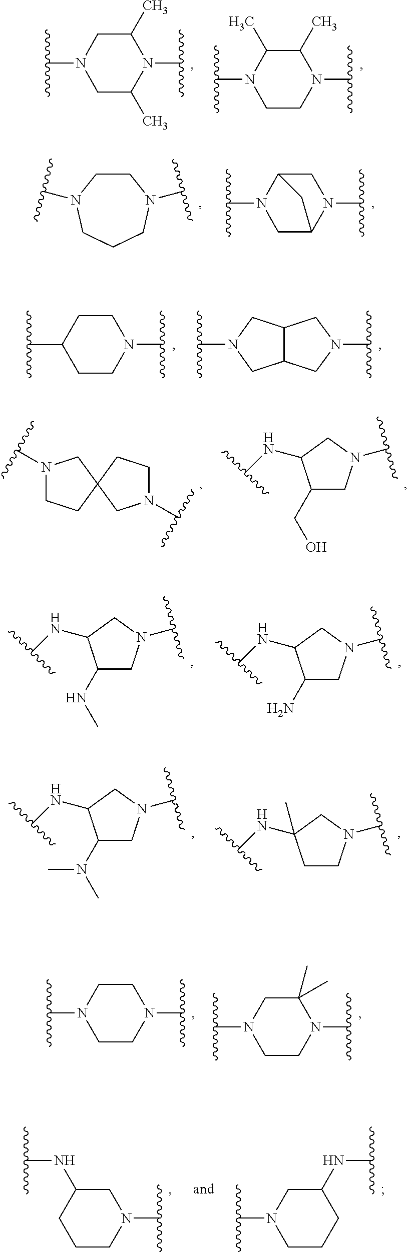
wherein each of R^{3a} and R^{3b} is independently selected from hydrogen, halogen, —OH, C1-C4 alkoxy, and C1-C4 alkyl; and
wherein R²³ is selected from hydrogen, halogen, —CN, SO₂NH₂, SO₂CH₃, SO₂CF₃, and NO₂, or a pharmaceutically acceptable salt thereof.

51. The method of claim 50, wherein the compound has a structure represented by a formula: 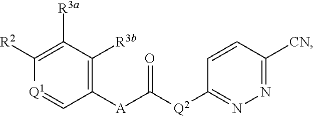
or a pharmaceutically acceptable salt thereof.

52. The method of claim 50, wherein the compound has a structure represented by a formula: 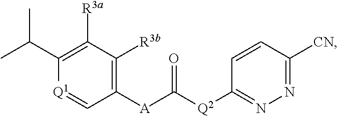
or a pharmaceutically acceptable salt thereof.

53. The method of claim 50, wherein the compound is selected from: 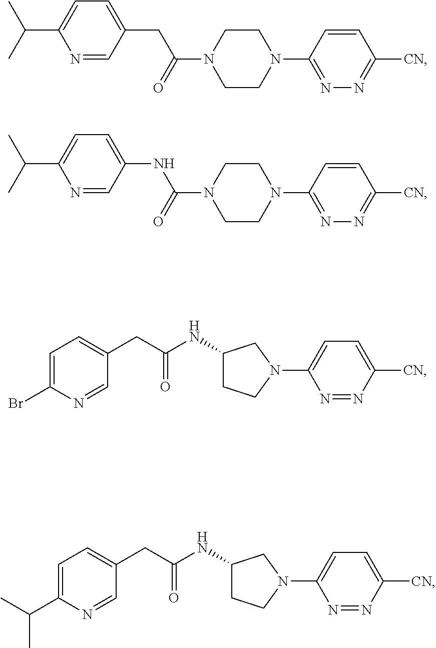 [SMILES: CC(C)c1ccc(CC(=O)N2CCN(c3ccc(C#N)nn3)CC2)cn1 ; CC(C)c1ccc(NC(=O)N2CCN(c3ccc(C#N)nn3)CC2)cn1 ; N#Cc1ccc(N2CC[C@H](NC(=O)Cc3ccc(Br)nc3)C2)nn1 ; CC(C)c1ccc(CC(=O)N[C@H]2CCN(c3ccc(C#N)nn3)C2)cn1]
or a pharmaceutically acceptable salt thereof.

54. The method of claim 50, wherein the compound is selected from: 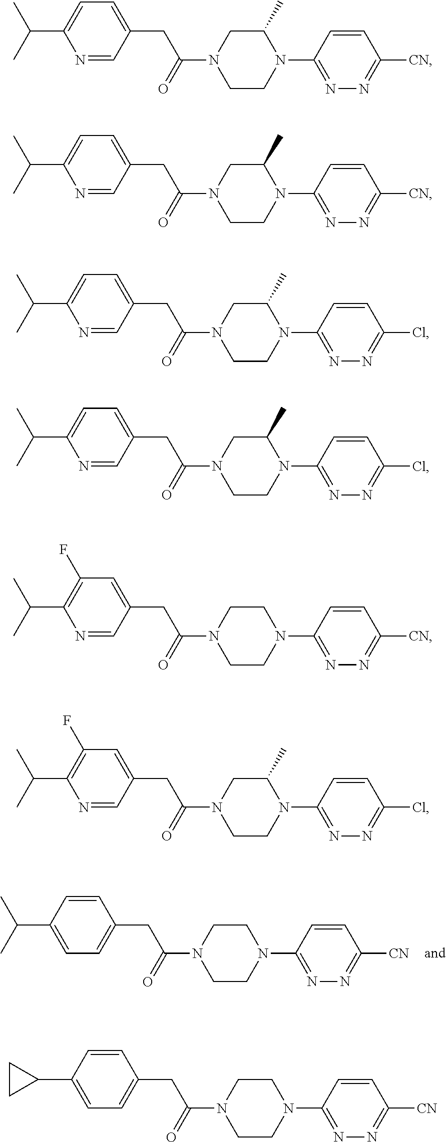
or a pharmaceutically acceptable salt thereof.

55. The method of claim 50, wherein the compound has a structure a structure represented by a formula: 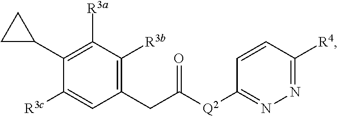
wherein Q² is a structure selected from: 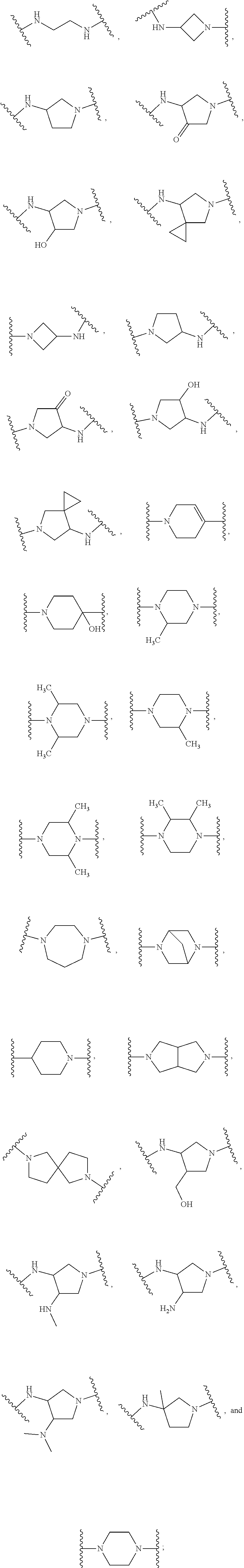
wherein each of R^{3a}, R^{3b}, and R^{3c} is independently selected from hydrogen, halogen, C1-C4 alkoxy and C1-C4 alkyl, provided at least one of R^{3a}, R^{3b}, and R^{3c} is halogen; and
wherein R⁴ is selected form hydrogen, halogen, —CN, SO₂NH₂, SO₂CH₃, SO₂CF₃, and NO₂, or a pharmaceutically acceptable salt thereof.

56. The method of claim 55, wherein the compound is selected from: 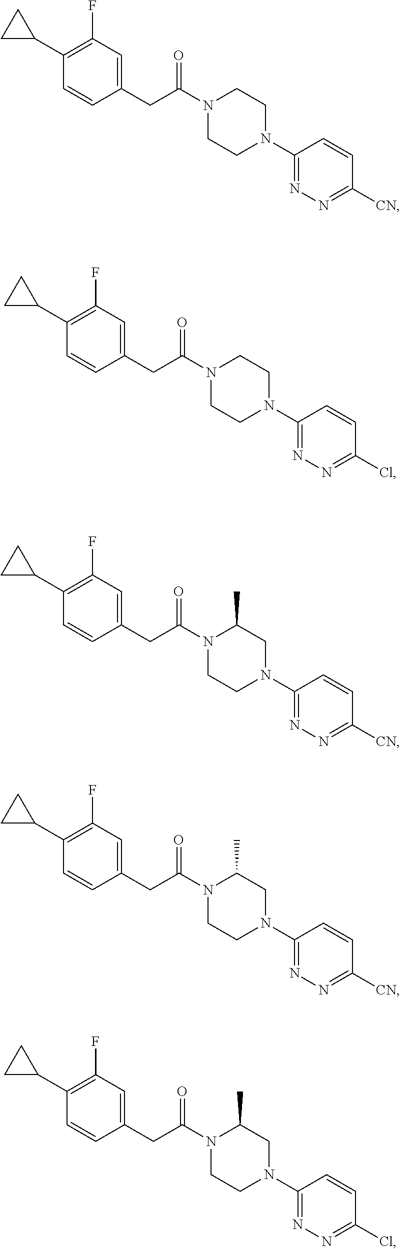 [SMILES: N#Cc1ccc(N2CCN(C(=O)Cc3ccc(C4CC4)c(F)c3)CC2)nn1 ; O=C(Cc1ccc(C2CC2)c(F)c1)N1CCN(c2ccc(Cl)nn2)CC1 ; C[C@H]1CN(c2ccc(C#N)nn2)CCN1C(=O)Cc1ccc(C2CC2)c(F)c1 ; C[C@@H]1CN(c2ccc(C#N)nn2)CCN1C(=O)Cc1ccc(C2CC2)c(F)c1 ; C[C@H]1CN(c2ccc(Cl)nn2)CCN1C(=O)Cc1ccc(C2CC2)c(F)c1] 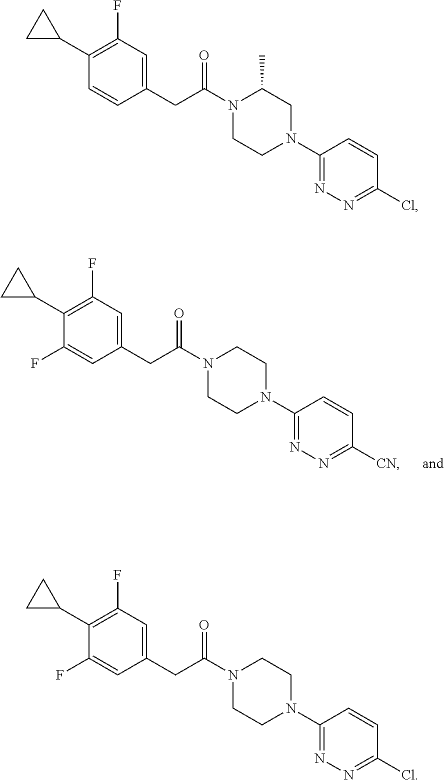 [SMILES: C[C@@H]1CN(c2ccc(Cl)nn2)CCN1C(=O)Cc1ccc(C2CC2)c(F)c1 ; N#Cc1ccc(N2CCN(C(=O)Cc3cc(F)c(C4CC4)c(F)c3)CC2)nn1 ; O=C(Cc1cc(F)c(C2CC2)c(F)c1)N1CCN(c2ccc(Cl)nn2)CC1]
or a pharmaceutically acceptable salt thereof.

57. The method of claim 49, wherein the compound has a structure represented by a formula: 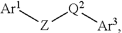
wherein Q² is a structure selected from: 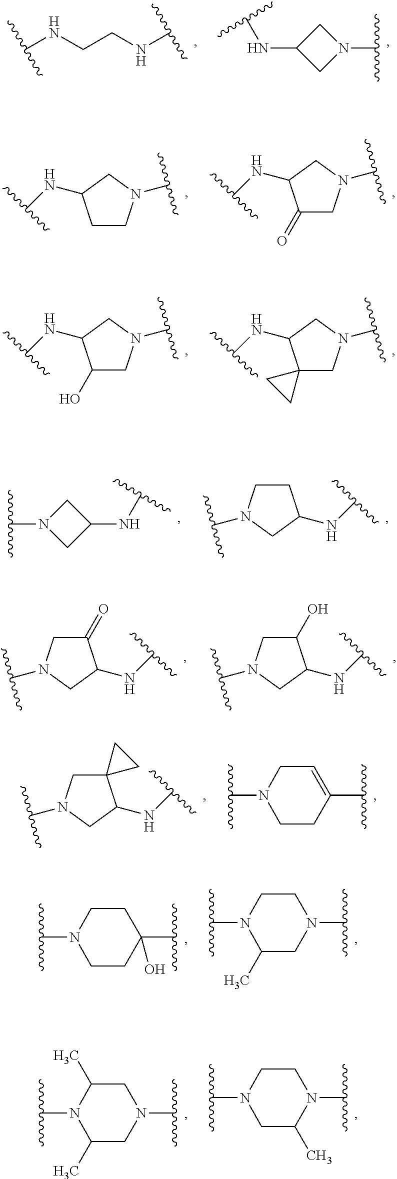 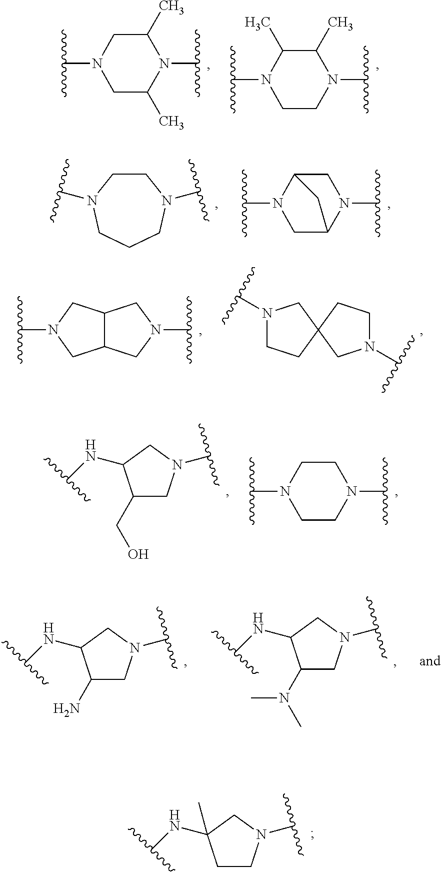
wherein Z is selected from O(C═O), CF₂CO, COCH₂, CH₂CO, 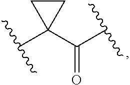 [SMILES: *C(=O)C1(*)CC1]
CO, CH₂SO₂, SO₂, NHCO, N(CH₃)CO, and CH(OH)CO;
wherein Ar¹ is selected from aryl and heteroaryl and substituted with 1, 2, or 3 groups independently selected from halogen, —NO₂, —CN, —OH, —SH, —NH₂, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), cyclopropyl, cyclobutyl, and oxetane, wherein the cyclopropyl, cyclobutyl, and oxetane are optionally substituted with 1, 2, or 3 groups independently selected from —OH, C1-C4 alkyl, and C1-C4 alkoxy; and
wherein Ar³ is a structure selected from: 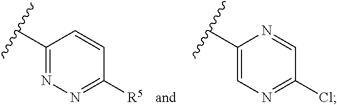
wherein R⁵, when present, is selected from CN, halogen, —NO₂, SO₂NH₂, and SO₂CH₃;
provided that if R⁵ is CN and Z is CO then Ar¹ is not substituted with C1-C8 monohaloalkyl or C1-C8 polyhaloalkyl; and
provided that if R⁵ is halogen then Ar¹ is selected from 5- and 6-membered heteroaryl and Z cannot be CO,
or a pharmaceutically acceptable salt thereof.

58. The method of claim 57, wherein Ar¹ is selected from aryl and heteroaryl and substituted with 1, 2, or 3 groups independently selected from halogen, —NO₂, —CN, —OH, —SH, —NH₂, C1-C8 acyclic alkyl, C2-C8 acyclic alkenyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, —CO(C1-C8 acyclic alkyl), and cyclopropyl.

59. The method of claim 57, wherein Ar³ is:

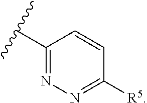

60. The method of claim 57, wherein R⁵ is CN or —Cl.

61. The method of claim 57, wherein R⁵ is selected from halogen, —NO₂, SO₂NH₂, and SO₂CH₃.

62. The method of claim 57, wherein the compound is:

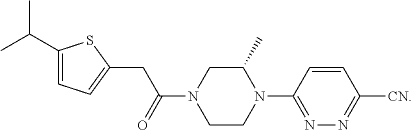 [SMILES: CC(C)c1ccc(CC(=O)N2CCN(c3ccc(C#N)nn3)[C@@H](C)C2)s1]

63. The method of claim 49, wherein the compound has a structure represented by a formula selected from: 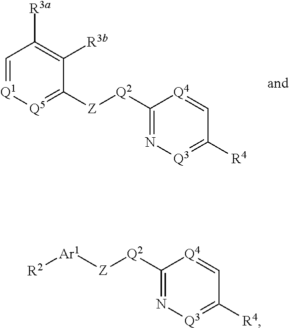
wherein Z is selected from A(C═O), C(O)CH₂, C(O), CH₂SO₂, and SO₂,
wherein A is selected from O, CH₂, CF₂, NH, N(CH₃), and CH(OH);
wherein each of Q¹ and Q⁵, when present, is independently selected from N and CH;
wherein Q³ is N and Q⁴ is CH or wherein Q⁴ is N and Q³ is CH;
wherein Q² is a structure selected from: 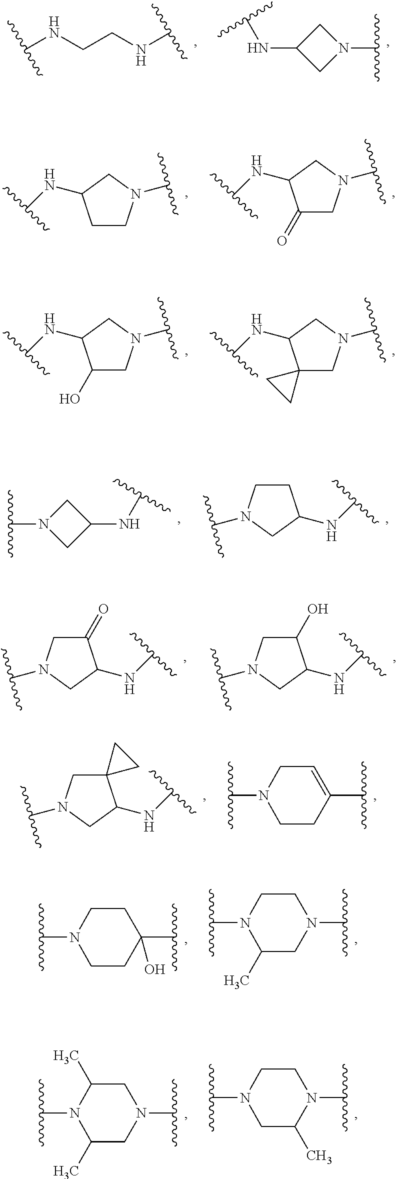 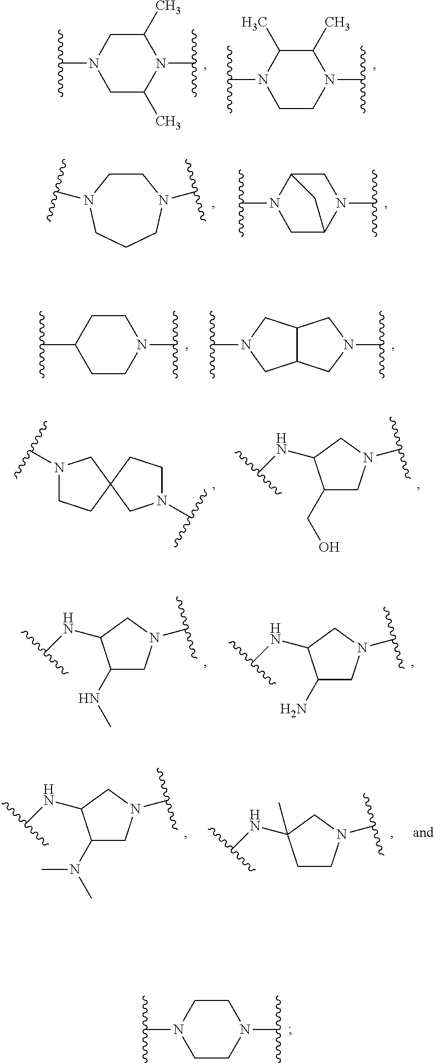
wherein R², when present, is selected from C1-C8 hydroxyalkyl, C1-C8 alkoxy, and cyclopropyl substituted with 1, 2, 3, or 4 groups independently selected from halogen, —NO₂, —CN, —OH, —SH, —NH₂, C1-C4 acyclic alkyl, C1-C4 hydroxyalkyl, C1-C4 monohaloalkyl, C1-C4 polyhaloalkyl, C1-C4 alkoxy, C1-C4 monohaloalkoxy, C1-C4 polyhaloalkoxy, C1-C4 acyclic alkylamino, (C1-C4)(C1-C4) dialkylamino, and —CO(C1-C4 acyclic alkyl), provided that cyclopropyl, when present, is substituted with at least one halogen group;
wherein each of R^{3a} and R^{3b}, when present, is independently selected from hydrogen, halogen, —OH, C1-C4 alkyl, C1-C4 thioalkyl, and C1-C4 alkoxy;
wherein R⁴ is selected from hydrogen, halogen, —CN, —NO₂, —SO₂NH₂, and —SO₂CH₃; and
wherein Ar¹, when present, is selected from aryl and heteroaryl and is substituted with 0, 1, 2, or 3 groups independently selected from halogen, —NO₂, —CN, —OH, —SH, —NH₂, C1-C8 acyclic alkyl, C1-C8 hydroxyalkyl, C1-C8 monohaloalkyl, C1-C8 polyhaloalkyl, C1-C8 alkoxy, C1-C8 monohaloalkoxy, C1-C8 polyhaloalkoxy, C1-C8 acyclic alkylamino, (C1-C8)(C1-C8) dialkylamino, and —CO(C1-C8 acyclic alkyl),
provided that when R³ is C1-C8 hydroxy or C1-C8 alkoxy then R² is not hydrogen,
or a pharmaceutically acceptable salt thereof.

64. The method of claim 63, wherein each of Q¹ and Q⁵ is CH.

65. The method of claim 63, wherein Q³ is N and Q⁴ is CH.

66. The method of claim 63, wherein Q² is a structure:

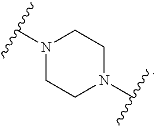 [SMILES: *N1CCN(*)CC1]

67. The method of claim 63, wherein R² is cyclopropyl substituted with 1, 2, or 3 groups independently selected from halogen and C1-C4 acyclic alkyl, provided that cyclopropyl is substituted with at least one halogen group.

68. The method of claim 63, wherein R² is a structure selected from:

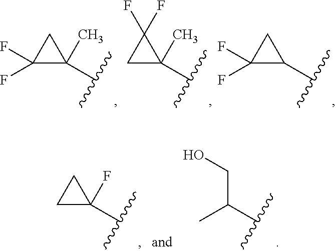

69. The method of claim 63, wherein each of R^{3a} and R^{3b} is hydrogen.

70. The method of claim 63, wherein R⁴ is CN.

71. The method of claim 63, wherein Ar¹ is a structure:

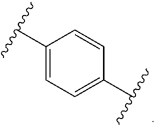 [SMILES: *c1ccc(*)cc1]

72. The method of claim 63, wherein the compound has a structure represented by a formula selected from:

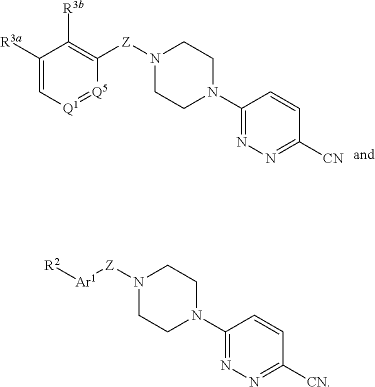

73. The method of claim 63, wherein the compound is selected from:

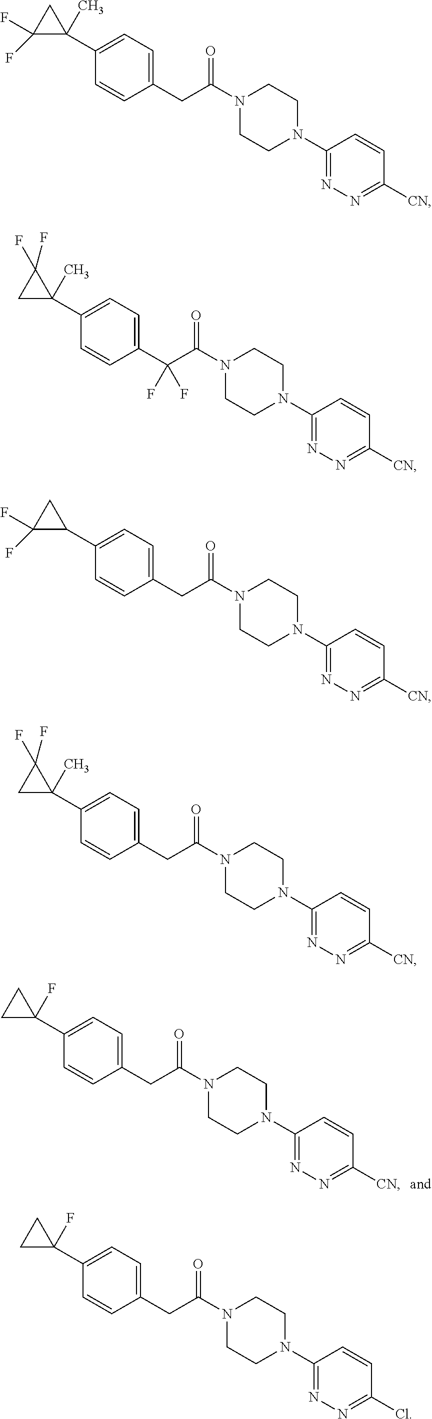 [SMILES: CC1(c2ccc(CC(=O)N3CCN(c4ccc(C#N)nn4)CC3)cc2)CC1(F)F ; CC1(c2ccc(C(F)(F)C(=O)N3CCN(c4ccc(C#N)nn4)CC3)cc2)CC1(F)F ; N#Cc1ccc(N2CCN(C(=O)Cc3ccc(C4CC4(F)F)cc3)CC2)nn1 ; CC1(c2ccc(CC(=O)N3CCN(c4ccc(C#N)nn4)CC3)cc2)CC1(F)F ; N#Cc1ccc(N2CCN(C(=O)Cc3ccc(C4(F)CC4)cc3)CC2)nn1 ; O=C(Cc1ccc(C2(F)CC2)cc1)N1CCN(c2ccc(Cl)nn2)CC1]

74. The method of claim 63, wherein the compound is: 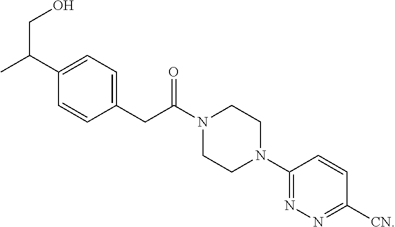 [SMILES: CC(CO)c1ccc(CC(=O)N2CCN(c3ccc(C#N)nn3)CC2)cc1]
